(19) United States
(12) Patent Application Publication
(10) Pub. No.: US 2023/0295118 A1
(43) Pub. Date: Sep. 21, 2023

(54) COMPOUNDS AND METHODS FOR THE TARGETED DEGRADATION OF CYCLIN DEPENDENT KINASES

(71) Applicants: EnhancedBio Inc., Seoul (KR); Endotarget Inc., New Orleans, LA (US)

(72) Inventors: Guangdi Wang, New Orleans, LA (US); Xianyou Peng, New Orleans, LA (US); Hongjoong Kim, Seoul (KR); Hyunjung Choi, Seoul (KR); Hunsoon Jung, Seoul (KR); Bongsu Park, Seoul (KR)

(21) Appl. No.: 17/813,161

(22) Filed: Jul. 18, 2022

Related U.S. Application Data
(60) Provisional application No. 63/321,165, filed on Mar. 18, 2022.

(51) Int. Cl. C07D 401/14; C07D 471/04; C07D 487/04; A61P 35/00

(52) U.S. Cl. CPC C07D 401/14; C07D 471/04; C07D 487/04; A61P 35/00

(57) ABSTRACT

Disclosed herein are compounds of Formula (I):

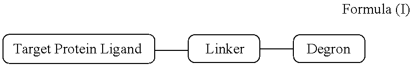

or a pharmaceutically acceptable salt, hydrate, solvate, prodrug, stereoisomer, or tautomer thereof, which act as protein degradation inducing moieties for cyclin-dependent kinase 4 (CDK4) and/or cyclin-dependent kinase 6 (CDK6). These compounds are useful in methods for the targeted degradation of CDK4 and/or CDK6 through the use of the bifunctional compounds that link a ubiquitin ligase-binding moiety to a ligand that is capable of binding to CDK4 and/or CDK6 which can be utilized in the treatment of disorders modulated by CDK4 and/or CDK6.

### CROSS-REFERENCE TO RELATED APPLICATIONS

This application claims priority to and the benefit of U.S. Provisional Application No. 63/321,165, filed Mar. 18, 2022, the disclosures of which are incorporated herein by reference in their entireties.

### FIELD OF THE INVENTION

Disclosed herein are methods, compounds and formulations directed to the modulation of cyclin dependent kinases.

### BACKGROUND OF THE INVENTION

Cyclin-dependent kinases 4 and 6 (CDK4/6) are Ser/Thr kinases that regulate G1-S cell cycle transition by forming complexes with D-type cyclins (D1, D2 and D3). CDK4/6-cyclin D complexes phosphorylate the retinoblastoma (Rb) proteins to release Rb-mediated repression on E2F target genes that drive G1 to S progression (Kent & Leone, 2019). Dysregulated CDK activity and loss of cell cycle control is a hallmark of cancerous cells (Hall and Peters, 1996; Sherr, 1996). To re-establish cell cycle control and block cancer cell proliferation, small molecule ATP competitive inhibitors have been used to selectively target cdk4/6 to induce G1 arrest in cells retaining wild-type Rb. However, the major obstacle to successful treatment with CDK4/6 inhibitors is acquired resistance that frequently occurs in patients who have received this therapy. It has been demonstrated that CDK4 was elevated in palbociclib resistant cell lines (Pancholi et al 2020). Amplification of CDK4 in melanoma, glioma, rhabdomyosarcoma, and lung cancer confers resistance to CDK4/6 inhibitors in these malignancies (Eran 2012, Ling 2012, Olanich et al 2015, Jiang et al 2020). In ER+ breast cancer, CDK6 amplification has been reported to confer acquired resistance to CDK4/6 inhibitors (Yang et al 2016). Overexpression of CDK6 not only mediates resistance to CDK4/6 inhibitors, but also leads to decreased expression of estrogen and progesterone receptors. Depletion of the individual CDK may provide a therapeutic strategy for overcoming resistance to CDK4/6 inhibition.

Thus, there is a need in the art for compositions and methods for modulating the cyclin dependent kinases.

### SUMMARY OF THE INVENTION

Disclosed herein are novel bifunctional compounds and compositions useful for the degradation of a target protein by recruiting the target protein to an E3 ubiquitin ligase for degradation by the endogenous cellular ubiquitin proteasome system (UPS). In particular, disclosed herein are bifunctional compounds, that facilitate targeted ubiquitination and degradation of cyclin dependent kinases (target protein), and/or exhibit inhibition of the cyclin dependent kinases. In addition, disclosed herein are methods of making such compounds and compositions; methods of using such compounds and compositions; pharmaceutical compositions comprising such compounds and compositions; and methods of using such pharmaceutical compositions, for the treatment or amelioration of a disease condition, such as cancer, especially breast cancer.

In an additional aspect, a method of ubiquitinating followed by degrading a target protein by bifunctional compounds attached by a chemical linker; therapeutic compositions comprising an effective amount of a compound disclosed herein or salt/solvate form thereof, and its delivery using a pharmaceutically acceptable carrier. In yet another aspect, the therapeutic compositions of a compound or multiple compounds that degrade and/or inhibit the target protein in a patient or subject, such as a human or animal, can be used for treating or ameliorating disease conditions/states, e.g., breast cancer, through modulation of wild-type CDK4/6 or mutant CDK4/6.

Provided here is a compound of the formula (I), or a tautomer, stereoisomer or a mixture of stereoisomers, pharmaceutically acceptable salt, or solvate thereof:

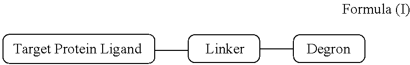

wherein:
the Target Protein Ligand is capable of binding to a target protein, such as a cyclin-dependent kinase (e.g., CDK4 and/or CDK6); the Target Protein Ligand is one of the following structures:

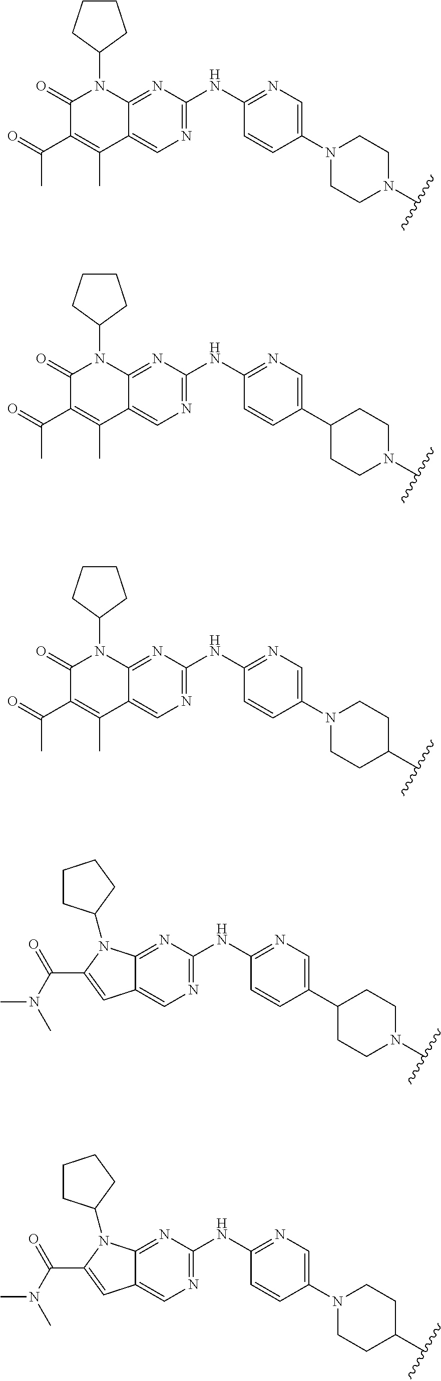 [SMILES: *N1CCN(c2ccc(Nc3ncc4c(C)c(C(C)=O)c(=O)n(C5CCCC5)c4n3)nc2)CC1 ; *N1CCC(c2ccc(Nc3ncc4c(C)c(C(C)=O)c(=O)n(C5CCCC5)c4n3)nc2)CC1 ; *C1CCN(c2ccc(Nc3ncc4c(C)c(C(C)=O)c(=O)n(C5CCCC5)c4n3)nc2)CC1 ; *N1CCC(c2ccc(Nc3ncc4cc(C(=O)N(C)C)n(C5CCCC5)c4n3)nc2)CC1 ; *C1CCN(c2ccc(Nc3ncc4cc(C(=O)N(C)C)n(C5CCCC5)c4n3)nc2)CC1]

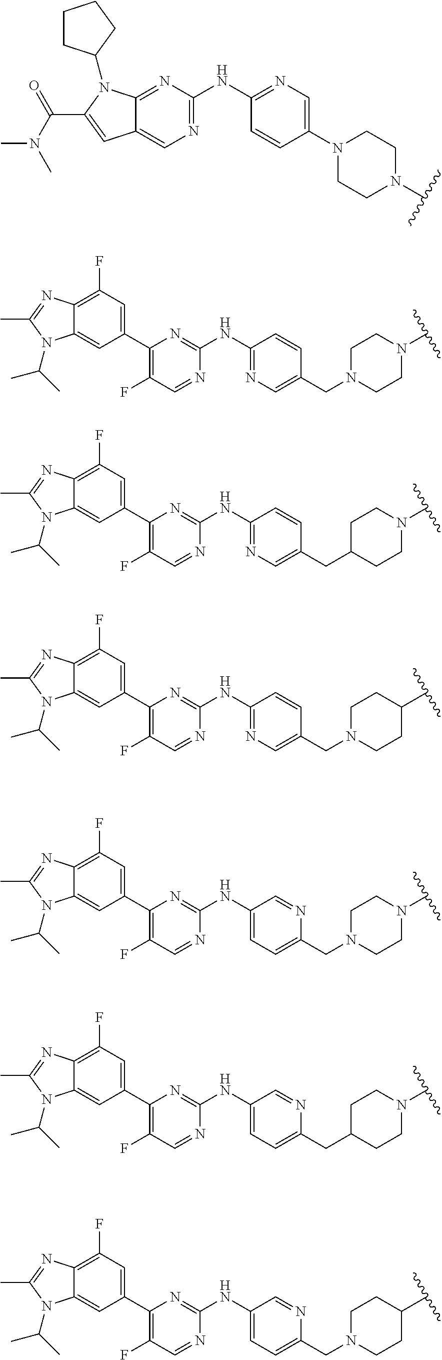

The Linker is a group that is covalently bonded to the Target Protein Ligand and the Degron; the Linker is an optionally substituted linking moiety comprising a branched or unbranched, cyclized or uncyclized, saturated or unsaturated chain of 5 to 16 carbon atoms in length.

In some embodiments, the linker is an optionally substituted linking moiety. In some embodiments, the linker comprises a branched or unbranched, cyclized or uncyclized, saturated or unsaturated chain of 5 to 16 carbon atoms in length, or any combination thereof; wherein 1 to 6 of the carbon atoms are optionally replaced with a heteroatom. In some embodiments, each occurrence of the heteroatom is independently O, N, and S.

In some embodiments, the linking moiety comprises a branched or linear C₅ to C₁₆ alkyl, branched or linear amino-C₅ to C₁₆ alkyl, branched or linear C₅ to C₁₆ alkoxy, branched or linear thio-C₅ to C₁₆ alkyl, C₅ to C₁₆ cycloalkyl, amino-C₅ to C₁₆ cycloalkyl, hydroxy-C₅ to C₁₆ cycloalky, thio-C₅ to C₁₆ cycloalkyl, or any combination thereof; wherein 1 to 6 of the carbon atoms are optionally replaced with a heteroatom. In some embodiments, each occurrence of the heteroatom is independently 0, N, and S.

The Degron is capable of binding to a ubiquitin ligase, such as an E3 ubiquitin ligase (e.g., CRBN). The Degron is selected from the following structures:

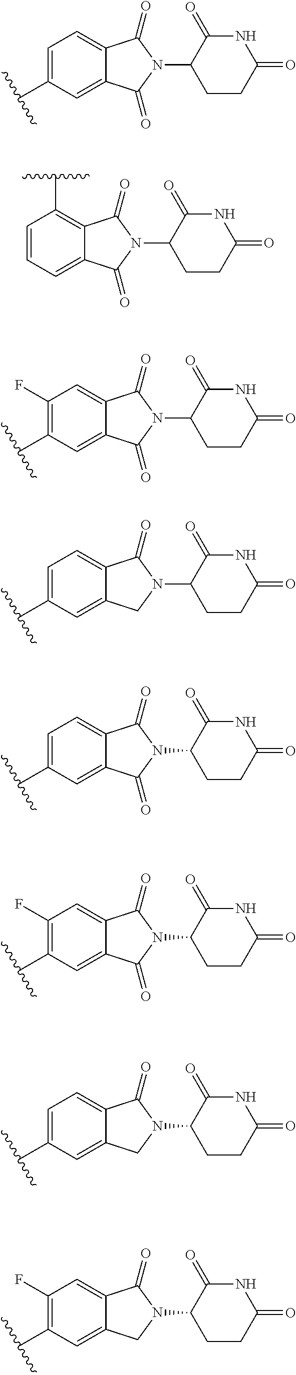

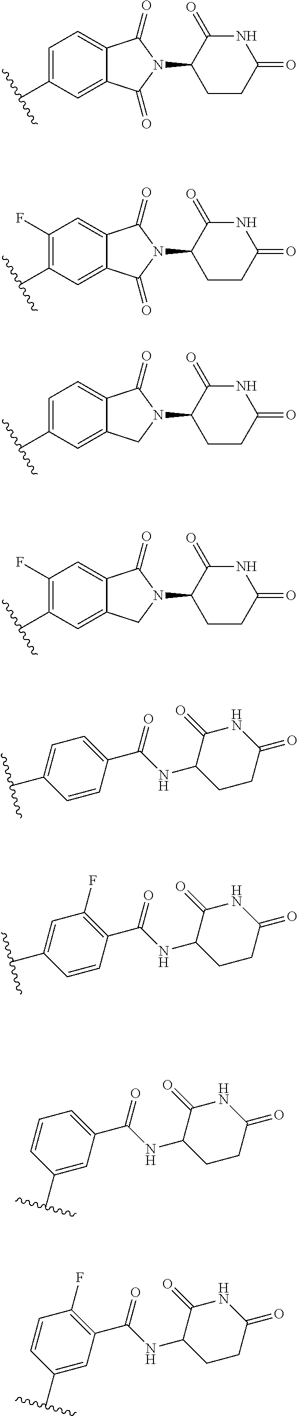

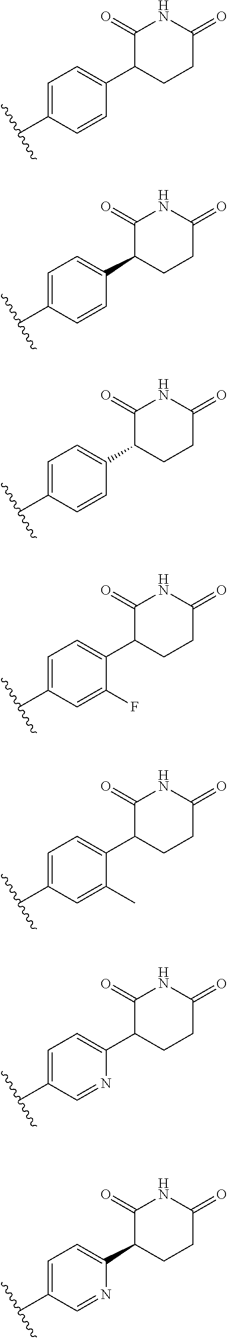

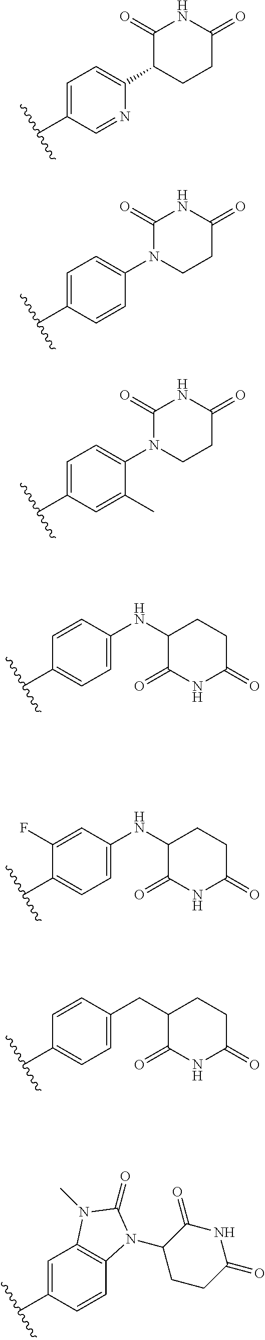

The targeted degradation of proteins through the use of bifunctional compounds, including bifunctional compounds that link an E3 ubiquitin ligase-binding moiety to a ligand that binds the targeted proteins is also disclosed herein.

Also disclosed is a composition comprising a compound of Formula I. In various embodiments, the composition is a pharmaceutical composition. In one embodiment, the pharmaceutical composition comprises at least one compound of Formula I and a pharmaceutically acceptable carrier.

In one embodiment, the pharmaceutical composition is suitable for oral administration. In one embodiment, the pharmaceutical composition is suitable for intravenous administration. In one embodiment, the pharmaceutical composition is suitable for intramuscular administration. In one embodiment, the pharmaceutical composition is suitable for parenteral administration.

Also disclosed is a method of preparing a compound of Formula I or composition or pharmaceutical composition thereof.

Also disclosed is a method of treating a disease or disorder in a subject in need thereof, the method comprising administering at least one compound of Formula I or composition or pharmaceutical composition thereof to the subject.

Also disclosed is a method for treating breast cancer in a subject in need thereof, the method comprising administering an effective amount of a compound of Formula I or composition or pharmaceutical composition thereof to the subject. In one embodiment, the breast cancer is an ER+, HER2− metastatic breast cancer chronic.

Also disclosed is a method for treating a breast cancer disease in a subject in need thereof, the method comprising administering an effective amount of a compound of Formula I or composition or pharmaceutical composition thereof in combination of an endocrine therapy to the subject.

Also disclosed is a method of reducing the level or activity of a target protein, the method comprising administering at least one compound of Formula I or composition or pharmaceutical composition thereof.

Also disclosed is a method of inhibiting a target protein, the method comprising administering at least one compound of Formula I or composition or pharmaceutical composition thereof.

In one embodiment, the target protein is CDK4/6.

### BRIEF DESCRIPTION OF THE DRAWINGS

For a further understanding of the nature, objects, and advantages of the compounds, compositions and methods disclosed herein, reference may be made to the following detailed description, read in conjunction with the following drawings, wherein like reference numerals denote like elements.

FIG. 1 shows A) the dose-dependent CDK4/6 degradation by exemplary compounds 104, 49, 73 in A375 melanoma cells; B) the dose-dependent CDK4/6 degradation by exemplary compounds 104, 49, 73 in A375 melanoma cells.

FIG. 2 shows the dose-dependent inhibition of cell proliferation of Mino cells by exemplary compound 47.

FIG. 3 shows the dose-dependent inhibition of cell proliferation of Mino cells by exemplary compound 49.

FIG. 4 shows the dose-dependent inhibition of cell proliferation of Mino cells by exemplary compound 65.

FIG. 5 shows the dose-dependent inhibition of cell proliferation of Mino cells by exemplary compound 76.

FIG. 6 shows the dose-dependent inhibition of cell proliferation of Mino cells by exemplary compound 63.

FIG. 7 shows the dose-dependent inhibition of cell proliferation of Mino cells by exemplary compound 103.

FIG. 8 shows the dose-dependent inhibition of cell proliferation of Mino cells by exemplary compound 104.

FIG. 9 shows the dose-dependent inhibition of cell proliferation of Mino cells by exemplary compound 50.

FIG. 10 shows the dose-dependent inhibition of cell proliferation of Mino cells by exemplary compound 73.

FIG. 11 shows the dose-dependent inhibition of cell proliferation of Mino cells by exemplary compound 90.

FIG. 12 shows the dose-dependent inhibition of cell proliferation of Mino cells by exemplary compound 92.

FIG. 13 shows the dose-dependent inhibition of cell proliferation of Mino cells by exemplary compound 94.

FIG. 14 shows the dose-dependent inhibition of cell proliferation of Mino cells by exemplary compound 100.

FIG. 15 shows the dose-dependent inhibition of cell proliferation of Mino cells by exemplary compounds 49, 90, and 94.

FIG. 16 shows the single oral dose (10 mg/kg) pharmacokinetic profile of compound 49 in Sprague Dawley rat.

FIG. 17 shows the efficacy of Compound 90 and Compound 94 in inhibiting Mino xenograft tumor growth in mice.

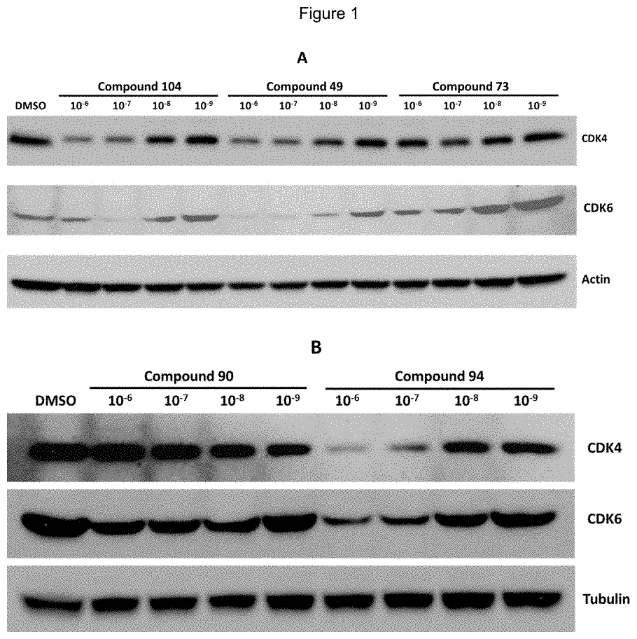

### DETAILED DESCRIPTION OF THE DISCLOSURE

The bifunctional compounds described below modulate a target protein, cyclin dependent kinases (CDK), by eliminating the kinase protein via ubiquitination and subsequent proteasomal degradation to block CDK signaling. The bifunctional compounds comprise one ligand that binds CDK and another ligand that binds to an E3 ubiquitin ligase. The two ligands are connected via a linker. The bifunctional compounds can simultaneously bind CDK (target protein) and a cereblon (CRBN) E3 ubiquitin ligase, which promotes ubiquitination of CDK and leads to degradation of CDK by the proteasome. The compounds bind competitively and/or non competitively to cyclin dependent kinases, and the E3 ubiquitin ligase, cereblon (CRBN) to effect ubiquitination and subsequent degradation of the CDK4/6 protein, thereby blocking the CDK signaling pathways and inhibiting the growth of CDK4/6 dependent cells. The disclosure also relates to pharmaceutical compositions comprising these CDK4/6 binding compounds, and methods for using the same for treatment of diseases and conditions mediated by the CDK4/6, including breast cancer.

Based on the ideas of the invention described above, the disclosed bifunctional compounds may be applied to targeted degradation of CDK4/6 and may be used to treat or prevent diseases where CDK4/6 is dysregulated.

It is to be understood that the disclosure is not limited to the particular embodiments of the disclosure described below, as variations of the particular embodiments may be made by those of ordinary skill in the art and still maintain the spirit and scope of the appended claims. It is also to be understood that the terminology employed is for the purpose of describing particular embodiments and is not intended to be limiting. Instead, the scope of the disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

In this specification and the claims reported herein, the phrase “and/or,” as used is construed to mean “either or both” of the elements, i.e., either the elements can be conjunctively present in some cases or the elements can be disjunctively present in other cases.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “cancer” refers to any of various types of malignant neoplasms, most of which invade surrounding tissues, may metastasize to several sites and are likely to recur after attempted removal and to cause death of the patient unless adequately treated. As used herein, neoplasia comprises cancer. Representative cancers include, for example, squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias, including non-acute and acute leukemias, such as acute myelogenous leukemia, acute lymphocytic leukemia, acute promyelocytic leukemia (APL), acute T-cell lymphoblastic leukemia, T-lineage acute lymphoblastic leukemia (T-ALL), adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas, among others, which may be treated by one or more compounds described herein.

A disease or disorder is “alleviated” if the severity of a sign or symptom of the disease or disorder, the frequency with which such a sign or symptom is experienced by a patient, or both, is reduced.

As used herein, the term “minimize” or “reduce”, or derivatives thereof, include a complete or partial degradation of a target protein (CDK4/6) and/or inhibition of a specified biological effect and/or reduction of CDK4/6 expression at the transcript or protein level. (which is apparent from the context in which the terms “minimize” or “reduce” are used).

The term “inhibit,” as used herein, means to suppress or block an activity or function by at least about ten percent relative to a control value. Preferably, the activity is suppressed or blocked by 50% compared to a control value, more preferably by 75%, and even more preferably by 95% or more.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell from a patient (e.g., for diagnosis or ex vivo applications), who has a disease or disorder contemplated herein, a sign or symptom of a disease or disorder contemplated herein or the potential to develop a disease or disorder contemplated herein, with the purpose to cure, heal, alleviate, relieve, alter, remedy, ameliorate, improve or affect a disease or disorder contemplated herein, the signs or symptoms of a disease or disorder contemplated herein or the potential to develop a disease or disorder contemplated herein. Such treatments may be specifically tailored or modified, based on knowledge obtained from the field of pharmacogenomics. To “treat” a disease as the term is used herein, means to reduce the frequency or severity of at least one sign or symptom of a disease or disorder experienced by a subject.

“Parenteral” administration of a composition includes, e.g., subcutaneous (s.c.), intravenous (i.v.), intramuscular (i.m.), or intrasternal injection, or infusion techniques.

The compounds according to the disclosure are isolated and purified in a manner known per se, e.g. by distilling off the solvent in vacuo and recrystallizing the residue obtained from a suitable solvent or subjecting it to one of the customary purification methods, such as chromatography on a suitable support material. Furthermore, reverse phase preparative HPLC of compounds of Formula I which possess a sufficiently basic or acidic functionality, may result in the formation of a salt, such as, in the case of a compound of Formula I which is sufficiently basic, a trifluoroacetate or formate salt for example, or, in the case of a compound of the disclosure which is sufficiently acidic, an ammonium salt for example. Salts of this type can either be transformed into its free base or free acid form, respectively, by various methods known to the person skilled in the art, or be used as salts in subsequent biological assays. Additionally, the drying process during the isolation of compounds of the disclosure may not fully remove traces of cosolvents, especially such as formic acid or trifluoroacetic acid, to give solvates or inclusion complexes. The person skilled in the art will recognize which solvates or inclusion complexes are acceptable to be used in subsequent biological assays. It is to be understood that the specific form (e.g., salt, free base, solvate, inclusion complex) of a compound of the disclosure as isolated as described herein is not necessarily the only form in which said compound can be applied to a biological assay in order to quantify the specific biological activity.

One aspect of the disclosure is salts of the compounds according to the disclosure including all inorganic and organic salts, especially all pharmaceutically acceptable inorganic and organic salts, particularly all pharmaceutically acceptable inorganic and organic salts customarily used in pharmacy.

Examples of salts include, but are not limited to, lithium, sodium, potassium, calcium, aluminum, magnesium, titanium, meglumine, ammonium, salts optionally derived from NH₃ or organic amines having from 1 to 16 C-atoms such as, e.g., ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, ethylendiamine, N-methylpiperindine, arginine, lysine, and guanidinium salts.

The salts of the disclosed compounds include pharmaceutically acceptable water-insoluble and, particularly, water-soluble salts.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, “pharmaceutically acceptable salts” refer to derivatives of the compounds disclosed herein wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt may be 1:1, or any ratio other than 1:1, e.g., 3:1, 2:1, 1:2, or 1:3.

It should be understood that all references to pharmaceutically acceptable salts include solvent addition forms (solvates) or crystal forms (polymorphs) as defined herein, of the same salt.

Salts of the compounds of formulas (I) according to the disclosure can be obtained by dissolving the free compound in a suitable solvent (for example a ketone such as acetone, methylethylketone or methylisobutylketone, an ether such as diethyl ether, tetrahydrofuran or dioxane, a chlorinated hydrocarbon such as methylene chloride or chloroform, or a low molecular weight aliphatic alcohol such as methanol, ethanol or isopropanol) which contains the desired acid or base, or to which the desired acid or base is then added. The acid or base can be employed in salt preparation, depending on whether a mono- or polybasic acid or base is concerned and depending on which salt is desired, in an equimolar quantitative ratio or one differing therefrom. The salts are obtained by filtering, reprecipitating, precipitating with a non-solvent for the salt or by evaporating the solvent. Salts obtained can be converted into the free compounds which, in turn, can be converted into salts. In this manner, pharmaceutically unacceptable salts, which can be obtained, for example, as process products in the manufacturing on an industrial scale, can be converted into pharmaceutically acceptable salts by processes known to the person skilled in the art.

According to the person skilled in the art the compounds of formula (I) as well as their salts may contain, e.g., when isolated in crystalline form, varying amounts of solvents. Included within the scope of the disclosure are therefore all solvates and in particular all hydrates of the compounds of formula (I) according to this disclosure as well as all solvates and in particular all hydrates of the salts of the compounds of formula (I) according to this disclosure.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

The compounds according to the disclosure and their salts can exist in the form of tautomers which are included in the embodiments of the disclosure.

The term “tautomer” refers to one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH conditions. The concept of tautomers that are interconvertible by tautomerizations is called tautomerism.

Where the specification depicts a compound prone to tautomerization, but only depicts one of the tautomers, it is understood that all tautomers are included as part of the meaning of the chemical depicted. It is to be understood that the compounds disclosed herein may be depicted as different tautomers. It should also be understood that when compounds have tautomeric forms, all tautomeric forms are intended to be included, and the naming of the compounds does not exclude any tautomer form.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine.

The compounds of the disclosure may, depending on their structure, exist in different stereoisomeric forms. These forms include configurational isomers or optically conformational isomers (enantiomers and/or diastereoisomers including those of atropisomers). The disclosure therefore includes enantiomers, diastereoisomers as well as mixtures thereof. From those mixtures of enantiomers and/or disastereoisomers pure stereoisomeric forms can be isolated with methods known in the art, preferably methods of chromatography, especially high performance liquid chromatography (HPLC) using achiral or chiral phase. The disclosure further includes all mixtures of the stereoisomers mentioned above independent of the ratio, including the racemates.

The compounds of the disclosure may, depending on their structure, exist in various stable isotopic forms. These forms include those in which one or more hydrogen atoms have been replaced with deuterium atoms, those in which one or more nitrogen atoms have been replaced with ¹⁵N atoms, or those in which one or more atoms of carbon, fluorine, chlorine, bromine, sulfur, or oxygen have been replaced by the stable isotope of the respective, original atoms.

Some of the compounds and salts according to the disclosure may exist in different crystalline forms (polymorphs) which are within the scope of the disclosure.

It is a further object of the disclosure to provide CDK4/6 degrading compounds, methods of synthesizing the CDK4/6 degrading bifunctional compounds, methods of manufacturing the CDK4/6 degrading compounds, and methods of using the CDK4/6 degrading compounds.

As used herein, the term “pharmaceutical composition” refers to a mixture of at least one compound of Formula I with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration. The term “pharmacological composition,” “therapeutic composition,” “therapeutic formulation” or “pharmaceutically acceptable formulation” can mean, but is in no way limited to, a composition or formulation that allows for the effective distribution of an agent provided by the invention, which is in a form suitable for administration to the physical location most suitable for their desired activity, e.g., systemic administration.

Non-limiting examples of agents suitable for formulation with the compounds of Formula I include: cinnamoyl, PEG, phospholipids or lipophilic moieties, phosphorothioates, P-glycoprotein inhibitors (such as Pluronic P85) which can enhance entry of drugs into various tissues, for example the CNS (Jolliet-Riant and Tillement, 1999); biodegradable polymers, such as poly (DL-lactide-coglycolide) microspheres for sustained release delivery after implantation (Emerich, D F et al, 1999) Alkermes, Inc. Cambridge, Mass.; and loaded nanoparticles, such as those made of polybutylcyanoacrylate, which can deliver drugs across the blood brain barrier and can alter neuronal uptake mechanisms (Schroeder et al, 1999).

A “therapeutic” treatment is a treatment administered to a subject who exhibits signs or symptoms of pathology disease or disorder, for the purpose of diminishing or eliminating those signs or symptoms.

As used herein, the terms “effective amount,” “pharmaceutically effective amount” and “therapeutically effective amount” refer to a sufficient amount of an agent to provide the desired biological or physiologic result. That result may be reduction and/or alleviation of a sign, a symptom, or a cause of a disease or disorder, or any other desired alteration of a biological system. An appropriate effective amount in any individual case may be determined by one of ordinary skill in the art using routine experimentation.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound of Formula I, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound of Formula I, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “halo” or “halogen” alone or as part of another substituent means, unless otherwise stated, a fluorine, chlorine, bromine, or iodine atom.

As used herein, the term “alkyl,” by itself or as part of another substituent means, unless otherwise stated, a straight or branched chain hydrocarbon having the number of carbon atoms designated (i.e., C₁₋₆ means one to six carbon atoms) and includes straight, branched chain, or cyclic substituent groups. Examples include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. The term “alkyl,” unless otherwise noted, is also meant to include those derivatives of alkyl defined in more detail below, such as “heteroalkyl”, “haloalkyl” and “homoalkyl”.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C═N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

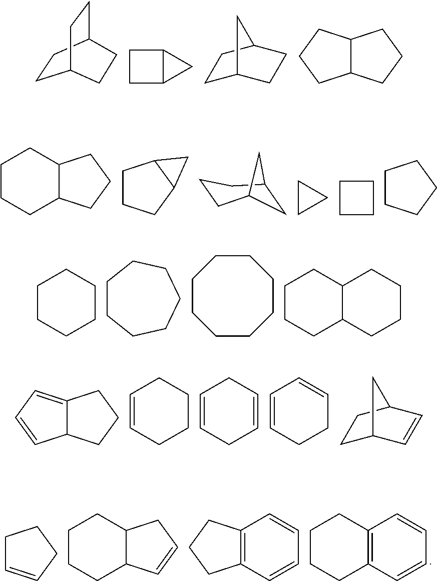

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon double bond or one carbon triple bond.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include:

—O—CH₂CH₂—CH₃, —CH₂CH₂—CH₂OH, —CH₂—CH₂—NH—CH₃, —CH₂—S—CH₂CH₃, and —CH₂CH₂—S(═O)—CH₃. Up to two heteroatoms may be consecutive, such as, for example, —CH₂—NH—OCH₃, or —CH₂—CH₂—S—S—CH₃. As used herein, the terms “heteroalkyl” refers to “alkoxy,” “alkylamino” and “alkylthio” that are used in their conventional sense, and refers to alkyl groups linked to molecules via an oxygen atom, an amino group, a sulfur atom, respectively.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers.

As used herein, the term “heterocycloalkyl” or “heterocyclyl” refers to a heteroalicyclic group containing one to four ring heteroatoms each selected from O, S and N. In one embodiment, each heterocycloalkyl group has from 4 to 10 atoms in its ring system, with the proviso that the ring of said group does not contain two adjacent O or S atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

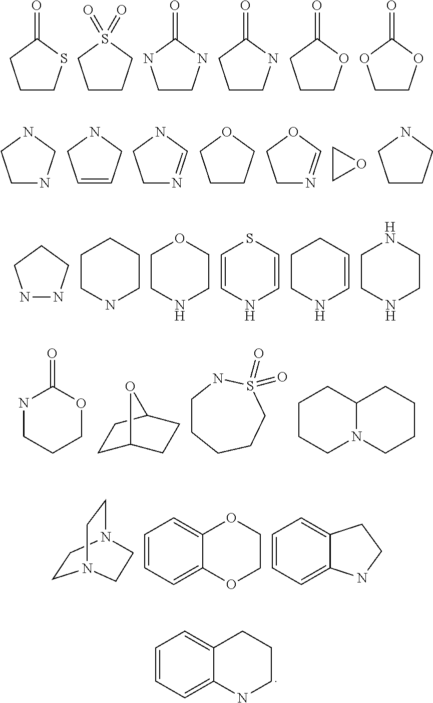

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

As used herein, the term “heteroaryl” or “heteroaromatic” refers to a heterocycle having aromatic character. A polycyclic heteroaryl may include one or more rings that are partially saturated. Examples include the following moieties:

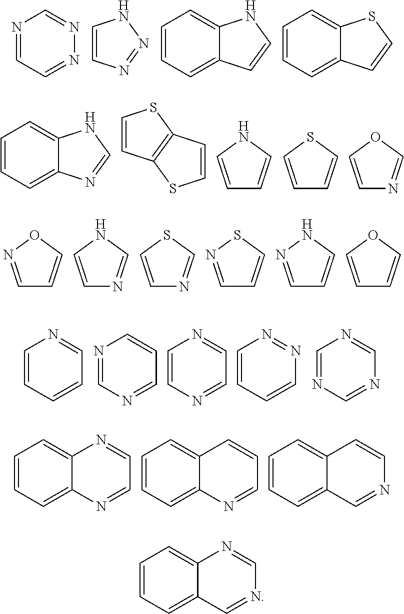

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. The substituents are independently selected, and substitution may be at any chemically accessible position. In one embodiment, the substituents vary in number between one and four. In another embodiment, the substituents vary in number between one and three.

In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, or other variant of the reference molecule.

As used herein, the term “potency” refers to the dose needed to produce half the maximal response (ED₅₀).

As used herein, the term “efficacy” refers to the maximal effect (E) achieved within an assay.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds

Provided herein are compounds of Formula I, or a tautomer, stereoisomer or a mixture of stereoisomers, pharmaceutically acceptable salt, or solvate thereof:

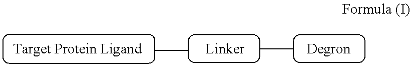

wherein:
the Target Protein Ligand is capable of binding to a target protein, such as a cyclin-dependent kinase (e.g., CDK4 and/or CDK6); the Target Protein Ligand is one of the following structures:

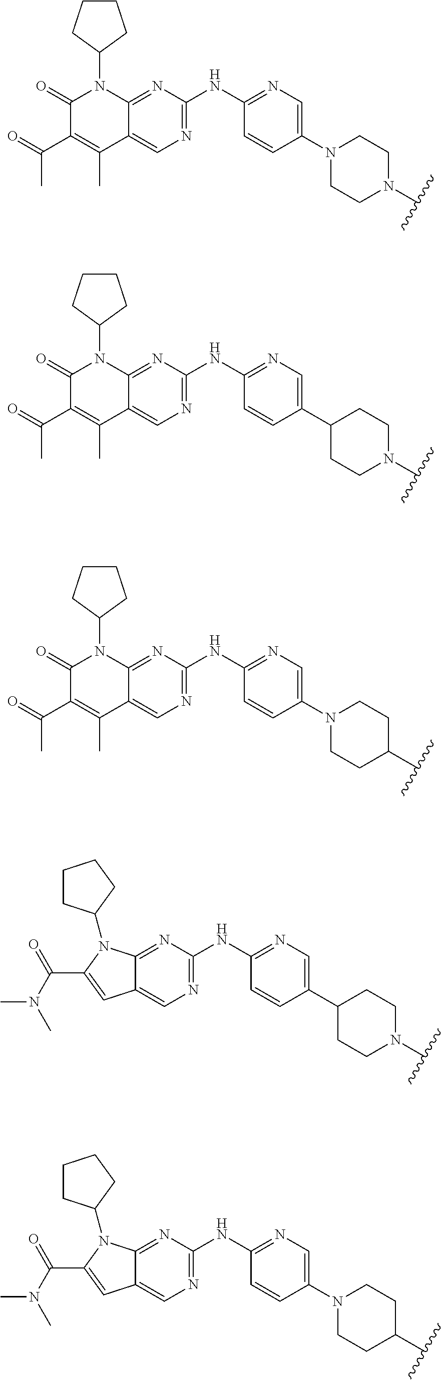 [SMILES: *N1CCN(c2ccc(Nc3ncc4c(C)c(C(C)=O)c(=O)n(C5CCCC5)c4n3)nc2)CC1 ; *N1CCC(c2ccc(Nc3ncc4c(C)c(C(C)=O)c(=O)n(C5CCCC5)c4n3)nc2)CC1 ; *C1CCN(c2ccc(Nc3ncc4c(C)c(C(C)=O)c(=O)n(C5CCCC5)c4n3)nc2)CC1 ; *N1CCC(c2ccc(Nc3ncc4cc(C(=O)N(C)C)n(C5CCCC5)c4n3)nc2)CC1 ; *C1CCN(c2ccc(Nc3ncc4cc(C(=O)N(C)C)n(C5CCCC5)c4n3)nc2)CC1]

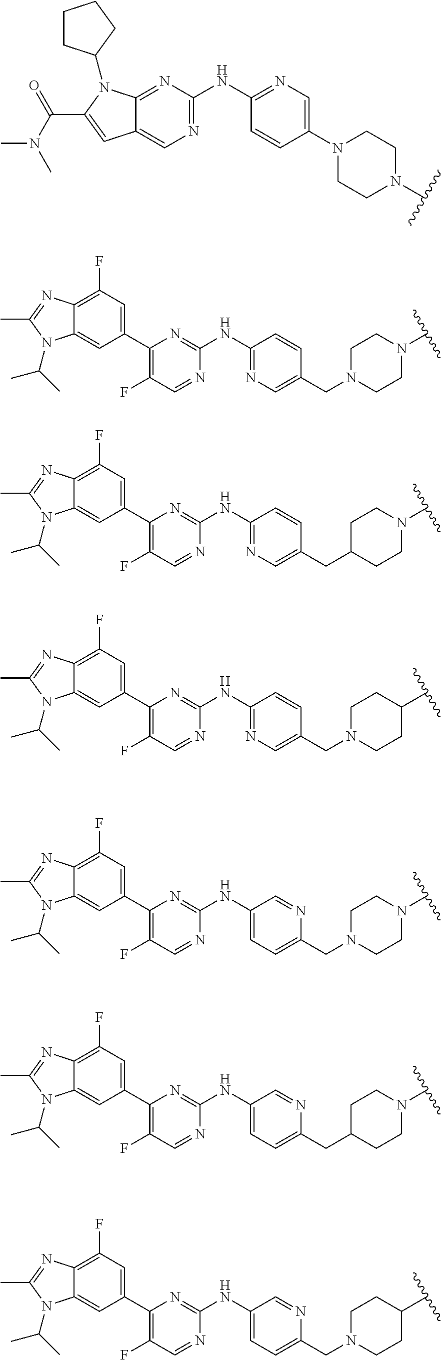

The Linker is a group that is covalently bonded to the Target Protein Ligand and the Degron; the Linker is an optionally substituted linking moiety comprising a branched or unbranched, cyclized or uncyclized, saturated or unsaturated chain of 5 to 16 carbon atoms in length, selected from:

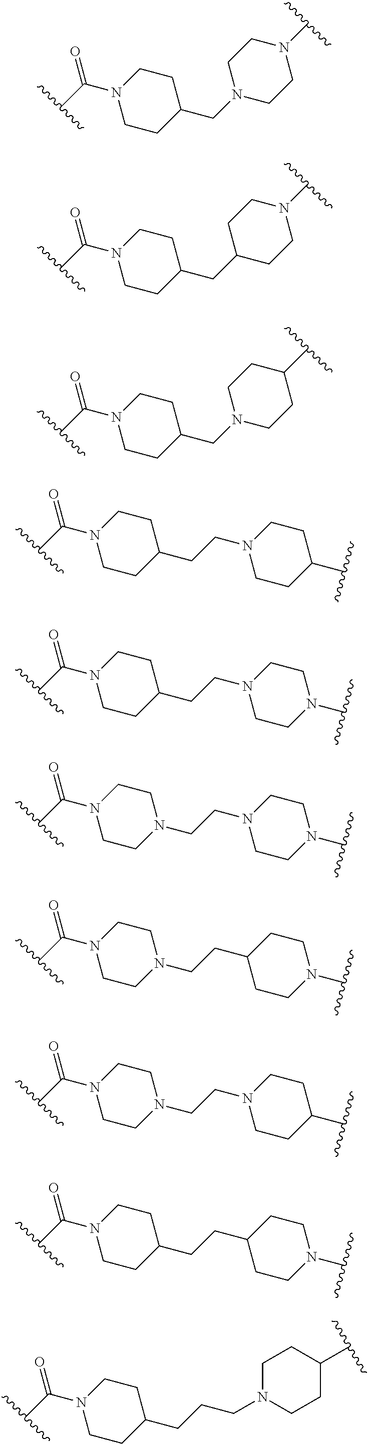

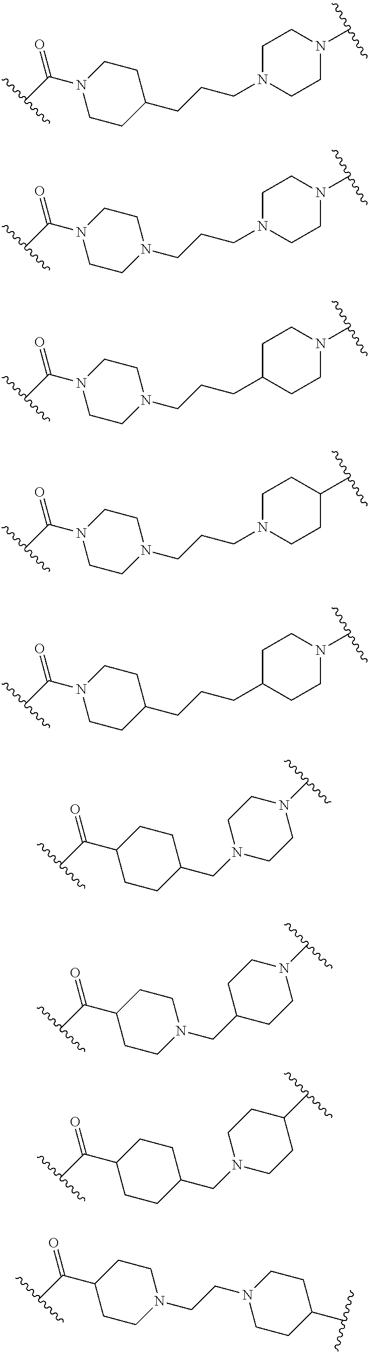

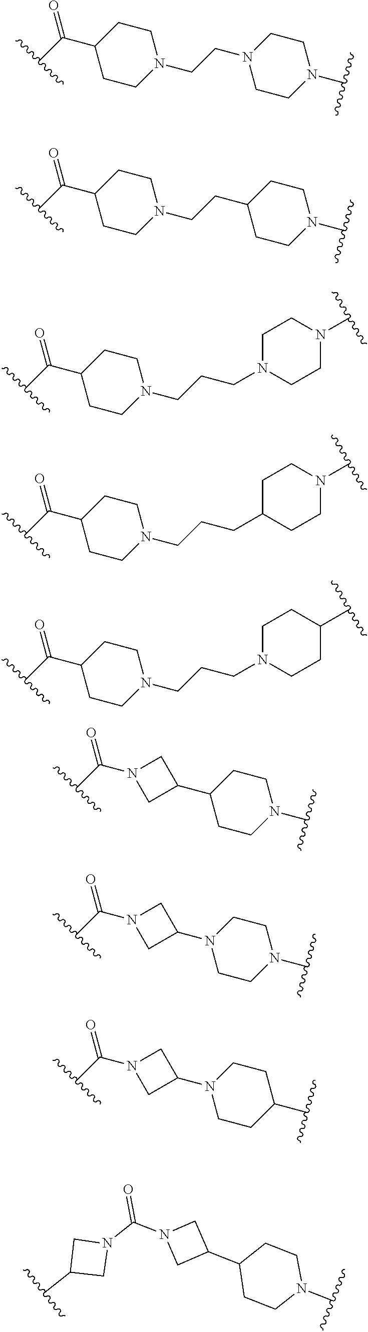

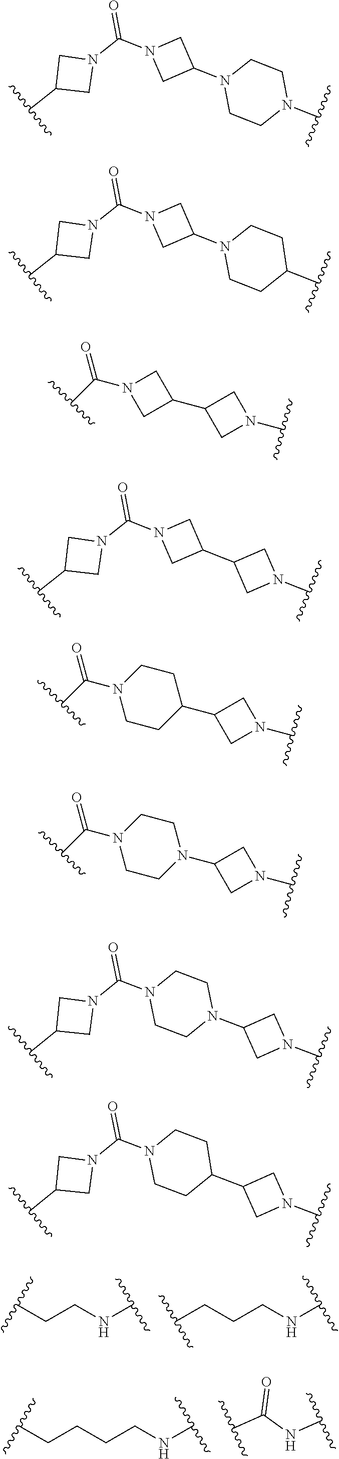

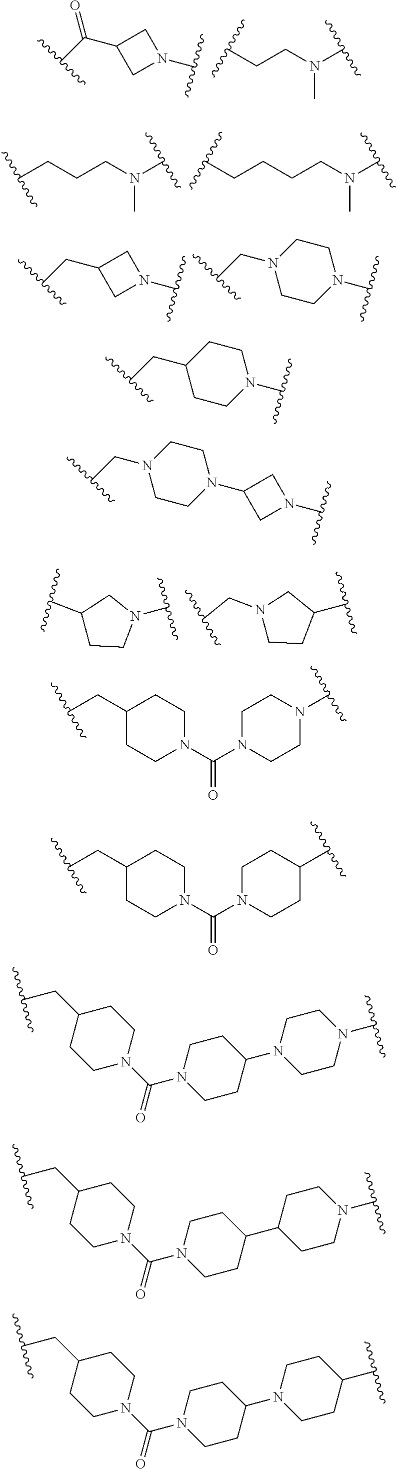

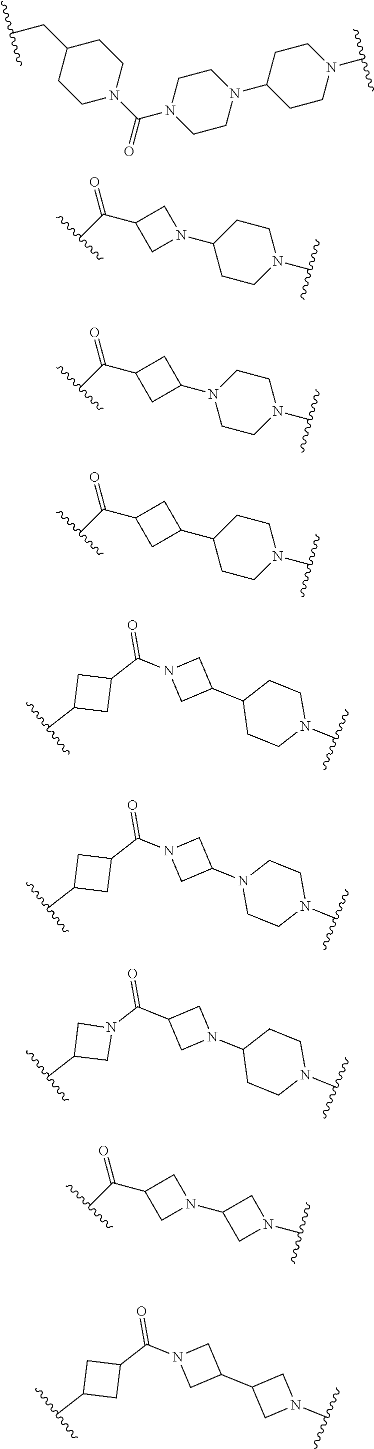

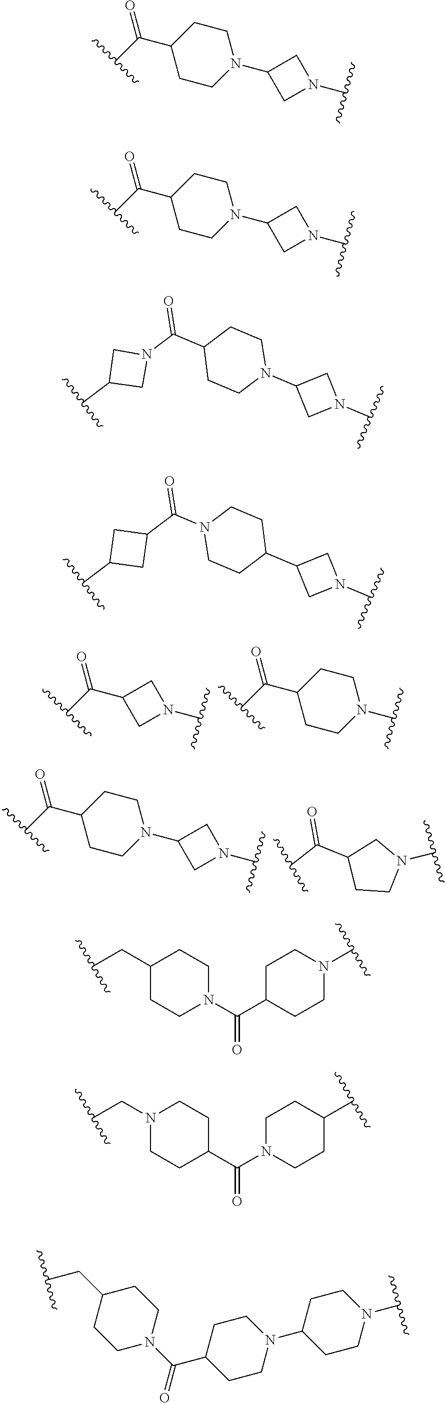

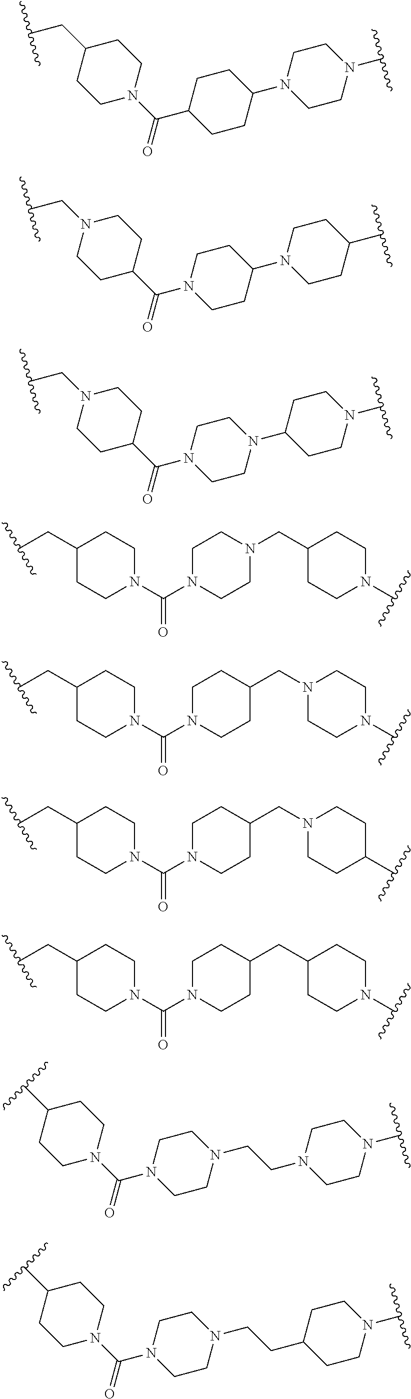

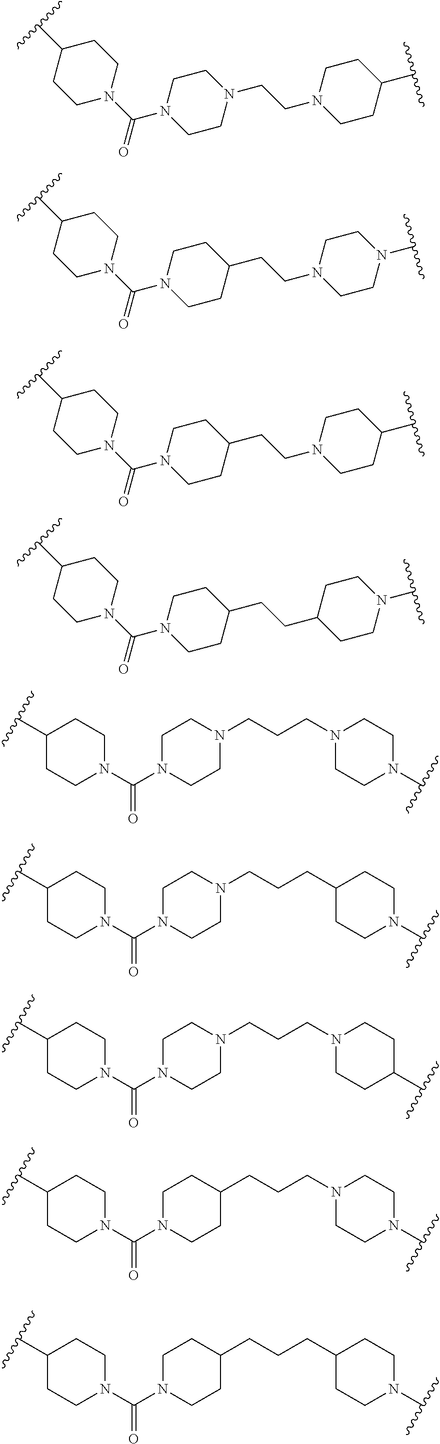

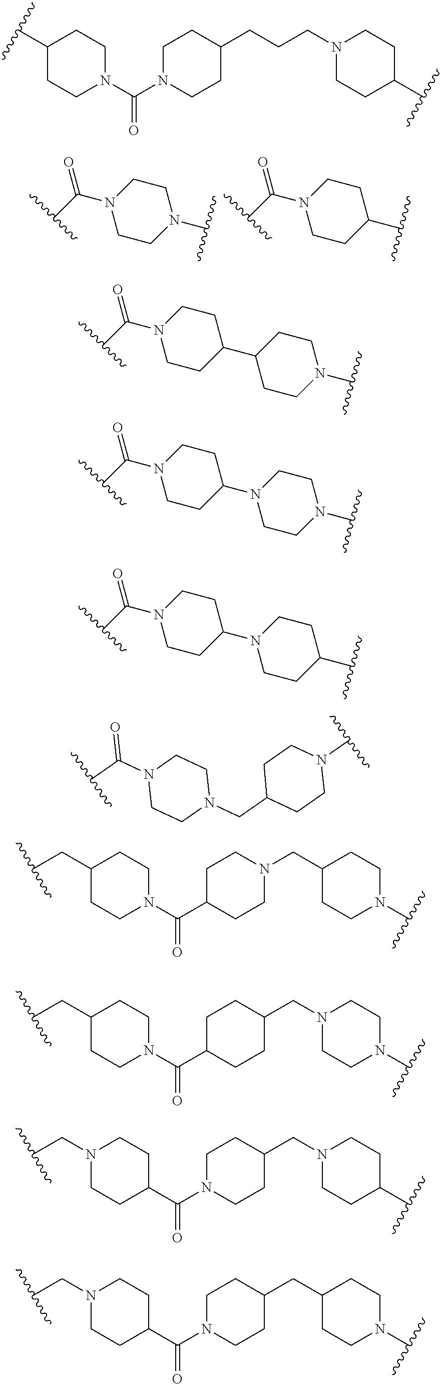

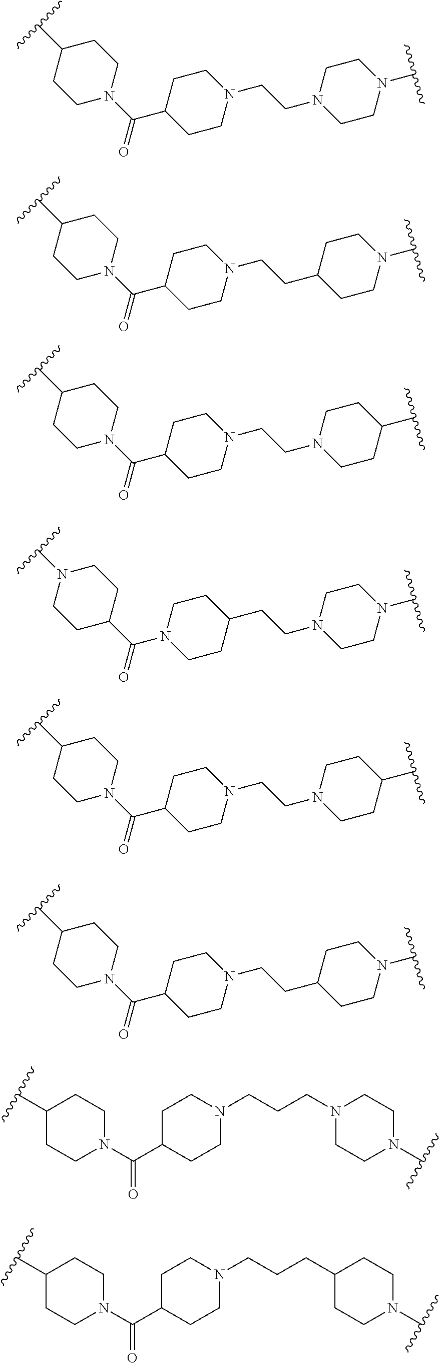

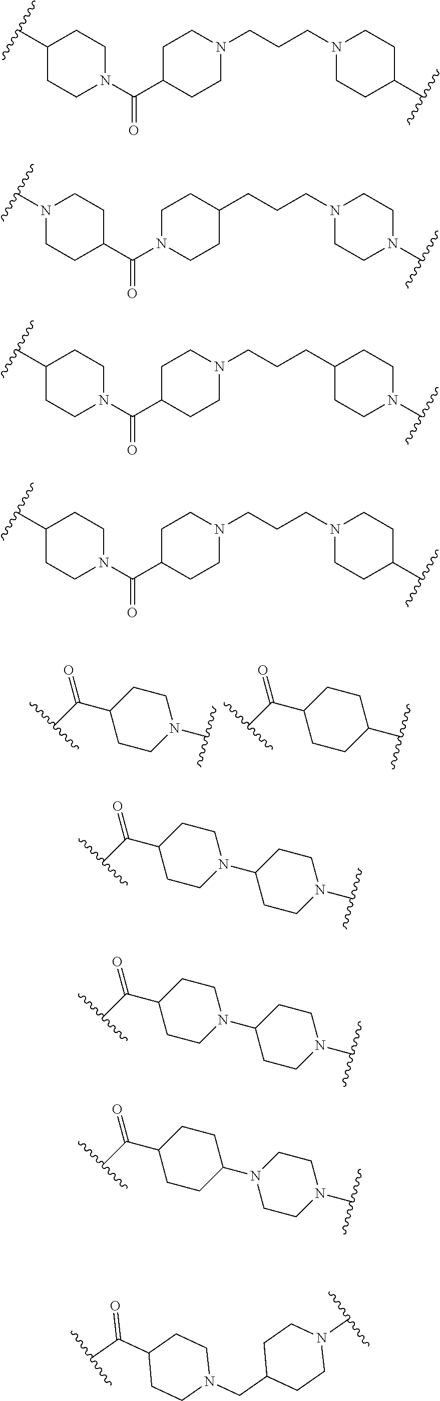

And the Degron is capable of binding to a ubiquitin ligase, such as an E3 ubiquitin ligase (e.g., CRBN). The Degron is selected from the following structures:

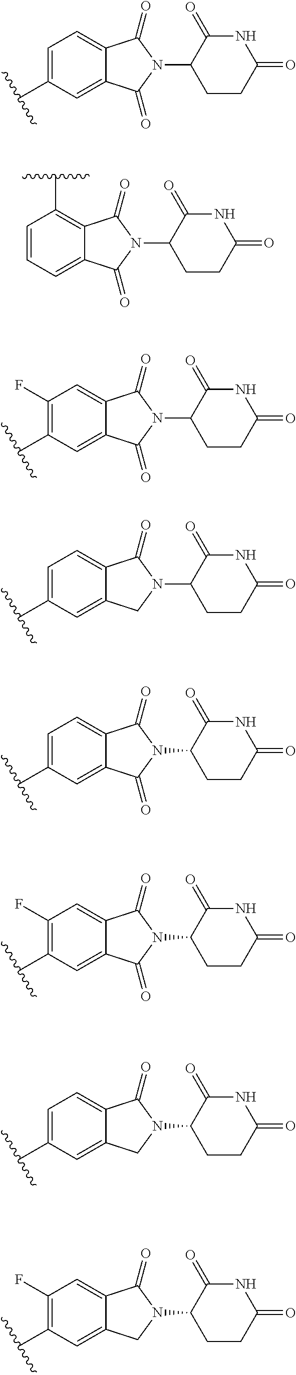

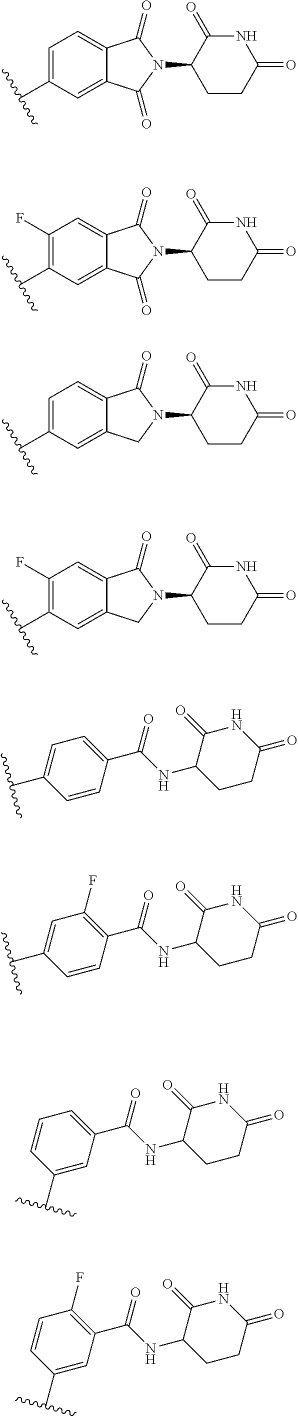

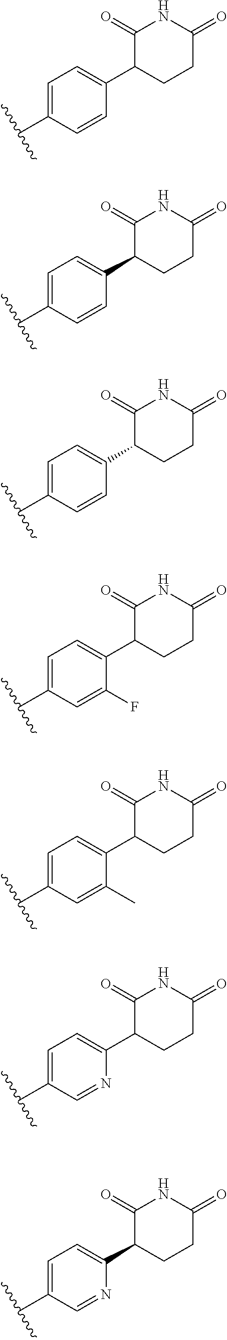

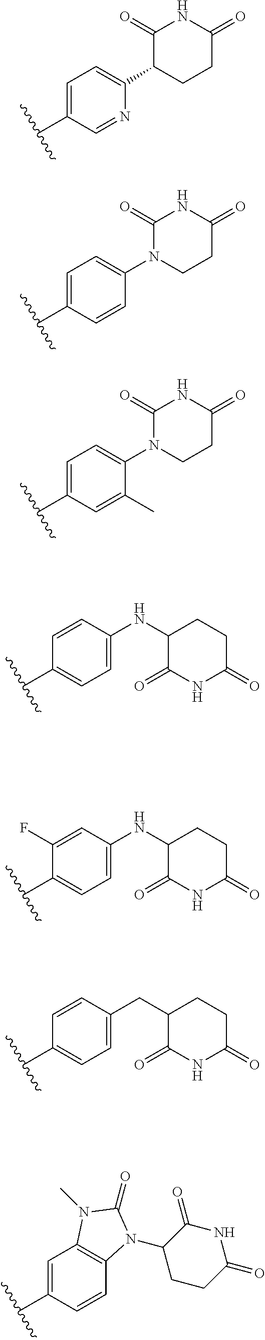

In some embodiments, provided herein is a compound, or tautomer, stereoisomer, pharmaceutically acceptable salt, or hydrate thereof is selected from the group consisting of compounds presented in Table 1 and any combination thereof. For example, in some embodiments, the compound of the present invention or pharmaceutically acceptable salt thereof is selected from the group consisting of compounds presented in Table 1 and any combination thereof.

TABLE 1
Exemplary Compounds of the Disclosure

| | | | | IC₅₀ | | | | |
|---|---|---|---|---|---|---|---|---|
| | | | | (anti- | | | | |
| | | | | pro- | | | | |
| | | | Exact | lifer- | DC₅₀ | Dₘₐₓ | DC₅₀ | Dₘₐₓ |
| # | Structure | IUPAC Name | mass | ation) | CDK4 | CDK4 | CDK6 | CDK6 |
| 1 | 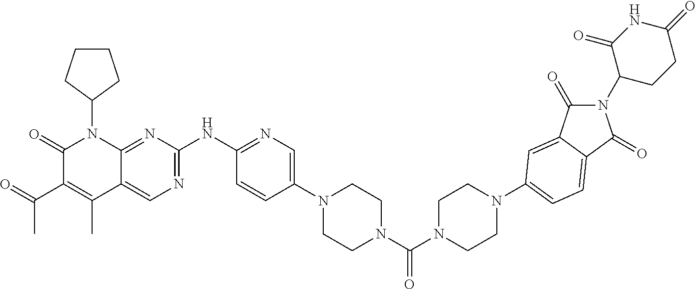 [SMILES: CC(=O)c1c(C)c2cnc(Nc3ccc(N4CCN(C(=O)N5CCN(c6ccc7c(c6)C(=O)N(C6CCC(=O)NC6=O)C7=O)CC5)CC4)cn3)nc2n(C2CCCC2)c1=O] | 5-(4-(4-(6-((6-acetyl- 8-cyclopentyl- 5-methyl-7-oxo-7,8- dihydropyrido[2,3- d]pyrimidin-2- yl)amino)pyridin- 3-yl)piperazine-1- carbonyl)piperazin- 1-yl)-2-(2,6- dioxopiperidin-3-yl) isoindoline-1,3- dione | 815.35 | A | B | B | A | B |
| 2 | 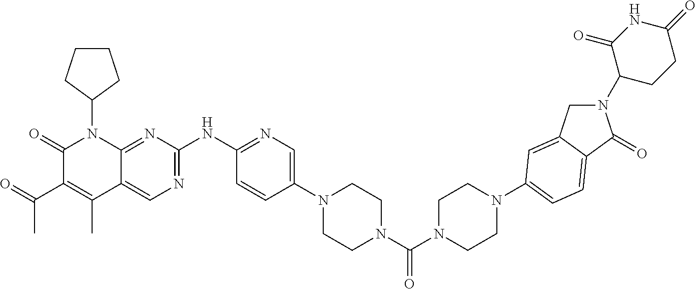 [SMILES: CC(=O)c1c(C)c2cnc(Nc3ccc(N4CCN(C(=O)N5CCN(c6ccc7c(c6)CN(C6CCC(=O)NC6=O)C7=O)CC5)CC4)cn3)nc2n(C2CCCC2)c1=O] | 3-(5-(4-(4-(6- ((6-acetyl-8- cyclopentyl-5- methyl-7-oxo-7,8- dihydropyrido[2,3- d]pyrimidin-2- yl)amino)pyridin-3- yl)piperazine-1- carbonyl)piperazin- 1-yl)-1- oxoisoindolin-2- yl)piperidine-2,6- dione | 801.37 | B | C | B | C | B |
| 3 | 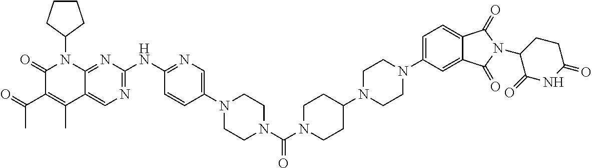 [SMILES: CC(=O)c1c(C)c2cnc(Nc3ccc(N4CCN(C(=O)N5CCC(N6CCN(c7ccc8c(c7)C(=O)N(C7CCC(=O)NC7=O)C8=O)CC6)CC5)CC4)cn3)nc2n(C2CCCC2)c1=O] | 5-(4-(1-(4-(6- ((6-acetyl-8- cyclopentyl-5- methyl-7-oxo-7,8- dihydropyrido[2,3- d]pyrimidin-2- yl)amino)pyridin-3- yl)piperazine-1- carbonyl)piperidin- 4-yl)piperazin-1- yl)-2-(2,6- dioxopiperidin-3- yl)isoindoline- 1,3-dione | 898.42 | B | C | C | C | C |
| 4 | 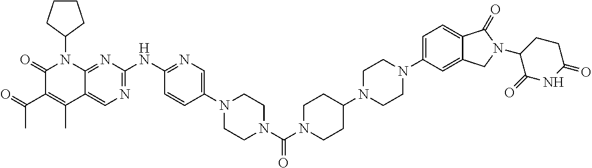 [SMILES: CC(=O)c1c(C)c2cnc(Nc3ccc(N4CCN(C(=O)N5CCC(N6CCN(c7ccc8c(c7)CN(C7CCC(=O)NC7=O)C8=O)CC6)CC5)CC4)cn3)nc2n(C2CCCC2)c1=O] | 3-(5-(4-(1-(4- (6-((6-acetyl-8- cyclopentyl-5- methyl-7-oxo-7,8- dihydropyrido[2,3- d]pyrimidin-2- yl)amino)pyridin-3- yl)piperazine-1- carbonyl)piperidin- 4-yl)piperazin-1- yl)-1-oxoisoindolin- 2-yl)piperidine- 2,6-dione | 884.44 | B | C | C | C | C |
| 5 | 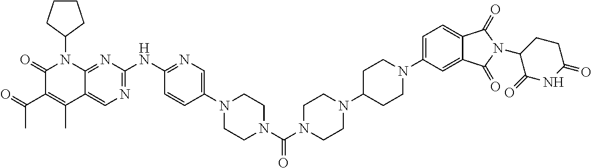 [SMILES: CC(=O)c1c(C)c2cnc(Nc3ccc(N4CCN(C(=O)N5CCN(C6CCN(c7ccc8c(c7)C(=O)N(C7CCC(=O)NC7=O)C8=O)CC6)CC5)CC4)cn3)nc2n(C2CCCC2)c1=O] | 5-(4-(4-(4-(6- ((6-acetyl-8- cyclopentyl-5- methyl-7-oxo-7,8- dihydropyrido[2,3- d]pyrimidin-2- yl)amino)pyridin-3- yl)piperazine-1- carbonyl)piperazin- 1-yl)piperidin-1- yl)-2-(2,6- dioxopiperidin-3- yl)isoindoline- 1,3-dione | 898.42 | B | C | B | C | B |
| 6 | 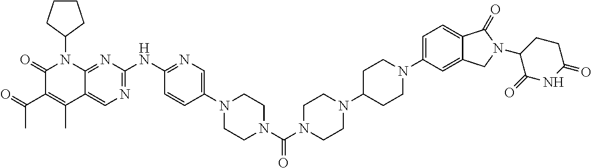 [SMILES: CC(=O)c1c(C)c2cnc(Nc3ccc(N4CCN(C(=O)N5CCN(C6CCN(c7ccc8c(c7)CN(C7CCC(=O)NC7=O)C8=O)CC6)CC5)CC4)cn3)nc2n(C2CCCC2)c1=O] | 3-(5-(4-(4-(4- (6-((6-acetyl-8- cyclopentyl-5- methyl-7-oxo-7,8- dihydropyrido[2,3- d]pyrimidin-2- yl)amino)pyridin-3- yl)piperazine-1- carbonyl)piperazin- 1-yl)piperidin-1- yl)-1-oxoisoindolin- 2-yl)piperidine- 2,6-dione | 884.44 | C | C | C | C | C |
| 7 | 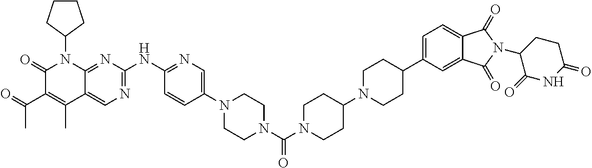 [SMILES: CC(=O)c1c(C)c2cnc(Nc3ccc(N4CCN(C(=O)N5CCC(N6CCC(c7ccc8c(c7)C(=O)N(C7CCC(=O)NC7=O)C8=O)CC6)CC5)CC4)cn3)nc2n(C2CCCC2)c1=O] | 5-(1′-(4-(6-((6-acetyl- 8-cyclopentyl- 5-methyl-7-oxo-7,8- dihydropyrido[2,3- d]pyrimidin-2- yl)amino)pyridin-3- yl)piperazine-1- carbonyl)-[1,4′- bipiperidin]-4-yl)-2- (2,6- dioxopiperidin-3- yl)isoindoline- 1,3-dione | 897.43 | B | B | B | B | B |
| 8 | 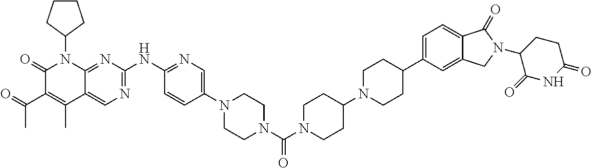 [SMILES: CC(=O)c1c(C)c2cnc(Nc3ccc(N4CCN(C(=O)N5CCC(N6CCC(c7ccc8c(c7)CN(C7CCC(=O)NC7=O)C8=O)CC6)CC5)CC4)cn3)nc2n(C2CCCC2)c1=O] | 3-(5-(1′-(4-(6- ((6-acetyl-8- cyclopentyl-5- methyl-7-oxo-7,8- dihydropyrido[2,3- d]pyrimidin-2- yl)amino)pyridin-3- yl)piperazine-1- carbonyl)-[1,4′- bipiperidin]-4-yl)-1- oxoisoindolin-2- yl)piperidine-2,6- dione | 883.45 | C | C | B | C | C |
| 9 | 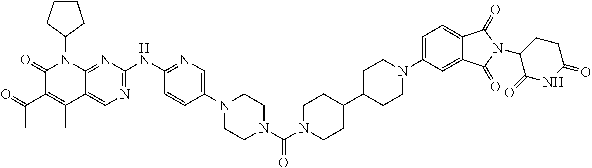 [SMILES: CC(=O)c1c(C)c2cnc(Nc3ccc(N4CCN(C(=O)N5CCC(C6CCN(c7ccc8c(c7)C(=O)N(C7CCC(=O)NC7=O)C8=O)CC6)CC5)CC4)cn3)nc2n(C2CCCC2)c1=O] | 5-(1′-(4-(6- ((6-acetyl- 8-cyclopentyl- 5-methyl-7-oxo-7,8- dihydropyrido[2,3- d]pyrimidin-2- yl)amino)pyridin-3- yl)piperazine-1- carbonyl)-[4,4′- bipiperidin]-1-yl)-2- (2,6- dioxopiperidin-3- yl)isoindoline- 1,3-dione | 897.43 | C | C | C | C | C |
| 10 | 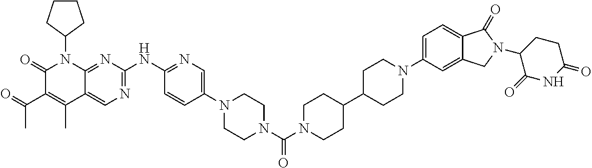 [SMILES: CC(=O)c1c(C)c2cnc(Nc3ccc(N4CCN(C(=O)N5CCC(C6CCN(c7ccc8c(c7)CN(C7CCC(=O)NC7=O)C8=O)CC6)CC5)CC4)cn3)nc2n(C2CCCC2)c1=O] | 3-(5-(1′-(4- (6-((6-acetyl-8- cyclopentyl-5- methyl-7-oxo-7,8- dihydropyrido[2,3- d]pyrimidin-2- yl)amino)pyridin-3- yl)piperazine-1- carbonyl)-[4,4′- bipiperidin]-1-yl)-1- oxoisoindolin-2- yl)piperidine-2,6- dione | 883.45 | C | C | C | C | C |
| 11 | 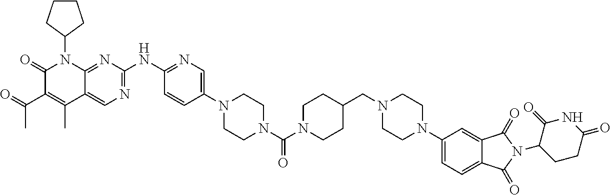 [SMILES: CC(=O)c1c(C)c2cnc(Nc3ccc(N4CCN(C(=O)N5CCC(CN6CCN(c7ccc8c(c7)C(=O)N(C7CCC(=O)NC7=O)C8=O)CC6)CC5)CC4)cn3)nc2n(C2CCCC2)c1=O] | 5-(4-((1-(4- (6-(((6-acetyl-8- cyclopentyl-5- methyl-7-oxo-7,8- dihydropyrido[2,3- d]pyrimidin-2- yl)amino)pyridin- 3-yl)piperazine-1- carbonyl) piperidin-4- yl)methyl)piperazin- 1-yl)-2-(2,6- dioxopiperidin-3- yl)isoindoline-1,3- dione | 912.44 | B | B | B | B | B |
| 12 | 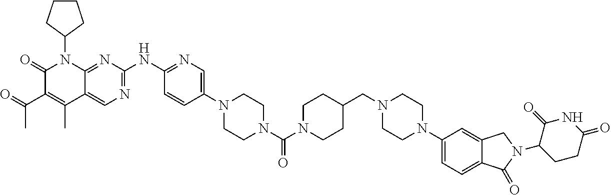 [SMILES: CC(=O)c1c(C)c2cnc(Nc3ccc(N4CCN(C(=O)N5CCC(CN6CCN(c7ccc8c(c7)CN(C7CCC(=O)NC7=O)C8=O)CC6)CC5)CC4)cn3)nc2n(C2CCCC2)c1=O] | 3-(5-(4-((1-(4- (6-((6-acetyl-8- cyclopentyl-5- methyl-7-oxo-7,8- dihydropyrido[2,3- d]pyrimidin-2- yl)amino)pyridin- 3-yl)piperazine-1- carbonyl) piperidin-4- yl)methyl) piperazin-1-yl)-1- oxoisoindolin-2- yl)piperidine-2,6- dione | 898.45 | B | C | B | C | B |
| 13 | 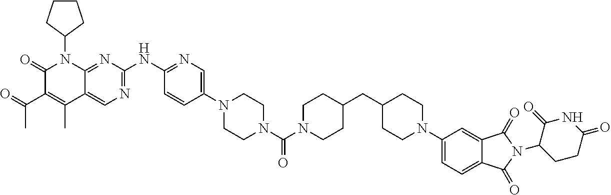 [SMILES: CC(=O)c1c(C)c2cnc(Nc3ccc(N4CCN(C(=O)N5CCC(CC6CCN(c7ccc8c(c7)C(=O)N(C7CCC(=O)NC7=O)C8=O)CC6)CC5)CC4)cn3)nc2n(C2CCCC2)c1=O] | 5-(4-((1-(4-(6- ((6-acetyl-8- cyclopentyl-5- methyl-7-oxo-7,8- dihydropyrido[2,3- d]pyrimidin-2- yl)amino)pyridin-3- yl)piperazine-1- carbonyl) piperidin-4- yl)methyl) piperidin-1- yl)-2-(2,6- dioxopiperidin-3- yl)isoindoline-1,3- dione | 911.44 | B | B | B | B | B |
| 14 | 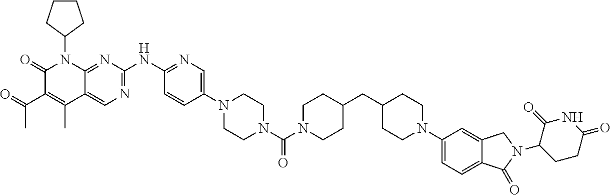 [SMILES: CC(=O)c1c(C)c2cnc(Nc3ccc(N4CCN(C(=O)N5CCC(CC6CCN(c7ccc8c(c7)CN(C7CCC(=O)NC7=O)C8=O)CC6)CC5)CC4)cn3)nc2n(C2CCCC2)c1=O] | 3-(5-(4-((1-(4- (6-((6-acetyl-8- cyclopentyl-5- methyl-7-oxo-7,8- dihydropyrido[2,3- d]pyrimidin-2- yl)amino)pyridin-3- yl)piperazine-1- carbonyl) piperidin-4- yl)methyl) piperidin-1-yl)-1- oxoisoindolin-2- yl)piperidine-2,6- dione | 897.46 | B | C | C | C | B |
| 15 | 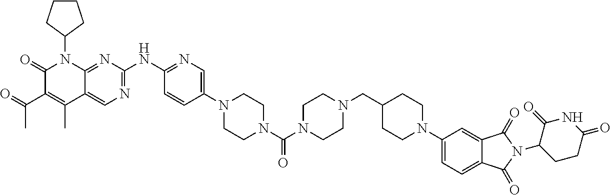 [SMILES: CC(=O)c1c(C)c2cnc(Nc3ccc(N4CCN(C(=O)N5CCN(CC6CCN(c7ccc8c(c7)C(=O)N(C7CCC(=O)NC7=O)C8=O)CC6)CC5)CC4)cn3)nc2n(C2CCCC2)c1=O] | 5-(4-((4-(4-(6- ((6-acetyl-8- cyclopentyl-5- methyl-7-oxo-7,8- dihydropyrido[2,3- d]pyrimidin-2- yl)amino)pyridin-3- yl)piperazine-1- carbonyl) piperazin-1- yl)methyl)piperidin- 1-yl)-2-(2,6- dioxopiperidin-3-yl) isoindoline-1,3- dione | 912.44 | C | C | C | C | C |
| 16 | 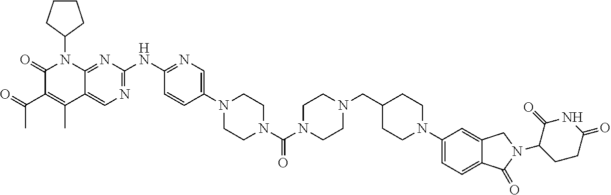 [SMILES: CC(=O)c1c(C)c2cnc(Nc3ccc(N4CCN(C(=O)N5CCN(CC6CCN(c7ccc8c(c7)CN(C7CCC(=O)NC7=O)C8=O)CC6)CC5)CC4)cn3)nc2n(C2CCCC2)c1=O] | 3-(5-(4-((4-(4- (6-((6-acetyl-8- cyclopentyl-5- methyl-7-oxo-7,8- dihydropyrido[2,3- d]pyrimidin-2- yl)amino)pyridin-3- yl)piperazine-1- carbonyl) piperazin-1- yl)methyl) piperidin-1-yl)-1- oxoisoindolin-2- yl)piperidine-2,6- dione | 898.46 | C | C | C | C | C |
| 17 | 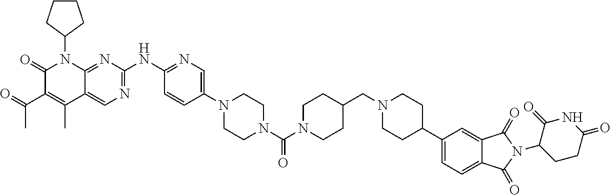 [SMILES: CC(=O)c1c(C)c2cnc(Nc3ccc(N4CCN(C(=O)N5CCC(CN6CCC(c7ccc8c(c7)C(=O)N(C7CCC(=O)NC7=O)C8=O)CC6)CC5)CC4)cn3)nc2n(C2CCCC2)c1=O] | 5-(1-((1-(4-(6- ((6-acetyl-8- cyclopentyl-5- methyl-7-oxo-7,8- dihydropyrido[2,3- d]pyrimidin-2- yl)amino)pyridin-3- yl)piperazine-1- carbonyl) piperidin-4- yl)methyl)piperidin- 4-yl)-2-(2,6- dioxopiperidin-3- yl)isoindoline-1,3- dione | 911.44 | B | B | C | B | C |
| 18 | 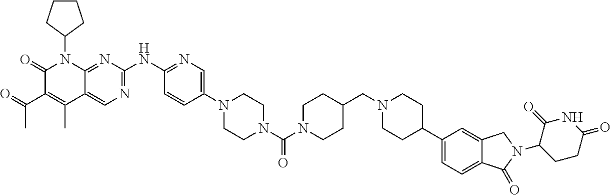 [SMILES: CC(=O)c1c(C)c2cnc(Nc3ccc(N4CCN(C(=O)N5CCC(CN6CCC(c7ccc8c(c7)CN(C7CCC(=O)NC7=O)C8=O)CC6)CC5)CC4)cn3)nc2n(C2CCCC2)c1=O] | 3-(5-(1-((1-(4- (6-((6-acetyl-8- cyclopentyl-5- methyl-7-oxo-7,8- dihydropyrido[2,3- d]pyrimidin-2- yl)amino)pyridin-3- yl)piperazine-1- carbonyl) piperidin-4- yl)methyl) piperidin-4-yl)-1- oxoisoindolin-2-yl) piperidine-2,6- dione | 897.46 | C | C | C | C | C |
| 19 | 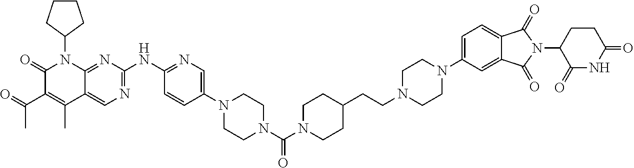 [SMILES: CC(=O)c1c(C)c2cnc(Nc3ccc(N4CCN(C(=O)N5CCC(CCN6CCN(c7ccc8c(c7)C(=O)N(C7CCC(=O)NC7=O)C8=O)CC6)CC5)CC4)cn3)nc2n(C2CCCC2)c1=O] | 5-(4-(2-(1-(4- (6-((6-acetyl-8- cyclopentyl-5- methyl-7-oxo-7,8- dihydropyrido[2,3- d]pyrimidin-2- yl)amino)pyridin-3- yl)piperazine-1- carbonyl) piperidin-4- yl)ethyl)piperazin- 1-yl)-2-(2,6- dioxopiperidin-3- yl)isoindoline-1,3- dione | 926.46 | A | B | B | B | B |
| 20 | 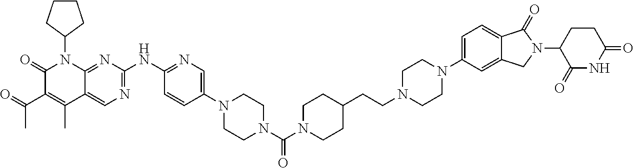 [SMILES: CC(=O)c1c(C)c2cnc(Nc3ccc(N4CCN(C(=O)N5CCC(CCN6CCN(c7ccc8c(c7)CN(C7CCC(=O)NC7=O)C8=O)CC6)CC5)CC4)cn3)nc2n(C2CCCC2)c1=O] | 3-(5-(4-(2-(1-(4- (6-((6-acetyl-8- cyclopentyl-5- methyl-7-oxo-7,8- dihydropyrido[2,3- d]pyrimidin-2- yl)amino)pyridin-3- yl)piperazine-1- carbonyl)piperidin-4- yl)ethyl) piperazin-1-yl)-1- oxoisoindolin-2- yl)piperidine-2,6- dione | 912.48 | B | B | B | B | B |
| 21 | 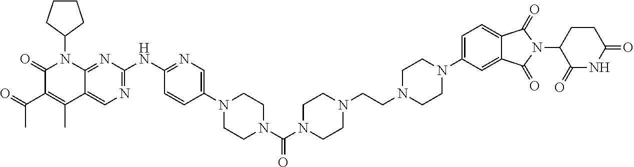 [SMILES: CC(=O)c1c(C)c2cnc(Nc3ccc(N4CCN(C(=O)N5CCN(CCN6CCN(c7ccc8c(c7)C(=O)N(C7CCC(=O)NC7=O)C8=O)CC6)CC5)CC4)cn3)nc2n(C2CCCC2)c1=O] | 5-(4-(2-(4-(4- (6-((6-acetyl-8- cyclopentyl-5- methyl-7-oxo-7,8- dihydropyrido[2,3- d]pyrimidin-2- yl)amino)pyridin-3- yl)piperazine-1- carbonyl) piperazin-1- yl)ethyl)piperazin- 1-yl)-2-(2,6- dioxopiperidin-3- yl)isoindoline-1,3- dione | 927.45 | B | B | B | B | B |
| 22 | 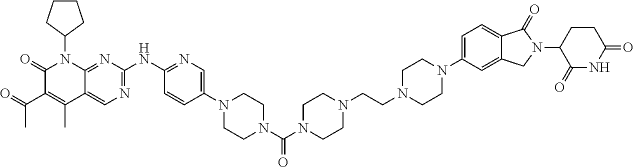 [SMILES: CC(=O)c1c(C)c2cnc(Nc3ccc(N4CCN(C(=O)N5CCN(CCN6CCN(c7ccc8c(c7)CN(C7CCC(=O)NC7=O)C8=O)CC6)CC5)CC4)cn3)nc2n(C2CCCC2)c1=O] | 3-(5-(4-(2-(4-(4- (6-((6-acetyl-8- cyclopentyl-5- methyl-7-oxo-7,8- dihydropyrido[2,3- d]pyrimidin-2- yl)amino)pyridin-3- yl)piperazine-1- carbonyl) piperazin-1- yl)ethyl) piperazin-1-yl)-1- oxoisoindolin-2- yl)piperidine-2,6- dione | 913.47 | C | C | B | C | B |
| 23 | 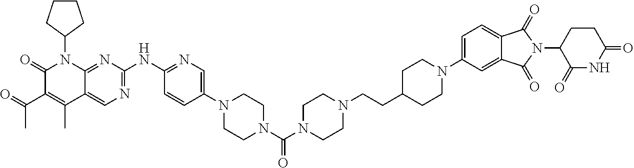 [SMILES: CC(=O)c1c(C)c2cnc(Nc3ccc(N4CCN(C(=O)N5CCN(CCC6CCN(c7ccc8c(c7)C(=O)N(C7CCC(=O)NC7=O)C8=O)CC6)CC5)CC4)cn3)nc2n(C2CCCC2)c1=O] | 5-(4-(2-(4-(4- (6-((6-acetyl-8- cyclopentyl-5- methyl-7-oxo-7,8- dihydropyrido[2,3- d]pyrimidin-2- yl)amino)pyridin-3- yl)piperazine-1- carbonyl) piperazin-1- yl)ethyl)piperidin- 1-yl)-2-(2,6- dioxopiperidin-3-yl) isoindoline-1,3- dione | 926.46 | C | C | B | C | B |
| 24 | 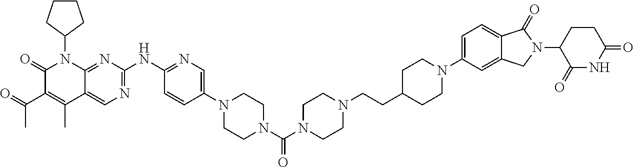 [SMILES: CC(=O)c1c(C)c2cnc(Nc3ccc(N4CCN(C(=O)N5CCN(CCC6CCN(c7ccc8c(c7)CN(C7CCC(=O)NC7=O)C8=O)CC6)CC5)CC4)cn3)nc2n(C2CCCC2)c1=O] | 3-(5-(4-(2-(4-(4- (6-((6-acetyl-8- cyclopentyl-5- methyl-7-oxo-7,8- dihydropyrido[2,3- d]pyrimidin-2- yl)amino)pyridin-3- yl)piperazine-1- carbonyl) piperazin-1- yl)ethyl)piperidin- 1-yl)-1- oxoisoindolin-2- yl)piperidine-2,6- dione | 912.48 | C | C | C | C | C |
| 25 | 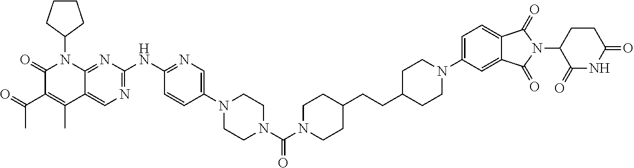 [SMILES: CC(=O)c1c(C)c2cnc(Nc3ccc(N4CCN(C(=O)N5CCC(CCC6CCN(c7ccc8c(c7)C(=O)N(C7CCC(=O)NC7=O)C8=O)CC6)CC5)CC4)cn3)nc2n(C2CCCC2)c1=O] | 5-(4-(2-(1-(4- (6-((6-acetyl-8- cyclopentyl-5- methyl-7-oxo-7,8- dihydropyrido[2,3- d]pyrimidin-2- yl)amino)pyridin-3- yl)piperazine-1- carbonyl) piperidin-4- yl)ethyl)piperidin- 1-yl)-2-(2,6- dioxopiperidin-3- yl)isoindoline-1,3- dione | 925.46 | B | C | B | B | B |
| 26 | 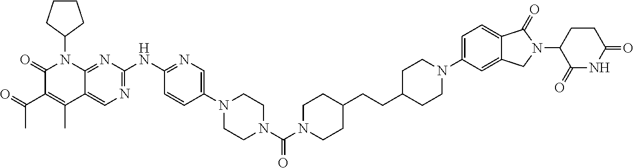 [SMILES: CC(=O)c1c(C)c2cnc(Nc3ccc(N4CCN(C(=O)N5CCC(CCC6CCN(c7ccc8c(c7)CN(C7CCC(=O)NC7=O)C8=O)CC6)CC5)CC4)cn3)nc2n(C2CCCC2)c1=O] | 3-(5-(4-(2-(1-(4- (6-((6-acetyl-8- cyclopentyl-5- methyl-7-oxo-7,8- dihydropyrido[2,3- d]pyrimidin-2- yl)amino)pyridin-3- yl)piperazine-1- carbonyl) piperidin-4- yl)ethyl) piperidin-1-yl)-1- oxoisoindolin-2- yl)piperidine-2,6- dione | 911.48 | B | C | C | C | C |
| 27 | 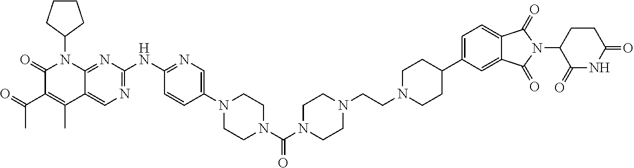 [SMILES: CC(=O)c1c(C)c2cnc(Nc3ccc(N4CCN(C(=O)N5CCN(CCN6CCC(c7ccc8c(c7)C(=O)N(C7CCC(=O)NC7=O)C8=O)CC6)CC5)CC4)cn3)nc2n(C2CCCC2)c1=O] | 5-(1-(2-(4-(4- (6-((6-acetyl-8- cyclopentyl-5- methyl-7-oxo-7,8- dihydropyrido[2,3- d]pyrimidin-2- yl)amino)pyridin-3- yl)piperazine-1- carbonyl) piperazin-1- yl)ethyl)piperidin- 4-yl)-2-(2,6- dioxopiperidin-3- yl)isoindoline-1,3- dione | 926.46 | B | C | B | C | B |
| 28 | 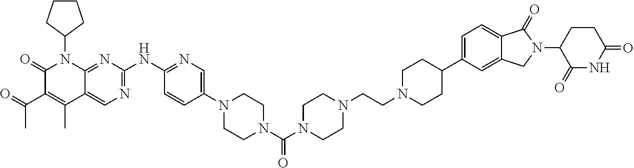 [SMILES: CC(=O)c1c(C)c2cnc(Nc3ccc(N4CCN(C(=O)N5CCN(CCN6CCC(c7ccc8c(c7)CN(C7CCC(=O)NC7=O)C8=O)CC6)CC5)CC4)cn3)nc2n(C2CCCC2)c1=O] | 3-(5-(1-(2-(4-(4- (6-((6-acetyl-8- cyclopentyl-5- methyl-7-oxo-7,8- dihydropyrido[2,3- d]pyrimidin-2- yl)amino)pyridin-3- yl)piperazine-1- carbonyl) piperazin-1- yl)ethyl)piperidin- 4-yl)-1- oxoisoindolin-2- yl)piperidine-2,6- dione | 912.48 | B | C | B | C | B |
| 29 | 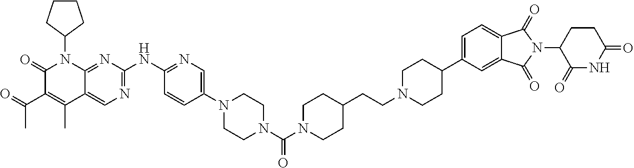 [SMILES: CC(=O)c1c(C)c2cnc(Nc3ccc(N4CCN(C(=O)N5CCC(CCN6CCC(c7ccc8c(c7)C(=O)N(C7CCC(=O)NC7=O)C8=O)CC6)CC5)CC4)cn3)nc2n(C2CCCC2)c1=O] | 5-(1-(2-(1-(4- (6-((6-acetyl-8- cyclopentyl-5- methyl-7-oxo-7,8- dihydropyrido[2,3- d]pyrimidin-2- yl)amino)pyridin-3- yl)piperazine-1- carbonyl)piperidin-4- yl)ethyl)piperidin- 4-yl)-2-(2,6- dioxopiperidin-3- yl)isoindoline-1,3- dione | 925.46 | C | C | C | C | C |
| 30 | 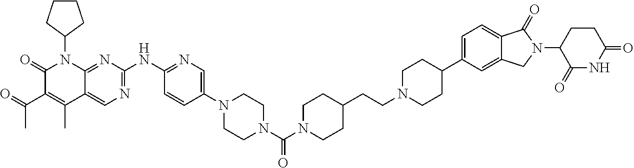 [SMILES: CC(=O)c1c(C)c2cnc(Nc3ccc(N4CCN(C(=O)N5CCC(CCN6CCC(c7ccc8c(c7)CN(C7CCC(=O)NC7=O)C8=O)CC6)CC5)CC4)cn3)nc2n(C2CCCC2)c1=O] | 3-(5-(1-(2-(1-(4- (6-((6-acetyl-8- cyclopentyl-5- methyl-7-oxo-7,8- dihydropyrido[2,3- d]pyrimidin-2- yl)amino)pyridin- 3-yl)piperazine-1- carbonyl) piperidin-4- yl)ethyl) piperidin-4-yl)-1- oxoisoindolin-2- yl)piperidine-2,6- dione | 911.48 | C | C | C | C | C |
| 31 | 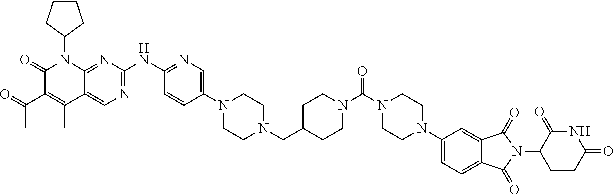 [SMILES: CC(=O)c1c(C)c2cnc(Nc3ccc(N4CCN(CC5CCN(C(=O)N6CCN(c7ccc8c(c7)C(=O)N(C7CCC(=O)NC7=O)C8=O)CC6)CC5)CC4)cn3)nc2n(C2CCCC2)c1=O] | 5-(4-(4-((4- (6-((6-acetyl-8- cyclopentyl-5- methyl-7-oxo-7,8- dihydropyrido[2,3- d]pyrimidin-2- yl)amino)pyridin- 3-yl)piperazin-1- yl)methyl) piperidine-1- carbonyl)piperazin- 1-yl)-2-(2,6- dioxopiperidin-3- yl)isoindoline-1,3- dione | 912.44 | A | B | A | B | A |
| 32 | 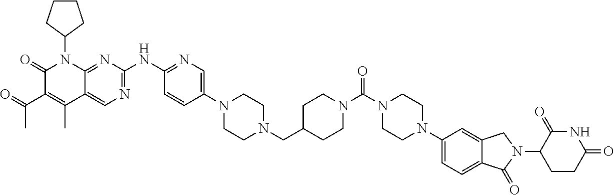 [SMILES: CC(=O)c1c(C)c2cnc(Nc3ccc(N4CCN(CC5CCN(C(=O)N6CCN(c7ccc8c(c7)CN(C7CCC(=O)NC7=O)C8=O)CC6)CC5)CC4)cn3)nc2n(C2CCCC2)c1=O] | 3-(5-(4-(4-((4- (6-((6-acetyl-8- cyclopentyl-5- methyl-7-oxo-7,8- dihydropyrido[2,3- d]pyrimidin-2- yl)amino)pyridin- 3-yl)piperazin-1 - yl)methyl) piperidine-1- carbonyl) piperazin-1-yl)-1- oxoisoindolin-2- yl)piperidine-2,6- dione | 898.46 | B | B | B | B | B |
| 33 | 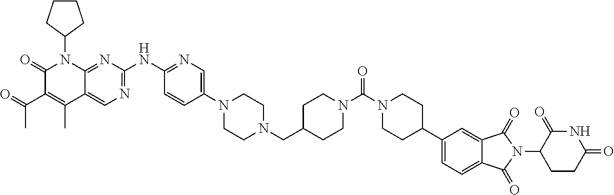 [SMILES: CC(=O)c1c(C)c2cnc(Nc3ccc(N4CCN(CC5CCN(C(=O)N6CCC(c7ccc8c(c7)C(=O)N(C7CCC(=O)NC7=O)C8=O)CC6)CC5)CC4)cn3)nc2n(C2CCCC2)c1=O] | 5-(1-(4-((4-(6- ((6-acetyl-8- cyclopentyl-5- methyl-7-oxo-7,8- dihydropyrido[2,3- d]pyrimidin-2- yl)amino)pyridin- 3-yl)piperazin-1- yl)methyl) piperidine-1- carbonyl)piperidin- 4-yl)-2-(2,6- dioxopiperidin-3- yl)isoindoline-1,3- dione | 911.44 | B | C | B | C | C |
| 34 | 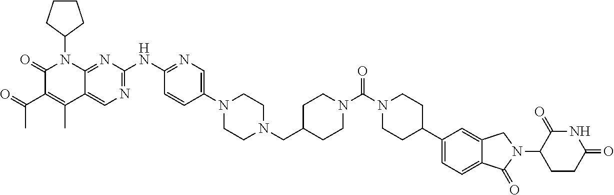 [SMILES: CC(=O)c1c(C)c2cnc(Nc3ccc(N4CCN(CC5CCN(C(=O)N6CCC(c7ccc8c(c7)CN(C7CCC(=O)NC7=O)C8=O)CC6)CC5)CC4)cn3)nc2n(C2CCCC2)c1=O] | 5-(1-(4-((4-(6- ((6-acetyl-8- cyclopentyl-5- methyl-7-oxo-7,8- dihydropyrido[2,3- d]pyrimidin-2- yl)amino)pyridin- 3-yl)piperazin-1- yl)methyl) piperidine-1- carbonyl)piperidin- 4-yl)-2-(2,6- dioxopiperidin-3- yl)isoindoline-1,3- dione | 897.46 | C | C | C | C | C |
| 35 | 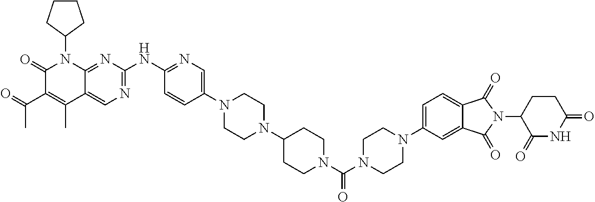 [SMILES: CC(=O)c1c(C)c2cnc(Nc3ccc(N4CCN(C5CCN(C(=O)N6CCN(c7ccc8c(c7)C(=O)N(C7CCC(=O)NC7=O)C8=O)CC6)CC5)CC4)cn3)nc2n(C2CCCC2)c1=O] | 5-(4-(4-(4- (6-((6-acetyl-8- cyclopentyl-5- methyl-7-oxo-7,8- dihydropyrido[2,3- d]pyrimidin-2- yl)amino)pyridin- 3-yl)piperazin-1- yl)piperidine-1- carbonyl)piperazin- 1-yl)-2-(2,6- dioxopiperidin-3- yl)isoindoline- 1,3-dione | 898.42 | B | B | B | C | B |
| 36 | 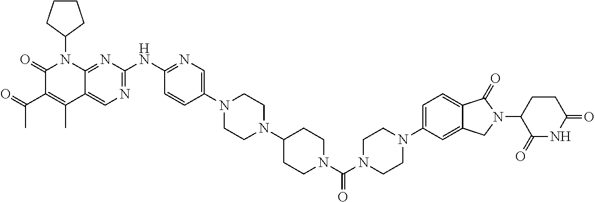 [SMILES: CC(=O)c1c(C)c2cnc(Nc3ccc(N4CCN(C5CCN(C(=O)N6CCN(c7ccc8c(c7)CN(C7CCC(=O)NC7=O)C8=O)CC6)CC5)CC4)cn3)nc2n(C2CCCC2)c1=O] | 3-(5-(4-(4-(4- (6-((6-acetyl-8- cyclopentyl-5- methyl-7-oxo-7,8- dihydropyrido[2,3- d]pyrimidin-2- yl)amino)pyridin- 3-yl)piperazin-1- yl)piperidine-1- carbonyl)piperazin- 1-yl)-1- oxoisoindolin-2- yl)piperidine- 2,6-dione | 884.44 | B | C | B | C | B |
| 37 | 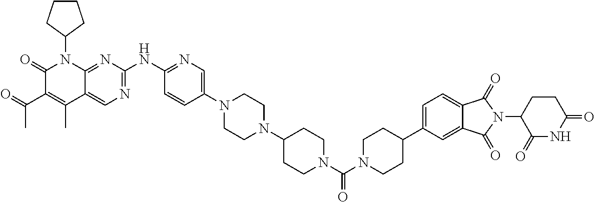 [SMILES: CC(=O)c1c(C)c2cnc(Nc3ccc(N4CCN(C5CCN(C(=O)N6CCC(c7ccc8c(c7)C(=O)N(C7CCC(=O)NC7=O)C8=O)CC6)CC5)CC4)cn3)nc2n(C2CCCC2)c1=O] | 5-(1-(4-(4- (6-((6-acetyl-8- cyclopentyl-5- methyl-7-oxo-7,8- dihydropyrido[2,3- d]pyrimidin-2- yl)amino)pyridin- 3-yl)piperazin-1- yl)piperidine-1- carbonyl)piperidin- 4-yl)-2-(2,6- dioxopiperidin-3- yl)isoindoline-1,3- dione | 897.43 | C | C | C | C | C |
| 38 | 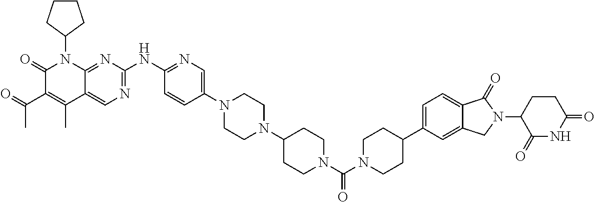 [SMILES: CC(=O)c1c(C)c2cnc(Nc3ccc(N4CCN(C5CCN(C(=O)N6CCC(c7ccc8c(c7)CN(C7CCC(=O)NC7=O)C8=O)CC6)CC5)CC4)cn3)nc2n(C2CCCC2)c1=O] | 3-(5-(1-(4-(4- (6-((6-acetyl-8- cyclopentyl-5- methyl-7-oxo-7,8- dihydropyrido[2,3- d]pyrimidin-2- yl)amino)pyridin- 3-yl)piperazin-1- yl)piperidine-1- carbonyl)piperidin- 4-yl)-1- oxoisoindolin-2- yl)piperidine-2,6- dione | 883.45 | C | C | C | C | C |
| 39 | 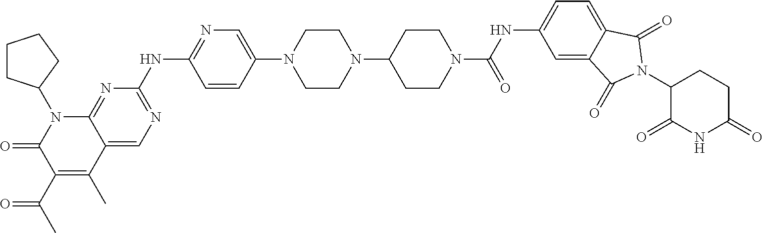 [SMILES: CC(=O)c1c(C)c2cnc(Nc3ccc(N4CCN(C5CCN(C(=O)Nc6ccc7c(c6)C(=O)N(C6CCC(=O)NC6=O)C7=O)CC5)CC4)cn3)nc2n(C2CCCC2)c1=O] | 4-(4-(6-((6-acetyl- 8-cyclopentyl-5- methyl-7-oxo-7,8- dihydropyrido[2,3- d]pyrimidin-2- yl)amino)pyridin- 3-yl)piperazin-1- yl)-N-(2-(2,6- dioxopiperidin-3-yl)- 1,3-dioxoisoindolin- 5-yl)piperidine- 1-carboxamide | 829.37 | A | B | A | B | A |
| 40 | 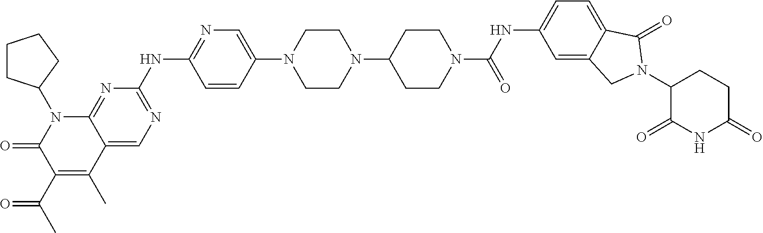 [SMILES: CC(=O)c1c(C)c2cnc(Nc3ccc(N4CCN(C5CCN(C(=O)Nc6ccc7c(c6)CN(C6CCC(=O)NC6=O)C7=O)CC5)CC4)cn3)nc2n(C2CCCC2)c1=O] | 4-(4-(6-((6-acetyl-8- cyclopentyl-5- methyl-7-oxo-7,8- dibydropyrido[2,3- d]pyrimidin-2- yl)amino)pyridin- 3-yl)piperazin-1- yl)-N-(2-(2,6- dioxopiperidin-3-yl)- 1-oxoisoindolin-5- yl)piperidine-1- carboxamide | 815.39 | A | B | B | B | A |
| 41 | 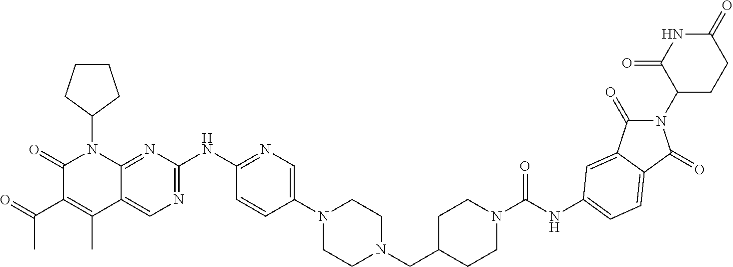 [SMILES: CC(=O)c1c(C)c2cnc(Nc3ccc(N4CCN(CC5CCN(C(=O)Nc6ccc7c(c6)C(=O)N(C6CCC(=O)NC6=O)C7=O)CC5)CC4)cn3)nc2n(C2CCCC2)c1=O] | 4-((4-(6-((6-acetyl- 8-cyclopentyl-5- methyl-7-oxo-7,8- dihydropyrido[2,3- d]pyrimidin-2- yl)amino)pyridin- 3-yl)piperazin-1- yl)methyl)-N-(2-(2,6- dioxopiperidin- 3-yl)-1,3- dioxoisoindolin-5- yl)piperidine-1- carboxamide | 844.38 | A | A | A | B | A |
| 42 | 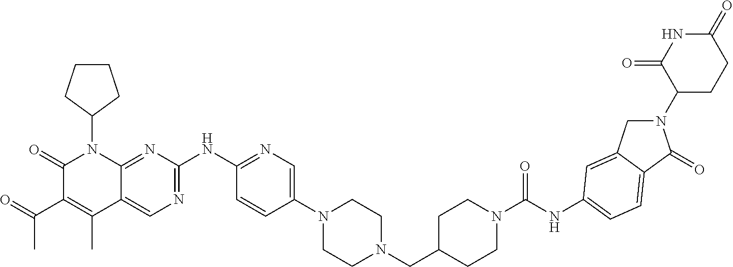 [SMILES: CC(=O)c1c(C)c2cnc(Nc3ccc(N4CCN(CC5CCN(C(=O)Nc6ccc7c(c6)CN(C6CCC(=O)NC6=O)C7=O)CC5)CC4)cn3)nc2n(C2CCCC2)c1=O] | 4-((4-(6-((6-acetyl- 8-cyclopentyl-5- methyl-7-oxo-7,8- dihydropyrido[2,3- d]pyrimidin-2- yl)amino)pyridin- 3-yl)piperazin-1- yl)methyl)-N-(2-(2,6- dioxopiperidin-3-yl)-1- oxoisoindolin-5- yl)piperidine-1- carboxamide | 830.40 | B | B | A | B | A |
| 43 | 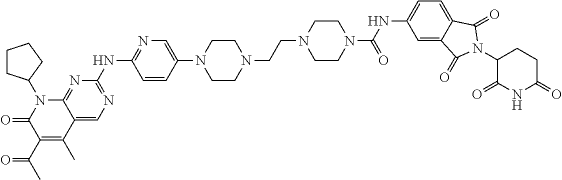 [SMILES: CC(=O)c1c(C)c2cnc(Nc3ccc(N4CCN(CCN5CCN(C(=O)Nc6ccc7c(c6)C(=O)N(C6CCC(=O)NC6=O)C7=O)CC5)CC4)cn3)nc2n(C2CCCC2)c1=O] | 4-(2-(4-(6-((6-acetyl- 8-cyclopentyl- 5-methyl-7-oxo-7,8- dihydropyrido[2,3- d]pyrimidin-2- yl)amino)pyridin- 3-yl)piperazin-1- yl)ethyl)-N-(2-(2,6- dioxopiperidin- 3-yl)-1,3- dioxoisoindolin-5- yl)piperazine-1- carboxamide | 859.39 | B | B | B | B | B |
| 44 | 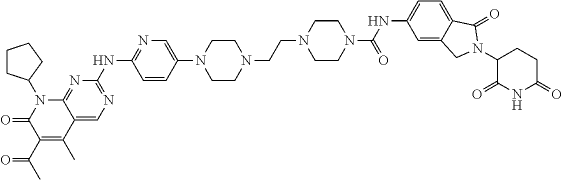 [SMILES: CC(=O)c1c(C)c2cnc(Nc3ccc(N4CCN(CCN5CCN(C(=O)Nc6ccc7c(c6)CN(C6CCC(=O)NC6=O)C7=O)CC5)CC4)cn3)nc2n(C2CCCC2)c1=O] | 4-(2-(4-(6-((6-acetyl- 8-cyclopentyl- 5-methyl-7-oxo-7,8- dihydropyrido[2,3- d]pyrimidin-2- yl)amino)pyridin- 3-yl)piperazin-1- ylethyl)-N-(2-(2,6- dioxopiperidin- 3-yl)-1- oxoisoindolin-5- yl)piperazine-1- carboxamide | 845.41 | B | B | B | B | B |
| 45 | 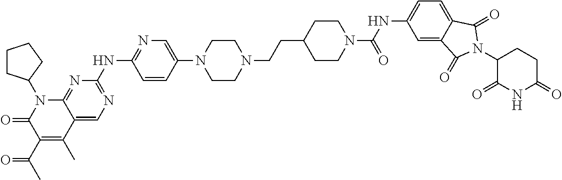 [SMILES: CC(=O)c1c(C)c2cnc(Nc3ccc(N4CCN(CCC5CCN(C(=O)Nc6ccc7c(c6)C(=O)N(C6CCC(=O)NC6=O)C7=O)CC5)CC4)cn3)nc2n(C2CCCC2)c1=O] | 4-(2-(4-(6-((6-acetyl- 8-cyclopentyl- 5-methyl-7-oxo-7,8- dihydropyrido[2,3- d]pyrimidin-2- yl)amino)pyridin- 3-yl)piperazin-1- yl)ethyl)-N-(2-(2,6- dioxopiperidin- 3-yl)-1,3- dioxoisoindolin-5- yl)piperidine- 1-carboxamide | 858.40 | B | B | B | B | B |
| 46 | 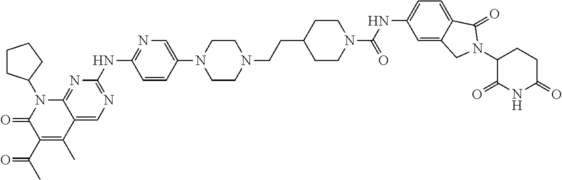 [SMILES: CC(=O)c1c(C)c2cnc(Nc3ccc(N4CCN(CCC5CCN(C(=O)Nc6ccc7c(c6)CN(C6CCC(=O)NC6=O)C7=O)CC5)CC4)cn3)nc2n(C2CCCC2)c1=O] | 4-(2-(4-(6-((6-acetyl- 8-cyclopentyl- 5-methyl-7-oxo-7,8- dihydropyrido[2,3- d]pyrimidin-2- yl)amino)pyridin-3- yl)piperazin-1- yl)ethyl)-N-(2-(2,6- dioxopiperidin- 3-yl)-1- oxoisoindolin-5- yl)piperidine-1- carboxamide | 843.42 | B | C | B | B | B |
| 47 | 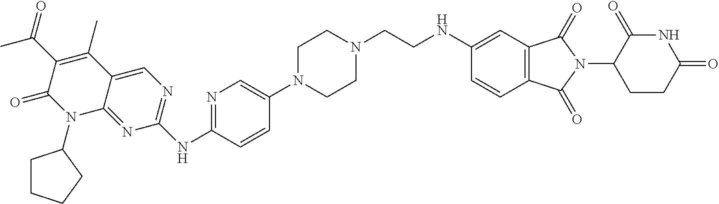 [SMILES: CC(=O)c1c(C)c2cnc(Nc3ccc(N4CCN(CCNc5ccc6c(c5)C(=O)N(C5CCC(=O)NC5=O)C6=O)CC4)cn3)nc2n(C2CCCC2)c1=O] | 5-((2-(4-(6- ((6-acetyl-8- cyclopentyl-5- methyl-7-oxo-7,8- dihydropyrido[2,3- d]pyrimidin-2- yl)amino)pyridin-3- yl)piperazin-1- yl)ethyl) amino)-2-(2,6- dioxopiperidin-3- yl)isoindoline-1,3- dione | 746.33 | A | A | A | A | A |
| 48 | 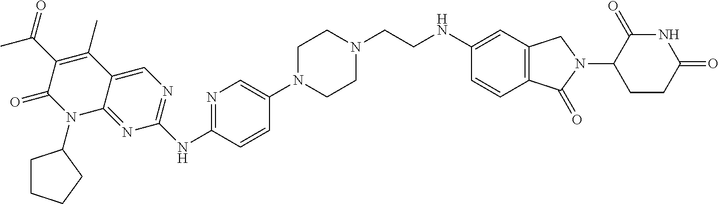 [SMILES: CC(=O)c1c(C)c2cnc(Nc3ccc(N4CCN(CCNc5ccc6c(c5)CN(C5CCC(=O)NC5=O)C6=O)CC4)cn3)nc2n(C2CCCC2)c1=O] | 3-(5-((2-(4-(6- ((6-acetyl-8- cyclopentyl-5- methyl-7-oxo-7,8- dihydropyrido[2,3- d]pyrimidin-2- yl)amino)pyridin-3- yl)piperazin-1- yl)ethyl)amino)-1- oxoisoindolin-2- yl)piperidine-2,6- dione | 732.35 | A | A | A | A | A |
| 49 | 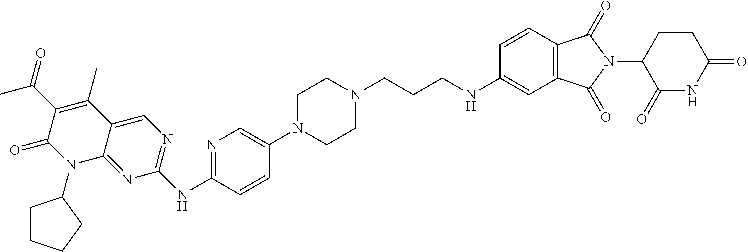 [SMILES: CC(=O)c1c(C)c2cnc(Nc3ccc(N4CCN(CCCNc5ccc6c(c5)C(=O)N(C5CCC(=O)NC5=O)C6=O)CC4)cn3)nc2n(C2CCCC2)c1=O] | 5-((3-(4-(6- ((6-acetyl-8- cyclopentyl-5- methyl-7-oxo-7,8- dihydropyrido[2,3- d]pyrimidin-2- yl)amino)pyridin-3- yl)piperazin-1- yl)propyl)amino)- 2-(2,6- dioxopiperidin-3-yl) isoindoline-1,3- dione | 760.34 | A | A | A | A | A |
| 50 | 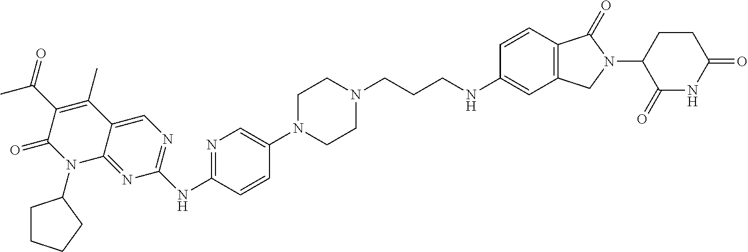 [SMILES: CC(=O)c1c(C)c2cnc(Nc3ccc(N4CCN(CCCNc5ccc6c(c5)CN(C5CCC(=O)NC5=O)C6=O)CC4)cn3)nc2n(C2CCCC2)c1=O] | 3-(5-((3-(4-(6- ((6-acetyl-8- cyclopentyl-5- methyl-7-oxo-7,8- dihydropyrido[2,3- d]pyrimidin-2- yl)amino)pyridin-3- yl)piperazin-1- yl)propyl)amino)-1- oxoisoindolin- 2-yl)piperidine-2,6- dione | 746.37 | A | A | A | A | A |
| 51 | 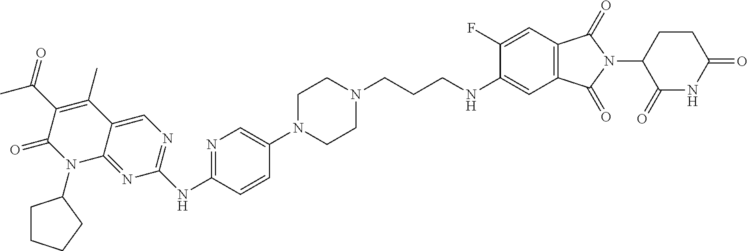 [SMILES: CC(=O)c1c(C)c2cnc(Nc3ccc(N4CCN(CCCNc5cc6c(cc5F)C(=O)N(C5CCC(=O)NC5=O)C6=O)CC4)cn3)nc2n(C2CCCC2)c1=O] | 5-((3-(4-(6- ((6-acetyl-8- cyclopentyl-5- methyl-7-oxo-7,8- dihydropyrido[2,3- d]pyrimidin-2- yl)amino)pyridin-3- yl)piperazin-1- yl)propyl) amino)-2-(2,6- dioxopiperidin- 3-yl)-6- fluoroisoindoline- 1,3-dione | 778.34 | A | B | A | B | A |
| 52 | 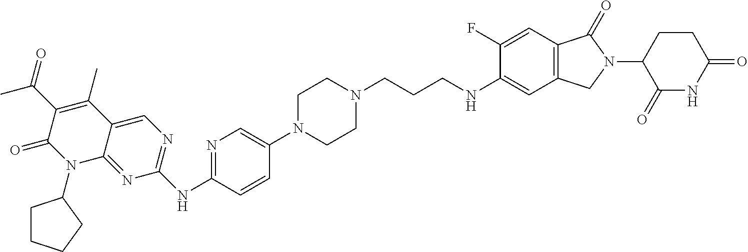 [SMILES: CC(=O)c1c(C)c2cnc(Nc3ccc(N4CCN(CCCNc5cc6c(cc5F)C(=O)N(C5CCC(=O)NC5=O)C6)CC4)cn3)nc2n(C2CCCC2)c1=O] | 3-(5-((3-(4-(6- ((6-acetyl-8- cyclopentyl-5- methyl-7-oxo-7,8- dihydropyrido[2,3- d]pyrimidin-2- yl)amino)pyridin-3- yl)piperazin-1- yl)propyl)amino)- 6-fluoro-1- oxoisoindolin-2-yl) piperidine-2,6- dione | 764.36 | A | B | A | B | A |
| 53 | 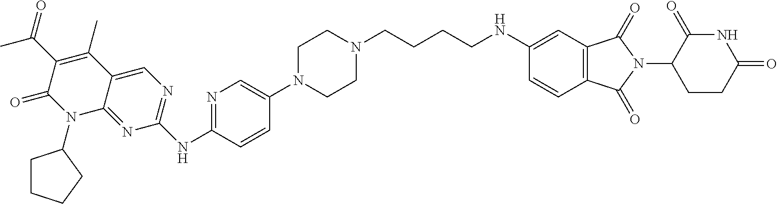 [SMILES: CC(=O)c1c(C)c2cnc(Nc3ccc(N4CCN(CCCCNc5ccc6c(c5)C(=O)N(C5CCC(=O)NC5=O)C6=O)CC4)cn3)nc2n(C2CCCC2)c1=O] | 5-((4-(4-(6- ((6-acetyl-8- cyclopentyl-5- methyl-7-oxo-7,8- dihydropyrido[2,3- d]pyrimidin-2- yl)amino)pyridin-3- yl)piperazin-1- yl)butyl) amino)-2-(2,6- dioxopiperidin-3- yl)isoindoline-1,3- dione | 774.36 | A | B | B | B | A |
| 54 | 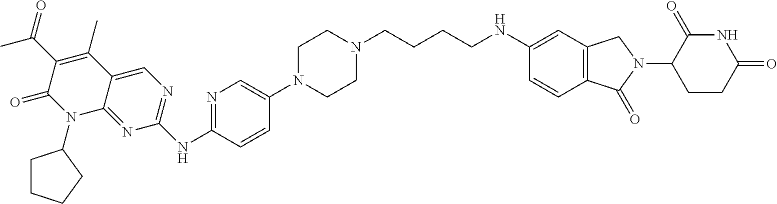 [SMILES: CC(=O)c1c(C)c2cnc(Nc3ccc(N4CCN(CCCCNc5ccc6c(c5)CN(C5CCC(=O)NC5=O)C6=O)CC4)cn3)nc2n(C2CCCC2)c1=O] | 3-(5-((4-(4-(6- ((6-acetyl-8- cyclopentyl-5- methyl-7-oxo-7,8- dihydropyrido[2,3- d]pyrimidin-2- yl)amino)pyridin-3- yl)piperazin-1- yl)butyl)amino)-1- oxoisoindolin-2- yl)piperidine-2,6- dione | 760.38 | A | B | B | B | B |
| 55 | 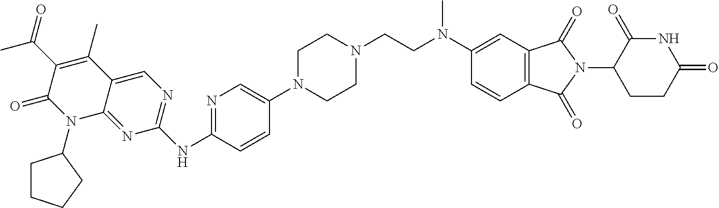 [SMILES: CC(=O)c1c(C)c2cnc(Nc3ccc(N4CCN(CCN(C)c5ccc6c(c5)C(=O)N(C5CCC(=O)NC5=O)C6=O)CC4)cn3)nc2n(C2CCCC2)c1=O] | 5-((2-(4-(6- ((6-acetyl-8- cyclopentyl-5- methyl-7-oxo-7,8- dihydropyrido[2,3- d]pyrimidin-2- yl)amino)pyridin-3- yl)piperazin-1- yl)ethyl)(methyl) amino)-2-(2,6- dioxopiperidin-3-yl) isoindoline-1,3- dione | 760.34 | B | B | B | B | B |
| 56 | 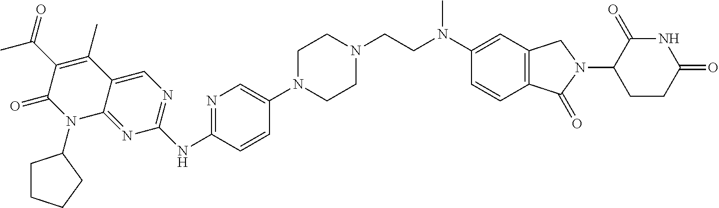 [SMILES: CC(=O)c1c(C)c2cnc(Nc3ccc(N4CCN(CCN(C)c5ccc6c(c5)CN(C5CCC(=O)NC5=O)C6=O)CC4)cn3)nc2n(C2CCCC2)c1=O] | 3-(5-((2-(4-(6- ((6-acetyl-8- cyclopentyl-5- methyl-7-oxo-7,8- dihydropyrido[2,3- d]pyrimidin-2- yl)amino)pyridin- 3-yl)piperazin-1- yl)ethyl)(methyl) amino)-1- oxoisoindolin-2- yl)piperidine-2,6- dione | 746.37 | B | B | B | B | B |
| 57 | 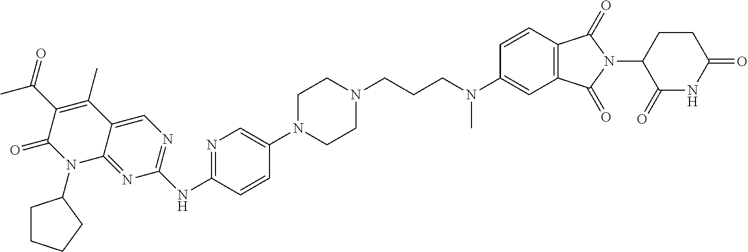 [SMILES: CC(=O)c1c(C)c2cnc(Nc3ccc(N4CCN(CCCN(C)c5ccc6c(c5)C(=O)N(C5CCC(=O)NC5=O)C6=O)CC4)cn3)nc2n(C2CCCC2)c1=O] | 5-((3-(4-(6- ((6-acetyl-8- cyclopentyl-5- methyl-7-oxo-7,8- dihydropyrido[2,3- d]pyrimidin-2- yl)amino)pyridin-3- yl)piperazin-1- yl)propyl)(methyl) amino)-2-(2,6- dioxopiperidin-3- yl)isoindoline-1,3- dione | 774.36 | B | B | B | B | B |
| 58 | 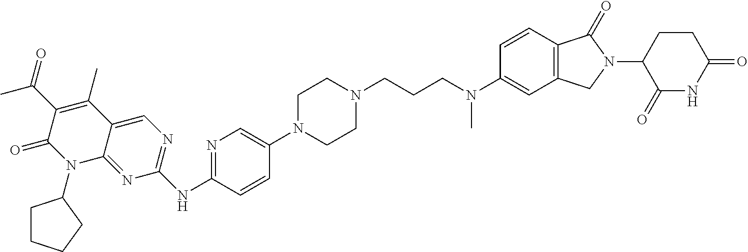 [SMILES: CC(=O)c1c(C)c2cnc(Nc3ccc(N4CCN(CCCN(C)c5ccc6c(c5)CN(C5CCC(=O)NC5=O)C6=O)CC4)cn3)nc2n(C2CCCC2)c1=O] | 3-(5-((3-(4-(6- ((6-acetyl-8- cyclopentyl-5- methyl-7-oxo-7,8- dihydropyrido[2,3- d]pyrimidin-2- yl)amino)pyridin- 3-yl)piperazin-1- yl)propyl) (methyl)amino)-1- oxoisoindolin-2-yl) piperidine-2,6- dione | 760.38 | B | B | B | B | B |
| 59 | 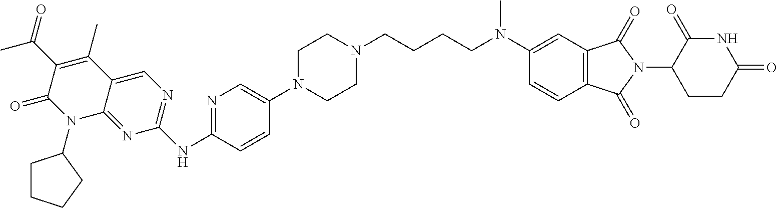 [SMILES: CC(=O)c1c(C)c2cnc(Nc3ccc(N4CCN(CCCCN(C)c5ccc6c(c5)C(=O)N(C5CCC(=O)NC5=O)C6=O)CC4)cn3)nc2n(C2CCCC2)c1=O] | 5-((4-(4-(6- ((6-acetyl-8- cyclopentyl-5- methyl-7-oxo-7,8- dihydropyrido[2,3- d]pyrimidin-2- yl)amino)pyridin-3- yl)piperazin-1- yl)butyl)(methyl) amino)-2-(2,6- dioxopiperidin-3-yl) isoindoline-1,3- dione | 788.38 | B | B | B | B | B |
| 60 | 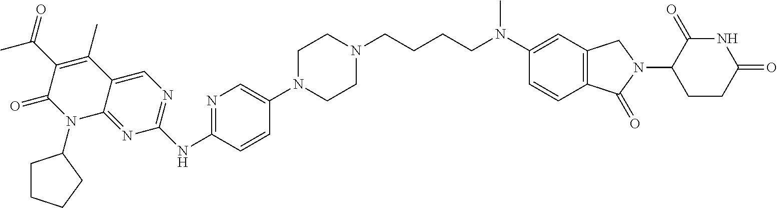 [SMILES: CC(=O)c1c(C)c2cnc(Nc3ccc(N4CCN(CCCCN(C)c5ccc6c(c5)CN(C5CCC(=O)NC5=O)C6=O)CC4)cn3)nc2n(C2CCCC2)c1=O] | 3-(5-((4-(4-(6- ((6-acetyl-8- cyclopentyl-5- methyl-7-oxo-7,8- dihydropyrido[2,3- d]pyrimidin-2- yl)amino)pyridin- 3-yl)piperazin-1- yl)butyl)(methyl) amino)-1- oxoisoindolin-2-yl) piperidine-2,6- dione | 774.40 | B | B | B | B | B |
| 61 | 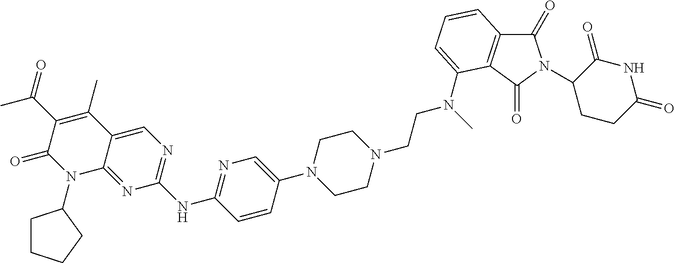 [SMILES: CC(=O)c1c(C)c2cnc(Nc3ccc(N4CCN(CCN(C)c5cccc6c5C(=O)N(C5CCC(=O)NC5=O)C6=O)CC4)cn3)nc2n(C2CCCC2)c1=O] | 4-((2-(4-(6- ((6-acetyl-8- cyclopentyl-5- methyl-7-oxo-7,8- dihydropyrido[2,3- d]pyrimidin-2- yl)amino)pyridin-3- yl)piperazin-1- yl)ethyl)(methyl) amino)-2-(2,6- dioxopiperidin-3-yl) isoindoline-1,3- dione | 760.34 | B | B | B | B | B |
| 62 | 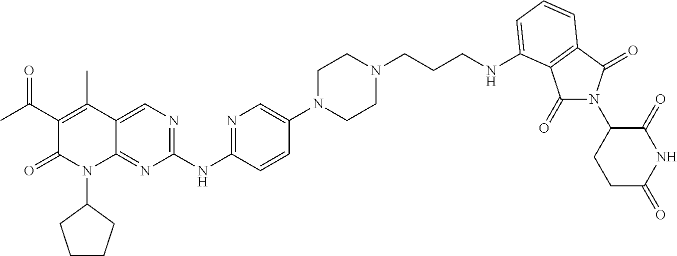 [SMILES: CC(=O)c1c(C)c2cnc(Nc3ccc(N4CCN(CCCNc5cccc6c5C(=O)N(C5CCC(=O)NC5=O)C6=O)CC4)cn3)nc2n(C2CCCC2)c1=O] | 4-((3-(4-(6- ((6-acetyl-8- cyclopentyl-5- methyl-7-oxo-7,8- dihydropyrido[2,3- d]pyrimidin-2- yl)amino)pyridin- 3-yl)piperazin-1- yl)propyl) amino)-2-(2,6- dioxopiperidin-3-yl) isoindoline-1,3- dione | 760.34 | A | B | A | B | A |
| 63 | 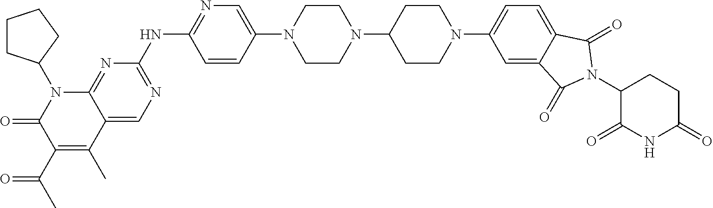 [SMILES: CC(=O)c1c(C)c2cnc(Nc3ccc(N4CCN(C5CCN(c6ccc7c(c6)C(=O)N(C6CCC(=O)NC6=O)C7=O)CC5)CC4)cn3)nc2n(C2CCCC2)c1=O] | 5-(4-(4-(6-((6-acetyl- 8-cyclopentyl- 5-methyl-7-oxo-7,8- dihydropyrido[2,3- d]pyrimidin-2- yl)amino)pyridin-3- yl)piperazin-1- yl)piperidin- 1-yl)-2-(2,6- dioxopiperidin-3- yl)isoindoline-1,3- dione | 786.36 | A | B | A | A | A |
| 64 | 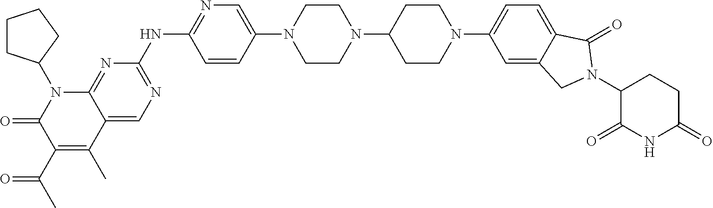 [SMILES: CC(=O)c1c(C)c2cnc(Nc3ccc(N4CCN(C5CCN(c6ccc7c(c6)CN(C6CCC(=O)NC6=O)C7=O)CC5)CC4)cn3)nc2n(C2CCCC2)c1=O] | 3-(5-(4-(4-(6- ((6-acetyl-8- cyclopentyl-5- methyl-7-oxo-7,8- dihydropyrido[2,3- d]pyrimidin-2- yl)amino)pyridin- 3-yl)piperazin-1- yl)piperidin-1-yl)- 1-oxoisoindolin- 2-yl)piperidine- 2,6-dione | 772.38 | A | B | A | B | A |
| 65 | 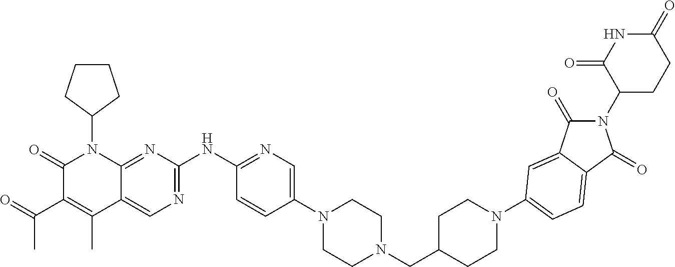 [SMILES: CC(=O)c1c(C)c2cnc(Nc3ccc(N4CCN(CC5CCN(c6ccc7c(c6)C(=O)N(C6CCC(=O)NC6=O)C7=O)CC5)CC4)cn3)nc2n(C2CCCC2)c1=O] | 5-(4-((4-(6- ((6-acetyl-8- cyclopentyl-5- methyl-7-oxo-7,8- dihydropyrido[2,3- d]pyrimidin-2- yl)amino)pyridin-3- yl)piperazin-1- yl)methyl)piperidin- 1-yl)-2-(2,6- dioxopiperidin-3-yl) isoindoline-1,3- dione | 800.38 | A | A | A | A | A |
| 66 | 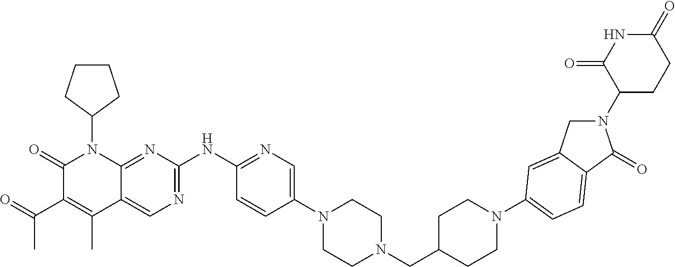 [SMILES: CC(=O)c1c(C)c2cnc(Nc3ccc(N4CCN(CC5CCN(c6ccc7c(c6)CN(C6CCC(=O)NC6=O)C7=O)CC5)CC4)cn3)nc2n(C2CCCC2)c1=O] | 3-(5-(4-((4-(6- ((6-acetyl-8- cyclopentyl-5- methyl-7-oxo-7,8- dihydropyrido[2,3- d]pyrimidin-2- yl)amino)pyridin- 3-yl)piperazin-1- yl)methyl) piperidin-1-yl)-1- oxoisoindolin-2- yl)piperidine-2,6- dione | 786.40 | A | B | A | A | A |
| 67 | 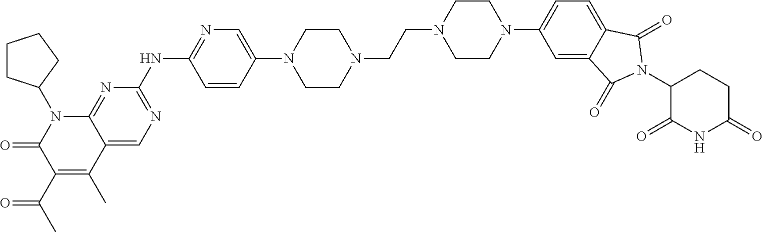 [SMILES: CC(=O)c1c(C)c2cnc(Nc3ccc(N4CCN(CCN5CCN(c6ccc7c(c6)C(=O)N(C6CCC(=O)NC6=O)C7=O)CC5)CC4)cn3)nc2n(C2CCCC2)c1=O] | 5-(4-(2-(4-(6- ((6-acetyl-8- cyclopentyl-5- methyl-7-oxo-7,8- dihydropyrido[2,3- d]pyrimidin-2- yl)amino)pyridin- 3-yl)piperazin-1- yl)ethyl)piperazin- 1-yl)-2-(2,6- dioxopiperidin-3-yl) isoindoline-1,3- dione | 815.39 | A | A | A | B | A |
| 68 | 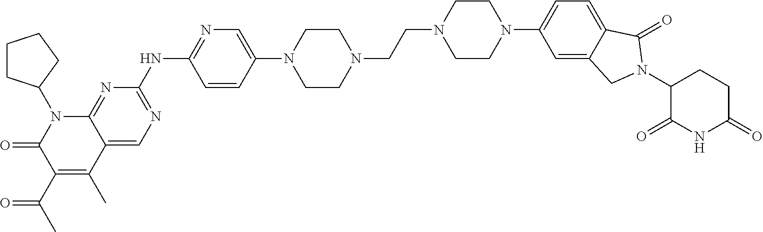 [SMILES: CC(=O)c1c(C)c2cnc(Nc3ccc(N4CCN(CCN5CCN(c6ccc7c(c6)CN(C6CCC(=O)NC6=O)C7=O)CC5)CC4)cn3)nc2n(C2CCCC2)c1=O] | 3-(5-(4-(2-(4- (6-((6-acetyl-8- cyclopentyl-5- methyl-7-oxo-7,8- dihydropyrido[2,3- d]pyrimidin-2- yl)amino)pyridin-3- yl)piperazin-1- yl)ethyl) piperazin-1-yl)-1- oxoisoindolin-2- yl)piperidine-2,6- dione | 801.41 | B | B | B | B | A |
| 69 | 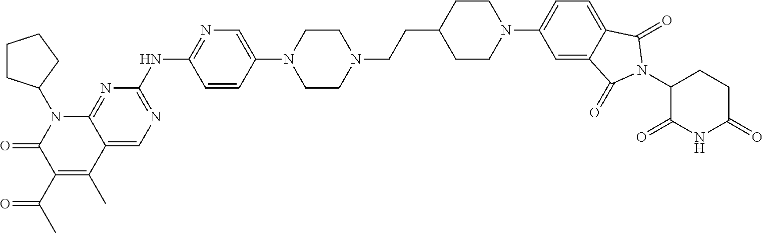 [SMILES: CC(=O)c1c(C)c2cnc(Nc3ccc(N4CCN(CCC5CCN(c6ccc7c(c6)C(=O)N(C6CCC(=O)NC6=O)C7=O)CC5)CC4)cn3)nc2n(C2CCCC2)c1=O] | 5-(4-(2-(4- (6-((6-acetyl-8- cyclopentyl-5- methyl-7-oxo-7,8- dihydropyrido[2,3- d]pyrimidin-2- yl)amino)pyridin- 3-yl)piperazin-1- yl)ethyl)piperidin- 1-yl)-2-(2,6- dioxopiperidin-3- yl)isoindoline-1,3- dione | 814.39 | B | B | B | B | B |
| 70 | 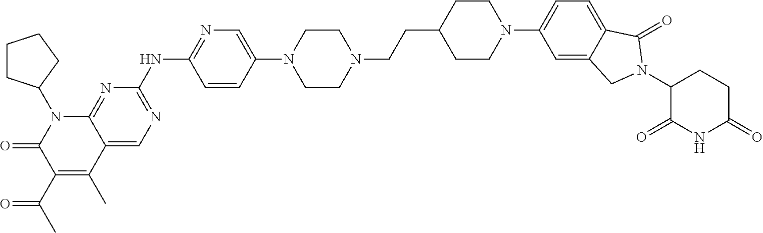 [SMILES: CC(=O)c1c(C)c2cnc(Nc3ccc(N4CCN(CCC5CCN(c6ccc7c(c6)CN(C6CCC(=O)NC6=O)C7=O)CC5)CC4)cn3)nc2n(C2CCCC2)c1=O] | 3-(5-(4-(2-(4- (6-((6-acetyl-8- cyclopentyl-5- methyl-7-oxo-7,8- dihydropyrido[2,3- d]pyrimidin-2- yl)amino)pyridin- 3-yl)piperazin-1- yl)ethyl) piperidin-1-yl)-1- oxoisoindolin-2-yl) piperidine-2,6- dione | 801.41 | B | B | C | B | B |
| 71 | 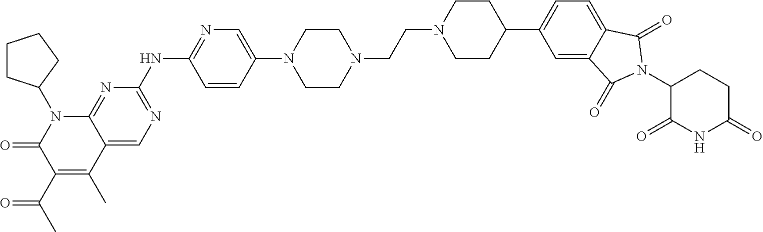 [SMILES: CC(=O)c1c(C)c2cnc(Nc3ccc(N4CCN(CCN5CCC(c6ccc7c(c6)C(=O)N(C6CCC(=O)NC6=O)C7=O)CC5)CC4)cn3)nc2n(C2CCCC2)c1=O] | 5-(1-(2-(4-(6- ((6-acetyl-8- cyclopentyl-5- methyl-7-oxo-7,8- dihydropyrido[2,3- d]pyrimidin-2- yl)amino)pyridin-3- yl)piperazin-1- yl)ethyl)piperidin- 4-yl)-2-(2,6- dioxopiperidin-3- yl)isoindoline-1,3- dione | 814.39 | B | C | B | C | B |
| 72 | 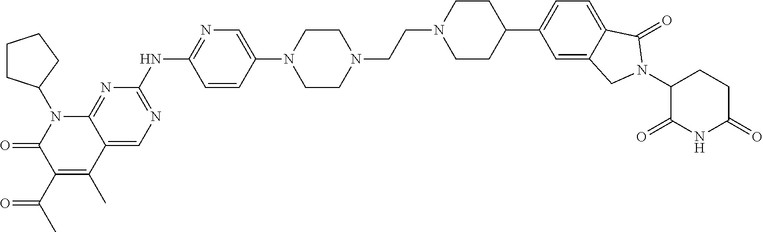 [SMILES: CC(=O)c1c(C)c2cnc(Nc3ccc(N4CCN(CCN5CCC(c6ccc7c(c6)CN(C6CCC(=O)NC6=O)C7=O)CC5)CC4)cn3)nc2n(C2CCCC2)c1=O] | 3-(5-(1-(2-(4- (6-((6-acetyl-8- cyclopentyl-5- methyl-7-oxo-7,8- dihydropyrido[2,3- d]pyrimidin-2- yl)amino)pyridin-3- yl)piperazin-1- yl)ethyl) piperidin-4-yl)-1- oxoisoindolin-2- yl)piperidine-2,6- dione | 800.41 | B | B | B | C | C |
| 73 | 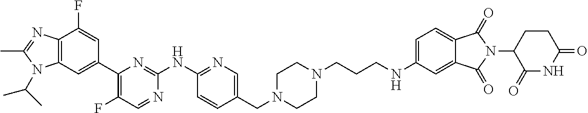 [SMILES: Cc1nc2c(F)cc(-c3nc(Nc4ccc(CN5CCN(CCCNc6ccc7c(c6)C(=O)N(C6CCC(=O)NC6=O)C7=O)CC5)cn4)ncc3F)cc2n1C(C)C] | 2-(2,6- dioxopiperidin- 3-yl)-5-((3- (4-((6-((5-fluoro- 4-(4-fluoro-1- isopropyl-2- methyl-1H- benzo[d]imidazol- 6-yl)pyrimidin-2- yl)amino)pyridin-3- yl)methyl) piperazin-1- yl)propyl)amino) isoindoline-1,3- dione | 791.35 | A | A | A | A | A |
| 74 | 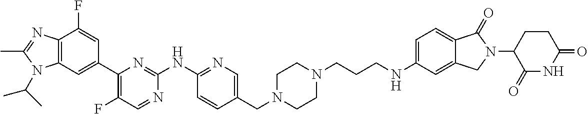 [SMILES: Cc1nc2c(F)cc(-c3nc(Nc4ccc(CN5CCN(CCCNc6ccc7c(c6)CN(C6CCC(=O)NC6=O)C7=O)CC5)cn4)ncc3F)cc2n1C(C)C] | 3-(5-((3-(4-((6- ((5-fluoro-4-(4- fluoro-1-isopropyl- 2-methyl-1H- benzo[d]imidazol- 6-yl)pyrimidin-2- yl)amino)pyridin-3- yl)methyl)piperazin-1- yl)propyl)amino)-1- oxoisoindolin- 2-yl)piperidine- 2,6-dione | 777.37 | A | A | A | A | A |
| 75 | 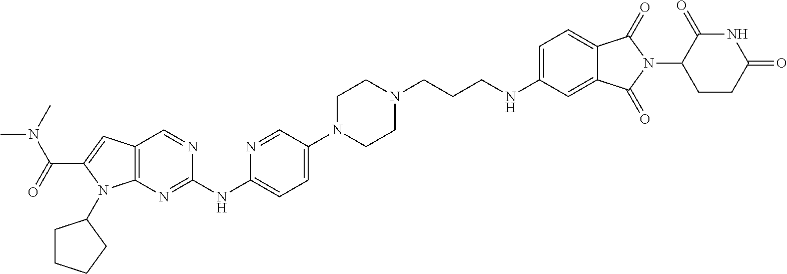 [SMILES: CN(C)C(=O)c1cc2cnc(Nc3ccc(N4CCN(CCCNc5ccc6c(c5)C(=O)N(C5CCC(=O)NC5=O)C6=O)CC4)cn3)nc2n1C1CCCC1] | 7-cyclopentyl-2- ((5-(4-(3-((2-(2,6- dioxopiperidin- 3-yl)-1,3- dioxoisoindolin-5- yl)amino)propyl) piperazin-1- yl)pyridin-2-yl) amino)-N,N- dimethyl-7H- pyrrolo[2,3- d]pyrimidine-6- carboxamide | 747.36 | A | A | A | A | A |
| 76 | 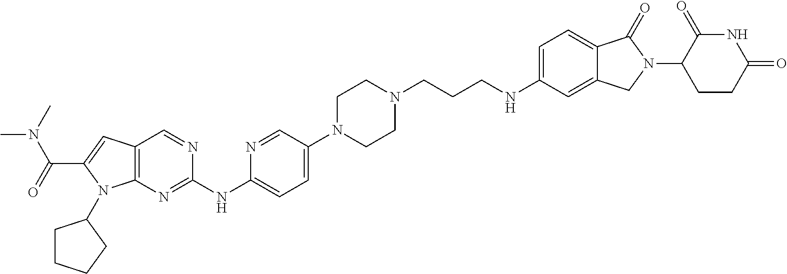 [SMILES: CN(C)C(=O)c1cc2cnc(Nc3ccc(N4CCN(CCCNc5ccc6c(c5)CN(C5CCC(=O)NC5=O)C6=O)CC4)cn3)nc2n1C1CCCC1] | 7-cyclopentyl-2- ((5-(4-(3-((2-(2,6- dioxopiperidin- 3-yl)-1- oxoisoindolin-5- yl)amino)propyl) piperazin-1- yl)pyridin-2-yl) amino)-N,N- dimethyl-7H- pyrrolo[2,3- d]pyrimidine-6- carboxamide | 733.38 | A | A | A | A | A |
| 77 | 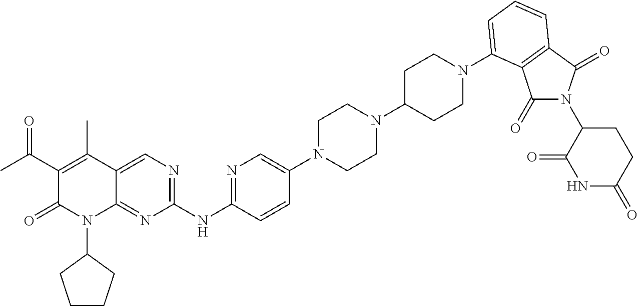 [SMILES: CC(=O)c1c(C)c2cnc(Nc3ccc(N4CCN(C5CCN(c6cccc7c6C(=O)N(C6CCC(=O)NC6=O)C7=O)CC5)CC4)cn3)nc2n(C2CCCC2)c1=O] | 4-(4-(4-(6-((6-acetyl- 8-cyclopentyl- 5-methyl-7-oxo-7,8- dihydropyrido[2,3- d]pyrimidin-2- yl)amino)pyridin-3- yl)piperazin-1- y1)piperidin-1- yl)-2-(2,6- dioxopiperidin-3-yl) isoindoline-1,3- dione | 786.36 | B | B | B | B | B |
| 78 | 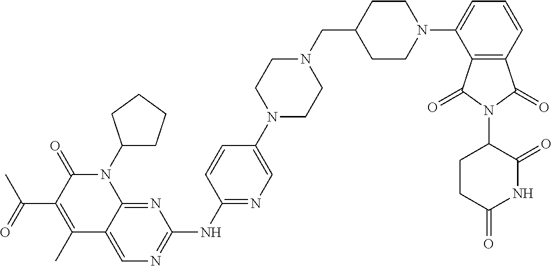 [SMILES: CC(=O)c1c(C)c2cnc(Nc3ccc(N4CCN(CC5CCN(c6cccc7c6C(=O)N(C6CCC(=O)NC6=O)C7=O)CC5)CC4)cn3)nc2n(C2CCCC2)c1=O] | 4-(4-((4-(6- ((6-acetyl-8- cyclopentyl-5- methyl-7-oxo-7,8- dihydropyrido[2,3- d]pyrimidin-2- yl)amino)pyridin-3- yl)piperazin-1- yl)methyl)piperidin- 1-yl)-2-(2,6- dioxopiperidin- 3-yl) isoindoline-1,3- dione | 800.38 | B | B | B | B | B |
| 79 | 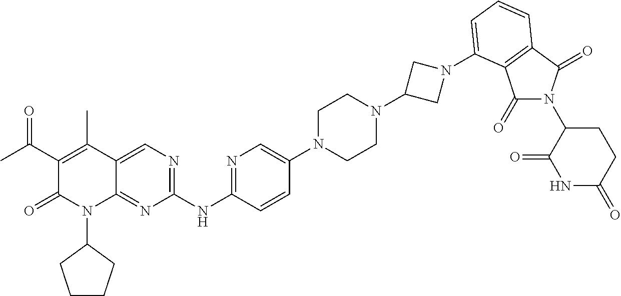 [SMILES: CC(=O)c1c(C)c2cnc(Nc3ccc(N4CCN(C5CN(c6cccc7c6C(=O)N(C6CCC(=O)NC6=O)C7=O)C5)CC4)cn3)nc2n(C2CCCC2)c1=O] | 4-(3-(4-(6-((6-acetyl- 8-cyclopentyl- 5-methyl-7-oxo-7,8- dihydropyrido[2,3- d]pyrimidin-2- yl)amino)pyridin-3- yl)piperazin-1- yl)azetidin-1- yl)-2-(2,6- dioxopiperidin-3- yl)isoindoline-1,3- dione | 758.33 | B | B | B | B | B |
| 80 | 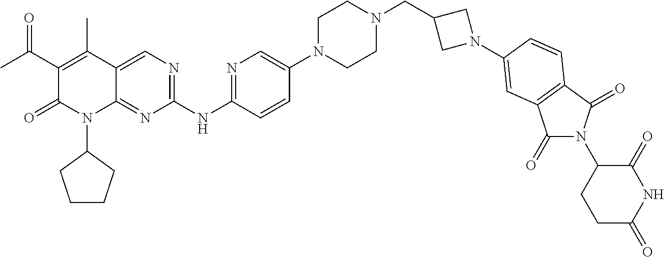 [SMILES: CC(=O)c1c(C)c2cnc(Nc3ccc(N4CCN(CC5CN(c6ccc7c(c6)C(=O)N(C6CCC(=O)NC6=O)C7=O)C5)CC4)cn3)nc2n(C2CCCC2)c1=O] | 5-(3-((4-(6- ((6-acetyl-8- cyclopentyl-5- methyl-7-oxo-7,8- dihydropyrido[2,3- d]pyrimidin-2- yl)amino)pyridin-3- yl)piperazin-1- yl)methyl)azetidin- 1-yl)-2-(2,6- dioxopiperidin-3-yl) isoindoline-1,3- dione | 772.34 | A | A | A | A | A |
| 81 | 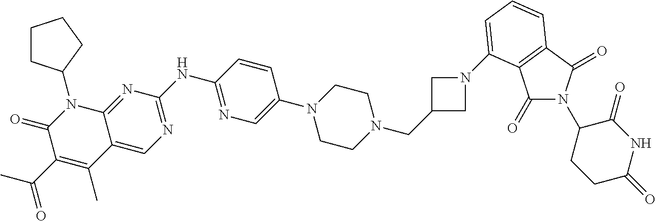 [SMILES: CC(=O)c1c(C)c2cnc(Nc3ccc(N4CCN(CC5CN(c6cccc7c6C(=O)N(C6CCC(=O)NC6=O)C7=O)C5)CC4)cn3)nc2n(C2CCCC2)c1=O] | 4-(3-((4-(6- ((6-acetyl-8- cyclopentyl-5- methyl-7-oxo-7,8- dihydropyrido[2,3- d]pyrimidin-2- yl)amino)pyridin-3- yl)piperazin-1- yl)methyl)azetidin- 1-yl)-2-(2,6- dioxopiperidin-3-yl) isoindoline-1,3- dione | 772.34 | A | B | A | B | A |
| 82 | 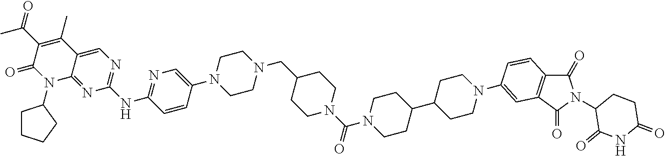 [SMILES: CC(=O)c1c(C)c2cnc(Nc3ccc(N4CCN(CC5CCN(C(=O)N6CCC(C7CCN(c8ccc9c(c8)C(=O)N(C8CCC(=O)NC8=O)C9=O)CC7)CC6)CC5)CC4)cn3)nc2n(C2CCCC2)c1=O] | 5-(1′-(4-((4-(6- ((6-acetyl-8- cyclopentyl-5- methyl-7-oxo-7,8- dihydropyrido[2,3- d]pyrimidin-2- yl)amino)pyridin-3- yl)piperazin-1- yl)methyl) piperidine- 1-carbonyl)- [4,4′-bipiperidin]- 1-yl)-2-(2,6- dioxopiperidin-3-yl) isoindoline-1,3- dione | 994.52 | C | C | C | C | C |
| 83 | 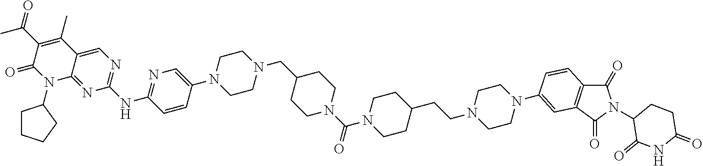 [SMILES: CC(=O)c1c(C)c2cnc(Nc3ccc(N4CCN(CC5CCN(C(=O)N6CCC(CCN7CCN(c8ccc9c(c8)C(=O)N(C8CCC(=O)NC8=O)C9=O)CC7)CC6)CC5)CC4)cn3)nc2n(C2CCCC2)c1=O] | 5-(4-(2-(1-(4-((4- (6-((6-acetyl-8- cyclopentyl-5-methyl- 7-oxo-7,8- dihydropyrido[2,3- d]pyrimidin-2- yl)amino)pyridin- 3-yl)piperazin-1- yl)methyl) piperidine-1- carbonyl) piperidin-4- yl)ethyl)piperazin- 1-yl)-2-(2,6- dioxopiperidin-3-yl) isoindoline-1,3- dione | 1023.54 | C | C | C | C | C |
| 84 | 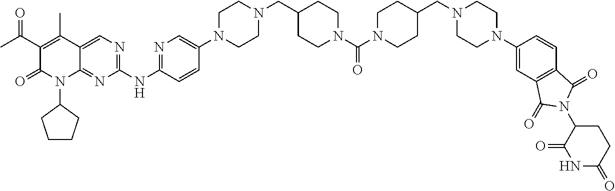 [SMILES: CC(=O)c1c(C)c2cnc(Nc3ccc(N4CCN(CC5CCN(C(=O)N6CCC(CN7CCN(c8ccc9c(c8)C(=O)N(C8CCC(=O)NC8=O)C9=O)CC7)CC6)CC5)CC4)cn3)nc2n(C2CCCC2)c1=O] | 5-(4-((1-(4-((4- (6-((6-acetyl-8- cyclopentyl-5- methyl-7-oxo-7,8- dihydropyrido[2,3- d]pyrimidin-2- yl)amino)pyridin-3- yl)piperazin-1- yl)methyl) piperidine-1- carbonyl) piperidin-4- yl)methyl) piperazin- 1-yl)-2-(2,6- dioxopiperidin-3-yl) isoindoline-1,3- dione | 1009.53 | A | B | A | B | A |
| 85 | 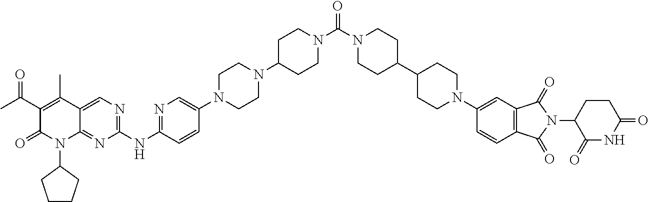 [SMILES: CC(=O)c1c(C)c2cnc(Nc3ccc(N4CCN(C5CCN(C(=O)N6CCC(C7CCN(c8ccc9c(c8)C(=O)N(C8CCC(=O)NC8=O)C9=O)CC7)CC6)CC5)CC4)cn3)nc2n(C2CCCC2)c1=O] | 5-(1′-(4-(4-(6- ((6-acetyl-8- cyclopentyl-5- methyl-7-oxo-7,8- dihydropyrido[2,3- d]pyrimidin-2- yl)amino)pyridin-3- yl)piperazin-1- yl)piperidine-1- carbonyl)-[4,4′- bipiperidin]-1- yl)-2-(2,6- dioxopiperidin-3-yl) isoindoline-1,3- dione | 980.50 | C | C | B | C | B |
| 86 | 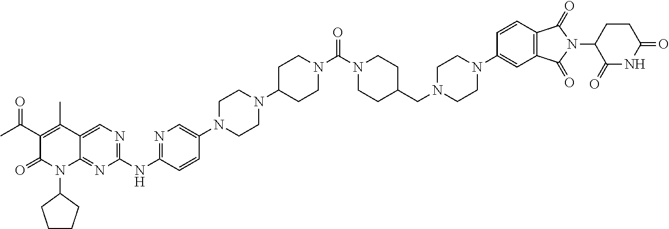 [SMILES: CC(=O)c1c(C)c2cnc(Nc3ccc(N4CCN(C5CCN(C(=O)N6CCC(CN7CCN(c8ccc9c(c8)C(=O)N(C8CCC(=O)NC8=O)C9=O)CC7)CC6)CC5)CC4)cn3)nc2n(C2CCCC2)c1=O] | 5-(4-((1-(4-(4- (6-((6-acetyl-8- cyclopentyl-5- methyl-7-oxo-7,8- dihydropyrido[2,3- d]pyrimidin-2- yl)amino)pyridin-3- yl)piperazin-1- yl)piperidine-1- carbonyl)piperidin- 4-yl)methyl) piperazin- 1-yl)-2-(2,6- dioxopiperidin-3-yl) isoindoline-1,3- dione | 995.51 | B | B | B | B | B |
| 87 | 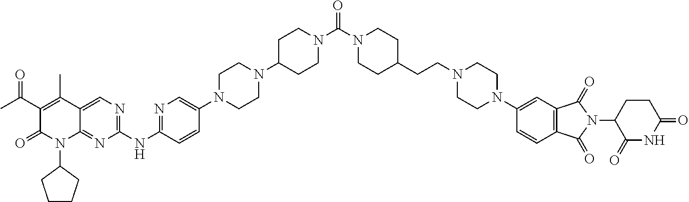 [SMILES: CC(=O)c1c(C)c2cnc(Nc3ccc(N4CCN(C5CCN(C(=O)N6CCC(CCN7CCN(c8ccc9c(c8)C(=O)N(C8CCC(=O)NC8=O)C9=O)CC7)CC6)CC5)CC4)cn3)nc2n(C2CCCC2)c1=O] | 5-(4-(2-(1-(4-(4- (6-((6-acetyl-8- cyclopentyl-5- methyl-7-oxo-7,8- dihydropyrido[2,3- d]pyrimidin-2- yl)amino)pyridin- 3-yl)piperazin-1- yl)piperidine-1- carbonyl)piperidin- 4-yl)ethyl)piperazin- 1-yl)-2-(2,6- dioxopiperidin-3-yl) isoindoline-1,3- dione | 1009.53 | B | B | B | B | B |
| 88 | 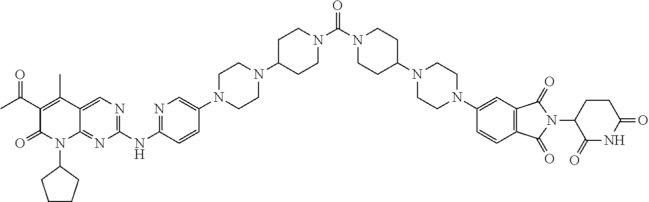 [SMILES: CC(=O)c1c(C)c2cnc(Nc3ccc(N4CCN(C5CCN(C(=O)N6CCC(N7CCN(c8ccc9c(c8)C(=O)N(C8CCC(=O)NC8=O)C9=O)CC7)CC6)CC5)CC4)cn3)nc2n(C2CCCC2)c1=O] | 5-(4-(1-(4-(4- (6-((6-acetyl-8- cyclopentyl-5- methyl-7-oxo-7,8- dihydropyrido[2,3- d]pyrimidin-2- yl)amino)pyridin-3- yl)piperazin-1- yl)piperidine-1- carbonyl)piperidin- 4-yl)piperazin- 1-yl)-2-(2,6- dioxopiperidin-3-yl) isoindoline-1,3- dione | 981.50 | C | C | B | C | C |
| 89 | 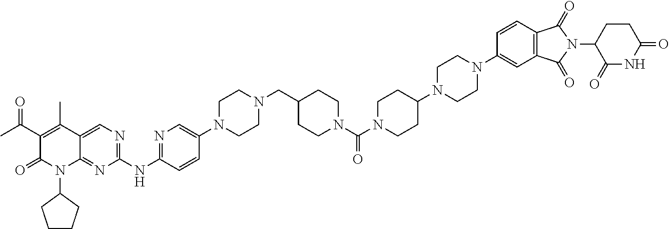 [SMILES: CC(=O)c1c(C)c2cnc(Nc3ccc(N4CCN(CC5CCN(C(=O)N6CCC(N7CCN(c8ccc9c(c8)C(=O)N(C8CCC(=O)NC8=O)C9=O)CC7)CC6)CC5)CC4)cn3)nc2n(C2CCCC2)c1=O] | 5-(4-(1-(4-((4- (6-((6-acetyl-8- cyclopentyl-5- methyl-7-oxo-7,8- dihydropyrido[2,3- d]pyrimidin-2- yl)amino)pyridin- 3-yl)piperazin-1- yl)methyl) piperidine-1- carbonyl)piperidin- 4-yl)piperazin-1- yl)-2-(2,6- dioxopiperidin-3- yl)isoindoline- 1,3-dione | 995.51 | B | B | B | C | B |
| 90 | 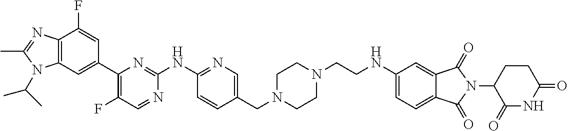 [SMILES: Cc1nc2c(F)cc(-c3nc(Nc4ccc(CN5CCN(CCNc6ccc7c(c6)C(=O)N(C6CCC(=O)NC6=O)C7=O)CC5)cn4)ncc3F)cc2n1C(C)C] | 2-(2,6- dioxopiperidin- 3-yl)-5-((2- (4-((6-((5-fluoro- 4-(4-fluoro-1- isopropyl-2- methyl-1H- benzo[d]imidazol- 6-yl)pyrimidin-2- yl)amino)pyridin-3- yl)methyl) piperazin-1- yl)ethyl)amino) isoindoline-1,3- dione | 777.33 | A | A | A | A | A |
| 91 | 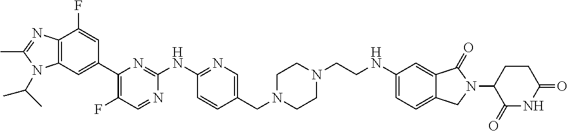 [SMILES: Cc1nc2c(F)cc(-c3nc(Nc4ccc(CN5CCN(CCNc6ccc7c(c6)C(=O)N(C6CCC(=O)NC6=O)C7)CC5)cn4)ncc3F)cc2n1C(C)C] | 3-(6-((2-(4-((6- ((5-fluoro-4-(4- fluoro-1-isopropyl- 2-methyl-1H- benzo[d]imidazol- 6-yl)pyrimidin-2- yl)amino)pyridin-3- yl)methyl) piperazin-1- yl)ethyl)amino)-1- oxoisoindolin-2- yl)piperidine- 2,6-dione | 763.35 | A | A | A | A | A |
| 92 | 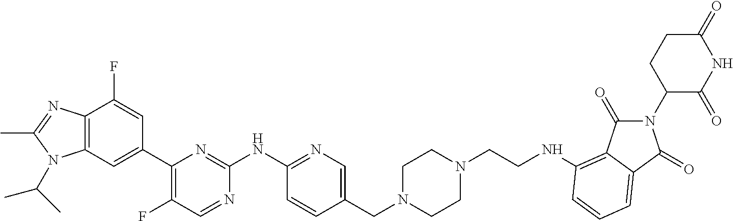 [SMILES: Cc1nc2c(F)cc(-c3nc(Nc4ccc(CN5CCN(CCNc6cccc7c6C(=O)N(C6CCC(=O)NC6=O)C7=O)CC5)cn4)ncc3F)cc2n1C(C)C] | 2-(2,6- dioxopiperidin- 3-yl)-4-((2- (4-((6-((5-fluoro- 4-(4-fluoro-1 isopropyl-2- methyl-1H- benzo[d]imidazol- 6-yl)pyrimidin-2- yl)amino)pyridin-3- yl)methyl) piperazin-1- yl)ethyl)amino) isoindoline-1,3- dione | 777.33 | A | B | A | A | A |
| 93 | 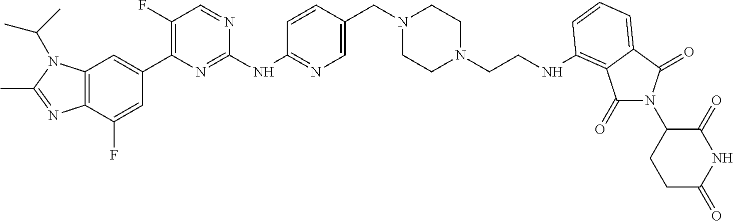 [SMILES: Cc1nc2c(F)cc(-c3nc(Nc4ccc(CN5CCN(CCNc6cccc7c6C(=O)N(C6CCC(=O)NC6=O)C7=O)CC5)cn4)ncc3F)cc2n1C(C)C] | 3-(4-((2-(4-((6- ((5-fluoro-4-(4- fluoro-1-isopropyl- 2-methyl-1H- benzo[d]imidazol-6- yl)pyrimidin-2- yl)amino)pyridin-3- yl)methyl) piperazin-1- ylethyl)amino)-1- oxoisoindolin-2- yl)piperidine-2,6- dione | 763.35 | A | B | A | B | A |
| 94 | 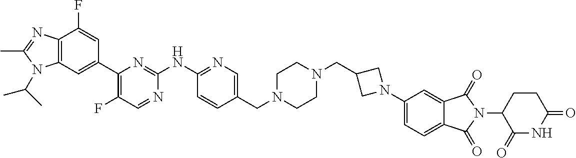 [SMILES: Cc1nc2c(F)cc(-c3nc(Nc4ccc(CN5CCN(CC6CN(c7ccc8c(c7)C(=O)N(C7CCC(=O)NC7=O)C8=O)C6)CC5)cn4)ncc3F)cc2n1C(C)C] | 2-(2,6- dioxopiperidin- 3-yl)-5-(3- ((4-((6-((5-fluoro- 4-(4-fluoro-1- isopropyl-2- methyl-1H- benzo[d]imidazol- 6-yl)pyrimidin-2- yl)amino)pyridin-3- yl)methyl) piperazin-1- yl)methyl)azetidin- 1-yl)isoindoline- 1,3-dione | 803.35 | A | A | A | A | A |
| 95 | 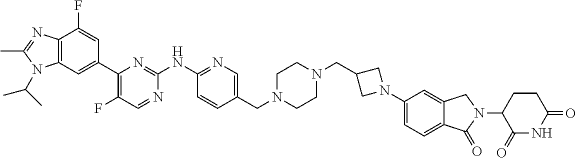 [SMILES: Cc1nc2c(F)cc(-c3nc(Nc4ccc(CN5CCN(CC6CN(c7ccc8c(c7)CN(C7CCC(=O)NC7=O)C8=O)C6)CC5)cn4)ncc3F)cc2n1C(C)C] | 3-(5-(3-((4-((6- ((5-fluoro-4-(4- fluoro-1-isopropyl- 2-methyl-1H- benzo[d]imidazol-6- yl)pyrimidin-2- yl)amino)pyridin-3- yl)methyl) piperazin-1- yl)methyl) azetidin-1-yl)-1- oxoisoindolin-2- yl)piperidine-2,6- dione | 789.37 | A | A | A | A | A |
| 96 | 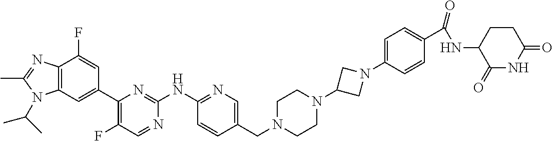 [SMILES: Cc1nc2c(F)cc(-c3nc(Nc4ccc(CN5CCN(C6CN(c7ccc(C(=O)NC8CCC(=O)NC8=O)cc7)C6)CC5)cn4)ncc3F)cc2n1C(C)C] | N-(2,6- dioxopiperidin- 3-yl)-4-(3-(4- ((6-((5-fluoro- 4-(4-fluoro-1- isopropyl-2- methyl-1H- benzo[d]imidazol-6- yl)pyrimidin-2- yl)amino)pyridin-3- yl)methyl)piperazin- 1-yl)azetidin-1- yi)benzamide | 763.35 | A | A | A | A | A |
| 97 | 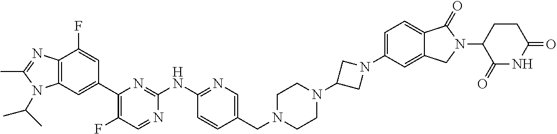 [SMILES: Cc1nc2c(F)cc(-c3nc(Nc4ccc(CN5CCN(C6CN(c7ccc8c(c7)CN(C7CCC(=O)NC7=O)C8=O)C6)CC5)cn4)ncc3F)cc2n1C(C)C] | 3-(5-(3-(4-((6- ((5-fluoro-4-(4- fluoro-1-isopropyl- 2-methyl-1H- benzo[d]imidazol-6- yl)pyrimidin-2- yl)amino) pyridin-3- yl)methyl)piperazin- 1-yl)azetidin-1- yl)-1-oxoisoindolin- 2-yl)piperidine- 2,6-dione | 775.35 | A | A | A | A | A |
| 98 | 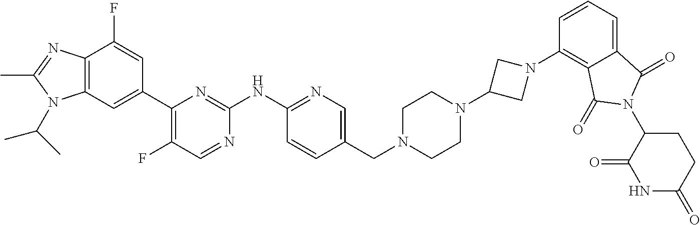 [SMILES: Cc1nc2c(F)cc(-c3nc(Nc4ccc(CN5CCN(C6CN(c7cccc8c7C(=O)N(C7CCC(=O)NC7=O)C8=O)C6)CC5)cn4)ncc3F)cc2n1C(C)C] | 2-(2,6- dioxopiperidin- 3-yl)-4-(3-(4- ((6-((5-fluoro- 4-(4-fluoro-1 isopropyl-2- methyl-1H- benzo[d]imidazol-6- yl)pyrimidin-2- yl)amino)pyridin-3- yl)methyl)piperazin- 1-yl)azetidin-1- yl)isoindoline- 1,3-dione | 789.33 | B | B | A | B | A |
| 99 | 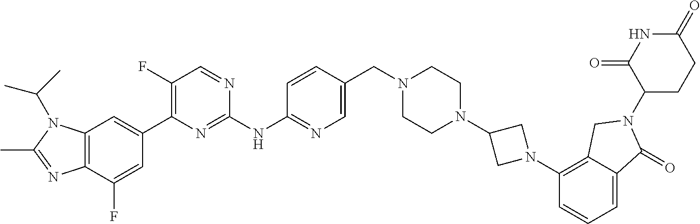 [SMILES: Cc1nc2c(F)cc(-c3nc(Nc4ccc(CN5CCN(C6CN(c7cccc8c7CN(C7CCC(=O)NC7=O)C8=O)C6)CC5)cn4)ncc3F)cc2n1C(C)C] | 3-(4-(3-(4-((6- ((5-fluoro-4-(4- fluoro-1-isopropyl- 2-methyl-1H- benzo[d]imidazol- 6-yl)pyrimidin-2- yl)amino)pyridin-3- yl)methyl)piperazin- 1-yl)azetidin-1- yl)-1-oxoisoindolin- 2-yl)piperidine- 2,6-dione | 775.35 | B | B | B | B | B |
| 100 | 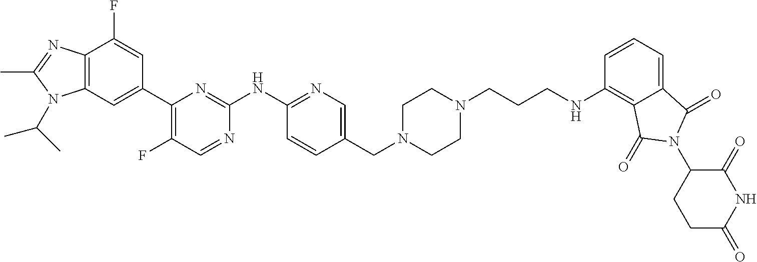 [SMILES: Cc1nc2c(F)cc(-c3nc(Nc4ccc(CN5CCN(CCCNc6cccc7c6C(=O)N(C6CCC(=O)NC6=O)C7=O)CC5)cn4)ncc3F)cc2n1C(C)C] | 2-(2,6- dioxopiperidin- 3-yl)-4-((3- (4-((6-((5-fluoro- 4-(4-fluoro-1 isopropyl-2- methyl-1H- benzo[d]imidazol- 6-yl)pyrimidin-2- yl)amino)pyridin-3- yl)methyl) piperazin-1- yl)propyl)amino) isoindoline-1,3- dione | 791.35 | A | B | A | B | A |
| 101 | 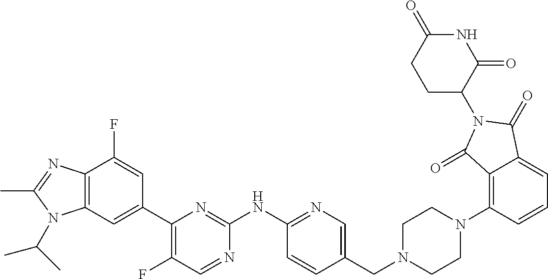 [SMILES: Cc1nc2c(F)cc(-c3nc(Nc4ccc(CN5CCN(c6cccc7c6C(=O)N(C6CCC(=O)NC6=O)C7=O)CC5)cn4)ncc3F)cc2n1C(C)C] | 2-(2,6- dioxopiperidin- 3-yl)-4-(4- ((6-((5-fluoro- 4-(4-fluoro-1- isopropyl-2- methyl-1H- benzo[d]imidazol- 6-yl)pyrimidin-2- yl)amino)pyridin- 3-yl)methyl) piperazin-1- yl)isoindoline-1,3- dione | 734.29 | B | B | B | B | A |
| 102 | 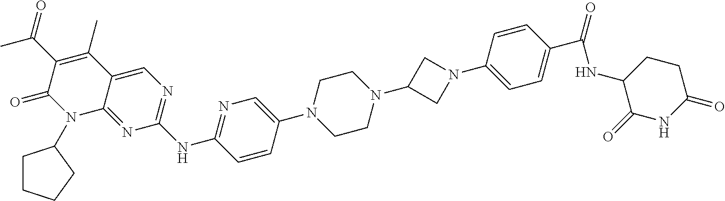 [SMILES: CC(=O)c1c(C)c2cnc(Nc3ccc(N4CCN(C5CN(c6ccc(C(=O)NC7CCC(=O)NC7=O)cc6)C5)CC4)cn3)nc2n(C2CCCC2)c1=O] | 4-(3-(4-(6-((6-acetyl- 8-cyclopentyl- 5-methyl- 7-oxo-7,8- dihydropyrido[2,3- d]pyrimidin-2- yl)amino)pyridin-3- yl)piperazin-1- yl)azetidin-1- yl)-N-(2,6- dioxopiperidin-3- yl)benzamide | 732.35 | B | B | A | B | B |
| 103 | 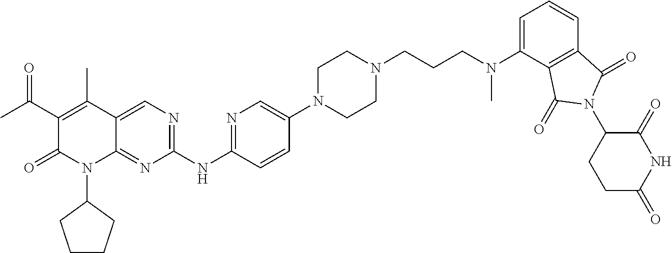 [SMILES: CC(=O)c1c(C)c2cnc(Nc3ccc(N4CCN(CCCN(C)c5cccc6c5C(=O)N(C5CCC(=O)NC5=O)C6=O)CC4)cn3)nc2n(C2CCCC2)c1=O] | 4-((3-(4-(6- ((6-acetyl-8- cyclopentyl-5- methyl-7-oxo-7,8- dihydropyrido[2,3- d]pyrimidin-2- yl)amino)pyridin- 3-yl)piperazin-1- yl)propyl)(methyl) amino)-2-(2,6- dioxopiperidin-3-yl) isoindoline-1,3- dione | 774.36 | A | B | B | B | B |
| 104 | 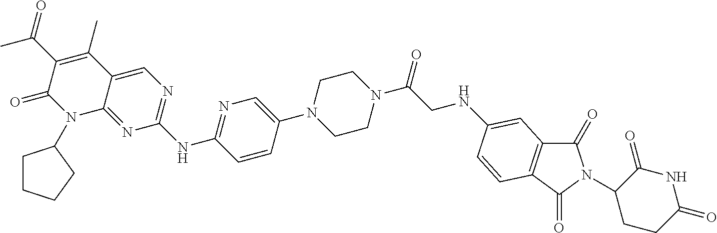 [SMILES: CC(=O)c1c(C)c2cnc(Nc3ccc(N4CCN(C(=O)CNc5ccc6c(c5)C(=O)N(C5CCC(=O)NC5=O)C6=O)CC4)cn3)nc2n(C2CCCC2)c1=O] | 5-((2-(4-(6- ((6-acetyl-8- cyclopentyl-5- methyl-7-oxo-7,8- dihydropyrido[2,3- d]pyrimidin-2- yl)amino)pyridin- 3-yl)piperazin-1- yl)-2-oxoethyl) amino)-2-(2,6- dioxopiperidin-3- yl)isoindoline-1,3- dione | 760.31 | A | B | B | B | B |
| 105 | 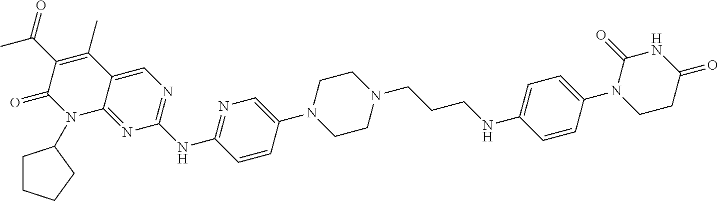 [SMILES: CC(=O)c1c(C)c2cnc(Nc3ccc(N4CCN(CCCNc5ccc(N6CCC(=O)NC6=O)cc5)CC4)cn3)nc2n(C2CCCC2)c1=O] | 1-(4-((3-(4- (6-((6-acetyl-8- cyclopentyl-5- methyl-7-oxo-7,8- dihydropyrido[2,3- d]pyrimidin-2- yl)amino)pyridin- 3-yl)piperazin-1- yl)propyl)amino) phenyl)dihydro- pyrimidine-2,4 (1H,3H)-dione | 692.35 | A | A | A | A | A |
| 106 | 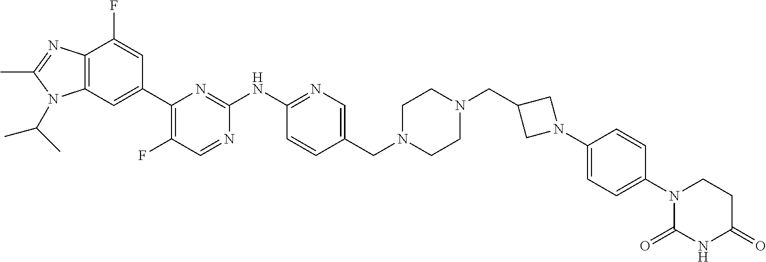 [SMILES: Cc1nc2c(F)cc(-c3nc(Nc4ccc(CN5CCN(CC6CN(c7ccc(N8CCC(=O)NC8=O)cc7)C6)CC5)cn4)ncc3F)cc2n1C(C)C] | 1-(4-(3-((4-((6- ((5-fluoro-4-(4- fluoro-1-isopropyl- 2-methyl-1H- benzo[d]imidazol-6- yl)pyrimidin-2- yl)amino)pyridin-3- yl)methyl) piperazin-1- yl)methyl) azetidin-1- yl)phenyl) dihydropyrimidine- 2,4(1H,3H)-dione | 735.36 | A | A | A | A | A |
| 107 | 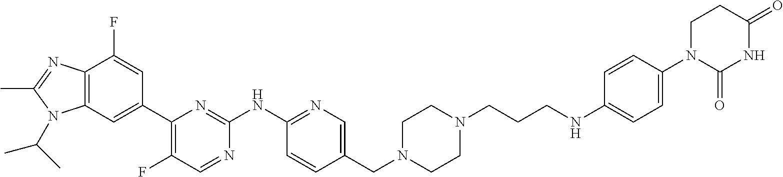 [SMILES: Cc1nc2c(F)cc(-c3nc(Nc4ccc(CN5CCN(CCCNc6ccc(N7CCC(=O)NC7=O)cc6)CC5)cn4)ncc3F)cc2n1C(C)C] | 1-(4-((3-(4-((6- ((5-fluoro-4-(4- fluoro-1-isopropyl- 2-methyl-1H- benzo[d]imidazol- 6-yl)pyrimidin-2- yl)amino) pyridin-3- yl)methyl) piperazin-1- yl)propyl)amino) phenyl)dihydro- pyrimidine-2,4 (1H,3H)-dione | 723.36 | A | A | A | A | A |
| 108 | 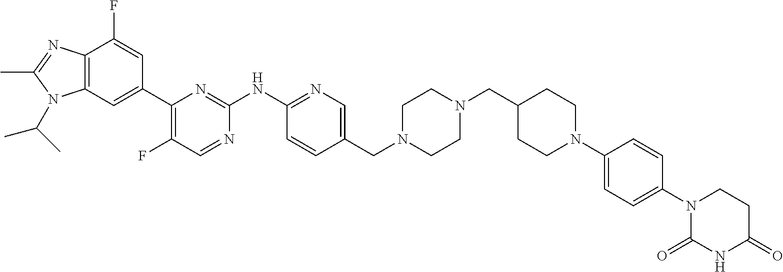 [SMILES: Cc1nc2c(F)cc(-c3nc(Nc4ccc(CN5CCN(CC6CCN(c7ccc(N8CCC(=O)NC8=O)cc7)CC6)CC5)cn4)ncc3F)cc2n1C(C)C] | 1-(4-(4-((4-((6- ((5-fluoro-4-(4- fluoro-1-isopropyl- 2-methyl-1H- benzo[d]imidazol- 6-yl)pyrimidin-2- yl)amino)pyridin-3- yl)methyl) piperazin-1- yl)methyl) piperidin-1- yl)phenyl) dihydropyrimidine- 2,4(1H,3H)-dione | 763.39 | A | A | A | A | A |
| 109 | 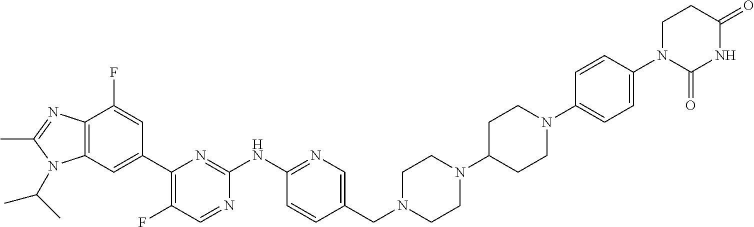 [SMILES: Cc1nc2c(F)cc(-c3nc(Nc4ccc(CN5CCN(C6CCN(c7ccc(N8CCC(=O)NC8=O)cc7)CC6)CC5)cn4)ncc3F)cc2n1C(C)C] | 1-(4-(4-(4-((6- ((5-fluoro-4-(4- fluoro-1-isopropyl- 2-methyl-1H- benzo[d]imidazol-6- yl)pyrimidin-2- yl)amino) pyridin-3- yl)methyl)piperazin- 1-yl)piperidin- 1-yl)phenyl) dihydropyrimidine- 2,4(1H,3H)-dione | 749.37 | A | A | A | A | A |
| 110 | 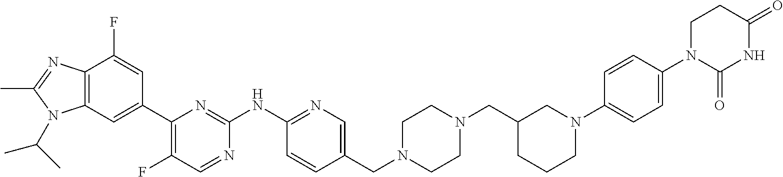 [SMILES: Cc1nc2c(F)cc(-c3nc(Nc4ccc(CN5CCN(CC6CCCN(c7ccc(N8CCC(=O)NC8=O)cc7)C6)CC5)cn4)ncc3F)cc2n1C(C)C] | 1-(4-(3-((4-((6- ((5-fluoro-4-(4- fluoro-1-isopropyl- 2-methyl-1H- benzo[d]imidazol- 6-yl)pyrimidin-2- yl)amino)pyridin-3- yl)methyl) piperazin-1- yl)methyl) piperidin-1- yl)phenyl) dihydropyrimidine- 2,4(1H,3H)-dione | 763.9 | A | A | A | A | A |
| 111 | 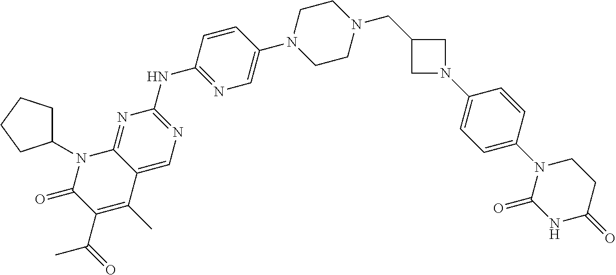 [SMILES: CC(=O)c1c(C)c2cnc(Nc3ccc(N4CCN(CC5CN(c6ccc(N7CCC(=O)NC7=O)cc6)C5)CC4)cn3)nc2n(C2CCCC2)c1=O] | 1-(4-(3-((4- (6-((6-acetyl-8- cyclopentyl-5- methyl-7-oxo-7,8- dihydropyrido[2,3- d]pyrimidin-2- yl)amino)pyridin- 3-yl)piperazin-1- yl)methyl) azetidin-1- yl)phenyl) dihydropyrimidine- 2,4(1H,3H)-dione | 704.35 | A | A | A | A | A |
| 112 | 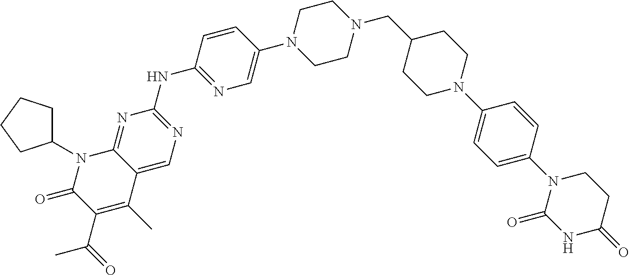 [SMILES: CC(=O)c1c(C)c2cnc(Nc3ccc(N4CCN(CC5CCN(c6ccc(N7CCC(=O)NC7=O)cc6)CC5)CC4)cn3)nc2n(C2CCCC2)c1=O] | 1-(4-(4-((4- (6-((6-acetyl-8- cyclopentyl-5- methyl-7-oxo-7,8- dihydropyrido[2,3- d]pyrimidin-2- yl)amino)pyridin- 3-yl)piperazin-1- yl)methyl) piperidin-1- yl)phenyl) dihydropyrimidine- 2,4(1H,3H)-dione | 732.39 | A | A | A | B | A |
| 113 | 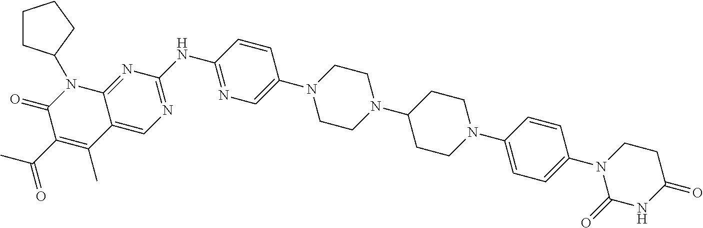 [SMILES: CC(=O)c1c(C)c2cnc(Nc3ccc(N4CCN(C5CCN(c6ccc(N7CCC(=O)NC7=O)cc6)CC5)CC4)cn3)nc2n(C2CCCC2)c1=O] | 1-(4-(4-(4- (6-((6-acetyl-8- cyclopentyl-5- methyl-7-oxo-7,8- dibydropyrido[2,3- d]pyrimidin-2- yl)amino)pyridin- 3-yl)piperazin-1- yl)piperidin-1- yi)phenyl) dihydropyrimidine- 2,4(1H,3H)-dione | 718.37 | A | B | A | A | A |
| 114 | 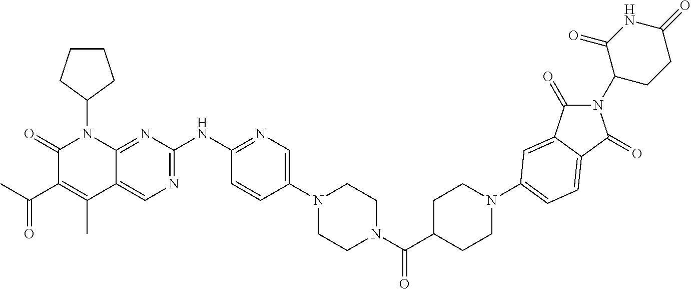 [SMILES: CC(=O)c1c(C)c2cnc(Nc3ccc(N4CCN(C(=O)C5CCN(c6ccc7c(c6)C(=O)N(C6CCC(=O)NC6=O)C7=O)CC5)CC4)cn3)nc2n(C2CCCC2)c1=O] | 5-(4-(4-(6-((6-acetyl- 8-cyclopentyl- 5-methyl- 7-oxo-7,8- dihydropyrido[2,3- d]pyrimidin-2- yl)amino)pyridin-3- yl)piperazine-1- carbonyl)piperidin- 1-yl)-2-(2,6- dioxopiperidin-3-yl) isoindoline-1,3- dione | 814.36 | A | B | A | B | A |
| 115 | 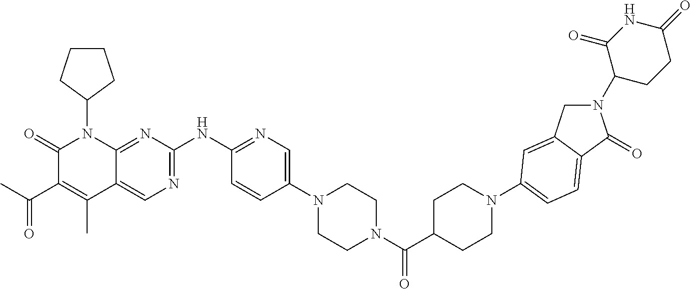 [SMILES: CC(=O)c1c(C)c2cnc(Nc3ccc(N4CCN(C(=O)C5CCN(c6ccc7c(c6)CN(C6CCC(=O)NC6=O)C7=O)CC5)CC4)cn3)nc2n(C2CCCC2)c1=O] | 3-(5-(4-(4-(6- ((6-acetyl-8- cyclopentyl-5- methyl-7-oxo-7,8- dihydropyrido[2,3- d]pyrimidin-2- yl)amino)pyridin- 3-yl)piperazine-1- carbonyl)piperidin- 1-yl)-1- oxoisoindolin-2- yl)piperidine-2,6- dione | 800.38 | A | B | B | B | B |
| 116 | 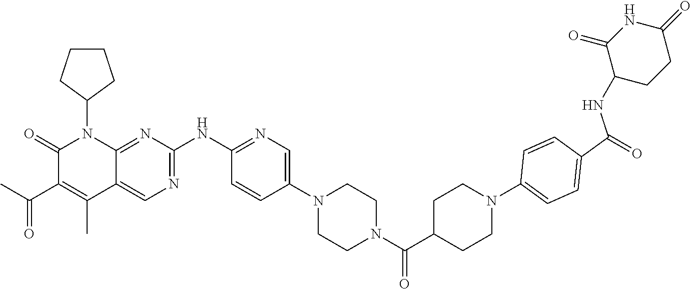 [SMILES: CC(=O)c1c(C)c2cnc(Nc3ccc(N4CCN(C(=O)C5CCN(c6ccc(C(=O)NC7CCC(=O)NC7=O)cc6)CC5)CC4)cn3)nc2n(C2CCCC2)c1=O] | 4-(4-(4-(6-((6-acetyl- 8-cyclopentyl- 5-methyl- 7-oxo-7,8- dihydropyrido[2,3- d]pyrimidin-2- yl)amino)pyridin-3- yl)piperazine-1- carbonyl)piperidin- 1-yl)-N-(2,6- dioxopiperidin-3- yl)benzamide | 788.38 | B | B | A | B | A |
| 117 | 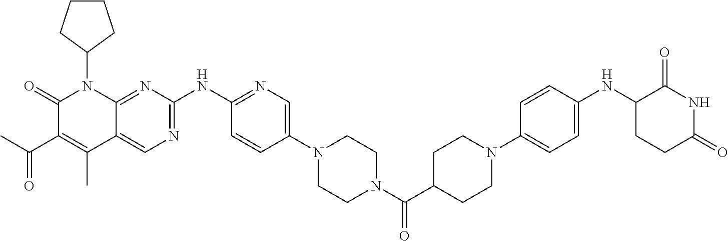 [SMILES: CC(=O)c1c(C)c2cnc(Nc3ccc(N4CCN(C(=O)C5CCN(c6ccc(NC7CCC(=O)NC7=O)cc6)CC5)CC4)cn3)nc2n(C2CCCC2)c1=O] | 3-((4-(4-(4-(6- ((6-acetyl-8- cyclopentyl-5- methyl-7-oxo-7,8- dihydropyrido[2,3- d]pyrimidin-2- yl)amino)pyridin-3- yl)piperazine-1- carbonyl) piperidin-1- yl)phenyl)amino) piperidine-2,6- dione | 760.38 | A | A | A | B | B |
| 118 | 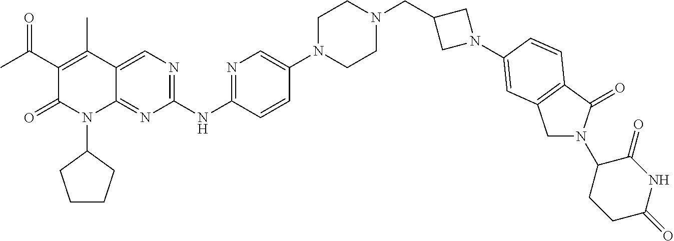 [SMILES: CC(=O)c1c(C)c2cnc(Nc3ccc(N4CCN(CC5CN(c6ccc7c(c6)CN(C6CCC(=O)NC6=O)C7=O)C5)CC4)cn3)nc2n(C2CCCC2)c1=O] | 3-(5-(3-((4-(6- ((6-acetyl-8- cyclopentyl-5- methyl-7-oxo-7,8- dihydropyrido[2,3- d]pyrimidin-2- yl)amino)pyridin-3- yl)piperazin-1- yl)methyl) azetidio-1-yl)-1- oxoisoindolin-2- yl)piperidine-2,6- dione | 758.37 | A | A | A | A | A |
| 119 | 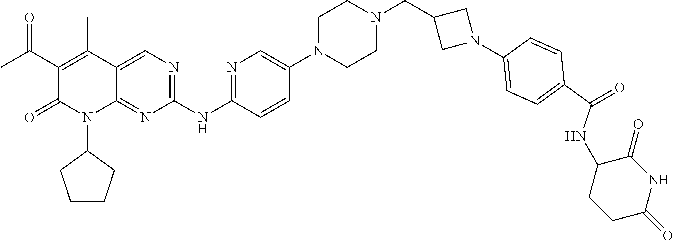 [SMILES: CC(=O)c1c(C)c2cnc(Nc3ccc(N4CCN(CC5CN(c6ccc(C(=O)NC7CCC(=O)NC7=O)cc6)C5)CC4)cn3)nc2n(C2CCCC2)c1=O] | 4-(3-((4-(6- ((6-acetyl-8- cyclopentyl-5- methyl-7-oxo-7,8- dihydropyrido[2,3- d]pyrimidin-2- yl)amino)pyridin-3- yl)piperazin-1- yl)methyl)azetidin- 1-yl)-N-(2,6- dioxopiperidin- 3-yl)benzamide | 746.37 | A | B | A | B | A |
| 120 | 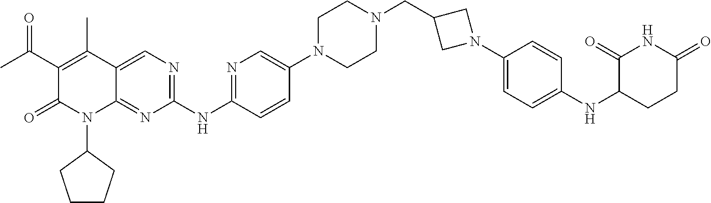 [SMILES: CC(=O)c1c(C)c2cnc(Nc3ccc(N4CCN(CC5CN(c6ccc(NC7CCC(=O)NC7=O)cc6)C5)CC4)cn3)nc2n(C2CCCC2)c1=O] | 3-((4-(3-((4- (6-((6-acetyl-8- cyclopentyl-5- methyl-7-oxo-7,8- dihydropyrido[2,3- d]pyrimidin-2- yl)amino)pyridin- 3-yl)piperazin-1- yl)methyl) azetidin-1- yl)phenyl)amino) piperidine-2,6- dione | 718.37 | A | A | A | B | A |
| 121 | 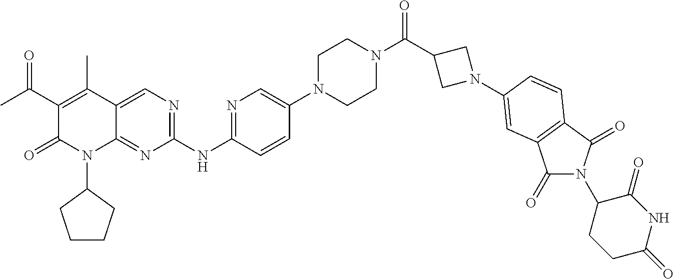 [SMILES: CC(=O)c1c(C)c2cnc(Nc3ccc(N4CCN(C(=O)C5CN(c6ccc7c(c6)C(=O)N(C6CCC(=O)NC6=O)C7=O)C5)CC4)cn3)nc2n(C2CCCC2)c1=O] | 5-(3-(4-(6- ((6-acetyl- 8-cyclopentyl- 5-methyl- 7-oxo-7,8- dihydropyrido[2,3- d]pyrimidin-2- yl)amino)pyridin-3- yl)piperazine-1- carbonyl)azetidin- 1-yl)-2-(2,6- dioxopiperidin-3- yl)isoindoline-1,3- dione | 786.32 | A | A | A | A | A |
| 122 | 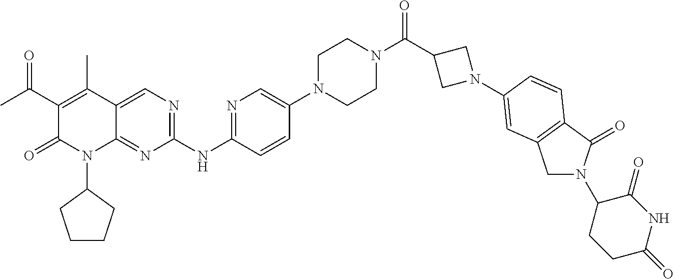 [SMILES: CC(=O)c1c(C)c2cnc(Nc3ccc(N4CCN(C(=O)C5CN(c6ccc7c(c6)CN(C6CCC(=O)NC6=O)C7=O)C5)CC4)cn3)nc2n(C2CCCC2)c1=O] | 3-(5-(3-(4-(6- ((6-acetyl-8- cyclopentyl-5- methyl-7-oxo-7,8- dihydropyrido[2,3- d]pyrimidin-2- yl)amino)pyridin-3- yl)piperazine-1- carbonyl)azetidin- 1-yl)-1- oxoisoindolin-2-yl) piperidine-2,6- dione | 772.34 | A | A | A | A | A |
| 123 | 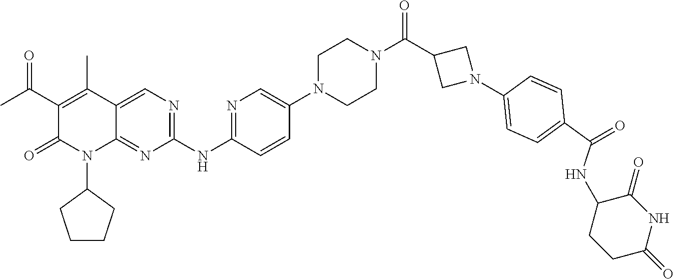 [SMILES: CC(=O)c1c(C)c2cnc(Nc3ccc(N4CCN(C(=O)C5CN(c6ccc(C(=O)NC7CCC(=O)NC7=O)cc6)C5)CC4)cn3)nc2n(C2CCCC2)c1=O] | 4-(3-(4-(6-((6-acetyl- 8-cyclopentyl- 5-methyl- 7-oxo-7,8- dihydropyrido[2,3- d]pyrimidin-2- yl)amino)pyridin-3- yl)piperazine-1- carbonyl)azetidin- 1-yl)-N-(2,6- dioxopiperidin-3- yl)benzamide | 760.34 | A | A | B | A | B |
| 124 | 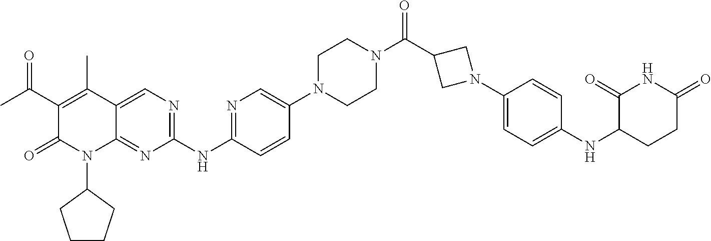 [SMILES: CC(=O)c1c(C)c2cnc(Nc3ccc(N4CCN(C(=O)C5CN(c6ccc(NC7CCC(=O)NC7=O)cc6)C5)CC4)cn3)nc2n(C2CCCC2)c1=O] | 3-((4-(3-(4-(6- ((6-acetyl-8- cyclopentyl-5- methyl-7-oxo-7,8- dihydropyrido[2,3- d]pyrimidin-2- yl)amino)pyridin-3- yl)piperazine-1- carbonyl)azetidin-1- yl)phenyl)amino) piperidine-2,6- dione | 732.35 | A | B | A | B | B |
| 125 | 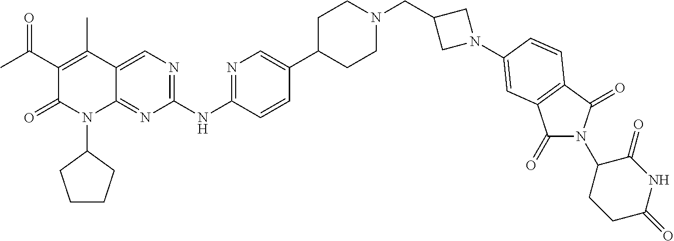 [SMILES: CC(=O)c1c(C)c2cnc(Nc3ccc(C4CCN(CC5CN(c6ccc7c(c6)C(=O)N(C6CCC(=O)NC6=O)C7=O)C5)CC4)cn3)nc2n(C2CCCC2)c1=O] | 5-(3-((4-(6- ((6-acetyl-8- cyclopentyl-5- methyl-7-oxo-7,8- dihydropyrido[2,3- d]pyrimidin-2- yl)amino)pyridin- 3-yl)piperidin-1- yl)methyl)azetidin- 1-yl)-2-(2,6- dioxopiperidin-3- yl)isoindoline-1,3- dione | 771.35 | A | A | A | A | A |
| 126 | 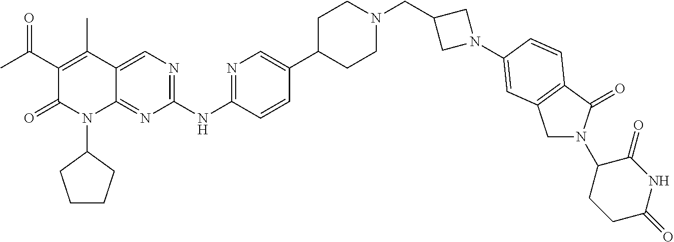 [SMILES: CC(=O)c1c(C)c2cnc(Nc3ccc(C4CCN(CC5CN(c6ccc7c(c6)CN(C6CCC(=O)NC6=O)C7=O)C5)CC4)cn3)nc2n(C2CCCC2)c1=O] | 3-(5-(3-((4-(6- ((6-acetyl-8- cyclopentyl-5- methyl-7-oxo-7,8- dihydropyrido[2,3- d]pyrimidin-2- yl)amino)pyridin-3- yl)piperidin-1- yl)methyl) azetidin-1-yl)-1- oxoisoindolin-2-yl) piperidine-2,6- dione | 757.37 | A | A | A | A | A |
| 127 | 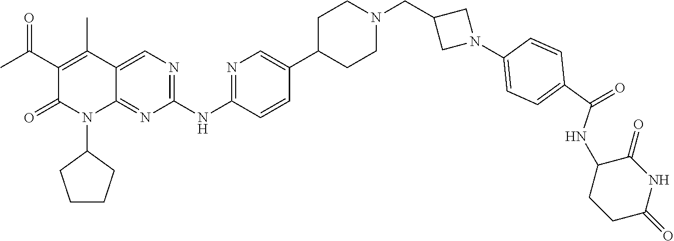 [SMILES: CC(=O)c1c(C)c2cnc(Nc3ccc(C4CCN(CC5CN(c6ccc(C(=O)NC7CCC(=O)NC7=O)cc6)C5)CC4)cn3)nc2n(C2CCCC2)c1=O] | 4-(3-((4-(6- ((6-acetyl-8- cyclopentyl-5- methyl-7-oxo-7,8- dihydropyrido[2,3- d]pyrimidin-2- yl)amino)pyridin- 3-yl)piperidin-1- y)methyl)azetidin- 1-yl)-N-(2,6- dioxopiperidin-3- yl)benzamide | 745.37 | A | B | A | B | A |
| 128 | 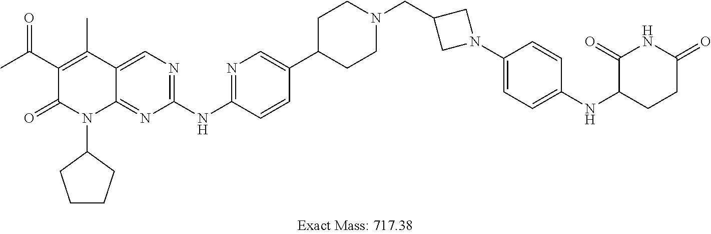 [SMILES: CC(=O)c1c(C)c2cnc(Nc3ccc(C4CCN(CC5CN(c6ccc(NC7CCC(=O)NC7=O)cc6)C5)CC4)cn3)nc2n(C2CCCC2)c1=O] | 3-((4-(3-((4-(6- ((6-acetyl-8- cyclopentyl-5- methyl-7-oxo-7,8- dihydropyrido[2,3- d]pyrimidin-2- yl)amino)pyridin- 3-yl)piperidin-1- yl)methyl) azetidin-1- yl)phenyl)amino) piperidine-2,6- dione | 717.38 | A | A | A | A | A |
| 129 | 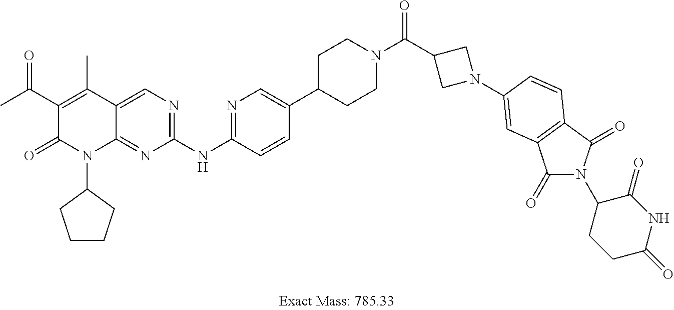 [SMILES: CC(=O)c1c(C)c2cnc(Nc3ccc(C4CCN(C(=O)C5CN(c6ccc7c(c6)C(=O)N(C6CCC(=O)NC6=O)C7=O)C5)CC4)cn3)nc2n(C2CCCC2)c1=O] | 5-(3-(4-(6-((6-acetyl- 8-cyclopentyl- 5-methyl- 7-oxo-7,8- dihydropyrido[2,3- d]pyrimidin-2- yl)amino)pyridin-3- yl)piperidine-1- carbonyl)azetidin- 1-yl)-2-(2,6- dioxopiperidin-3- yl)isoindoline-1,3- dione | 785.33 | B | B | A | B | A |
| 130 | 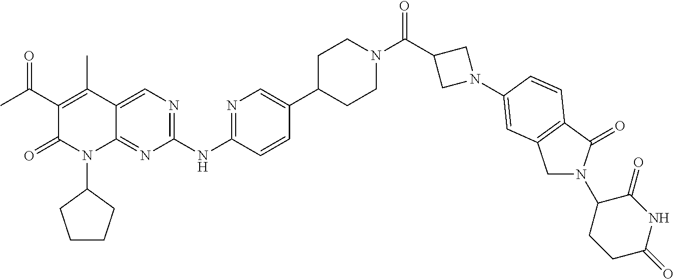 [SMILES: CC(=O)c1c(C)c2cnc(Nc3ccc(C4CCN(C(=O)C5CN(c6ccc7c(c6)CN(C6CCC(=O)NC6=O)C7=O)C5)CC4)cn3)nc2n(C2CCCC2)c1=O] | 3-(5-(3-(4-(6- ((6-acetyl-8- cyclopentyl-5- methyl-7-oxo-7,8- dihydropyrido[2,3- d]pyrimidin-2- yl)amino)pyridin- 3-yl)piperidine-1- carbonyl) azetidin-1-yl)-1- oxoisoindolin-2- yl)piperidine-2,6- dione | 771.35 | A | B | A | A | B |
| 131 | 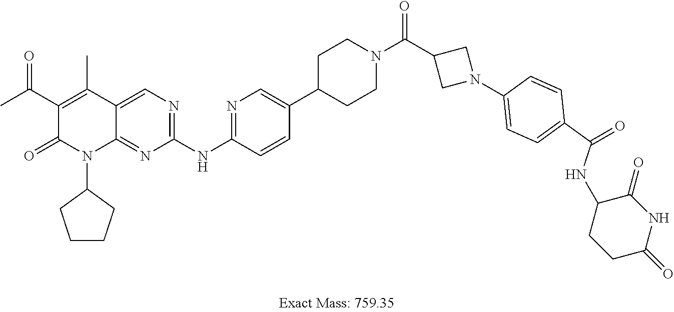 [SMILES: CC(=O)c1c(C)c2cnc(Nc3ccc(C4CCN(C(=O)C5CN(c6ccc(C(=O)NC7CCC(=O)NC7=O)cc6)C5)CC4)cn3)nc2n(C2CCCC2)c1=O] | 4-(3-(4-(6-((6-acetyl- 8-cyclopentyl- 5-methyl- 7-oxo-7,8- dihydropyrido[2,3- d]pyrimidin-2- yl)amino)pyridin- 3-yl)piperidine-1- carbonyl)azetidin- 1-yl)-N-(2,6- dioxopiperidin-3- yl)benzamide | 759.35 | B | B | B | B | B |
| 132 | 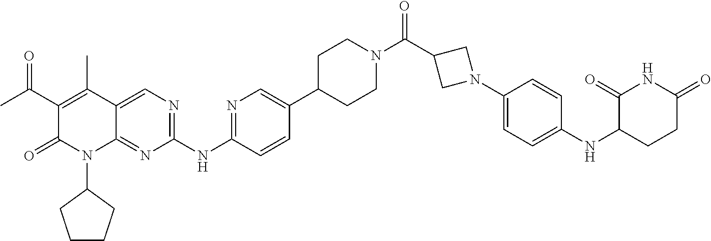 [SMILES: CC(=O)c1c(C)c2cnc(Nc3ccc(C4CCN(C(=O)C5CN(c6ccc(NC7CCC(=O)NC7=O)cc6)C5)CC4)cn3)nc2n(C2CCCC2)c1=O] | 3-((4-(3-(4-(6- ((6-acetyl-8- cyclopentyl-5- methyl-7-oxo-7,8- dibydropyrido[2,3- d]pyrimidin-2- yl)amino)pyridin-3- yl)piperidine-1- carbonyl) azetidin-1- yl)phenyl)amino) piperidine-2,6- dione | 731.35 | A | B | A | A | A |
| 133 | 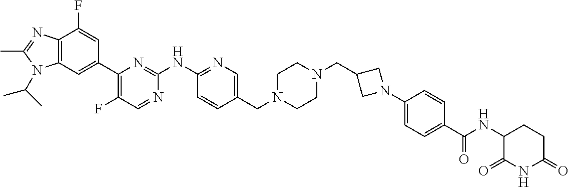 [SMILES: Cc1nc2c(F)cc(-c3nc(Nc4ccc(CN5CCN(CC6CN(c7ccc(C(=O)NC8CCC(=O)NC8=O)cc7)C6)CC5)cn4)ncc3F)cc2n1C(C)C] | N-(2,6- dioxopiperidin- 3-yl)-4-(3- ((4-((6-((5-fluoro- 4-4-fluoro-1- isopropyl-2- methyl-1H- benzo[d]imidazol- 6-yl)pyrimidin-2- ylamino)pyridin-3- yl)methyl) piperazin-1- yl)methyl) azetidin-1- yl)benzamide | 777.37 | A | A | A | A | A |
| 134 | 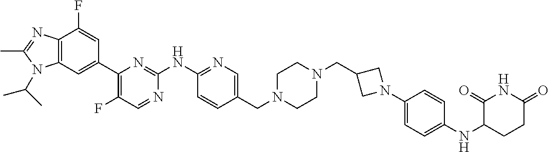 [SMILES: Cc1nc2c(F)cc(-c3nc(Nc4ccc(CN5CCN(CC6CN(c7ccc(NC8CCC(=O)NC8=O)cc7)C6)CC5)cn4)ncc3F)cc2n1C(C)C] | 3-((4-(3-((4-((6- ((5-fluoro-4-(4- fluoro-1-isopropyl- 2-methyl-1H- benzo[d]imidazol- 6-yl)pyrimidin-2- ylamino)pyridin- 3-yl)methyl) piperazin-1-yl) methyl) azetidin-1-yl) phenyl)amino) piperidine-2,6- dione | 749.37 | A | A | A | A | A |
| 135 | 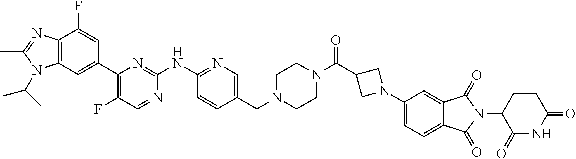 [SMILES: Cc1nc2c(F)cc(-c3nc(Nc4ccc(CN5CCN(C(=O)C6CN(c7ccc8c(c7)C(=O)N(C7CCC(=O)NC7=O)C8=O)C6)CC5)cn4)ncc3F)cc2n1C(C)C] | 2-(2,6- dioxopiperidin- 3-yl)-5-(3-(4- ((6-((5-fluoro- 4-(4-fluoro-1- isopropyl-2- methyl-1H- benzo[d]imidazol- 6-yl)pyrimidin-2- yl)amino)pyridin-3- yl)methyl) piperazine-1- carbonyl)azetidin- 1-yl)isoindoline- 1,3-dione | 817.33 | A | A | A | B | B |
| 136 | 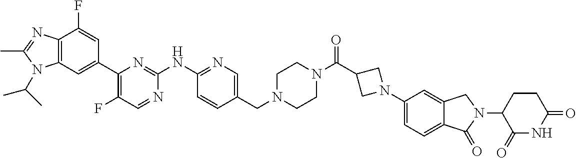 [SMILES: Cc1nc2c(F)cc(-c3nc(Nc4ccc(CN5CCN(C(=O)C6CN(c7ccc8c(c7)CN(C7CCC(=O)NC7=O)C8=O)C6)CC5)cn4)ncc3F)cc2n1C(C)C] | 3-(5-(3-(4-((6- ((5-fluoro-4-(4- fluoro-1-isopropyl- 2-methyl-1H- benzo[d]imidazol-6- yl)pyrimidin-2- yl)amino)pyridin-3- yl)methyl) piperazine-1- carbonyl) azetidin-1-yl)-1- oxoisoindolin-2-yl) piperidine-2,6- dione | 803.35 | A | B | A | B | A |
| 137 | 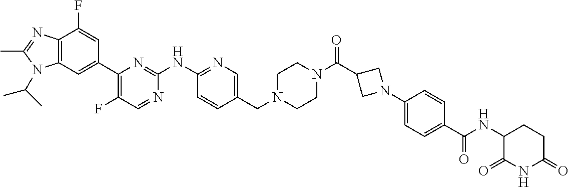 [SMILES: Cc1nc2c(F)cc(-c3nc(Nc4ccc(CN5CCN(C(=O)C6CN(c7ccc(C(=O)NC8CCC(=O)NC8=O)cc7)C6)CC5)cn4)ncc3F)cc2n1C(C)C] | N-(2,6- dioxopiperidin- 3-yl)-4-(3-(4- ((6-((5-fluoro- 4-(4-fluoro-1- isopropyl-2- methyl-1H- benzo[d]imidazol- 6-yl)pyrimidin-2- yl)amino) pyridin-3- yl)methyl) piperazine-1- carbonyl)azetidin- 1-yl)benzamide | 791.35 | B | B | B | B | B |
| 138 | 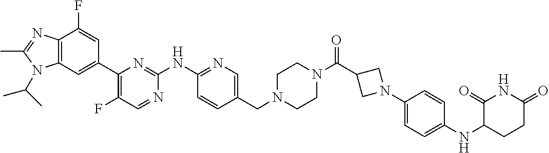 [SMILES: Cc1nc2c(F)cc(-c3nc(Nc4ccc(CN5CCN(C(=O)C6CN(c7ccc(NC8CCC(=O)NC8=O)cc7)C6)CC5)cn4)ncc3F)cc2n1C(C)C] | 3-((4-(3-(4-((6- ((5-fluoro-4-(4- fluoro-1-isopropyl- 2-methyl-1H- benzo[d]imidazol- 6-yl)pyrimidin-2- yl)amino) pyridin-3- yl)methyl) piperazine-1- carbonyl)azetidin-1- yl)phenyl)amino) piperidine-2,6- dione | 763.35 | B | B | B | B | B |
| 139 | 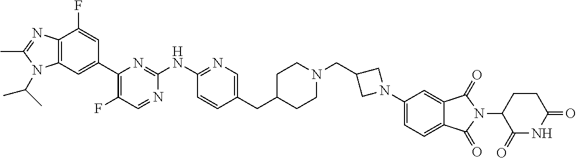 [SMILES: Cc1nc2c(F)cc(-c3nc(Nc4ccc(CC5CCN(CC6CN(c7ccc8c(c7)C(=O)N(C7CCC(=O)NC7=O)C8=O)C6)CC5)cn4)ncc3F)cc2n1C(C)C] | 2-(2,6- dioxopiperidin- 3-yl)-5-(3- ((4-((6-((5- fluoro-4-(4-fluoro- 1-isopropyl- 2-methyl-1H- benzo[d]imidazol- 6-yl)pyrimidin-2- yl)amino)pyridin-3- yl)methyl) piperidin-1- yl)methyl)azetidin- 1-yl)isoindoline- 1,3-dione | 802.35 | A | A | A | A | A |
| 140 | 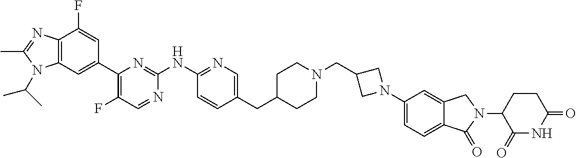 [SMILES: Cc1nc2c(F)cc(-c3nc(Nc4ccc(CC5CCN(CC6CN(c7ccc8c(c7)CN(C7CCC(=O)NC7=O)C8=O)C6)CC5)cn4)ncc3F)cc2n1C(C)C] | 3-(5-(3-((4-((6- ((5-fluoro-4-(4- fluoro-1-isopropyl- 2-methyl-1H- benzo[d]imidazol-6- yl)pyrimidin-2- yl)amino)pyridin- 3-yl)methyl) piperidin-1- yl)methyl) azetidin-1-yl)-1- oxoisoindolin-2- yl)piperidine-2,6- dione | 788.37 | A | A | A | A | A |
| 141 | 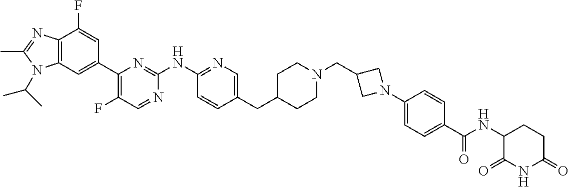 [SMILES: Cc1nc2c(F)cc(-c3nc(Nc4ccc(CC5CCN(CC6CN(c7ccc(C(=O)NC8CCC(=O)NC8=O)cc7)C6)CC5)cn4)ncc3F)cc2n1C(C)C] | N-(2,6- dioxopiperidin- 3-yl)-4-(3- ((4-((6-{(5-fluoro- 4-(4-fluoro-1- isopropyl-2- methyl-1H- benzo[d]imidazol-6- yl)pyrimidin-2- yl)amino) pyridin-3- yl)methyl) piperidin-1- yl)methyl)azetidin- 1-yl)benzamide | 776.37 | A | B | A | B | A |
| 142 | 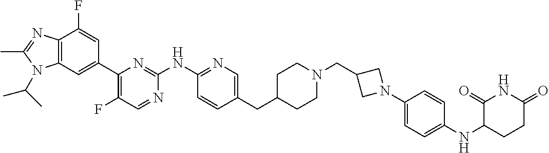 [SMILES: Cc1nc2c(F)cc(-c3nc(Nc4ccc(CC5CCN(CC6CN(c7ccc(NC8CCC(=O)NC8=O)cc7)C6)CC5)cn4)ncc3F)cc2n1C(C)C] | 3-((4-(3-((4-((6- ((5-fluoro-4-(4- fluoro-1-isopropyl- 2-methyl-1H- benzo[d]imidazol- 6-yl)pyrimidin-2- yl)amino)pyridin-3- yl)methyl) piperidin-1- yl)methyl) azetidin-1- yl)phenyl)amino) piperidine-2,6- dione | 748.38 | A | B | A | B | A |
| 143 | 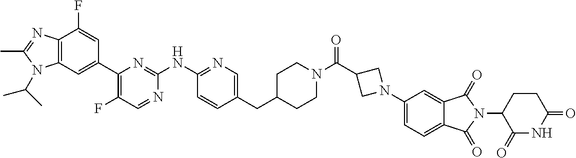 [SMILES: Cc1nc2c(F)cc(-c3nc(Nc4ccc(CC5CCN(C(=O)C6CN(c7ccc8c(c7)C(=O)N(C7CCC(=O)NC7=O)C8=O)C6)CC5)cn4)ncc3F)cc2n1C(C)C] | 2-(2,6- dioxopiperidin- 3-yl)-5-(3-(4- ((6-((5-fluoro- 4-(4-fluoro-1- isopropyl-2- methyl-1H- benzo[d]imidazol- 6-yl)pyrimidin-2- yl)amino) pyridin-3- yl)methyl) piperidine-1- carbonyl)azetidin- 1-yl)isoindoline- 1,3-dione | 816.33 | A | B | A | B | B |
| 144 | 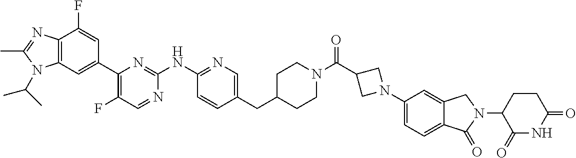 [SMILES: Cc1nc2c(F)cc(-c3nc(Nc4ccc(CC5CCN(C(=O)C6CN(c7ccc8c(c7)CN(C7CCC(=O)NC7=O)C8=O)C6)CC5)cn4)ncc3F)cc2n1C(C)C] | 3-(5-(3-(4-((6- ((5-fluoro-4-(4- fluoro-1-isopropyl- 2-methyl-1H- benzo[d]imidazol- 6-yl)pyrimidin-2- yl)amino)pyridin-3- yl)methyl) piperidine-1- carbonyl)azetidin- 1-yl)-1- oxoisoindolin-2-yl) piperidine-2,6- dione | 802.35 | A | B | A | B | A |
| 145 | 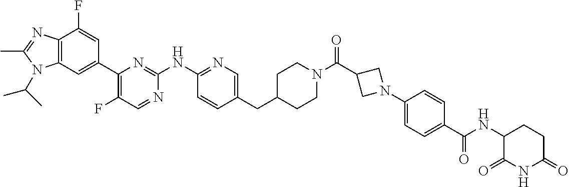 [SMILES: Cc1nc2c(F)cc(-c3nc(Nc4ccc(CC5CCN(C(=O)C6CN(c7ccc(C(=O)NC8CCC(=O)NC8=O)cc7)C6)CC5)cn4)ncc3F)cc2n1C(C)C] | N-(2,6- dioxopiperidin- 3-yl)-4-(3-(4- ((6-((5-fluoro- 4-(4-fluoro-1- isopropyl-2- methyl-1H- benzo[d]imidazol- 6-yl)pyrimidin-2- yl)amino)pyridin-3- yl)methyl) piperidine-1- carbonyl)azetidin-1- yl)benzamide | 790.35 | A | B | B | B | B |
| 146 | 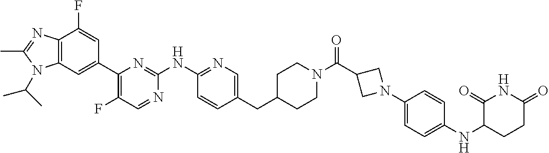 [SMILES: Cc1nc2c(F)cc(-c3nc(Nc4ccc(CC5CCN(C(=O)C6CN(c7ccc(NC8CCC(=O)NC8=O)cc7)C6)CC5)cn4)ncc3F)cc2n1C(C)C] | 3-((4-(3-(4-((6- ((5-fluoro-4-(4- fluoro-1-isopropyl- 2-methyl-1H- benzo[d]imidazol- 6-yl)pyrimidin-2- yl)amino)pyridin-3- yl)methyl) piperidine-1- carbonyl)azetidin-1- yl)phenyl)amino) piperidine-2,6- dione | 762.36 | B | B | C | B | B |
| 147 | 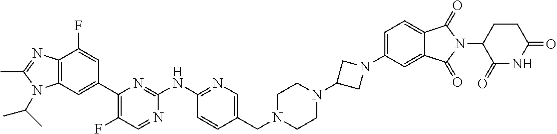 [SMILES: Cc1nc2c(F)cc(-c3nc(Nc4ccc(CN5CCN(C6CN(c7ccc8c(c7)C(=O)N(C7CCC(=O)NC7=O)C8=O)C6)CC5)cn4)ncc3F)cc2n1C(C)C] | 2-(2,6- dioxopiperidin- 3-yl)-5-(3-(4- ((6-((5-fluoro- 4-(4-fluoro-1- isopropyl-2- methyl-1H- benzo[d]imidazol-6- yl)pyrimidin-2- yl)amino)pyridin-3- yl)methyl)piperazin- 1-yl)azetidin-1- yl)isoindoline- 1,3-dione | 789.33 | B | A | B | B | B |
| 148 | 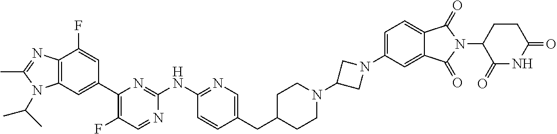 [SMILES: Cc1nc2c(F)cc(-c3nc(Nc4ccc(CC5CCN(C6CN(c7ccc8c(c7)C(=O)N(C7CCC(=O)NC7=O)C8=O)C6)CC5)cn4)ncc3F)cc2n1C(C)C] | 2-(2,6- dioxopiperidin- 3-yl)-5-(3-(4- ((6-((5-fluoro- 4-(4-fluoro-1- isopropyl-2- methyl-1H- benzo[d]imidazol- 6-yl)pyrimidin-2- yl)amino)pyridin-3- yl)methyl)piperidin- 1-yl)azetidin-1- yl)isoindoline-1,3- dione | 788.34 | B | A | B | B | B |
| 149 | 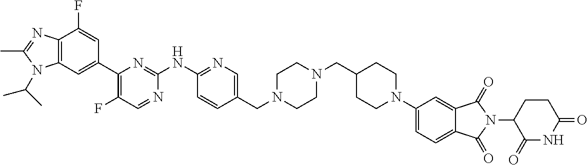 [SMILES: Cc1nc2c(F)cc(-c3nc(Nc4ccc(CN5CCN(CC6CCN(c7ccc8c(c7)C(=O)N(C7CCC(=O)NC7=O)C8=O)CC6)CC5)cn4)ncc3F)cc2n1C(C)C] | 2-(2,6- dioxopiperidin- 3-yl)-5-(4- ((4-((6-((5-fluoro- 4-(4-fluoro-1- isopropyl-2-methyl- 1H-benzo[d] imidazol-6-yl) pyrimidin-2-yl) amino)pyridin- 3-yl)methyl) piperazin-1-yl) methyl)piperidin-1- yl)isoindoline- 1,3-dione | 831.38 | A | A | A | A | A |
| 150 | 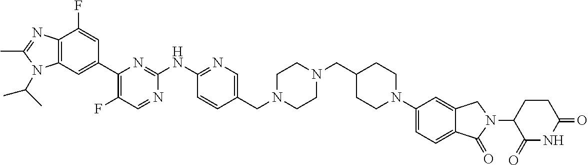 [SMILES: Cc1nc2c(F)cc(-c3nc(Nc4ccc(CN5CCN(CC6CCN(c7ccc8c(c7)CN(C7CCC(=O)NC7=O)C8=O)CC6)CC5)cn4)ncc3F)cc2n1C(C)C] | N-(2,6- dioxopiperidin- 3-yl)-4-(4-(4- ((6-((5-fluoro- 4-(4-fluoro-1- isopropyl-2- methyl-1H- benzo[d]imidazol- 6-yl)pyrimidin-2- yl)amino)pyridin-3- yl)methyl)piperazin- 1-yl)piperidin- 1-yl)benzamide | 791.38 | A | A | A | A | A |
| 151 | 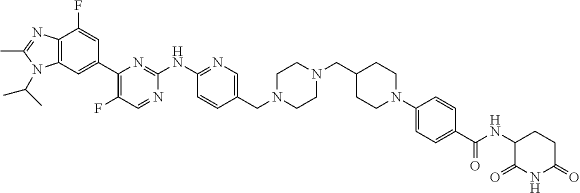 [SMILES: Cc1nc2c(F)cc(-c3nc(Nc4ccc(CN5CCN(CC6CCN(c7ccc(C(=O)NC8CCC(=O)NC8=O)cc7)CC6)CC5)cn4)ncc3F)cc2n1C(C)C] | N-(2,6- dioxopiperidin- 3-yl)-4-(4- ((4-((6-((5-fluoro- 4-(4-fluoro-1- isopropyl-2- methyl-1H- benzo[d]imidazol- 6-yl)pyrimidin-2- yl)amino)pyridin-3- yl)methyl) piperazin-1- yl)methyl)piperidin- 1-yl)benzamide | 805.40 | A | B | A | B | B |
| 152 | 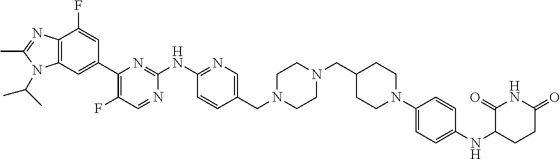 [SMILES: Cc1nc2c(F)cc(-c3nc(Nc4ccc(CN5CCN(CC6CCN(c7ccc(NC8CCC(=O)NC8=O)cc7)CC6)CC5)cn4)ncc3F)cc2n1C(C)C] | 3-((4-(4-((4-((6- ((5-fluoro-4-(4- fluoro-1-isopropyl- 2-methyl-1H- benzo[d]imidazol- 6-yl)pyrimidin-2- yl)amino)pyridin-3- yl)methyl) piperazin-1- yl)methyl) piperidin-1- yl)phenyl)amino) piperidine-2,6- dione | 777.40 | A | A | A | B | A |
| 163 | 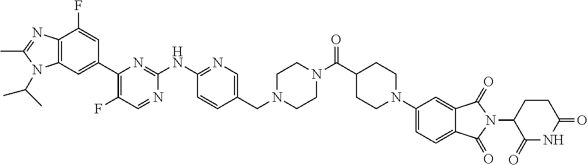 [SMILES: Cc1nc2c(F)cc(-c3nc(Nc4ccc(CN5CCN(C(=O)C6CCN(c7ccc8c(c7)C(=O)N(C7CCC(=O)NC7=O)C8=O)CC6)CC5)cn4)ncc3F)cc2n1C(C)C] | 2-(2,6- dioxopiperidin- 3-yl)-5-(4-(4- ((6-((5-fluoro- 4-(4-fluoro-1- isopropyl-2- methyl-1H- benzo[d]imidazol- 6-yl)pyrimidin-2- yl)amino)pyridin- 3-yl)methyl) piperazine-1- carbonyl)piperidin- 1-yl)isoindoline- 1,3-dione | 845.36 | B | B | B | C | B |
| 154 | 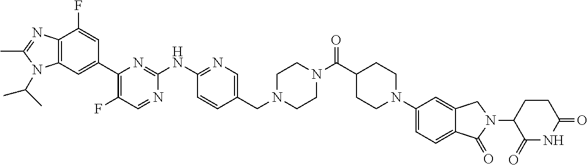 [SMILES: Cc1nc2c(F)cc(-c3nc(Nc4ccc(CN5CCN(C(=O)C6CCN(c7ccc8c(c7)CN(C7CCC(=O)NC7=O)C8=O)CC6)CC5)cn4)ncc3F)cc2n1C(C)C] | 3-(5-(4-(4-((6- ((5-fluoro-4-(4- fluoro-1-isopropyl- 2-methyl-1H- benzo[d]imidazol- 6-yl)pyrimidin-2- ylamino)pyridin-3- yl)methyl) piperazine-1- carbonyl) piperidin-1-yl)-1- oxoisoindolin-2- yl)piperidine-2,6- dione | 831.38 | B | B | B | C | B |
| 155 | 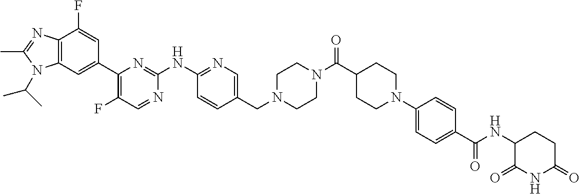 [SMILES: Cc1nc2c(F)cc(-c3nc(Nc4ccc(CN5CCN(C(=O)C6CCN(c7ccc(C(=O)NC8CCC(=O)NC8=O)cc7)CC6)CC5)cn4)ncc3F)cc2n1C(C)C] | N-(2,6- dioxopiperidin- 3-yl)-4-(4-(4- ((6-((5-fluoro- 4-(4-fluoro-1- isopropyl-2- methyl-1H- benzo[d]imidazol- 6-yl)pyrimidin-2- yl)amino)pyridin-3- yl)methyl) piperazine-1- carbonyl)piperidin- 1-yl)benzamide | 819.38 | B | B | C | C | C |
| 156 | 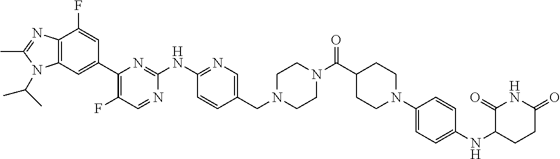 [SMILES: Cc1nc2c(F)cc(-c3nc(Nc4ccc(CN5CCN(C(=O)C6CCN(c7ccc(NC8CCC(=O)NC8=O)cc7)CC6)CC5)cn4)ncc3F)cc2n1C(C)C] | 3-((4-(4-(4-((6- ((5-fluoro-4-(4- fluoro-1-isopropyl- 2-methyl-1H- benzo[d]imidazol- 6-yl)pyrimidin-2- yl)amino)pyridin- 3-yl)methyl) piperazine-1- carbonyl) piperidin-1- yl)phenyl)amino) piperidine-2,6- dione | 791.38 | B | B | B | B | A |
| 157 | 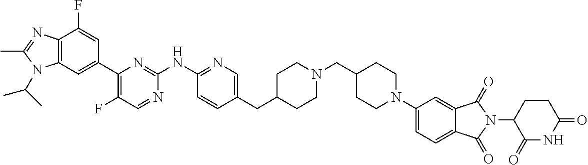 [SMILES: Cc1nc2c(F)cc(-c3nc(Nc4ccc(CC5CCN(CC6CCN(c7ccc8c(c7)C(=O)N(C7CCC(=O)NC7=O)C8=O)CC6)CC5)cn4)ncc3F)cc2n1C(C)C] | 2-(2,6- dioxopiperidin- 3-yl)-5-(4- ((4-((6-((5-fluoro- 4-(4-fluoro-1- isopropyl-2- methyl-1H- benzo[d]imidazol- 6-yl)pyrimidin-2- yl)amino)pyridin- 3-yl)methyl) piperidin-1-yl) methyl)piperidin-1- yl)isoindoline- 1,3-dione | 831.38 | A | A | A | A | A |
| 158 | 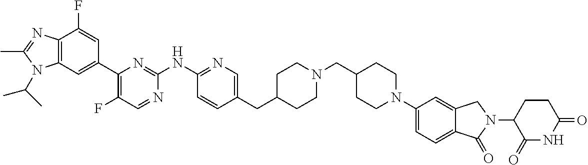 [SMILES: Cc1nc2c(F)cc(-c3nc(Nc4ccc(CC5CCN(CC6CCN(c7ccc8c(c7)CN(C7CCC(=O)NC7=O)C8=O)CC6)CC5)cn4)ncc3F)cc2n1C(C)C] | 3-(5-(4-((4-((6- ((5-fluoro-4-(4- fluoro-1-isopropyl- 2-methyl-1H- benzo[d]imidazol-6- yl)pyrimidin-2- yi)amino)pyridin- 3-yl)methyl) piperidin-1-yl) methyl)piperidin-1- yl)-1-oxoisoindolin- 2-yl)piperidine- 2,6-dione | 816.40 | A | B | A | B | A |
| 159 | 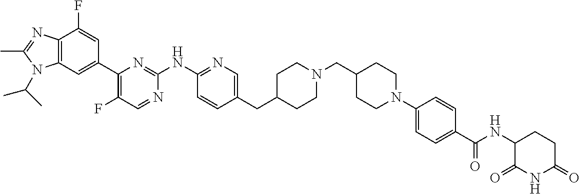 [SMILES: Cc1nc2c(F)cc(-c3nc(Nc4ccc(CC5CCN(CC6CCN(c7ccc(C(=O)NC8CCC(=O)NC8=O)cc7)CC6)CC5)cn4)ncc3F)cc2n1C(C)C] | N-(2,6- dioxopiperidin- 3-yl)-4-(4- ((4-((6-((5-fluoro- 4-(4-fluoro-1- isopropyl-2- methyl-1H- benzo[d]imidazol- 6-yl)pyrimidin-2- yl)amino)pyridin-3- yl)methyl) piperidin-1- yl)methyl)piperidin- 1-yl)benzamide | 804.40 | A | A | A | A | A |
| 160 | 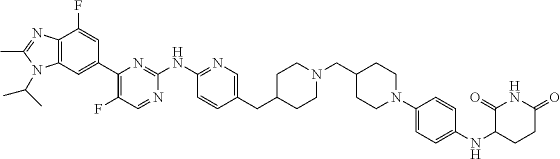 [SMILES: Cc1nc2c(F)cc(-c3nc(Nc4ccc(CC5CCN(CC6CCN(c7ccc(NC8CCC(=O)NC8=O)cc7)CC6)CC5)cn4)ncc3F)cc2n1C(C)C] | 3-((4-(4-((4-((6- ((5-fluoro-4-(4- fluoro-1-isopropyl- 2-methyl-1H- benzo[d]imidazol- 6-yl)pyrimidin-2- yl)amino)pyridin- 3-yl)methyl) piperidin-1- yl)methyl) piperidin-1- yl)phenyl)amino) piperidine-2,6- dione | 776.41 | A | A | A | A | A |
| 161 | 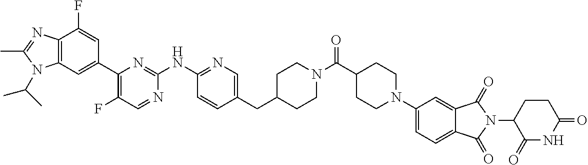 [SMILES: Cc1nc2c(F)cc(-c3nc(Nc4ccc(CC5CCN(C(=O)C6CCN(c7ccc8c(c7)C(=O)N(C7CCC(=O)NC7=O)C8=O)CC6)CC5)cn4)ncc3F)cc2n1C(C)C] | 2-(2,6- dioxopiperidin- 3-yl)-5-(4-(4- ((6-((5-fluoro- 4-(4-fluoro-1- isopropyl-2- methyl-1H- benzo[d]imidazol- 6-yl)pyrimidin-2- yl)amino)pyridin- 3-yl)methyl) piperidine-1- carbonyl) piperidin-1- yl)isoindoline- 1,3-dione | 844.36 | B | B | B | C | B |
| 162 | 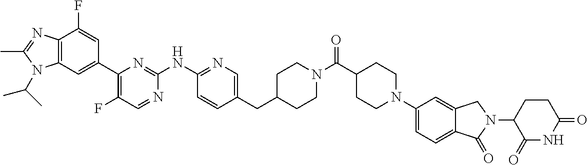 [SMILES: Cc1nc2c(F)cc(-c3nc(Nc4ccc(CC5CCN(C(=O)C6CCN(c7ccc8c(c7)CN(C7CCC(=O)NC7=O)C8=O)CC6)CC5)cn4)ncc3F)cc2n1C(C)C] | 3-(5-(4-(4-((6-((5- fluoro-4-(4-fluoro- 1-isopropyl- 2-methyl- 1H-benzo[d] imnidazol-6-yl) pyrimidin-2- yl)amino) pyridin-3-yl)methyl) piperidine-1- carbonyl)piperidin- 1-yl)-1-oxoiso indolin-2- yl)piperidine- 2,6-dione | 830.38 | B | B | B | C | B |
| 163 | 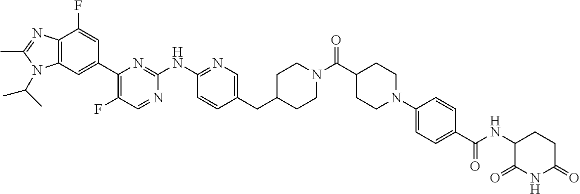 [SMILES: Cc1nc2c(F)cc(-c3nc(Nc4ccc(CC5CCN(C(=O)C6CCN(c7ccc(C(=O)NC8CCC(=O)NC8=O)cc7)CC6)CC5)cn4)ncc3F)cc2n1C(C)C] | N-(2,6- dioxopiperidin- 3-yl)-4-(4-(4- ((6-((5-fluoro- 4-(4-fluoro-1- isopropyl-2- methyl-1H- benzo[d]imidazol-6- yl)pyrimidin-2- yl)amino)pyridin-3- yl)methyl) piperidine-1- carbonyl)piperidin- 1-yl)benzamide | 818.38 | B | B | C | C | C |
| 164 | 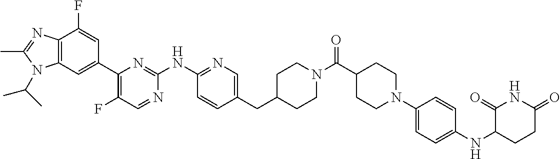 [SMILES: Cc1nc2c(F)cc(-c3nc(Nc4ccc(CC5CCN(C(=O)C6CCN(c7ccc(NC8CCC(=O)NC8=O)cc7)CC6)CC5)cn4)ncc3F)cc2n1C(C)C] | 3-((4-(4-(4-((6- ((5-fluoro-4-(4- fluoro-1-isopropyl- 2-methyl-1H- benzo[d]imidazol-6- yl)pyrimidin-2- yl)amino)pyridin- 3-yl)methyl) piperidine- 1-carbonyl) piperidin-1- yl)phenyl)amino) piperidine-2,6- dione | 790.39 | B | B | B | B | A |
| 165 | 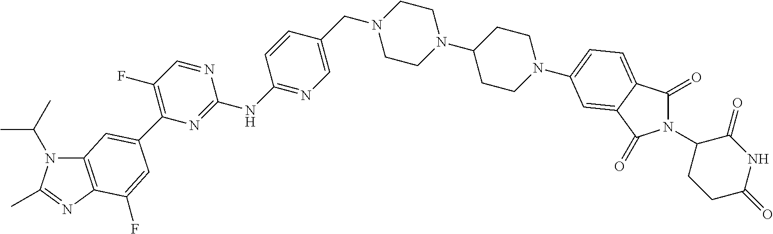 [SMILES: Cc1nc2c(F)cc(-c3nc(Nc4ccc(CN5CCN(C6CCN(c7ccc8c(c7)C(=O)N(C7CCC(=O)NC7=O)C8=O)CC6)CC5)cn4)ncc3F)cc2n1C(C)C] | 2-(2,6- dioxopiperidin- 3-yl)-5-(4-(4- ((6-((5-fluoro- 4-(4-fluoro-1- isopropyl-2- methyl-1H- benzo[d]imidazol- 6-yl)pyrimidin-2- yl)amino)pyridin-3- yl)methyl)piperazin- 1-yl)piperidin- 1-yl)isoindoline- 1,3-dione | 817.36 | A | B | A | B | A |
| 166 | 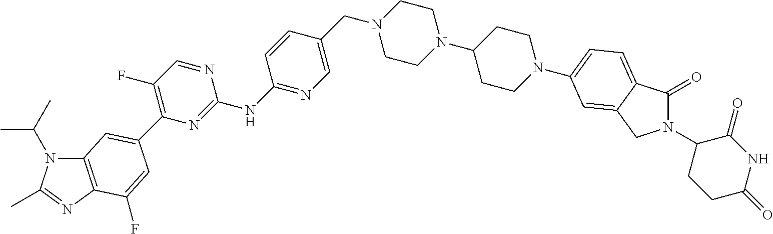 [SMILES: Cc1nc2c(F)cc(-c3nc(Nc4ccc(CN5CCN(C6CCN(c7ccc8c(c7)CN(C7CCC(=O)NC7=O)C8=O)CC6)CC5)cn4)ncc3F)cc2n1C(C)C] | 3-(5-(4-(4-((6- ((5-fluoro-4-(4- fluoro-1-isopropyl- 2-methyl-1H- benzo[d]imidazol-6- yl)pyrimidin-2- yl)amino)pyridin-3- yl)methyl)piperazin- 1-yl)piperidin-1- yl)-1-oxoisoindolin- 2-yl)piperidine- 2,6-dione | 803.38 | A | B | A | B | B |
| 167 | 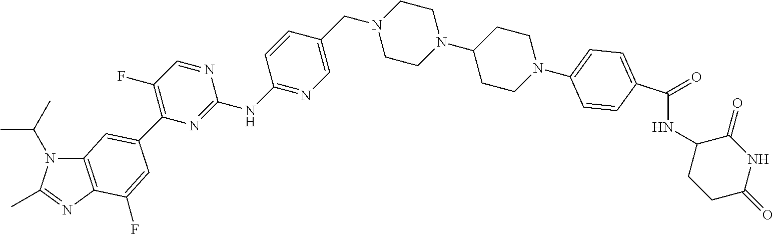 [SMILES: Cc1nc2c(F)cc(-c3nc(Nc4ccc(CN5CCN(C6CCN(c7ccc(C(=O)NC8CCC(=O)NC8=O)cc7)CC6)CC5)cn4)ncc3F)cc2n1C(C)C] | N-(2,6- dioxopiperidin- 3-yl)-4-(4-(4- ((6-((5-fluoro- 4-(4-fluoro-1- isopropyl-2- methyl-1H- benzo[d]imidazol- 6-yl)pyrimidin-2- yl)amino)pyridin-3- yl)methyl)piperazin- 1-yl)piperidin- 1-yl)benzamide | 791.38 | B | B | B | B | B |
| 168 | 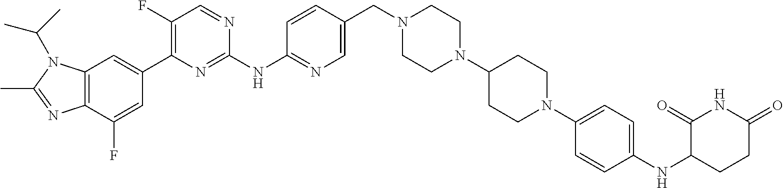 [SMILES: Cc1nc2c(F)cc(-c3nc(Nc4ccc(CN5CCN(C6CCN(c7ccc(NC8CCC(=O)NC8=O)cc7)CC6)CC5)cn4)ncc3F)cc2n1C(C)C] | 3-((4-(4-(4-((6- ((5-fluoro-4-(4- fluoro-1-isopropyl- 2-methyl-1H- benzo[d]imidazol- 6-yl)pyrimidin-2- yl)amino)pyridin-3- yl)methyl)piperazin- 1-yl)piperidin- 1-yl)phenyl)amino) piperidine-2,6- dione | 763.39 | A | B | A | A | A |
| 169 | 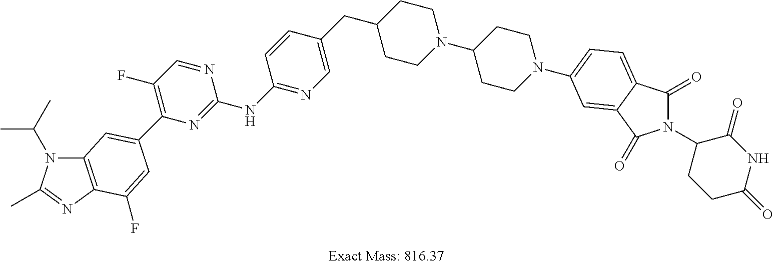 [SMILES: Cc1nc2c(F)cc(-c3nc(Nc4ccc(CC5CCN(C6CCN(c7ccc8c(c7)C(=O)N(C7CCC(=O)NC7=O)C8=O)CC6)CC5)cn4)ncc3F)cc2n1C(C)C] | 2-(2,6- dioxopiperidin- 3-yl)-5-(4- ((6-((5-fluoro- 4-(4-fluoro-1- isopropyl-2- methyl-1H- benzo[d]imidazol-6- yl)pyrimidin-2- yl)amino)pyridin-3- yl)methyl)-[1,4′- bipiperidin]-1′- yl)isoindoline-1,3- dione | 816.37 | A | B | A | B | A |
| 170 | 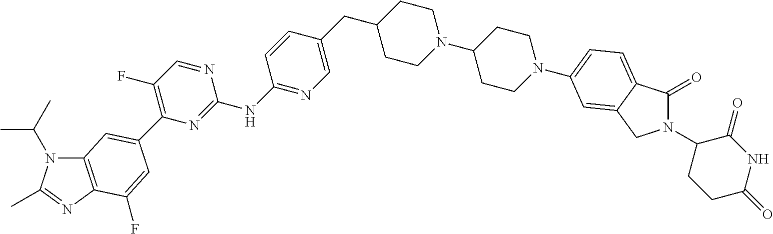 [SMILES: Cc1nc2c(F)cc(-c3nc(Nc4ccc(CC5CCN(C6CCN(c7ccc8c(c7)CN(C7CCC(=O)NC7=O)C8=O)CC6)CC5)cn4)ncc3F)cc2n1C(C)C] | 3-(5-(4-((6- ((5-fluoro- 4-(4-fluoro-1- isopropyl-2- methyl-1H- benzo[d]imidazol-6- yl)pyrimidin-2- yl)amino)pyridin-3- yl)methyl)-[1,4′- bipiperidin]-1′-yl)- 1-oxoisoindolin- 2-yl)piperidine- 2,6-dione | 802.39 | A | A | A | A | B |
| 171 | 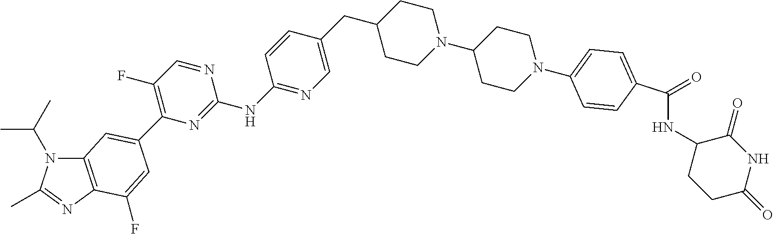 [SMILES: Cc1nc2c(F)cc(-c3nc(Nc4ccc(CC5CCN(C6CCN(c7ccc(C(=O)NC8CCC(=O)NC8=O)cc7)CC6)CC5)cn4)ncc3F)cc2n1C(C)C] | N-(2,6- dioxopiperidin- 3-yl)-4-(4- ((6-((5-fluoro- 4-(4-fluoro-1- isopropyl-2- methyl-1H- benzo[d]imidazol-6- yl)pyrimidin-2- yl)amino)pyridin-3- yl)methyl)-[1,4′- bipiperidin]-1′-yl) benzamide | 790.39 | B | B | B | B | B |
| 172 | 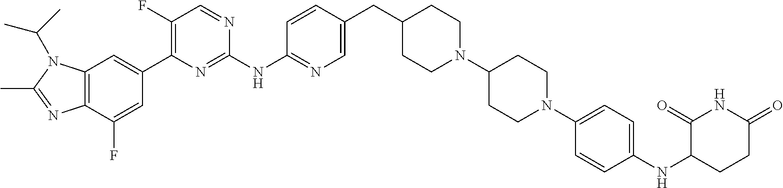 [SMILES: Cc1nc2c(F)cc(-c3nc(Nc4ccc(CC5CCN(C6CCN(c7ccc(NC8CCC(=O)NC8=O)cc7)CC6)CC5)cn4)ncc3F)cc2n1C(C)C] | 3-((4-(4-((6-((5- fluoro-4-(4-fluoro- 1-isopropyl-2- methyl-1H- benzo[d]imidazol- 6-yl)pyrimidin-2- yl)amino)pyridin-3- yl)methyl)-[1,4′- bipiperidin]-1′- yl)phenyl)amino) piperidine-2,6- dione | 762.39 | A | B | A | B | A |

Antiproliferation in mantle cell lymphoma cell (Mino or JeKo-1): A: IC₅₀ <50 nM; B: 50 nM <1C₅₀ <200 nM; C: IC₅₀ >200 nM
CDK4/6 degradation (DC₅₀): A: <50 nM; B: 50-200 nM; C: >200 nM
CDK4/6 degradation (Dₘₐₓ): A: >80%; B: >60%; C >50%; D <50%

### Pharmaceutical Compositions

This specification also describes, in part, a composition which comprises a compound of Formula (I), or a derivative, tautomer, stereoisomer, mixture of stereoisomers, pharmaceutically acceptable salt, or solvate thereof.

This specification also describes, in part, a pharmaceutical composition which comprises a compound of Formula (I), or a pharmaceutically acceptable salt thereof, and at least one pharmaceutically acceptable excipient.

This specification also describes, in part, a compound of Formula (I), or a pharmaceutically acceptable salt thereof, for use in reducing the level or activity of a target protein (e.g., a cyclin dependent kinase).

This specification also describes, in part, a compound of Formula (I), or a pharmaceutically acceptable salt thereof, for use in inhibiting a target protein (e.g., a cyclin dependent kinase).

This specification also describes, in part, a compound of Formula (I), or a pharmaceutically acceptable salt thereof, for use in therapy.

This specification also describes, in part, a compound of Formula (I), or a pharmaceutically acceptable salt thereof, for use in the treatment of cancer.

This specification also describes, in part, a method for treating cancer in a warm-blooded animal in need of such treatment, which comprises administering to the warm-blooded animal a therapeutically effective amount of a compound of Formula (I), or a pharmaceutically acceptable salt thereof.

In one embodiment, the disclosure provides for a pharmaceutical composition comprising at least one compound of one of formula (I) or a pharmaceutically acceptable salt or solvate thereof. In an embodiment, the pharmaceutical compound is for use in treatment of a proliferative disease, such as a cancer, for example, breast cancer. A further embodiment may provide a method of treating breast cancer comprising administering to a subject in need of treatment or amelioration a compound according to any one of the preceding paragraphs. In some embodiments a compound as presented above is used in the preparation of a medicament for treatment of breast cancer in a patient or subject, such as a human or animal.

The pharmaceutical compositions of the disclosure can be in any form known to those of skill in the art, and a suitable dosage form of the compound(s) can be administered by an appropriate route. For instance, in some embodiments the pharmaceutical compositions are in a form of a product for oral delivery, said product form being selected from a group consisting of a concentrate, dried powder, liquid, capsule, pellet, and pill. In other embodiments, the pharmaceutical compositions of the disclosure are in the form of a product for parenteral administration including intravenous, intradermal, intramuscular, intraarticular, intra-synovial, intrasternal, intrathecal and subcutaneous administration. The compounds described herein may be administered as a single dose or a divided dose over a period of time. The pharmaceutical compositions disclosed herein may also further comprise carriers, binders, diluents, and excipients. The described carriers, diluents and excipients may include dried corn starch or lactose, the binder may include microcrystalline cellulose, gum tragacanth or gelatin, in addition, the excipients may also include a dispersing agent, a lubricant, a glidant, a sweetening agent or a flavoring agent.

Also, in other aspects, the disclosure provides a method of modulating a kinase, comprising contacting the kinase with a bifunctional compound disclosed herein, or a pharmaceutically acceptable salt, hydrate, solvate, prodrug, stereoisomer, or tautomer thereof, or with a pharmaceutical composition disclosed herein. In some embodiments, the kinase is CDK4. In other embodiments, the kinase is CDK6. In other embodiments, the kinase is CDK4 and CDK6.

The disclosed compounds can be used to slow the rate of primary tumor growth. The disclosed compounds can also be used to prevent, abate, minimize, control, and/or lessen tumor metastasis in humans and animals. The disclosed compounds when administered to a subject in need of treatment can be used to stop the spread of cancer cells. As such, the compounds disclosed herein can be administered as part of a combination therapy with one or more drugs or other pharmaceutical agents. When used as part of the combination therapy, the decrease in metastasis and reduction in primary tumor growth afforded by the disclosed compounds allows for a more effective and efficient use of any pharmaceutical or drug therapy being used to treat the patient. In addition, control of metastasis by the disclosed compound affords the subject a greater ability to concentrate the disease in one location.

The following are non-limiting examples of cancers that can be treated by the disclosed methods and compositions: Acute Lymphoblastic; Acute Myeloid Leukemia; Adrenocortical Carcinoma; Adrenocortical Carcinoma, Childhood; Appendix Cancer; Basal Cell Carcinoma; Bile Duct Cancer, Extrahepatic; Bladder Cancer; Bone Cancer; Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Central Nervous System Embryonal Tumors; Cerebellar Astrocytoma; Cerebral Astrocytotna/Malignant Glioma; Craniopharyngioma; Ependymoblastoma; Ependymoma; Medulloblastoma; Medulloepithelioma; Pineal Parenchymal Tumors of intermediate Differentiation; Supratentorial Primitive Neuroectodermal Tumors and Pineoblastoma; Visual Pathway and Hypothalamic Glioma; Brain and Spinal Cord Tumors; Breast Cancer, Bronchial Tumors; Burkitt Lymphoma; Carcinoid Tumor, Carcinoid Tumor, Gastrointestinal; Central Nervous System Atypical Teratoid/Rhabdoid Tumor; Central Nervous System Embryonal Tumors; Central Nervous System Lymphoma; Cerebellar Astrocytoma Cerebral Astrocytoma/Malignant Glioma, Childhood; Cervical Cancer; Chordoma, Childhood; Chronic Lymphocytic Leukemia; Chronic Myelogenous Leukemia; Chronic Myeloproliferative Disorders; Colon Cancer; Colorectal Cancer; Craniopharyngioma; Cutaneous T-Cell Lymphoma; Esophageal Cancer; Ewing Family of Tumors; Extragonadal Germ Cell Tumor; Extrahepatic Bile Duct Cancer; Eye Cancer, intraocular Melanoma; Eye Cancer, Retinoblastoma; Gallbladder Cancer; Gastric (Stomach) Cancer; Gastrointestinal Carcinoid Tumor; Gastrointestinal Stromal Tumor (GIST); Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer, Histiocytosis, Langerhans Cell; Hodgkin Lymphoma; Hypopharyngeal Cancer; Hypothalamic and Visual Pathway Glioma; intraocular Melanoma; Islet Cell Tumors; Kidney (Renal Cell) Cancer; Langerhans Cell Histiocytosis; Laryngeal Cancer; Leukemia, Acute Lymphoblastic; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin; Lymphoma, Non-Hodgkin; Lymphoma, Primary Central Nervous System; Macroglobulinemia, Waldenstrom; Malignant Fibrous Histiocytoma of Bone and Osteosarcoma; Medulloblastoma; Melanoma; Melanoma, intraocular (Eye); Merkel Cell Carcinoma; Mesothelioma; Metastatic Squamous Neck Cancer with Occult Primary; Mouth Cancer; Multiple Endocrine Neoplasia Syndrome, (Childhood); Multiple Myeloma/Plasma Cell Neoplasm; Mycosis; Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Neuroblastoma; Non-Small Cell Lung Cancer; Oral Cancer; Oral Cavity Cancer; Oropharyngeal Cancer Osteosarcoma and Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell Tumors; Papillomatosis; Parathyroid Cancer; Penile Cancer; Pharyngeal Cancer; Pheochromocytoma; Pineal Parenchymal Tumors of Intermediate Differentiation; Pineoblastoma and Supratentorial Primitive Neuroectodermal Tumors; Pituitary Tumor; Plasma Celt Neoplasm/Multiple Myeloma; Pleuropulmonary Blastoma; Primary Central Nervous System Lymphoma; Prostate Cancer; Rectal Cancer; Renal Cell (Kidney) Cancer Renal Pelvis and Ureter, Transitional Cell Cancer; Respiratory Tract Carcinoma Involving the NUT Gene on Chromosome 15; Retinoblastoma; Rhabdomyosarcoma; Salivary Gland Cancer; Sarcoma, Ewing Family of Tumors; Sarcoma, Kaposi; Sarcoma, Soft Tissue; Sarcoma, Uterine; Sezary Syndrome; Skin Cancer (Nonmelanoma); Skin Cancer (Melanoma); Skin Carcinoma, Merkel Cell; Small Cell Lung Cancer; Small Intestine Cancer; Soft Tissue Sarcoma; Squamous Cell Carcinoma, Squamous Neck Cancer with Occult Primary, Metastatic; Stomach (Gastric) Cancer: Supratentorial Primitive Neuroectodermal Tumors; T-Cell Lymphoma, Cutaneous; Testicular Cancer; Throat Cancer; Thymoma and Thymic Carcinoma; Thyroid Cancer; Transitional Cell Cancer of the Renal Pelvis and Ureter; Trophoblastic Tumor, Gestational; Urethral Cancer; Uterine Cancer, Endometrial; Uterine Sarcoma; Vaginal Cancer; Vulvar Cancer; Waldenstrom Macroglobulinemia; and Wilms Tumor.

### Methods of Treatment

The methods for treating a clinical indication by the CDK4/6 degrading compounds disclosed herein, may be effectuated by administering a therapeutically effective amount of the CDK4/6 degrading compounds to a patient in need thereof, this therapeutically effective amount may comprise administration of the prodrug to the patient at 1 mg/kg/day, 2 mg/kg/day, 3 mg/kg/day, 4 mg/kg/day, 5 mg/kg/day, 10 mg/kg/day and 20 mg/kg/day. Alternatively, amounts ranging from about 0.001 mg/kg/day to about 0.01 mg/kg/day, or about 0.01 mg/kg/day to about 0.1 mg/kg/day, or about 0.1 mg/kg/day to about 1 mg/kg/day, or about 1 mg/kg/day to 10 mg/kg/day, or about 10 mg/kg/day to about 100 mg/kg/day are also contemplated.

A further object of the disclosure is a kit, comprising a composition containing at least one CDK4/6 degrading compound for treatment and prevention of cancer and cancer related morbidities. The composition of the kit may comprise at least one carrier, at least one binder, at least one diluent, at least one excipient, at least one other therapeutic agent, or mixtures thereof. The kit may be designed, developed, distributed, or sold as a unit for performing the methods of the invention and to deliver the drugs to the targeted cells for the treatment and prevention of cancer and related diseases. The kits may also include instructions to customers for proper usage of the kit to treat patients exhibiting the symptoms of the desired disease, e.g., breast cancer.

One aspect of the disclosure is the compounds disclosed herein as well as the intermediates as used for their synthesis, and the synthetic scheme for the preparation of the disclosed final compounds and the intermediates resulted before the final compound is generated.

While certain features of this invention shown and described below are pointed out in the annexed claims, the invention is not intended to be limited to the details specified, since a person of ordinary skill in the relevant art will understand that various omissions, modifications, substitutions, and changes in the forms and details of the invention illustrated and in its operation may be made without departing in any way from the spirit of the invention. No feature of the invention is critical or essential unless it is specifically stated as being “critical” or “essential”.

These and other features, aspects, and advantages of embodiments of the disclosure will become more evident with regard to the following descriptions, claims, and accompanying drawings explained below.

Another object of the disclosure is to provide a composition, for example a pharmaceutical composition, comprising at least one CDK4/6 degrader compound in an amount effective for the indication of proliferative diseases such as cancer, including but not limited to breast cancer. In an embodiment, the cancer is a breast cancer, such as metastatic ER+, HER2-breast cancer.

In an embodiment, the object of such treatment is to degrade CDK4/6 and/or inhibit CDK4/6-dependent proliferation of a cell. In a further embodiment, said object is to inhibit CDK4/6-induced proliferation of a cell by a mechanism selected from CDK4/6 degradation.

As used herein, “treating” means administering to a subject a pharmaceutical composition to ameliorate, reduce or lessen the symptoms of a disease. As used herein, “treating” or “treat” describes the management and care of a subject for the purpose of combating a disease, condition, or disorder and includes the administration of a compound disclosed herein, or a pharmaceutically acceptable salt, polymorph or solvate thereof, to alleviate the symptoms or complications of a disease, condition or disorder, or to eliminate the disease, condition or disorder. The term “treat” may also include treatment of a cell in vitro or an animal model. As used herein, “subject” or “subjects” refers to any animal, such as mammals including rodents (e.g., mice or rats), dogs, primates, lemurs or humans.

Treating cancer may result in a reduction in size of a tumor. A reduction in size of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater, even more preferably, reduced by 50% or greater; and most preferably, reduced by greater than 75% or greater. Size of a tumor may be measured by any reproducible means of measurement. The size of a tumor may be measured as a diameter of the tumor.

Treating cancer may result in a reduction in tumor volume. Preferably, after treatment, tumor volume is reduced by 5% or greater relative to its size prior to treatment; more preferably, tumor volume is reduced by 10% or greater; more preferably, reduced by 20% or greater more preferably, reduced by 30% or greater, more preferably, reduced by 40% or greater; even more preferably, reduced by 50% or greater, and most preferably, reduced by 75% or greater. Tumor volume may be measured by any reproducible means of measurement.

Treating cancer may result in a decrease in number of tumors. Preferably, after treatment, tumor number is reduced by 5% or greater relative to number prior to treatment; more preferably, tumor number is reduced by 10% or greater; more preferably, reduced by 20% or greater, more preferably, reduced by 30% or greater, more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer may result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater more preferably, reduced by 20% or greater; more preferably, reduced by 30% or greater, more preferably, reduced by 40% or greater, even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer may result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in increase in average survival time of a population of treated subjects in comparison to a population receiving monotherapy with a drug that is not a compound disclosed herein, or a pharmaceutically acceptable salt thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in a decrease in the mortality rate of a population of treated subjects in comparison to a population receiving carrier alone. Treating cancer may result in a decrease in the mortality rate of a population of treated subjects in comparison to an untreated population. Treating cancer may result in a decrease in the mortality rate of a population of treated subjects in comparison to a population receiving monotherapy with a drug that is not a compound disclosed herein, or a pharmaceutically acceptable salt, prodrug, metabolite, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer may result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate may be measured according to a change in tumor diameter per unit time.

Treating cancer may result in a decrease in tumor regrowth, for example, following attempts to remove it surgically. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder may result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder may result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells may be equivalent to the mitotic index.

Treating or preventing a cell proliferative disorder may result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder may result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology may be measured by microscopy. e.g., using an inverted tissue culture microscope. An abnormal cellular morphology may take the form of nuclear pleiomorphism.

### EXAMPLES

Hereby are provided non-limiting examples of embodiments of compounds disclosed herein. The examples and preparations provided below further illustrate and exemplify the compounds as disclosed herein and methods of preparing such compounds. It is to be understood that the scope of the disclosure is not limited in any way by the scope of the following examples and preparations. Unless stated otherwise, starting materials were commercially available. All solvents and commercial reagents were of laboratory grade and were used as received.

### Example 1; General Synthesis

The chemical entities described herein can be synthesized according to one or more illustrative schemes herein and/or techniques well known in the art. Unless specified to the contrary, the reactions described herein take place at atmospheric pressure, generally within a temperature range from about −10° C. to about 200° C. Further, except as otherwise specified, reaction times and conditions are intended to be approximate, e.g., taking place at about atmospheric pressure within a temperature range of about −10° C. to about 200° C. over a period that can be, for example, about 1 to about 24 hours; reactions left to run overnight in some embodiments can average a period of about 16 hours. Isolation and purification of the chemical entities and intermediates described herein can be implemented, if desired, by any suitable separation or purification procedure such as, for example, filtration, extraction, crystallization, column chromatography, thin-layer chromatography or thick-layer chromatography, or a combination of these procedures. See, e.g., Carey et al. Advanced Organic Chemistry, 3rd Ed., 1990 New York: Plenum Press; Mundy et al., Name Reaction and Reagents in Organic Synthesis, 2nd Ed., 2005 Hoboken, N.J.: J. Wiley & Sons. Specific illustrations of suitable separation and isolation procedures are given by reference to the examples hereinbelow. However, other equivalent separation or isolation procedures can also be used.

In all of the methods, it is well understood that protecting groups for sensitive or reactive groups may be employed where necessary, in accordance with general principles of chemistry. Protecting groups are manipulated according to standard methods of organic synthesis (T. W. Greene and P. G. M. Wuts (1999) Protective Groups in Organic Synthesis, 3rd Ed., John Wiley & Sons). These groups may be removed at a convenient stage of the compound synthesis using methods that are readily apparent to those skilled in the art.

In some embodiments, disclosed compounds can generally be synthesized by an appropriate combination of generally well-known synthetic methods. Techniques useful in synthesizing these chemical entities are both readily apparent and accessible to those of skill in the relevant art, based on the instant disclosure. Many of the optionally substituted starting compounds and other reactants are commercially available, or can be readily prepared by those skilled in the art using commonly employed synthetic methodology.

The discussion below is offered to illustrate certain of the diverse methods available for use in making the disclosed compounds and is not intended to limit the scope of reactions or reaction sequences that can be used in preparing the compounds provided herein. The skilled artisan will under-stand that standard atom valencies apply to all compounds disclosed herein in genus or named compound for unless otherwise specified.

### Example 2. Preparation of Compound 1, 3, 9, 11 & 19

As illustrative examples of compounds of formula (I), the following compounds, denoted Compounds 1, 3, 9, 11 & 19, were synthesized:

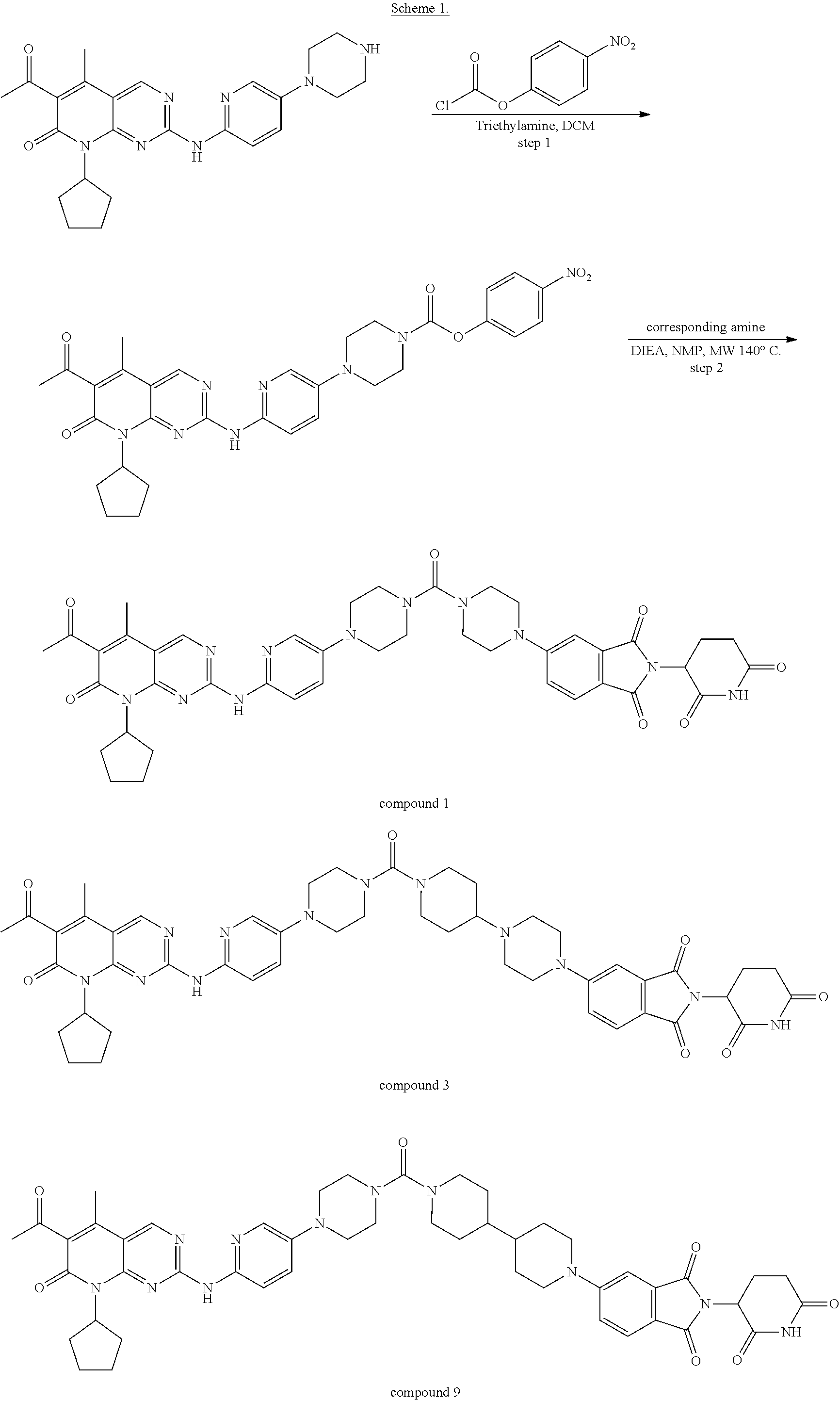

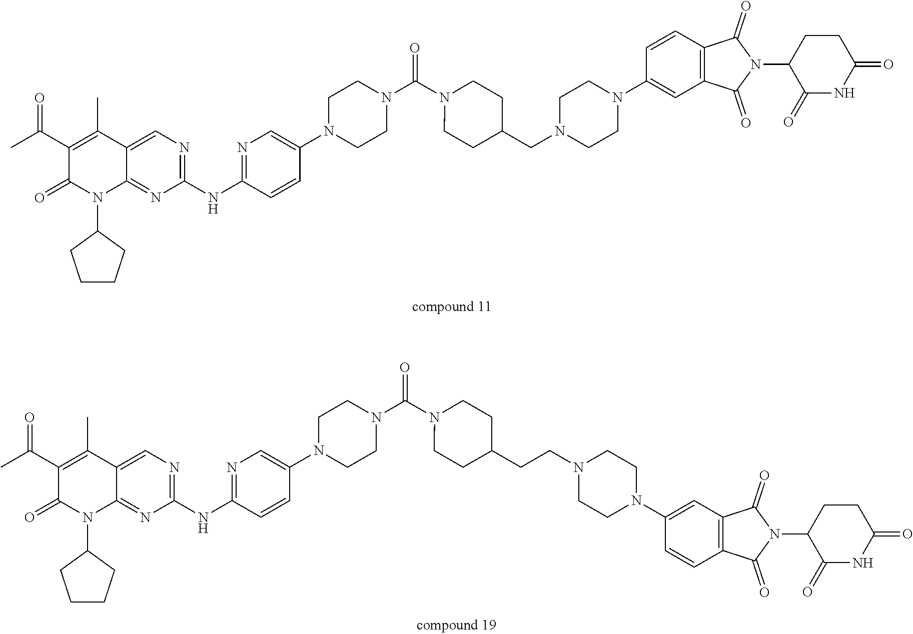 [SMILES: CC(=O)c1c(C)c2cnc(Nc3ccc(N4CCN(C(=O)N5CCC(CN6CCN(c7ccc8c(c7)C(=O)N(C7CCC(=O)NC7=O)C8=O)CC6)CC5)CC4)cn3)nc2n(C2CCCC2)c1=O ; CC(=O)c1c(C)c2cnc(Nc3ccc(N4CCN(C(=O)N5CCC(CCN6CCN(c7ccc8c(c7)C(=O)N(C7CCC(=O)NC7=O)C8=O)CC6)CC5)CC4)cn3)nc2n(C2CCCC2)c1=O]

### Step 1. 4-nitrophenyl 4-(6-((6-acetyl-8-cyclopentyl-5-methyl-7-oxo-7,8-dihydropyrido[2,3-d]pyrimidin-2-yl)amino)pyridin-3-yl)piperazine-1-carboxylate

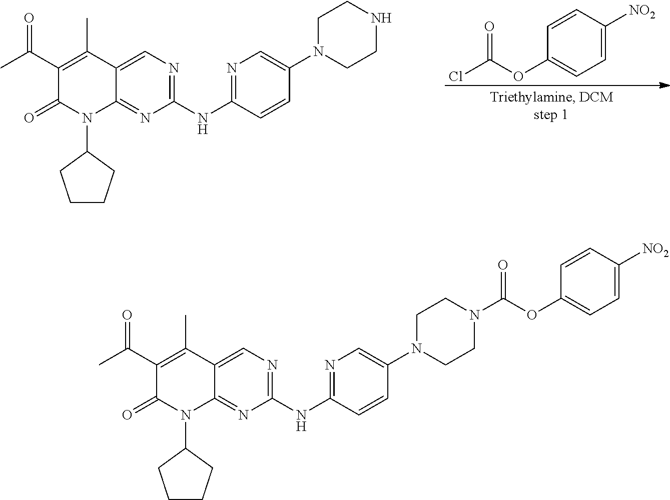 [SMILES: CC(=O)c1c(C)c2cnc(Nc3ccc(N4CCNCC4)cn3)nc2n(C2CCCC2)c1=O ; O=C(Cl)Oc1ccc([N+](=O)[O-])cc1 ; CC(=O)c1c(C)c2cnc(Nc3ccc(N4CCN(C(=O)Oc5ccc([N+](=O)[O-])cc5)CC4)cn3)nc2n(C2CCCC2)c1=O]

To a stirred suspension of 6-acetyl-8-cyclopentyl-5-methyl-2-((5-(piperazin-1-yl)pyridin-2-yl)amino)pyrido[2,3-d]pyrimidin-7(8H)-one (0.894 grams (g), 2 millimoles (mmol)) and triethylamine (0.40 g, 4 mmol) in 10 mL of dichloromethane (DCM) was added 4-nitrophenyl carbonochloridate (0.24 g, 2.4 mmol) in one portion at room temperature (rt). After stirring for 1 hour (h), the suspension became a clear yellow solution. The solvent was removed in vacuo. The residue was subjected to silica gel column chromatography to provide 0.55 g of 4-nitrophenyl 4-(6-((6-acetyl-8-cyclopentyl-5-methyl-7-oxo-7,8-dihydropyrido[2,3-d]pyrimidin-2-yl)amino)pyridin-3-yl)piperazine-1-carboxylate as red solid. LCMS [M+H] 613. ¹H NMR (CDCl3, 300 MHz): δ 1.63-2.05 (8H, mm, 2.39 (3H, s), 2.51 (3H, s), 3.01 (4H, d), 3.62 (4H, d), 4.72 (1H, d), 7.14 (1H, d), 7.38 (1H, d), 7.50-7.68 (3H, m), 8.16 (2H, d), 8.46 (1H, s).

### Step 2. 5-(4-(4-(6-((6-acetyl-8-cyclopentyl-5-methyl-7-oxo-7,8-dihydropyrido[2,3-d]pyrimidin-2-yl)amino)pyridin-3-yl)piperazine-1 carbonyl)piperazin-1-yl)-2-(2,6-dioxopiperidin-3-yl)isoindoline-1,3-dione (Compound 1)

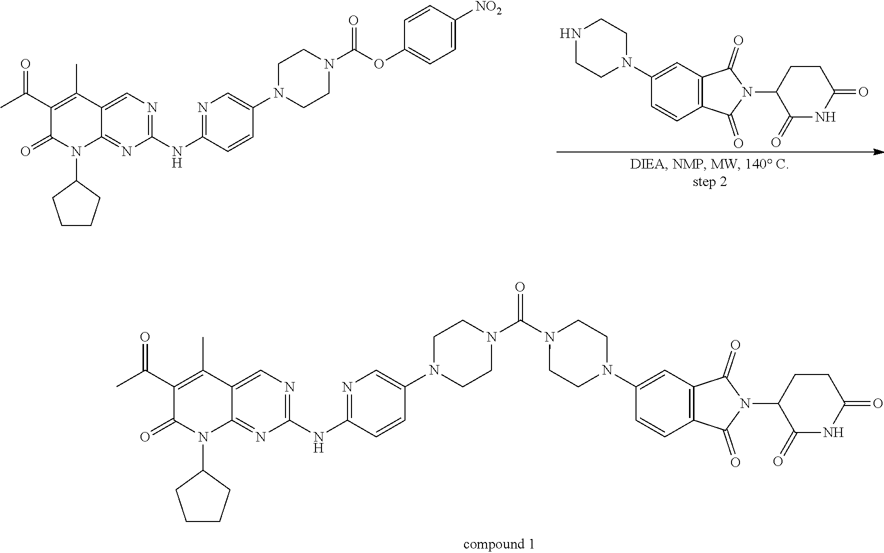 [SMILES: CC(=O)c1c(C)c2cnc(Nc3ccc(N4CCN(C(=O)Oc5ccc([N+](=O)[O-])cc5)CC4)cn3)nc2n(C2CCCC2)c1=O ; O=C1CCC(N2C(=O)c3ccc(N4CCNCC4)cc3C2=O)C(=O)N1 ; CC(=O)c1c(C)c2cnc(Nc3ccc(N4CCN(C(=O)N5CCN(c6ccc7c(c6)C(=O)N(C6CCC(=O)NC6=O)C7=O)CC5)CC4)cn3)nc2n(C2CCCC2)c1=O]

A mixture of 4-nitrophenyl 4-(6-((6-acetyl-8-cyclopentyl-5-methyl-7-oxo-7,8-dihydropyrido[2,3-d]pyrimidin-2-yl)amino)pyridin-3-yl)piperazine-1-carboxylate (122 milligrams (mg), 0.2 mmol), diisopropylethylamine (DIEA) (130 mg, 1 mmol) and 2-(2,6-dioxopiperidin-3-yl)-5-(piperazin-1-yl)isoindoline-1,3-dione (68 mg, 0.2 mmol) in 3 milliliters (mL) of n-methyl pyrrole (NMP) was stirred in a microwave (MW) reactor and heated to 140° C. for 1 h. After cooling to room temperature (rt), the mixture was subjected onto silica gel column chromatography to provide crude product. The crude product was further purified by prep-high pressure liquid chromatography (HPLC) by using C18 column eluted with 0.5% formic acid (FA) in water/MeCN. After lyophilization, 35 mg of 5-(4-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperazin-1-yl)-2-(2,6-dioxopiperidin-3-yl)isoindoline-1,3-dione (compound 1) was obtained as yellow solid.

LCMS [M+H] 816. ¹H NMR (DMSO-d6, 300 MHz): δ 1.62-2.14 (10H, m), 2.19-2.45 (5H, m), 2.51 (3H, s), 2.93-3.11 (4H, m), 3.12-3.29 (4H, m), 3.50-3.67 (8H, m), 4.72 (1H, m), 5.05 (1H, d), 7.00-7.20 (2H, m), 7.32-7.56 (2H, m), 7.63 (1H, d), 8.33-8.51 (2H, m).

Following the above process, 5-(4-(1-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-4-yl)piperazin-1-yl)-2-(2,6-dioxopiperidin-3-yl)isoindoline-1,3-dione (Compound 3), 5-(1′-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)-[4,4′-bipiperidin]-1-yl)-2-(2,6-dioxopiperidin-3-yl)isoindoline-1,3-dione (compound 9), 5-(4-((1-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-4-yl)methyl)piperazin-1-yl)-2-(2,6-dioxopiperidin-3-yl)isoindoline-1,3-dione (compound 11), 5-(4-(2-(1-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-4-yl)ethyl)piperazin-1-yl)-2-(2,6-dioxopiperidin-3-yl)isoindoline-1,3-dione (compound 19) were synthesized by using corresponding amines.

### Example 3. Preparation of Compound 31, 81, 82, 83, 84 & 88

As illustrative examples of compounds of formula (I), the following compounds, denoted Compounds 31, 81, 82, 83 & 88, were synthesized:

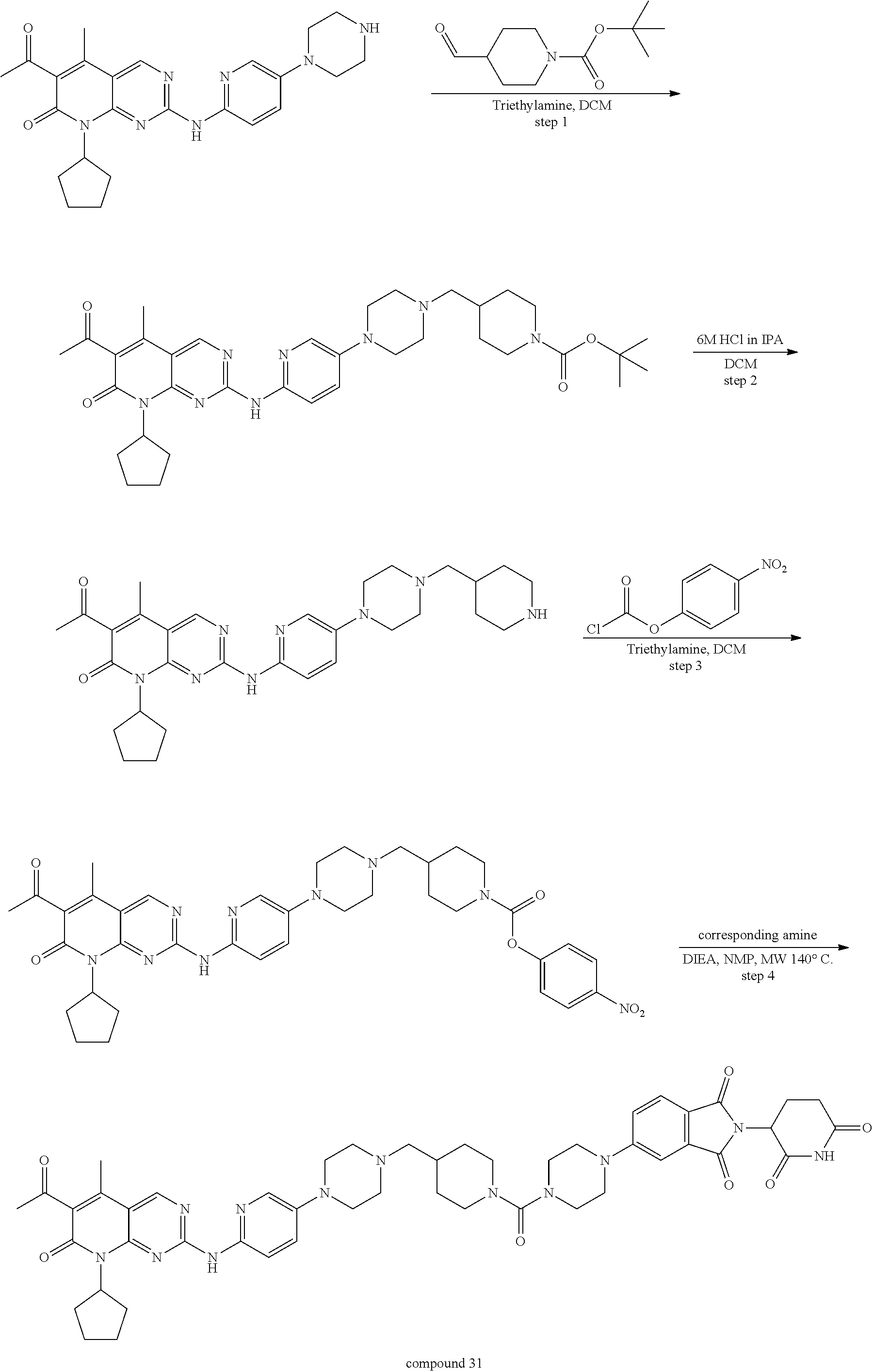

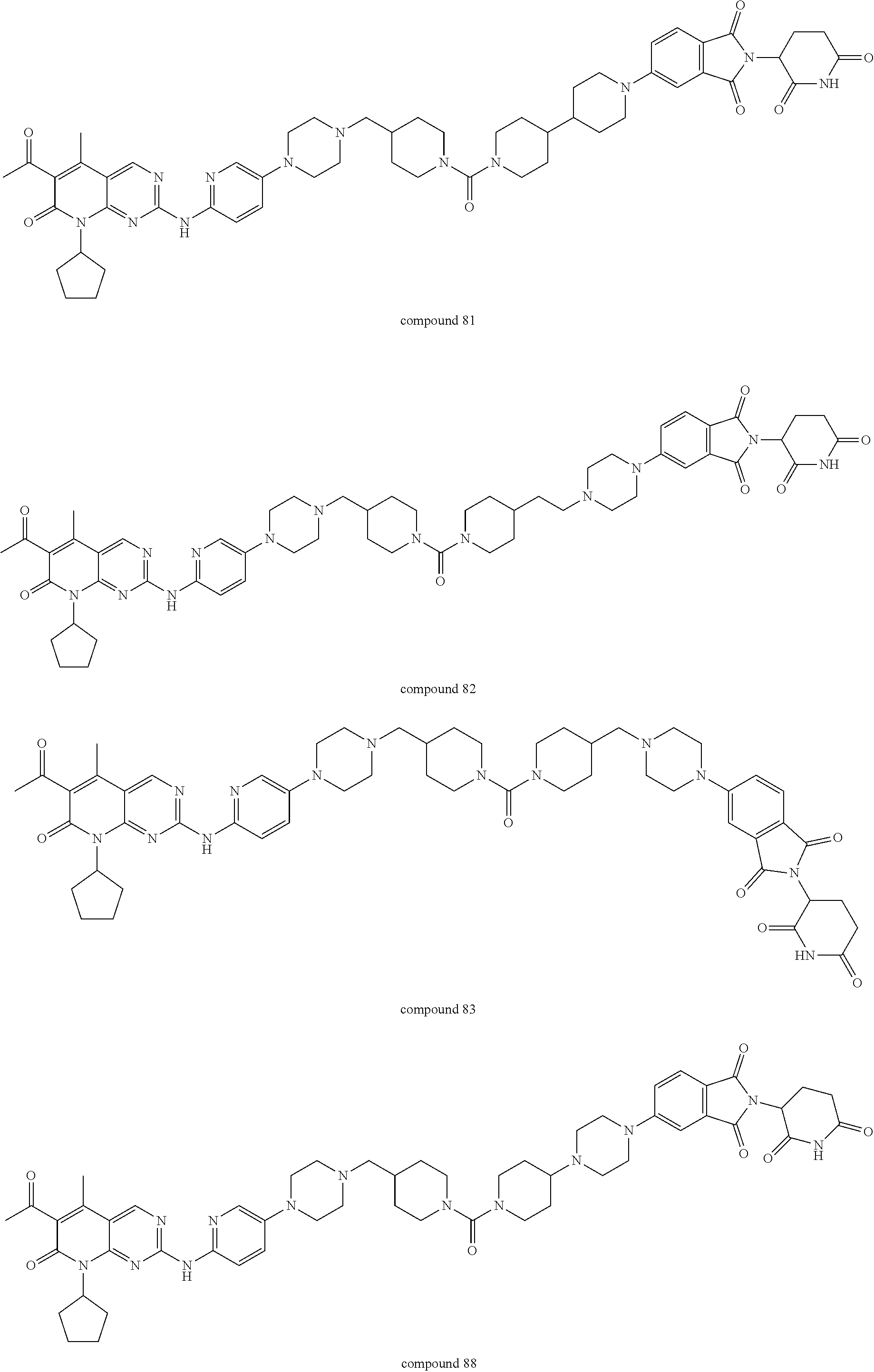

### Step 1. tert-butyl 4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carboxylate

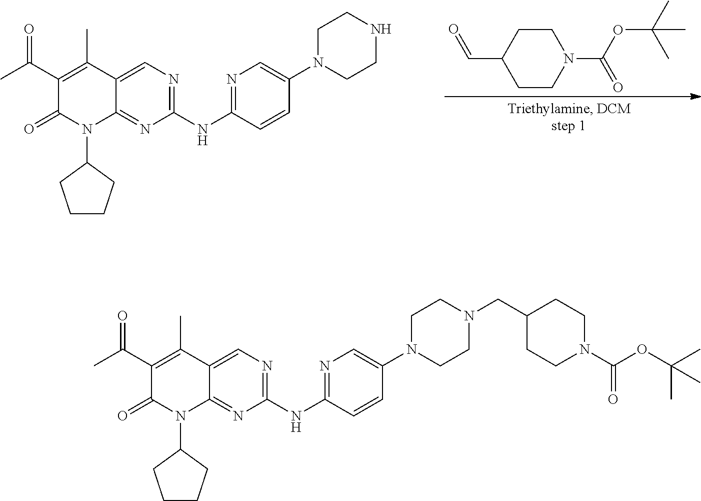 [SMILES: CC(=O)c1c(C)c2cnc(Nc3ccc(N4CCNCC4)cn3)nc2n(C2CCCC2)c1=O ; CC(C)(C)OC(=O)N1CCC(C=O)CC1 ; CC(=O)c1c(C)c2cnc(Nc3ccc(N4CCN(CC5CCN(C(=O)OC(C)(C)C)CC5)CC4)cn3)nc2n(C2CCCC2)c1=O]

To a stirred suspension of 6-acetyl-8-cyclopentyl-5-methyl-2-((5-(piperazin-1-yl)pyridin-2-yl)amino)pyrido[2,3-d]pyrimidin-7(8H)-one (0.894 g, 2 mmol) and triethylamine (0.40 g, 4 mmol) in 10 mL of DCM was added tert-butyl 4-formylpiperidine-1-carboxylate (0.51 g, 2.4 mmol) and sodium triacetoxyborohydride (0.80 g, 4 mmol) portionwise at rt. After stirring for 1 h, the solvent was removed in vacuo. The residue was subjected to silica gel column chromatography to provide 0.79 g of tert-butyl 4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carboxylate as yellow solid. LCMS [M+H] 645.

### Step 2. 6-acetyl-8-cyclopentyl-5-methyl-2-(5-(4-(piperidin-4-ylmethyl)piperazin-1-yl)pyridin-2-yl)amino)pyrido[2,3-d]pyrimidin-7(8H)-one

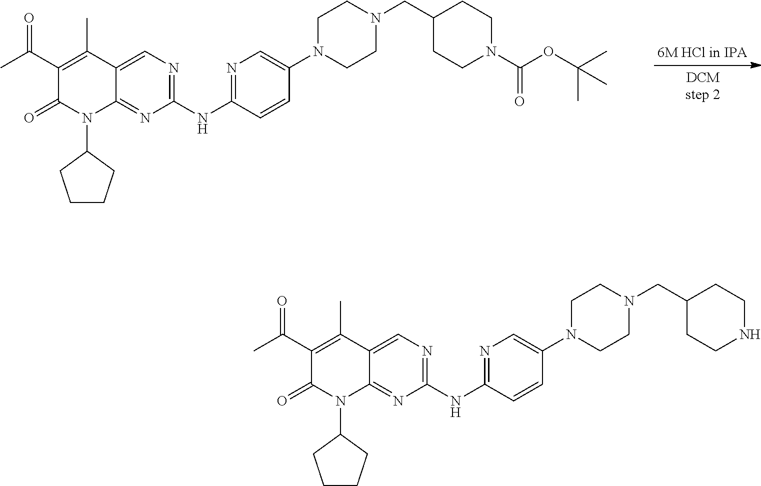

To a stirred suspension of above obtained tert-butyl 4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carboxylate (0.87 g, 2 mmol) in 10 mL of DCM was added 5 mL of 6M hydrochloric acid (HCl) in isopropyl alcohol (IPA) at rt. After stirring for 5 h, the solvent was removed in vacuo. 0.64 g of 6-acetyl-8-cyclopentyl-5-methyl-2-((5-(4-(piperidin-4-ylmethyl)piperazin-1-yl)pyridin-2-yl)amino)pyrido[2,3-d]pyrimidin-7(8H)-one was obtained as yellow solid and used in the next step without further purification. LCMS [M+H] 545.

### Step 3. 4-nitrophenyl 4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carboxylate

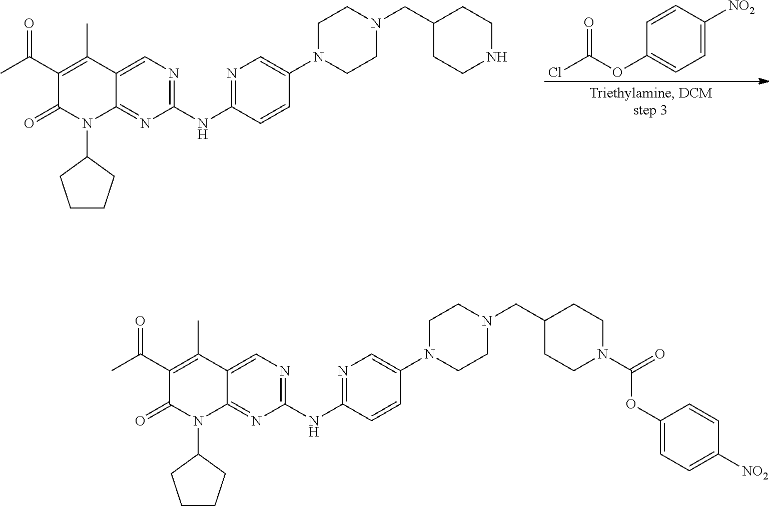 [SMILES: CC(=O)c1c(C)c2cnc(Nc3ccc(N4CCN(CC5CCNCC5)CC4)cn3)nc2n(C2CCCC2)c1=O ; O=C(Cl)Oc1ccc([N+](=O)[O-])cc1 ; CC(=O)c1c(C)c2cnc(Nc3ccc(N4CCN(CC5CCN(C(=O)Oc6ccc([N+](=O)[O-])cc6)CC5)CC4)cn3)nc2n(C2CCCC2)c1=O]

Following the procedure in example 2 step 1.

To a stirred suspension of 6-acetyl-8-cyclopentyl-5-methyl-2-((5-(4-(piperidin-4-ylmethyl)piperazin-1-yl)pyridin-2-yl amino)pyrido[2,3-d]pyrimidin-7(8H)-one (0.54 g, 1 mmol) and triethylamine (0.40 g, 4 mmol) in 10 mL of DCM was added 4-nitrophenyl carbonochloridate (0.24 g, 1.2 mmol) in one portion at rt. After stirring for 1 h, the solvent was removed in vacuo. The residue was subjected to silica gel column chromatography to provide 0.36 g of 4-nitrophenyl 4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carboxylate as an orange solid. LCMS [M+H] 710.

### Step 4. 5-(4-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carbonyl)piperazin-1-yl)-2-(2,6-dioxopiperidin-3-yl)isoindoline-1,3-dione (Compound 31)

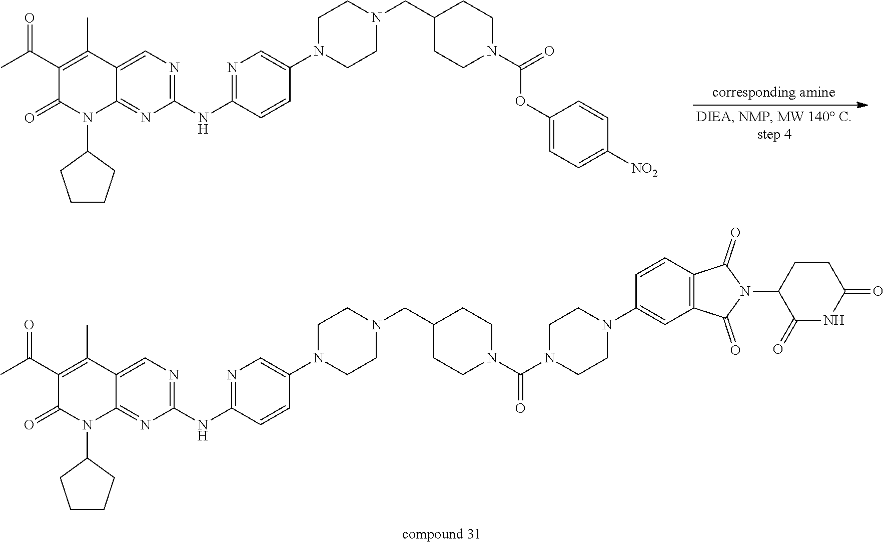 [SMILES: CC(=O)c1c(C)c2cnc(Nc3ccc(N4CCN(CC5CCN(C(=O)Oc6ccc([N+](=O)[O-])cc6)CC5)CC4)cn3)nc2n(C2CCCC2)c1=O ; CC(=O)c1c(C)c2cnc(Nc3ccc(N4CCN(CC5CCN(C(=O)N6CCN(c7ccc8c(c7)C(=O)N(C7CCC(=O)NC7=O)C8=O)CC6)CC5)CC4)cn3)nc2n(C2CCCC2)c1=O]

Following the procedure in example 2 step 2, with the corresponding amines, 5-(4-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carbonyl)piperazin-1-yl)-2-(2,6-dioxopiperidin-3-yl)isoindoline-1,3-dione (compound 31), 5-(1′-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carbonyl)-[4,4′-bipiperidin]-1-yl)-2-(2,6-dioxopiperidin-3-yl)isoindoline-1,3-dione (compound 81), 5-(4-(2-(1-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carbonyl)piperidin-4-yl)ethyl)piperazin-1-yl)-2-(2,6-dioxopiperidin-3-yl)isoindoline-1,3-dione (compound 82), 5-(4-((1-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carbonyl)piperidin-4-yl)methyl)piperazin-1-yl)-2-(2,6-dioxopiperidin-3-yl)isoindoline-1,3-dione (compound 83), & 5-(4-(1-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carbonyl)piperidin-4-yl)piperazin-1-yl)-2-(2,6-dioxopiperidin-3-yl)isoindoline-1,3-dione (compound 88) were obtained as yellow solids.

### Example 4. Preparation of Compounds 35, 84, 85, 86 & 87

As illustrative examples of compounds of formula (I), the following compounds, denoted Compounds 35, 84, 85, 86 & 87, were synthesized:

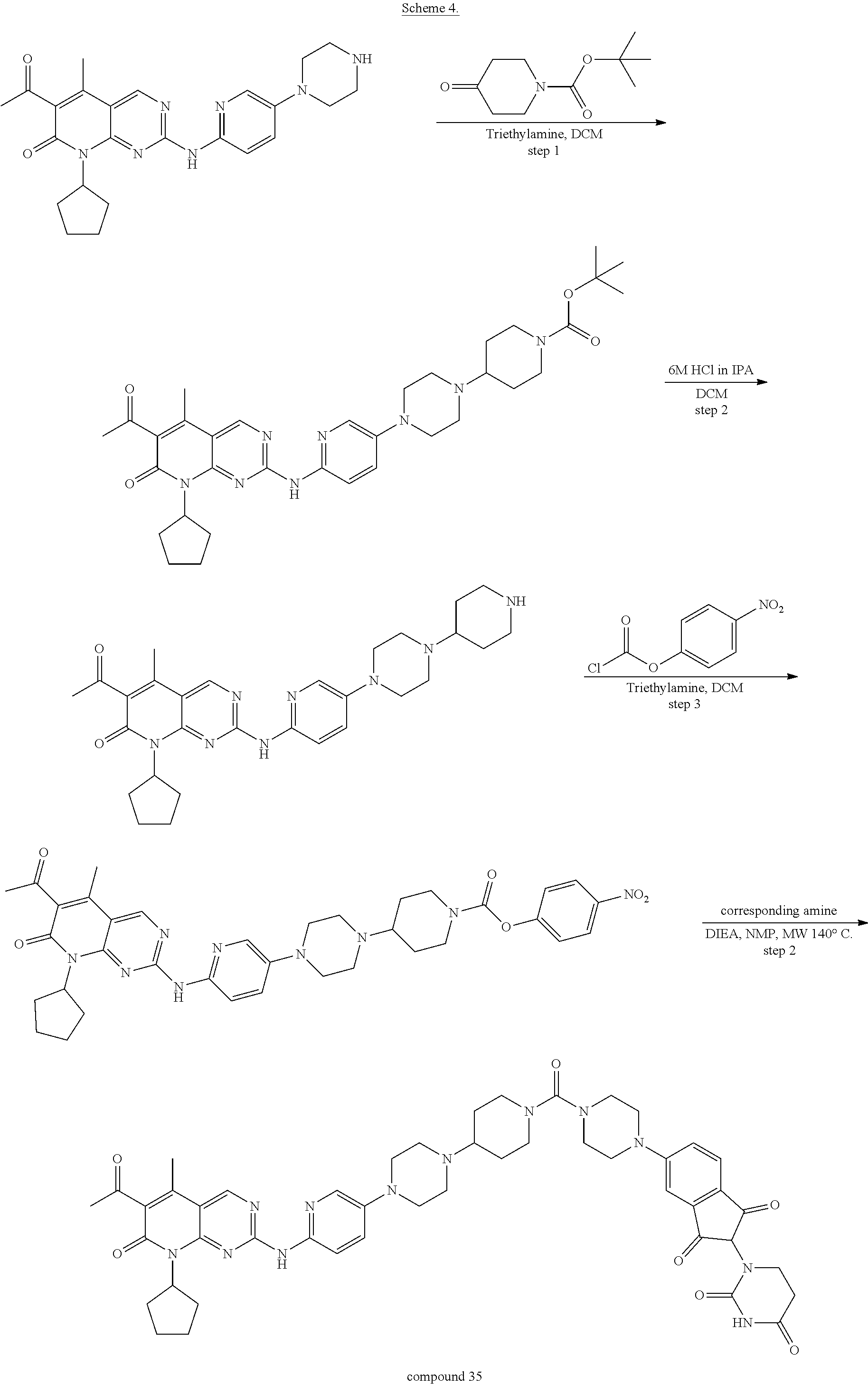

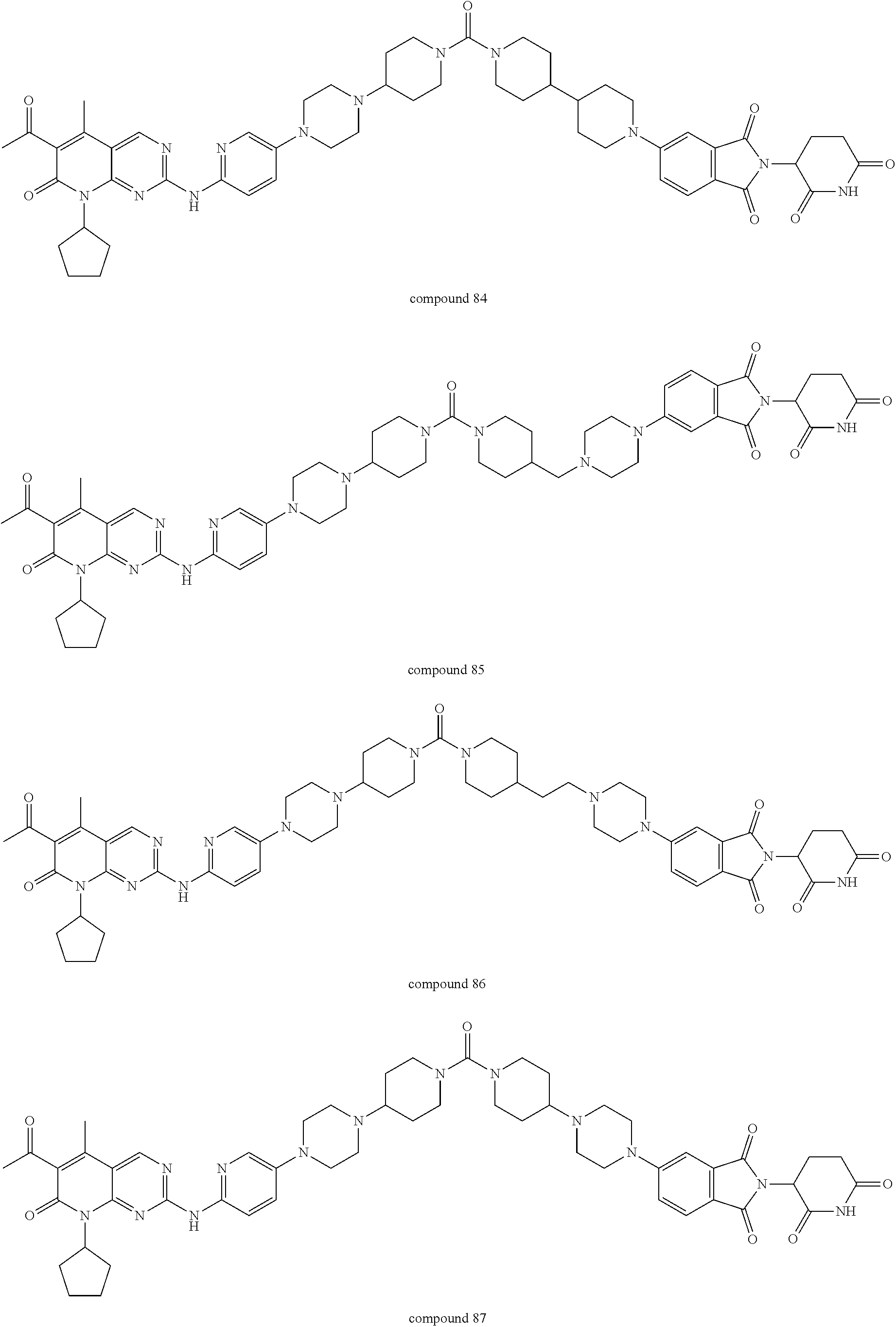

### Step 1. tert-butyl 4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carboxylate

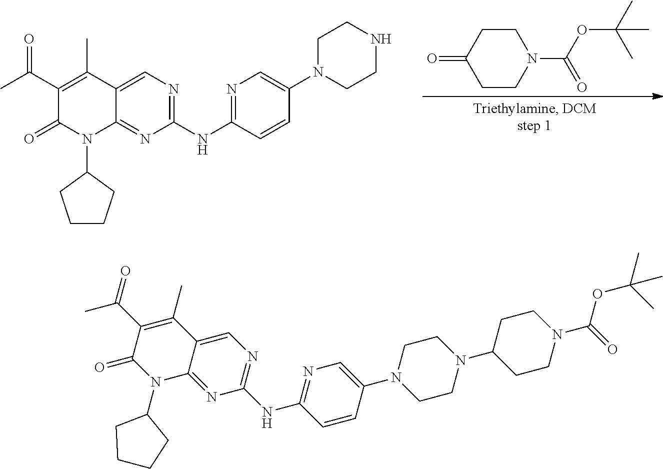 [SMILES: CC(=O)c1c(C)c2cnc(Nc3ccc(N4CCNCC4)cn3)nc2n(C2CCCC2)c1=O ; CC(C)(C)OC(=O)N1CCC(=O)CC1 ; CC(=O)c1c(C)c2cnc(Nc3ccc(N4CCN(C5CCN(C(=O)OC(C)(C)C)CC5)CC4)cn3)nc2n(C2CCCC2)c1=O]

To a stirred suspension of 6-acetyl-8-cyclopentyl-5-methyl-2-((5-(piperazin-1-yl)pyridin-2-yl)amino)pyrido[2,3-d]pyrimidin-7(8H)-one (0.894 g, 2 mmol) and triethylamine (0.40 g, 4 mmol) in 10 mL of DCM was added tert-butyl 4-oxopiperidine-1-carboxylate (0.8 g, 4 mmol) followed by sodium triacetoxyborohydride (0.80 g, 4 mmol) portionwise at it After stirring for 1 h, the solvent was removed in vacuo. The residue was subjected onto silica gel column chromatography to provide 0.87 g of tert-butyl 4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carboxylate as yellow solid. LCMS [M+H] 631.

### Step 2. 6-acetyl-8-cyclopentyl-5-methyl-2-((5-(4-piperidin-4-yl)piperazin-1-yl)pyridin-2-yl)amino)pyrido[2,3-d]pyrimidin-7(8H)-one

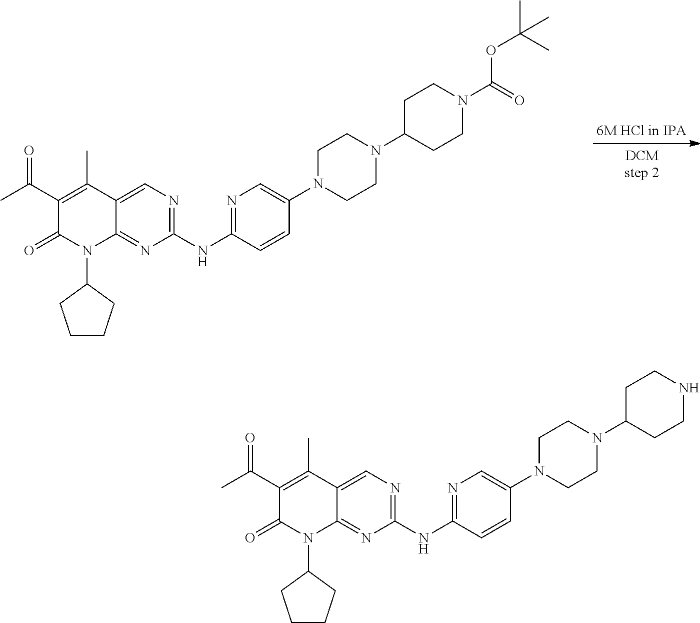

To a stirred suspension of above obtained tert-butyl 4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carboxylate (0.87 g, 2 mmol) in 10 mL of DCM was added 5 mL of 6M HCl in isopropyl alcohol (IPA) at rt. After stirring for 5 h, the solvent was removed in vacuo. 0.58 g of 6-acetyl-8-cyclopentyl-5-methyl-2-((5-(4-(piperidin-4-yl)piperazin-1-yl)pyridin-2-yl)amino)pyrido[2,3-d]pyrimidin-7(8H)-one was obtained as yellow solid and used in the next step without further purification. LCMS [M+H] 531.

### Step 3. 4-nitrophenyl 4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carboxylate

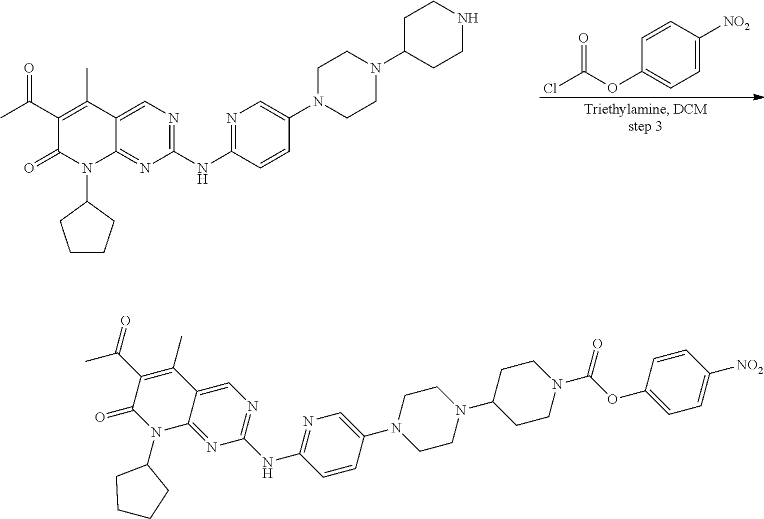 [SMILES: CC(=O)c1c(C)c2cnc(Nc3ccc(N4CCN(C5CCNCC5)CC4)cn3)nc2n(C2CCCC2)c1=O ; O=C(Cl)Oc1ccc([N+](=O)[O-])cc1 ; CC(=O)c1c(C)c2cnc(Nc3ccc(N4CCN(C5CCN(C(=O)Oc6ccc([N+](=O)[O-])cc6)CC5)CC4)cn3)nc2n(C2CCCC2)c1=O]

Following the procedure in example 2 step 1.

To a stirred suspension of 6-acetyl-8-cyclopentyl-5-methyl-2-((5-(4-(piperidin-4-yl)piperazin-1-yl)pyridin-2-yl)amino)pyrido[2,3-d]pyrimidin-7(8H)-one (0.53 g, 1 mmol) and triethylamine (0.40 g, 4 mmol) in 10 mL of DCM was added 4-nitrophenyl carbonochloridate (0.48 g, 2.4 mmol) in one portion at rt. After stirring for 1 h, the solvent was removed in vacuo. The residue was subjected to silica gel column chromatography to provide 0.34 g of 4-nitrophenyl 4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carboxylate as a yellow solid. LCMS [M+H] 696.

### Step 4. 5 (4-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carbonyl)piperazin-1-yl)-2-(2,6-dioxopiperidin-3-yl)isoindoline-1,3-dione (Compound 35)

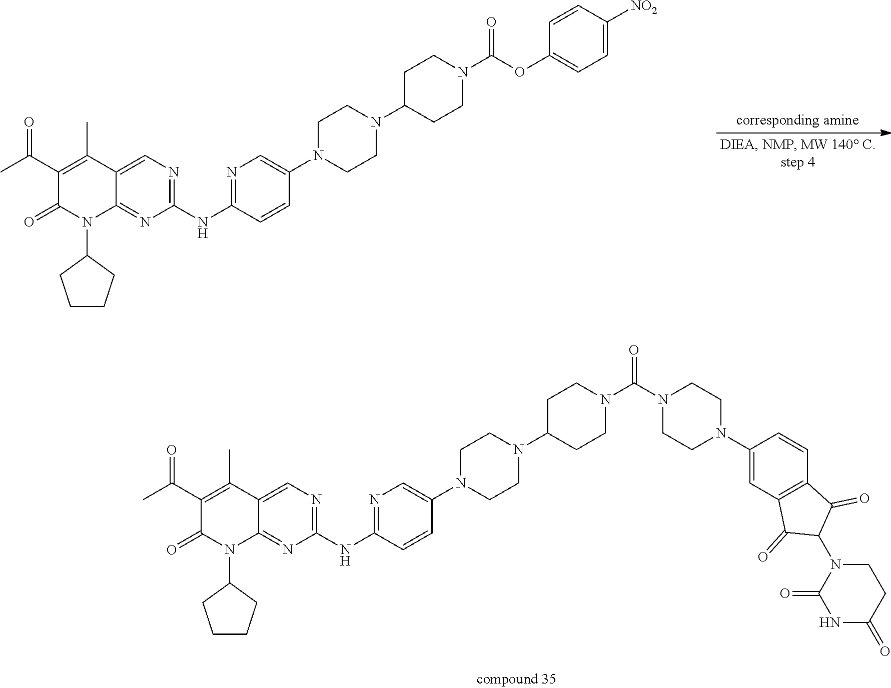 [SMILES: CC(=O)c1c(C)c2cnc(Nc3ccc(N4CCN(C5CCN(C(=O)Oc6ccc([N+](=O)[O-])cc6)CC5)CC4)cn3)nc2n(C2CCCC2)c1=O ; CC(=O)c1c(C)c2cnc(Nc3ccc(N4CCN(C5CCN(C(=O)N6CCN(c7ccc8c(c7)C(=O)C(N7CCC(=O)NC7=O)C8=O)CC6)CC5)CC4)cn3)nc2n(C2CCCC2)c1=O]

Following the procedure in example 2 step 2, with the corresponding amines, 5-(4-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carbonyl)piperazin-1-yl)-2-(2,6-dioxopiperidin-3-yl)isoindoline-1,3-dione (compound 35), 5-(1′-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carbonyl)-[4,4′-bipiperidin]-1-yl)-2-(2,6-dioxopiperidin-3-yl)isoindoline-1,3-dione (compound 84), 5-(4-((1-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carbonyl)piperidin-4-yl)methyl)piperazin-1-yl)-2-(2,6-dioxopiperidin-3-yl)isoindoline-1,3-dione (compound 85), 5-(4-(2-(1-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carbonyl)piperidin-4-yl)ethyl)piperazin-1-yl)-2-(2,6-dioxopiperidin-3-yl)isoindoline-1,3-dione (compound 86), 5-(4-(1-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carbonyl)piperidin-4-yl)piperazin-1-yl)-2-(2,6-dioxopiperidin-3-yl)isoindoline-1,3-dione (compound 87) were obtained as yellow solids.

### Example 5. Preparation of Compound 49, 50, 51, 47, 55, 57

As illustrative examples of compound of formula (I), the following compounds, denoted Compounds 49, 50, 51, 47, 55, 57, were synthesized:

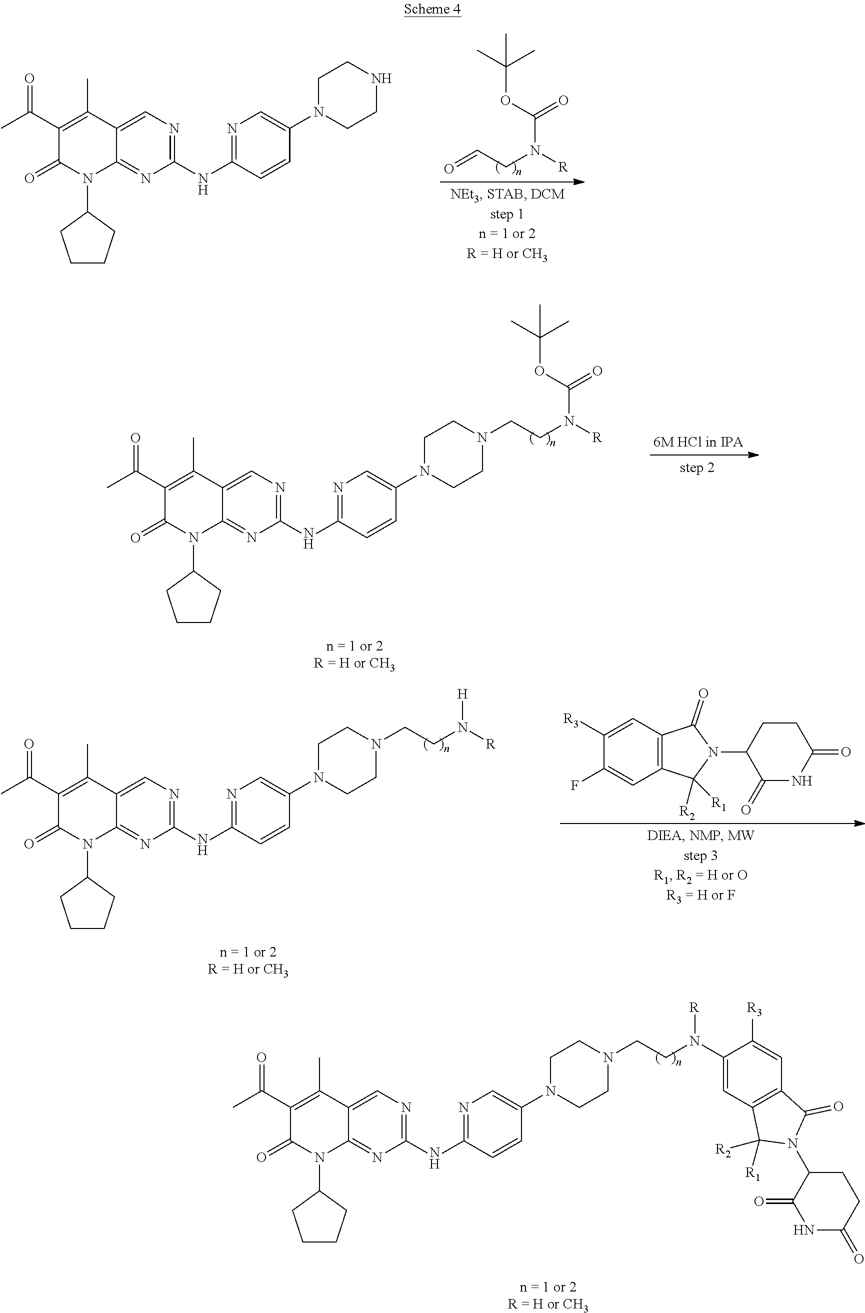

### Step 1. tert-butyl (3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propyl)carbamate

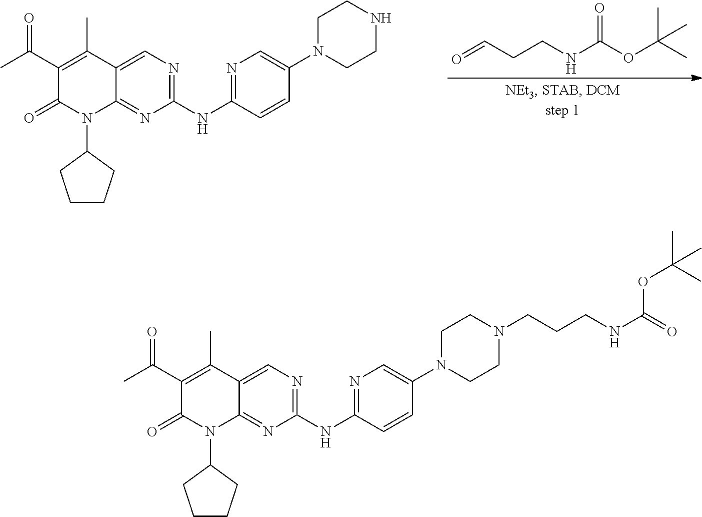 [SMILES: CC(=O)c1c(C)c2cnc(Nc3ccc(N4CCNCC4)cn3)nc2n(C2CCCC2)c1=O ; CC(C)(C)OC(=O)NCCC=O ; CC(=O)c1c(C)c2cnc(Nc3ccc(N4CCN(CCCNC(=O)OC(C)(C)C)CC4)cn3)nc2n(C2CCCC2)c1=O]

To a stirred suspension of 6-acetyl-8-cyclopentyl-5-methyl-2-((5-(piperazin-1-yl)pyridin-2-yl)amino)pyrido[2,3-d]pyrimidin-7(8H)-one (224 mg, 0.5 mmol) and triethylamine (0.40 g, 4 mmol) in 5 mL of DCM was added tert-butyl (3-oxopropyl)carbamate (0.10 g, 0.6 mmol) followed by sodium triacetoxyborohydride (0.16 g, 0.75 mmol) in one portion at rt. After stirring for 1 h, the solvent was removed in vacuo. The residue was subjected to silica gel column chromatography to provide 0.30 g of tert-butyl (3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propyl)carbamate as a yellow solid. LCMS [M+H] 605.

### Step 2. 6-acetyl-2-((5-(4-(3-aminopropyl)piperazin-1-yl)pyridin-2-yl)amino)-8-cyclopentyl-5-methylpyrido[2,3-d]pyrimidin-7(8H)-one

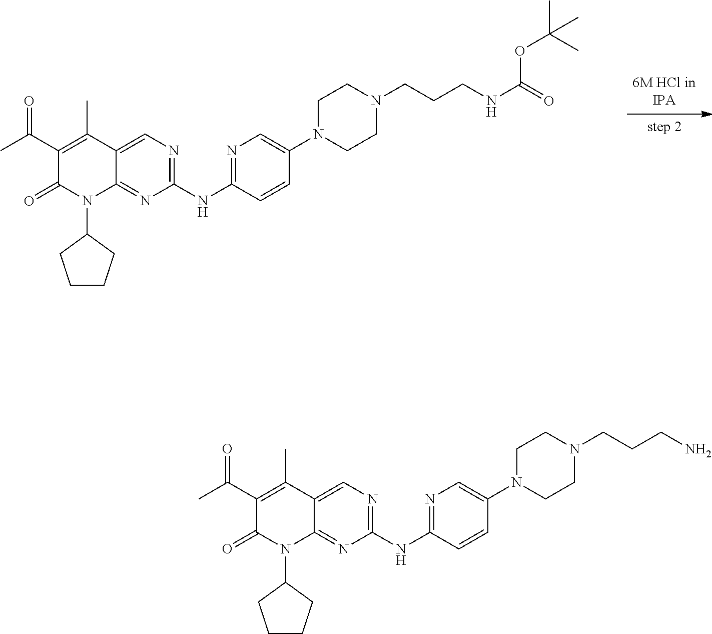

To a stirred suspension of above obtained tert-butyl 4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carboxylate (0.3 g, 0.5 mmol) in 5 mL of DCM was added 1 mL of 6M HCl in IPA at rt. After stirring for 5 h, the solvent was removed in vacuo. 0.35 g of 6-acetyl-2-((5-(4-(3-aminopropyl)piperazin-1-yl)pyridin-2-yl)amino)-8-cyclopentyl-5-methylpyrido[2,3-d]pyrimidin-7(8H)-one HCl salt was obtained as a yellow solid and used in the next step without further purification. LCMS [M+H] 505.

### Step 3. 5-((3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propyl)amino)-2-(2,6-dioxopiperidin-3-yl)isoindoline-1,3-dione (Compound 49)

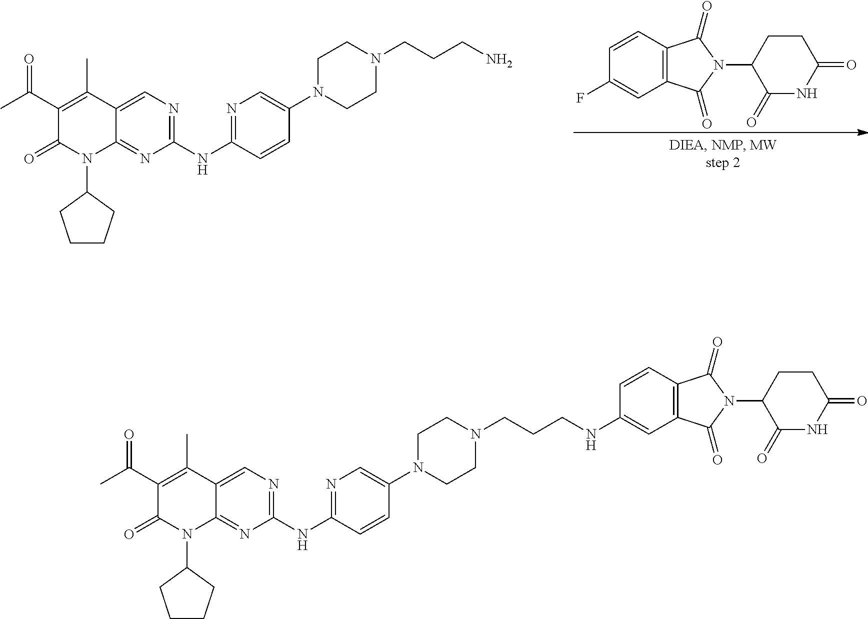 [SMILES: CC(=O)c1c(C)c2cnc(Nc3ccc(N4CCN(CCCN)CC4)cn3)nc2n(C2CCCC2)c1=O ; O=C1CCC(N2C(=O)c3ccc(F)cc3C2=O)C(=O)N1 ; CC(=O)c1c(C)c2cnc(Nc3ccc(N4CCN(CCCNc5ccc6c(c5)C(=O)N(C5CCC(=O)NC5=O)C6=O)CC4)cn3)nc2n(C2CCCC2)c1=O]

A mixture of 6-acetyl-2-((5-(4-(3-aminopropyl)piperazin-1-yl)pyridin-2-yl)amino)-8-cyclopentyl-5-methylpyrido[2,3-d]pyrimidin-7(8H)-one (0.35 g), 2-(2,6-dioxopiperidin-3-yl)-5-fluoroisoindoline-1,3-dione (0.28 g, 1 mmol), DIEA (0.65 g, 5 mmol) in 4 mL of NMP was heated and stirred in a microwave MW irradiator at 120° C. for 1 h. After cooling to rt, the product was purified by silica gel column chromatography with ethyl acetate (EA): MeOH as eluent. The crude product was further purified by prep-HPLC, C18 with 0.5% formic acid (FA) in water and MeCN as eluent. 45 mg of 5-((3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propyl)amino)-2-(2,6-dioxopiperidin-3-yl)isoindoline-1,3-dione was obtained as a yellow solid. LCMS [M+H] 761. ¹H NMR: δ 1.62-2.14 (12H, m), 2.19-2.45 (5H, m), 2.45-2.61 (9H, m), 2.92-3.07 (4H, m), 3.24-3.36 (2H, m), 4.96 (1H, m), 5.75 (1H, dd), 6.93-8.46 (6H, m), 11.87 (s, 1H).

Following the above procedure, with the corresponding starting materials, 3-(5-((3-(4(6-((6-acetyl-8-cyclopentyl-5-methyl-7-oxo-7,8-dihydropyrido[2,3-d]pyrimidin-2-yl)amino)pyridin-3-yl)piperazin-1-yl)propyl)amino)-1-oxoisoindolin-2-yl)piperidine-2,6-dione (compound 50), 5-((3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propyl)amino)-2-(2,6-dioxopiperidin-3-yl)-6-fluoroisoindoline-1,3-dione (compound 51), 5-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)amino)-2-(2,6-dioxopiperidin-3-yl)isoindoline-1,3-dione (compound 46), 5-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)(methyl)amino)-2-(2,6-dioxopiperidin-3-yl)isoindoline-1,3-dione (compound 55), 5-((3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propyl)(methyl)amino)-2-(2,6-dioxopiperidin-3-yl)isoindoline-1,3-dione (compound 57) were obtained as yellow solids.

### Example 6. Preparation of Compounds 73.100

As illustrative examples of compounds of formula (I), the following compounds, denoted Compound 73 and compound 100 were synthesized:

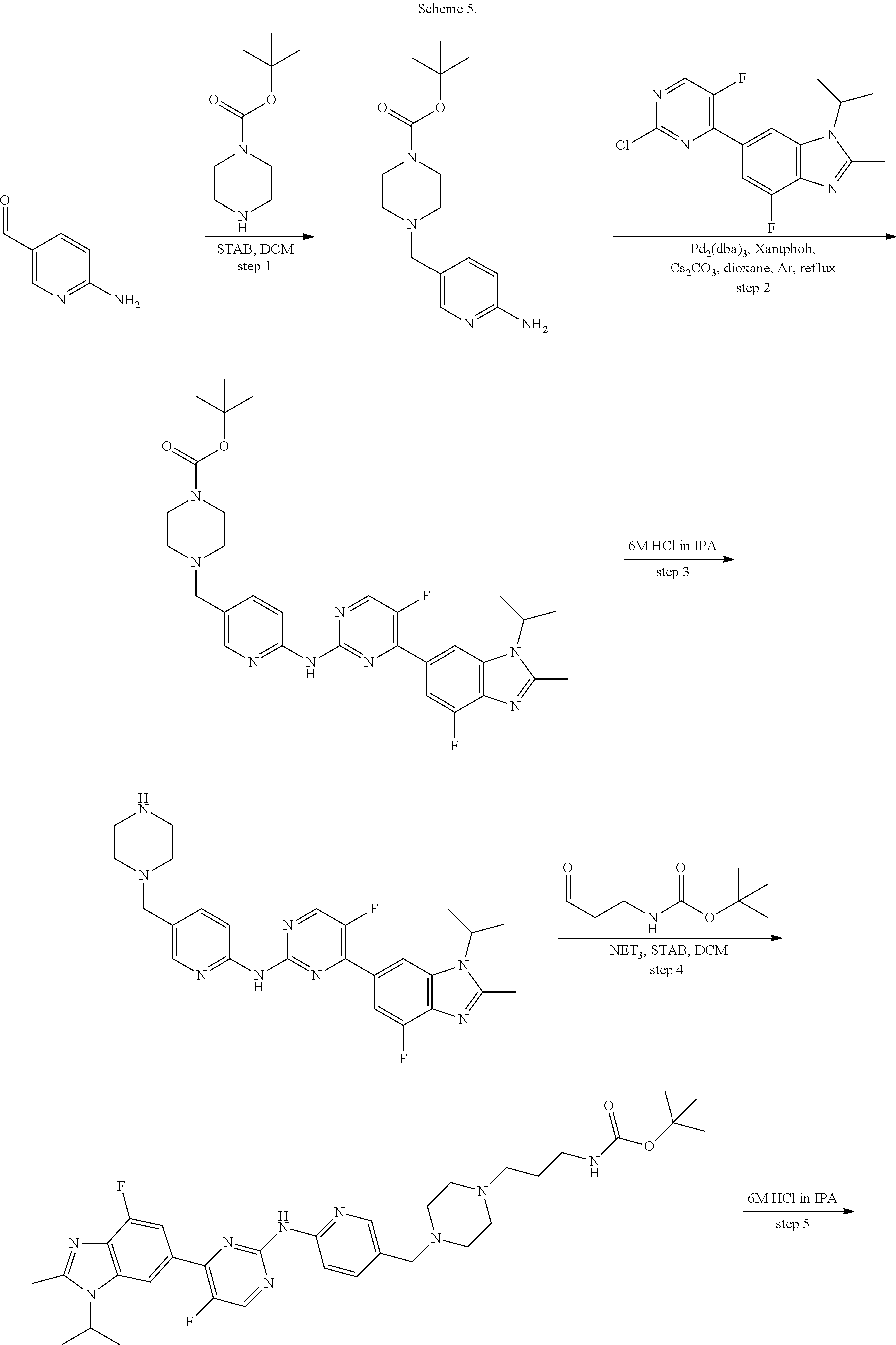

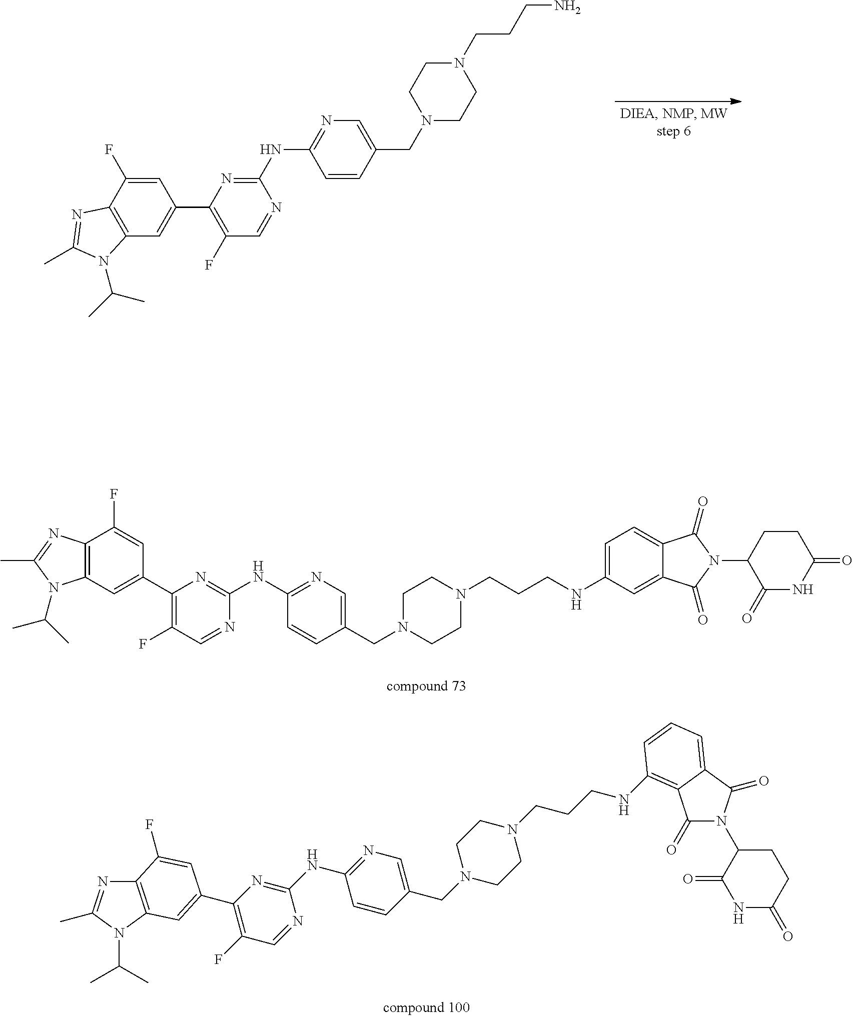 [SMILES: Cc1nc2c(F)cc(-c3nc(Nc4ccc(CN5CCN(CCCN)CC5)cn4)ncc3F)cc2n1C(C)C ; Cc1nc2c(F)cc(-c3nc(Nc4ccc(CN5CCN(CCCNc6ccc7c(c6)C(=O)N(C6CCC(=O)NC6=O)C7=O)CC5)cn4)ncc3F)cc2n1C(C)C ; Cc1nc2c(F)cc(-c3nc(Nc4ccc(CN5CCN(CCCNc6cccc7c6C(=O)N(C6CCC(=O)NC6=O)C7=O)CC5)cn4)ncc3F)cc2n1C(C)C]

### Step 1. tert-butyl 4-((6-aminopyridin-3-yl)methyl)piperazine-1-carboxylate

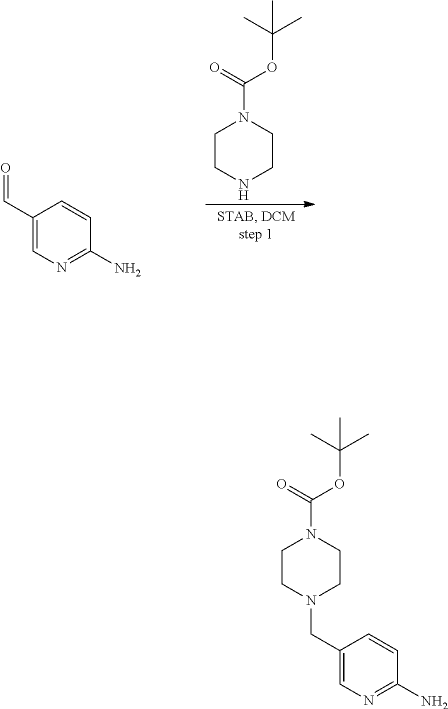 [SMILES: Nc1ccc(C=O)cn1 ; CC(C)(C)OC(=O)N1CCNCC1 ; CC(C)(C)OC(=O)N1CCN(Cc2ccc(N)nc2)CC1]

To a stirred suspension of 6-aminonicotinaldehyde (4.92 g, 40 mmol) and tert-butyl piperazine-1-carboxylate (9.0 g, 48 mmol) in 200 mL of DCM was added sodiumtriacetoxyborohydride (STAB) (12.7 g, 60 mmol) portionwise at rt. The resulting mixture was stirred at rt for 5 h. Ice-water was added to quench the reaction and the product was extracted with DCM (2×200 mL). The combined organic layer was dried over Na₂SO₄, filtered, and evaporated to dryness. The residue was subjected to silica gel column chromatography with EA/MeOH as eluent. 3.5 g of tert-butyl 4-((6-aminopyridin-3-yl)methyl)piperazine-1-carboxylate was obtained as white solid. LCMS [M+H] 293.

### Step 2. tert-butyl 4-((6-((5-fluoro-4-(4-fluoro-1-isopropyl-2-methyl-1H-benzo[d]imidazol-6-yl)pyrimidin-2-yl)amino)pyridin-3-yl)methyl)piperazine-1-carboxylate

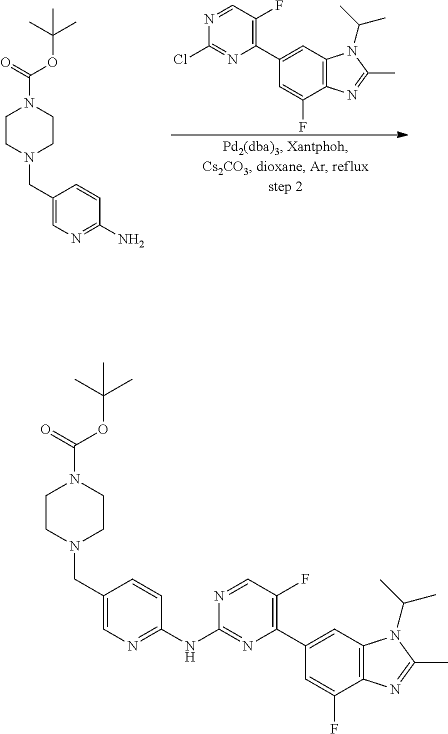 [SMILES: CC(C)(C)OC(=O)N1CCN(Cc2ccc(N)nc2)CC1 ; Cc1nc2c(F)cc(-c3nc(Cl)ncc3F)cc2n1C(C)C ; Cc1nc2c(F)cc(-c3nc(Nc4ccc(CN5CCN(C(=O)OC(C)(C)C)CC5)cn4)ncc3F)cc2n1C(C)C]

A mixture of tert-butyl 4-((6-aminopyridin-3-yl)methyl)piperazine-1-carboxylate (1.5 g, 5 mmol), 6-(2-chloro-5-fluoropyrimidin-4-yl)-4-fluoro-1-isopropyl-2-methyl-1H-benzo[d]imidazole (1.6 g, 5 mmol), Pd₂(dba)₃ (0.14 g, 0.25 mmol), 4,5-bis(diphenylphosphino)-9,9-dimethylxanthene (Xantphos) (0.15 g, 0.5 mmol) and Cs₂CO₃ (3.25 g, 10 mmol) in 100 mL of 1,4-dioxane was stirred at reflux under Ar for 2 h. While cooled to about 50° C., the solids were filtered through celite. The filtrate cake was further washed twice with tetrahydrofuran (THF). The combined filtrates were evaporated in vacuo until solids formed. Ethylacetate (EA) was added to the obtained crude product and stirred for about 1 h. The solids were collected with filtration, washed with water twice and then n-hexane. The solids were dried at 40° C. overnight to provide 2.5 g of tert-butyl 4-((6-((5-fluoro-4-(4-fluoro-1-isopropyl-2-methyl-1H-benzo[d]imidazol-6-yl)pyrimidin-2-yl)amino)pyridin-3-yl)methyl)piperazine-1-carboxylate as white solid. LCMS [M+H] 293.

### Step 3. 5-fluoro-4-(4-fluoro-1-isopropyl-2-methyl-1H-benzo[d]imidazol-6-yl)-N-(5-(piperazin-1-ylmethyl)pyridin-2-yl)pyrimidin-2-amine

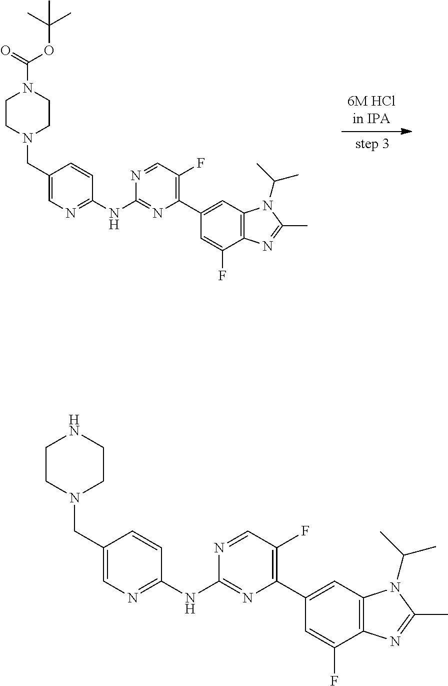 [SMILES: Cc1nc2c(F)cc(-c3nc(Nc4ccc(CN5CCN(C(=O)OC(C)(C)C)CC5)cn4)ncc3F)cc2n1C(C)C ; Cc1nc2c(F)cc(-c3nc(Nc4ccc(CN5CCNCC5)cn4)ncc3F)cc2n1C(C)C]

To a stirred suspension of above obtained tert-butyl 4-((6-((5-fluoro-4-(4-fluoro-1-isopropyl-2-methyl-1H-benzo[d]imidazol-6-yl)pyrimidin-2-yl)amino)pyridin-3-yl)methyl)piperazine-1-carboxylate (0.58 g, 1 mmol) in 5 mL of DCM was added 1 mL of 6M HCl in IPA at rt. After stirring for 5 h, the solvent was removed in vacuo. 0.7 g of 5-fluoro-4-(4-fluoro-1-isopropyl-2-methyl-1H-benzo[d]imidazol-6-yl)-N-(5-(piperazin-1-ylmethyl)pyridin-2-yl)pyrimidin-2-amine with HCl salt was obtained as yellow solid and used in the next step without further purification. LCMS [M+H] 479.

### Step 4. tert-butyl (3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)propyl)carbamate

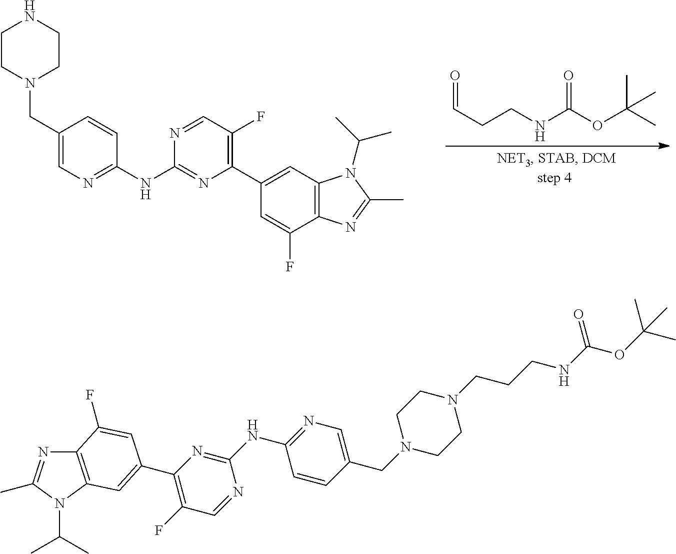 [SMILES: Cc1nc2c(F)cc(-c3nc(Nc4ccc(CN5CCNCC5)cn4)ncc3F)cc2n1C(C)C ; CC(C)(C)OC(=O)NCCC=O ; Cc1nc2c(F)cc(-c3nc(Nc4ccc(CN5CCN(CCCNC(=O)OC(C)(C)C)CC5)cn4)ncc3F)cc2n1C(C)C]

To a stirred suspension of tert-butyl (3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)propyl)carbamate (479 mg, 1 mmol) and triethylamine (0.5 g, 5 mmol) in 5 mL of DCM was added tert-butyl (3-oxopropyl)carbamate (0.21 g, 1.2 mmol) followed by sodium triacetoxyborohydride (0.42 g, 2 mmol) in one portion at rt. After stirring for 1 h, the solvent was removed in vacuo. The residue was subjected to silica gel column chromatography to provide 0.55 g of tert-butyl (3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)propyl)carbamate as yellow solid. LCMS [M+H] 636.

### Step 5. N-(5-((4-(3-aminopropyl)piperazin-1-yl)methyl)pyridin-2-yl)-5-fluoro-4-(4-fluoro-1-isopropyl-2-methyl-1H-benzo[d]imidazol-6-yl)pyrimidin-2-amine

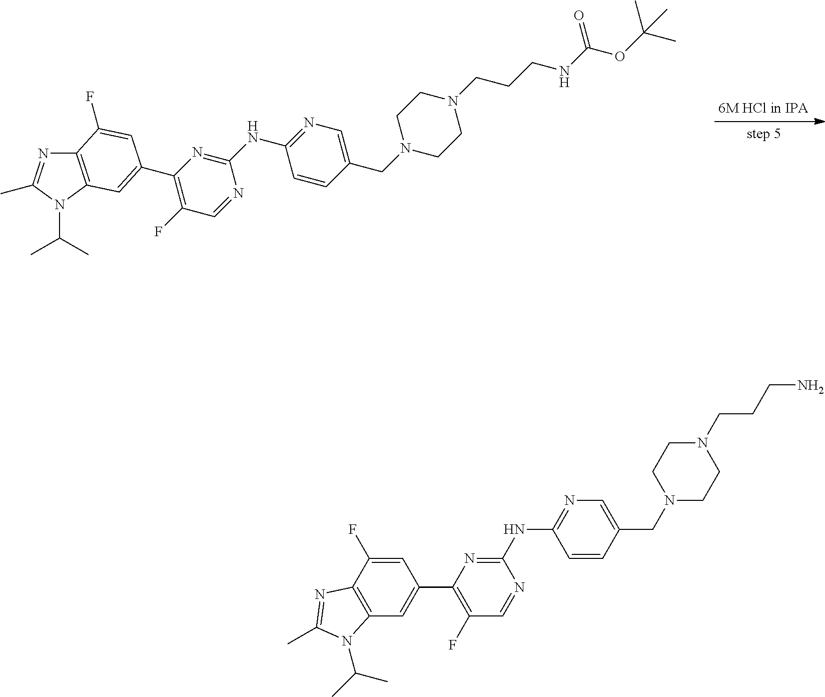

To a stirred suspension of above obtained tert-butyl (3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)propyl)carbamate (0.5 g, 0.5 mmol) in 5 mL of DCM was added 1 mL of 6M HCl in IPA at it After stirring for 5 h, the solvent was removed in vacuo. 0.61 g of N-(5-((4-(3-aminopropyl)piperazin-1-yl)methyl)pyridin-2-yl)-5-fluoro-4-(4-fluoro-1-isopropyl-2-methyl-1H-benzo[d]imidazol-6-yl)pyrimidin-2-amine was obtained as white solid and used in the next step without further purification. LCMS [M+H] 536.

### Step 6. 2-(2,6-dioxopiperidin-3-yl)-5-((3-(4-((6-((5-fluoro-4-(4-fluoro-1-Isopropyl-2-methyl-1H-benzo[d]imidazol-1-yl)pyrimidin-2-yl)amino)pyridin-3-yl)methyl)piperazin-1-yl)propyl)amino)isoindoline-1,3-dione (Compound 73)

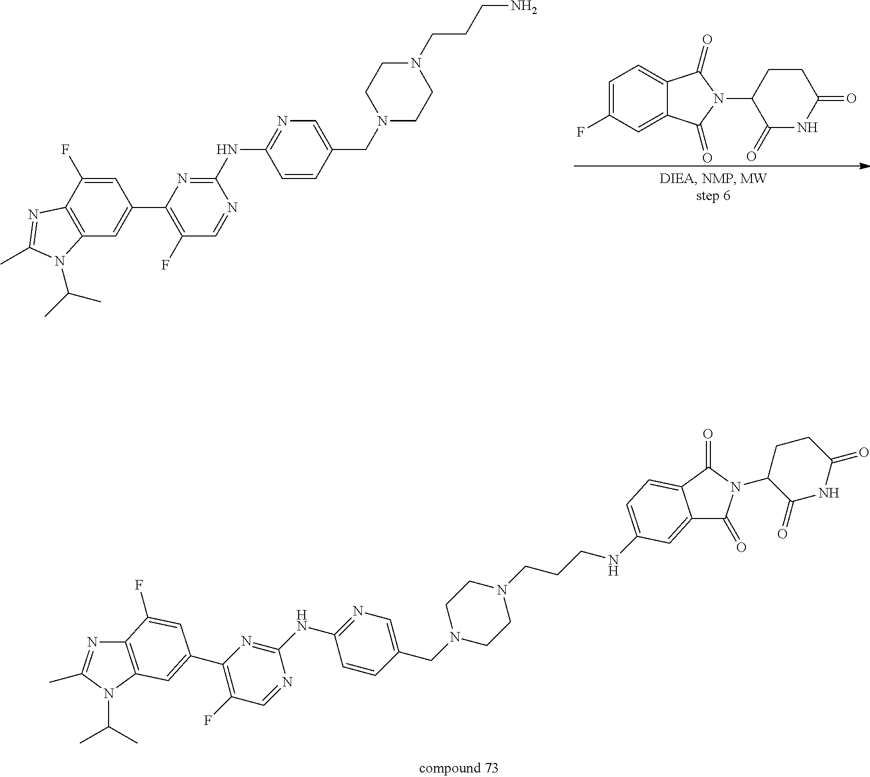 [SMILES: Cc1nc2c(F)cc(-c3nc(Nc4ccc(CN5CCN(CCCN)CC5)cn4)ncc3F)cc2n1C(C)C ; O=C1CCC(N2C(=O)c3ccc(F)cc3C2=O)C(=O)N1 ; Cc1nc2c(F)cc(-c3nc(Nc4ccc(CN5CCN(CCCNc6ccc7c(c6)C(=O)N(C6CCC(=O)NC6=O)C7=O)CC5)cn4)ncc3F)cc2n1C(C)C]

A mixture of N-(5-((4-(3-aminopropyl)piperazin-1-yl)methyl)pyridin-2-yl)-5-fluoro-4-(4-fluoro-1-isopropyl-2-methyl-1H-benzo[d]imidazol-6-yl)pyrimidin-2-amine (0.12 g), 2-(2,6-dioxopiperidin-3-yl)-5-fluoroisoindoline-1,3-dione (0.12 g, 0.5 mmol), DIEA (0.65 g, 5 mmol) in 4 mL of NMP was heated and stirred in a MW irradiator at 120° C. for 1 h. After cooling to rt, the product was purified by silica gel column chromatography with EA: MeOH as eluent. The crude product was further purified by prep-HPLC, C18 with 0.5% FA in water and MeCN as eluent. 62 mg of 2-(2,6-dioxopiperidin-3-yl)-5-((3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)propyl)amino)isoindoline-1,3-dione as pale solid. LCMS [M+H] 792. ¹H NMR: δ 1.56-2.14 (10H, m), 2.19-2.69 (15H, m), 3.24-3.36 (2H, m), 3.65-3.75 (2H, m), 4.92 (1H, m), 5.05 (1H, d), 6.87-7.09 (2H, m), 7.51 (1H, d). 7.79 (1H, d), 8.10-8.46 (5H, m), 11.06 (1H, m).

Following the above procedure, with the corresponding starting materials, 2-(2,6-dioxopiperidin-3-yl)-4-((3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)propyl)amino)isoindoline-1,3-dione (compound 100) was obtained as brown solid. LCMS [M+H] 792. ¹H NMR: δ 1.56-1.68 (6H, m), 1.74-1.86 (2H, m), 1.95-2.15 (2H, m), 2.19-2.46 (4H, m), 2.47-2.69 (11H, m), 3.26-3.38 (2H, m), 3.65-3.75 (2H, m), 4.92 (1H, t), 5.12 (1H, d), 6.92-7.09 (2H, m), 7.50 (1H, d), 7.68-7.85 (2H, m), 8.10-8.33 (3H, m), 8.41 (1H, d), 11.05 (1H, s).

Following the above procedure, with the corresponding starting materials, 7-cyclopentyl-2-((5-(4-(3-((2-(2,6-dioxopiperidin-3-yl)-1,3-dioxoisoindolin-5-yl)amino)propyl)piperazin-1-yl)pyridin-2-yl)amino)-N,N-dimethyl-7H-pyrrolo[2,3-d]pyrimidine-6-carboxamide (compound 75) was obtained as brown solid.

### Example 7. Preparation of Compound 94

As an illustrative example of a compound of formula (I), the following compound, denoted Compound 94, was synthesized:

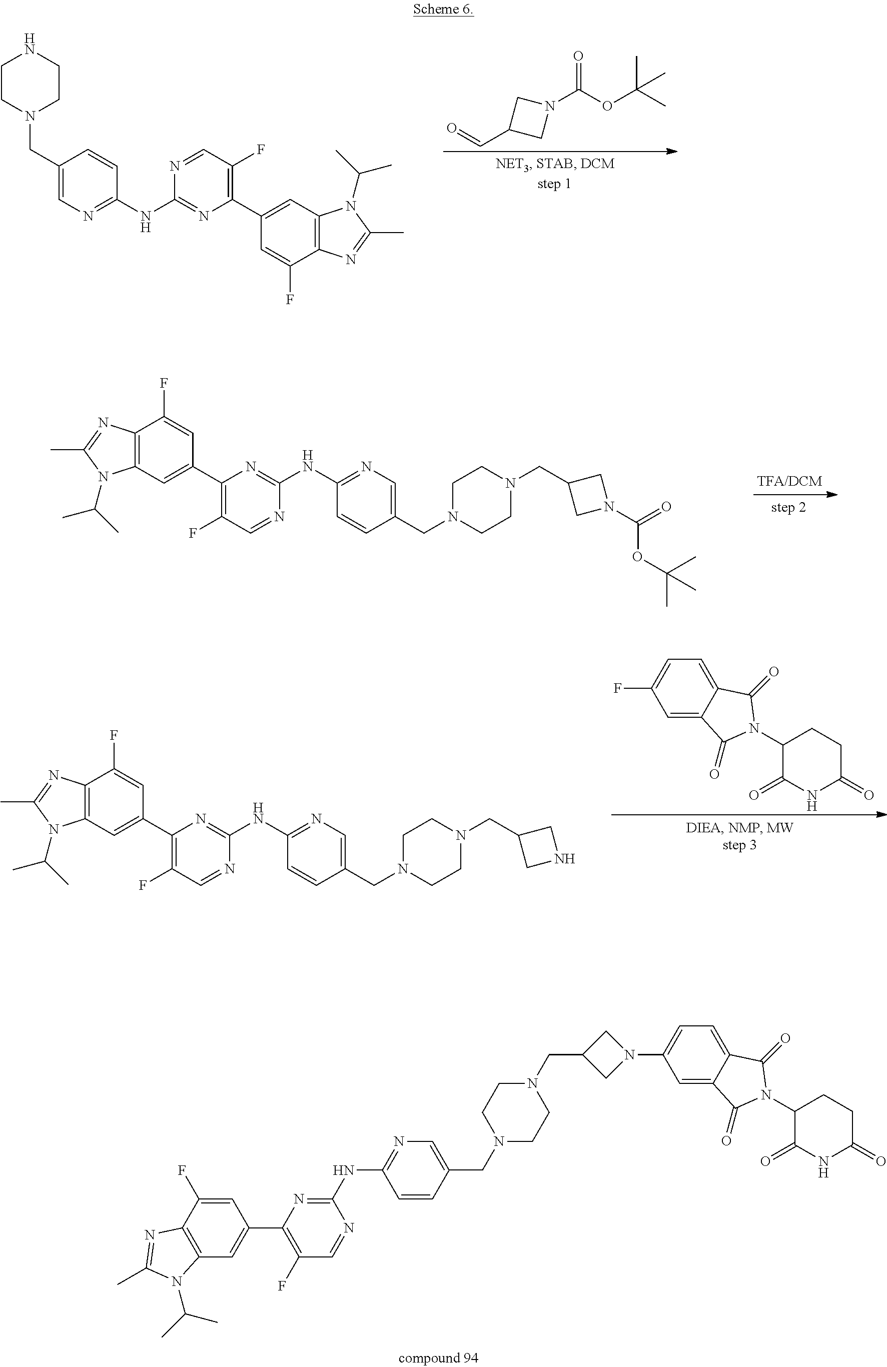

### Step 1. tert-butyl 3-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)azetidine-1-carboxylate

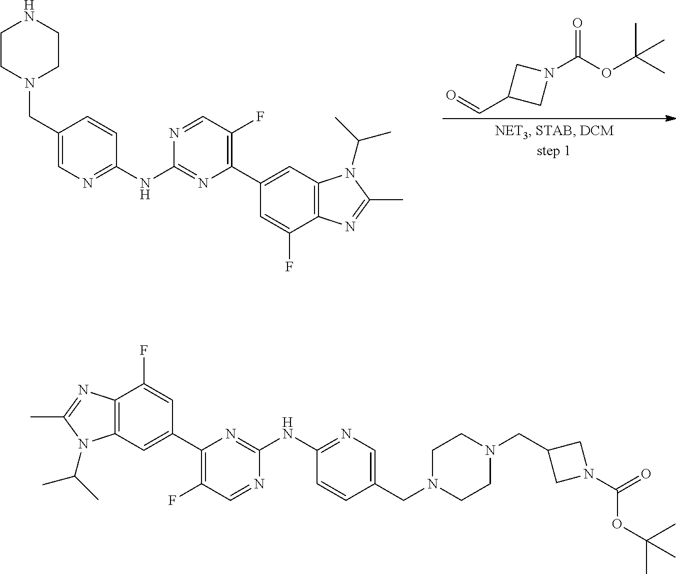 [SMILES: Cc1nc2c(F)cc(-c3nc(Nc4ccc(CN5CCNCC5)cn4)ncc3F)cc2n1C(C)C ; CC(C)(C)OC(=O)N1CC(C=O)C1 ; Cc1nc2c(F)cc(-c3nc(Nc4ccc(CN5CCN(CC6CN(C(=O)OC(C)(C)C)C6)CC5)cn4)ncc3F)cc2n1C(C)C]

To a stirred suspension of tert-butyl (3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)propyl)carbamate (479 mg, 1 mmol) and triethylamine (0.5 g, 5 mmol) in 5 mL of DCM was added tert-butyl 3-formylazetidine-1-carboxylate (0.22 g, 1.2 mmol) followed by sodium triacetoxyborohydride (0.42 g, 2 mmol) in one portion at rt. After stirring for 1 h, the solvent was removed in vacuo. The residue was subjected to silica gel column chromatography to provide 0.45 g of tert-butyl (3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)propyl)carbamate as yellow solid. LCMS [M+H] 648.

### Step 2. N-(5 ((4-(azetidin-3-ylmethyl)piperazin-1-yl)methyl)pyridin-2-yl)-5-fluoro-4-(4-fluoro-1-isopropyl-2-methyl-1H-benzo[d]imidazol-6-yl)pyrimidin-2-amine

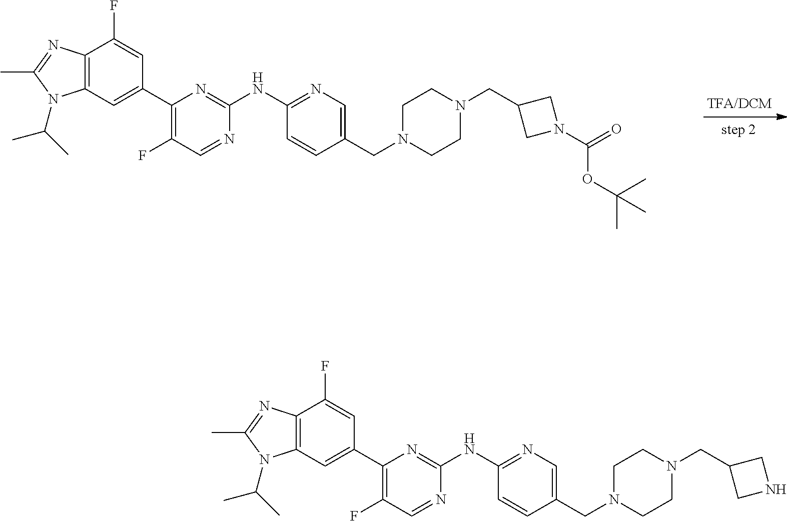

To a stirred suspension of above obtained tert-butyl (3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)propyl)carbamate (0.32 g, 0.5 mmol) in 5 mL of DCM was added 1 mL of trifluoroacetic acid (TFA) at rt. After stirring for 2 h, the solvent was removed in vacuo. 0.44 g of N-(5-((4-(3-aminopropyl)piperazin-1-yl)methyl)pyridin-2-yl)-5-fluoro-4-(4-fluoro-1-isopropyl-2-methyl-1H-benzo[d]imidazol-6-yl)pyrimidin-2-amine nTFA salt was obtained as brown oil and used in the next step without further purification. LCMS [M+H] 548.

### Step 3. 2-(2,6-dioxopiperidin-3-yl)-5-(3-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)azetidin-1-yl)isoindoline-1,3-dione (Compound 94)

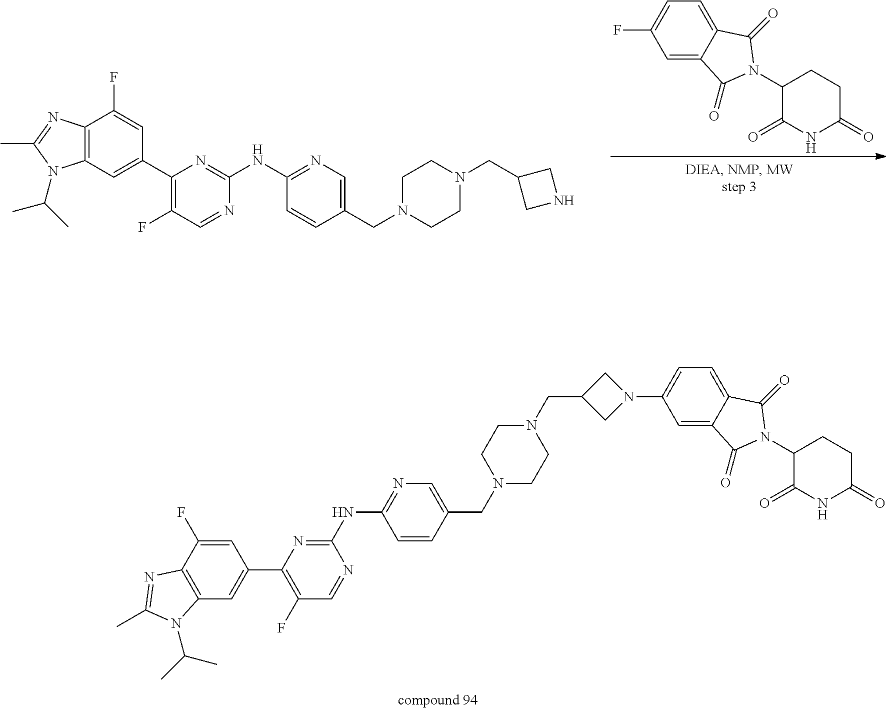 [SMILES: Cc1nc2c(F)cc(-c3nc(Nc4ccc(CN5CCN(CC6CNC6)CC5)cn4)ncc3F)cc2n1C(C)C ; O=C1CCC(N2C(=O)c3ccc(F)cc3C2=O)C(=O)N1 ; Cc1nc2c(F)cc(-c3nc(Nc4ccc(CN5CCN(CC6CN(c7ccc8c(c7)C(=O)N(C7CCC(=O)NC7=O)C8=O)C6)CC5)cn4)ncc3F)cc2n1C(C)C]

A mixture of N-(5-((4-(3-aminopropyl)piperazin-1-yl)methyl)pyridin-2-yl)-5-fluoro-4-(4-fluoro-1-isopropyl-2-methyl-1H-benzo[d]imidazol-6-yl)pyrimidin-2-amine nTFA salt (0.44 g), 2-(2,6-dioxopiperidin-3-yl)-5-fluoroisoindoline-1,3-dione (0.12 g, 0.5 mmol), DIEA (0.65 g, 5 mmol) in 4 mL of NMP was heated and stirred in a MW irradiator at 120° C. for 1 h. After cooling to rt, the product was purified by silica gel column chromatography with EA: MeOH as eluent. The crude product was further purified by prep-HPLC, C18 with 0.5% FA in water and MeCN as eluent. 33 mg of 2-(2,6-dioxopiperidin-3-yl)-5-(3-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)azetidin-1-yl)isoindoline-1,3-dione as light yellow solid. LCMS [M+H] 804. ¹H NMR: δ 1.56-1.68 (6H, m), 1.95-2.15 (2H, m), 2.19-2.69 (16H, m), 3.61-3.76 (4H, m), 3.89-4.05 (2H, m), 4.92 (1H, t), 5.12 (1H, d), 7.03 (1H, d), 7.34 (1H, d), 7.50 (1H, d), 7.79 (1H, d), 8.04 (1H, d), 8.10-8.33 (3H, m), 8.41 (1H, d), 11.05 (1H, s).

### Example 8. Antiproliferative Effect

Antiproliferative effect of the exemplary compounds was assessed in B-cell malignancy models.

To assess the effect of the exemplary compounds on cell viability in B-cell malignancy models by degradation of CDK4/6 proteins, Mino cells were treated with varying concentrations of exemplary compounds (0, 0.0001, 0.001, 0.01, 0.1, 1 μM) or positive control (palbociclib and abemaciclib). Under these conditions, cell viability was determined by an MTS assay. Cytotoxicity was assayed by the determination of reduced tetrazolium (formazan) created by metabolically active cells as detected at 490 nanometers (nm) absorbance using a microplate reader (BMG Labtech, Cary, NC, USA).

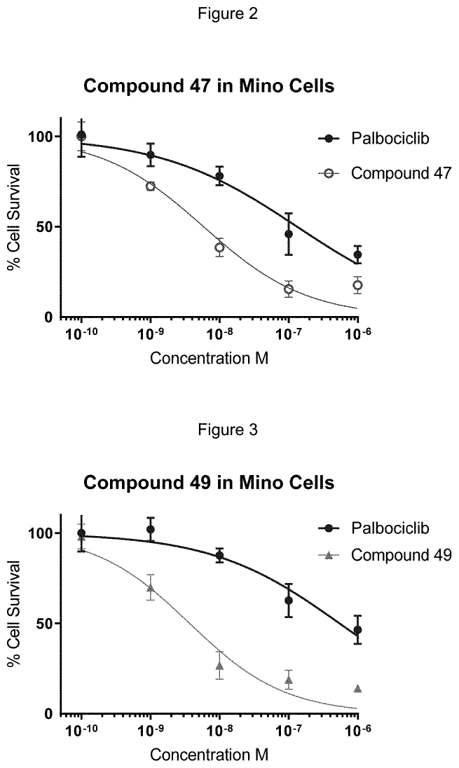

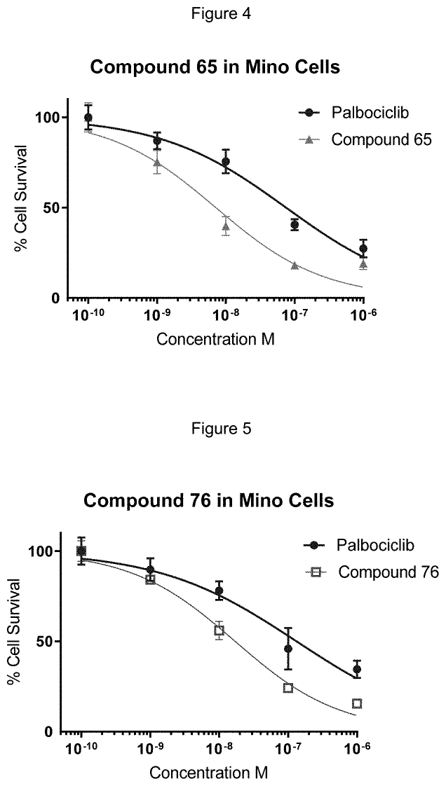

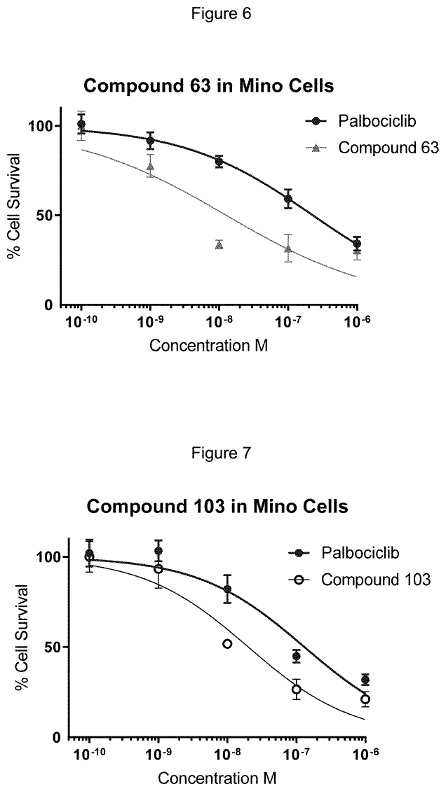

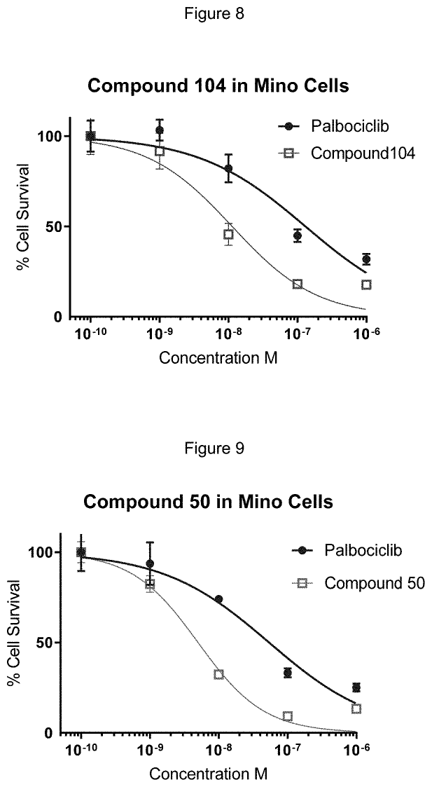

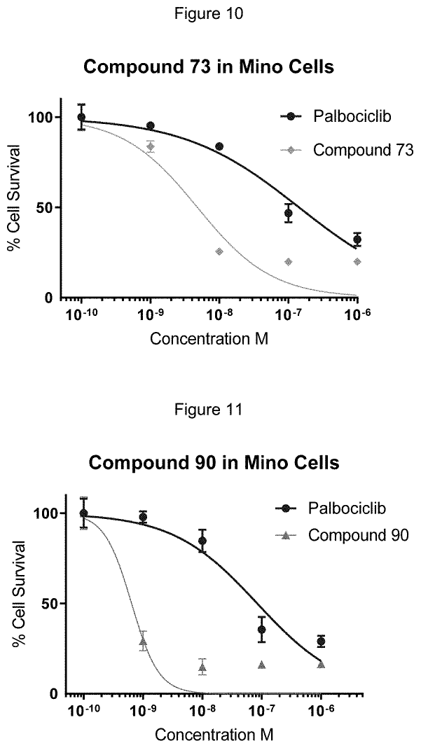

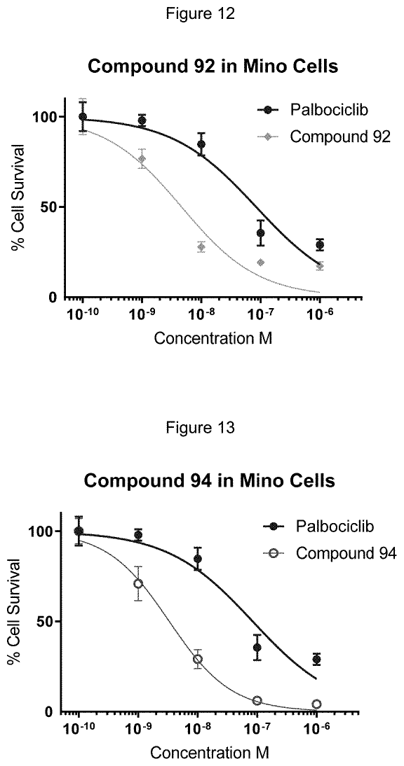

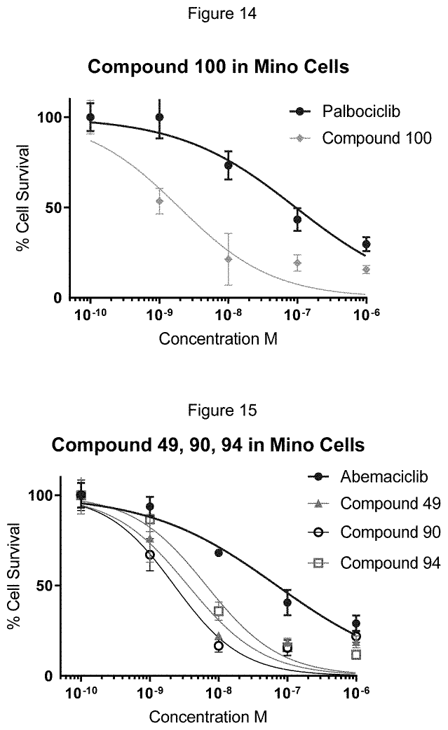

The exemplary compounds attenuated the viability of Mino cells in a concentration-dependent manner at 72 h post treatment. Results are shown in Table 1 and FIGS. 2-15 for each exemplary compound.

### Example 9. Pharmacokinetics

Pharmacokinetic properties of exemplary compounds 49 were assessed in Female Sprague Dawley rats.

Sprague Dawley rats (n=3), weighing between 350 and 400 g (Charles River Laboratories, Portage. MI) were given oral gavage containing compound 49 suspended in propylene glycol (PG):solutol:40% HP-b-CD in DI water (20:5:75 v:v) at a single dose of 10 mg/kg. After drug administration, blood samples were collected from the tail vein of the rats at various time points into 1.5 mL microcentrifuge tubes containing 0.1 mL of 10% EDTA anticoagulant. Plasma was then separated from cell pellets by centrifugation in a refrigerated centrifuge at 4° C. and transferred to a separate tube. Plasma samples were frozen at −80° C. until analysis.

HPLC-MS/MS Analysis of Plasma Samples. Plasma samples were extracted with chloroform/methanol (2:1) using traditional Folch method for lipid extraction. Methanol (1 mL) and chloroform (2 mL) were added to each plasma sample followed by addition of 5 nanograms (ng) trans-Tamoxifen-13C2, 15N to each sample as the internal standard. The mixtures were stored at −20° C. overnight. Next the samples were sonicated for 5 min and centrifuged with a Thermo Scientific Heraeus Megafuge 16 Centrifuge. The top layer was transferred to another test tube. The bottom layer was washed with 1 mL chloroform/methanol (2:1), centrifuged, and the solvent was transferred and combined with previous washings. Eight tenth of a milliliter HPLC grade water was added to the extracts. After vortexing, the mixture was centrifuged. The bottom layer was dried out with nitrogen and re-suspended in 100 μL HPLC grade acetonitrile. An aliquot of 10 microliter (μL) sample was injected onto a Hypersil Gold column (50×2.1 mm; particle size 1.9 micrometer (μm), Thermo Scientific) on a Dionex Ultimate 3000 UPLC system equipped with a TSQ Vantage triple quadrupole mass spectrometer for analysis. A binary mobile phase (A: water with 0.05% formic acid, B: acetonitrile with 0.05% formic acid) was used to achieve the gradient of initial 30% B for 1 min and then to 80% B at 8 min, to 100% B at 9 min, and returned to 30% B for 4 min. The flow rate was controlled at 0.6 mL/min. The settings of HESI source were as follows: spray voltage (3200 volt); vaporizer temperature (365° C.); sheath gas pressure (45 psi); auxiliary gas pressure (10 psi); capillary temperature (330° C.). Nitrogen was used as the sheath gas and axillary gas. Argon was used as the collision gas.

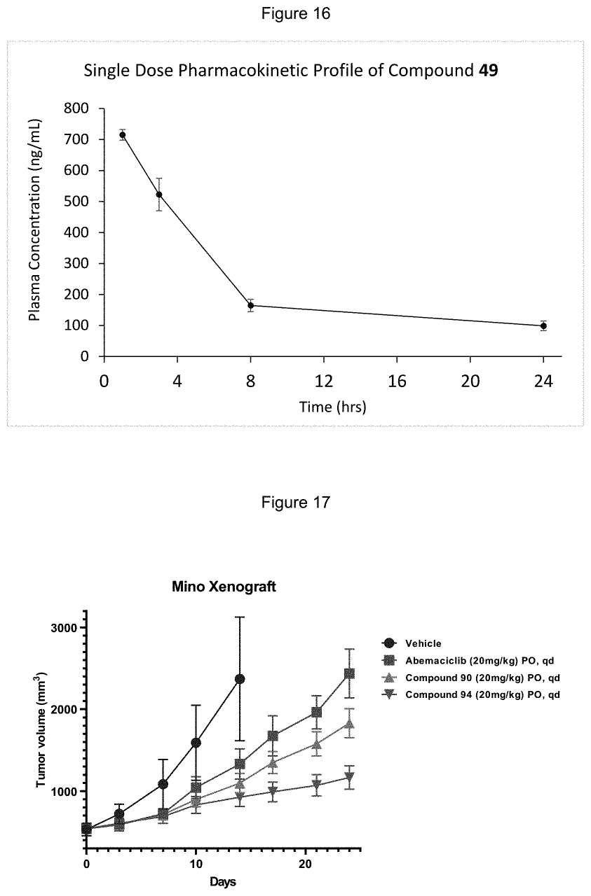

FIG. 16 shows the pharmacokinetic profile of exemplary compounds 49 in SD rats.

### Example 10. In Vivo Tumor Inhibition Efficacy

Mino cells were obtained from American Type Culture Collection (ATCC, Manassas, VA). Cells were maintained in RPM 1640 (Life Technologies, Grand Island, NY) supplemented with 10% heat-inactivated fetal bovine serum (56° C. for 30 minutes), penicillin (10 000 units/mL; Sigma, St Louis, Mo), streptomycin (10 mg/mL; Sigma), gentamicin (50 mg/mL; Life Technologies), amphotericin B (25 mg/mL; Sigma), and L-glutamine (200 mM, 29.2 mg/mL; Life Technologies), culture, as previously described (29, 30). Mino cells (10 million) were subcutaneously implanted into the flank of NOD/SCID mice. When the average tumor volume reached approximately 300 cubic millimeters (mm³), the following treatments were administered in cohorts of 5 mice for each treatment: vehicle alone, 20 mg/kg abemaciclib, 20 mg/kg Compound 90, or 20 mg/kg Compound 94 via oral gavage for 3 weeks. Tumor growth was monitored twice weekly by calipers and tumor volume was calculated using the equation: (½ (length×width²)).

FIG. 17 shows the efficacy of Compound 90 and Compound 94 in inhibiting Mino xenograft tumor growth in mice.

All references cited in this specification are herein incorporated by reference as though each reference was specifically and individually indicated to be incorporated by reference. The citation of any reference is for its disclosure prior to the filing date and should not be construed as an admission that the disclosure is not entitled to antedate such reference by virtue of prior invention.

It will be understood that each of the elements described above, or multiple elements together may also find a useful application in other types of methods differing from the type described above, as well as in other types of diseases differing from the type described herein. Without further analysis, the foregoing will so fully reveal the gist of the disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the disclosure is not intended to be limiting only by the following claims.

### REFERENCES CITED

Emerich D F, Tracy M A, Ward K L, Figueiredo M, Qian R. Henschel C, Bartus R T. Biocompatibility of poly (DL-lactide-co-glycolide) microspheres implanted into the brain. Cell transplantation. 1999 January; 8(1):47-58.
Eran H. A Landscape of Driver Mutations in Melanoma. Cell (2012) 2 (150):251-63.
Hall M, Peters G (1996) Genetic alterations of cyclins, cyclin-dependent kinases, and cdk inhibitors in human cancer. Adv Cancer Res 68: 67-108.
Jiang H, Luo N, Zhang X, Song C, Zang J. EGFR L861Q and CDK4 Amplification Responding to Afatinib Combined with Palbociclib Treatment in a Patient With Advanced Lung Squamous Cell Carcinoma. Lung Cancer (2020) 145:216-218.
Jolliet-Riant P, Tillement J P. Drug transfer across the blood-brain barrier and improvement of brain delivery. Fundam Clin Pharmacol. 1999; 13(1):16-26.
Kent, L. N., & Leone, G. (2019). The broken cycle: E2F dysfunction in cancer. Nature Reviews Cancer, 19, 326-338.
Ling C. p16-Cdk4-Rb Axis Controls Sensitivity to a Cyclin-Dependent Kinase Inhibitor PD0332991 in Glioblastoma Xenograft Cells. Neurooncology (2012) 7(14):810-81.
Malumbres M, Barbacid M (2009) Cell cycle, CDKs and cancer: a changing paradigm. Nat Rev Cancer 9: 153-166.
Meinhart A, Kamenski T, Hoeppner S et al. (2005) structural perspective of CTD function. Genes Dev 19: 1401-1415.
Meyerson M, Enders G H, Wu C L et al. (1992) A family of human cdc2-related protein kinases. EMBO J 11: 2909-2917.
Olanich M E, Sun W, Hewitt S M, Abdullaev Z, Pack S D, Barr F G. CDK4 Amplification Reduces Sensitivity to CDK4/6 Inhibition in Fusion-Positive Rhabdomyosarcoma. Clin Cancer Res (2015) 21(21):4947-59.
Pancholi S, Ribas R, Simigdala N, Schuster E, Nikitorowicz-Buniak J, Ressa A, et al. Tumour Kinome Re-Wiring Governs Resistance to Palbociclib in Oestrogen Receptor Positive Breast Cancers, Highlighting New Therapeutic Modalities. Oncogene (2020) 39(25):4781-97.
Pines J (1994) The cell cycle kinases. Semin Cancer Biol 5: 305-313.
Sausville E A (2002) Complexities in the development of cyclin-dependent kinase inhibitor drugs. Trends Mol Med 8: S32-S37.
Shapiro G I (2006) Cyclin-dependent kinase pathways as targets for cancer treatment. J Clin Oncol 24: 1770-1783.
Sherr C J, Roberts J M (1999) CDK inhibitors: positive and negative regulators of G1-phase progression. Genes Dev 13: 1501-1512.
Schroeder U, Sabel B A, Schroeder H. Diffusion enhancement of drugs by loaded nanoparticles in vitro. Prog Neuropsychopharmacol Biol Psychiatry. 1999 July: 23(5):941-9.
Yang, C. et al. Acquired CDK6 amplification promotes breast cancer resistance to CDK4/6 inhibitors and loss of ER signaling and dependence. Oncogene. 2017 Apr. 20; 36(16):2255-2264.

What is claimed is:

1. A compound represented by Formula (I) or a tautomer, stereoisomer, mixture of stereoisomers, pharmaceutically acceptable salt, or solvate thereof: 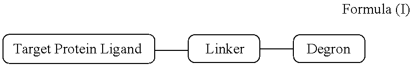
wherein:
the Target Protein Ligand is capable of binding to a target protein, such as a cyclin-dependent kinase (e.g., CDK4 and/or CDK6); the Target Protein Ligand is one of the following structures: 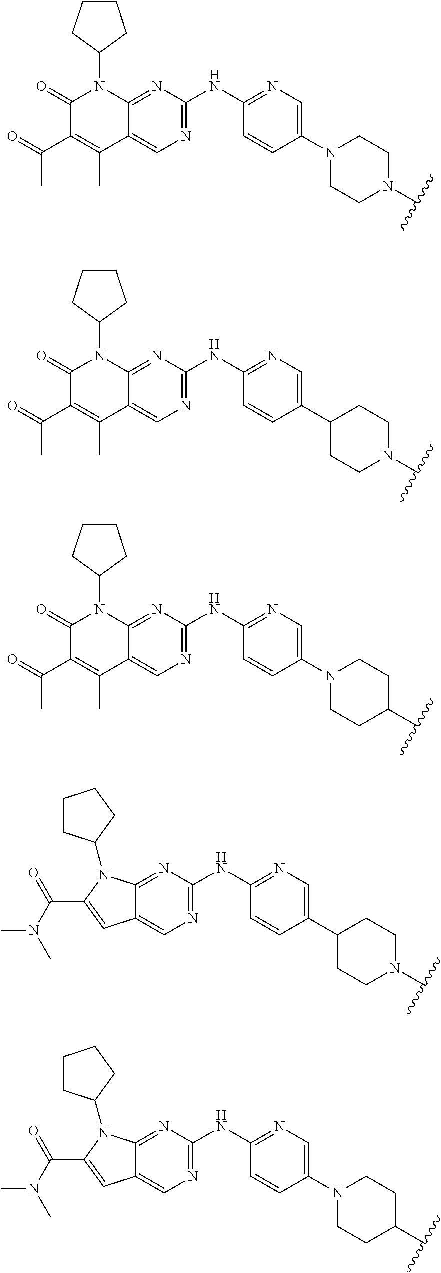 [SMILES: *N1CCN(c2ccc(Nc3ncc4c(C)c(C(C)=O)c(=O)n(C5CCCC5)c4n3)nc2)CC1 ; *N1CCC(c2ccc(Nc3ncc4c(C)c(C(C)=O)c(=O)n(C5CCCC5)c4n3)nc2)CC1 ; *C1CCN(c2ccc(Nc3ncc4c(C)c(C(C)=O)c(=O)n(C5CCCC5)c4n3)nc2)CC1 ; *N1CCC(c2ccc(Nc3ncc4cc(C(=O)N(C)C)n(C5CCCC5)c4n3)nc2)CC1 ; *C1CCN(c2ccc(Nc3ncc4cc(C(=O)N(C)C)n(C5CCCC5)c4n3)nc2)CC1] 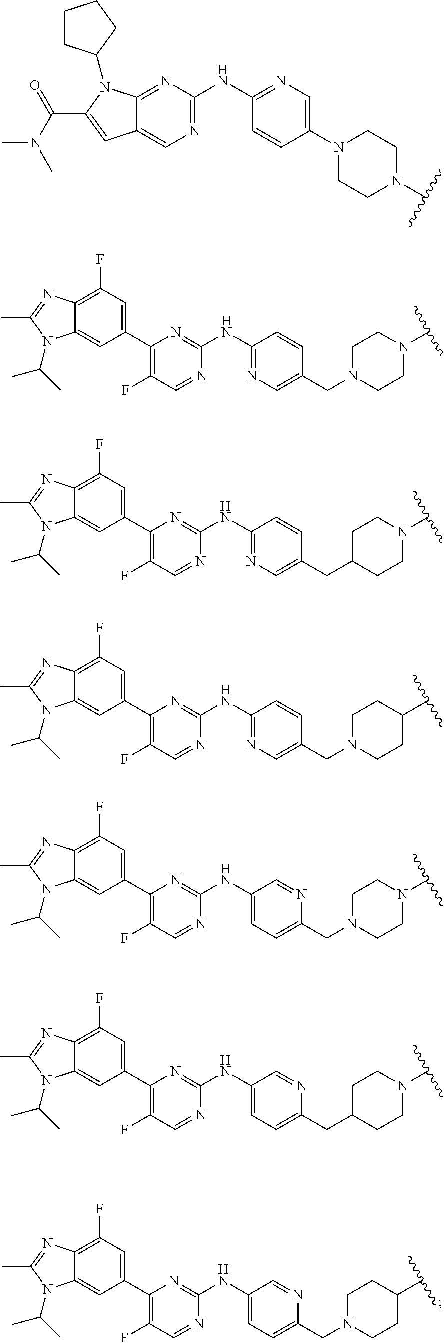
the Linker is an optionally substituted linking moiety comprising a branched or unbranched, cyclized or uncyclized, saturated or unsaturated chain of 5 to 16 carbon atoms in length, wherein 1 to 6 of the carbon atoms are optionally replaced with a heteroatom independently selected from O, N and S.
wherein the linker is an optionally substituted linking moiety comprising a branched or linear C₅-C₁₆ alkyl, branched or linear amino-C₅-C₁₆ alkyl, branched or linear C₅-C₁₆ alkoxy, branched or linear thio-C₅-C₁₆ alkyl, C₅-C₁₆ cycloalkyl, amino-C₅-C₁₆ cycloalkyl, hydroxy-C₅-C₁₆ cycloalky, thio-C₅-C₁₆ cycloalkyl;
wherein 1 to 6 of the carbon atoms are optionally replaced with a heteroatom independently selected from O, N and S.

2. The compound of claim 1, wherein the linker is selected from the group consisting of:

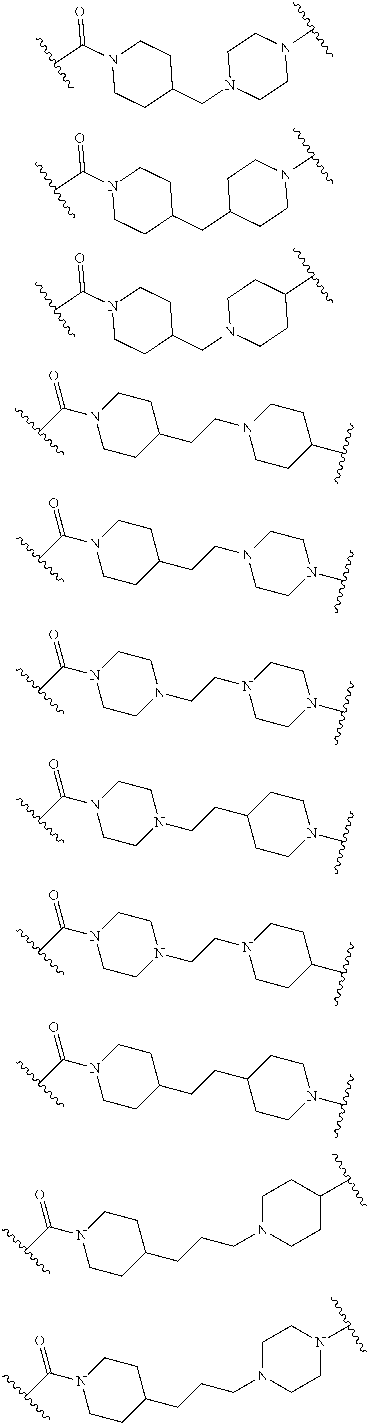

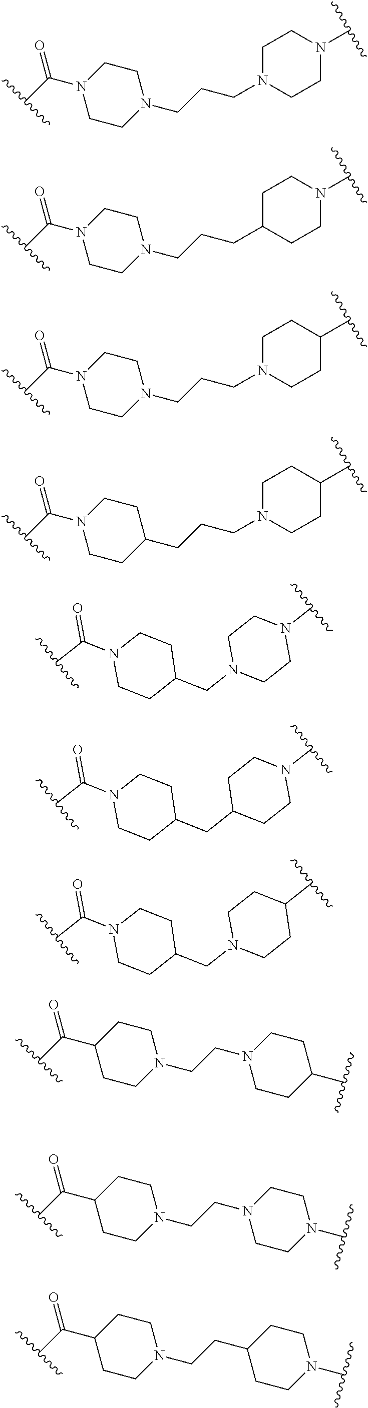

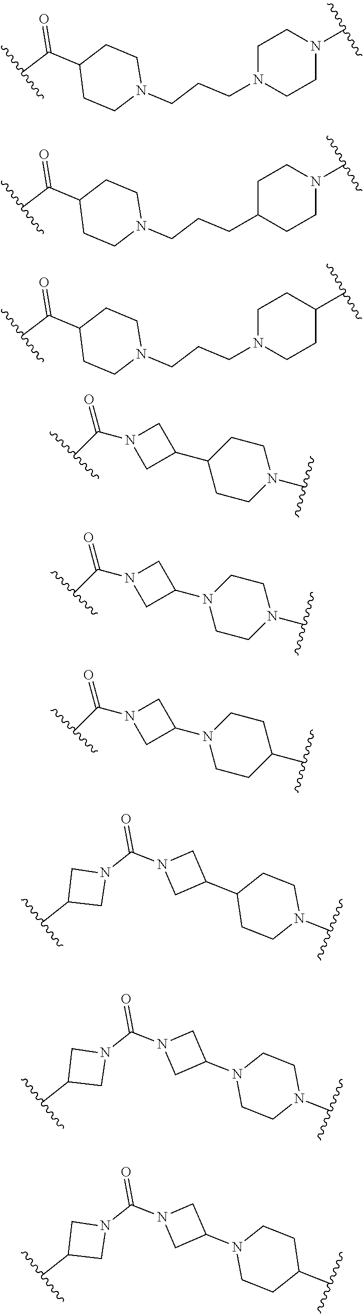

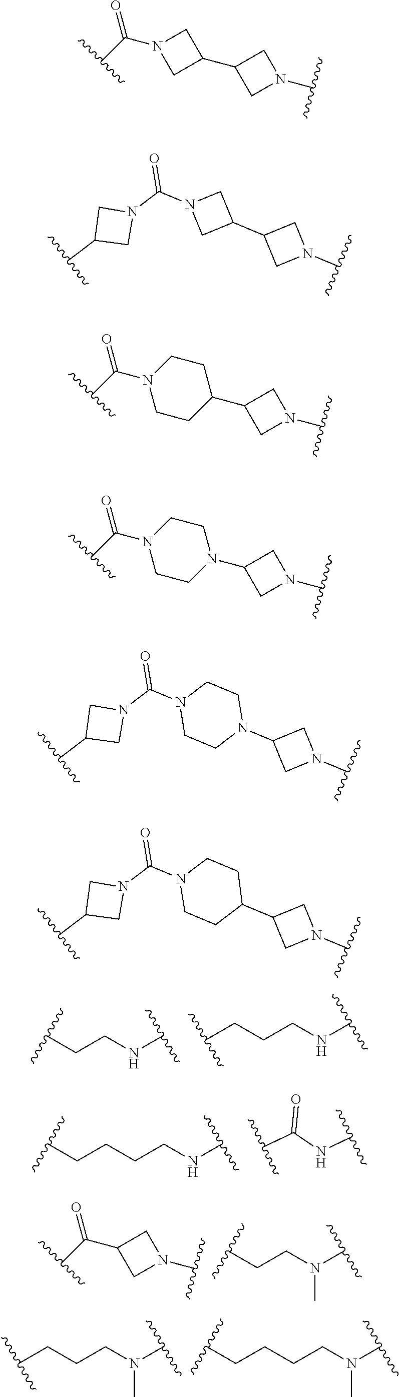

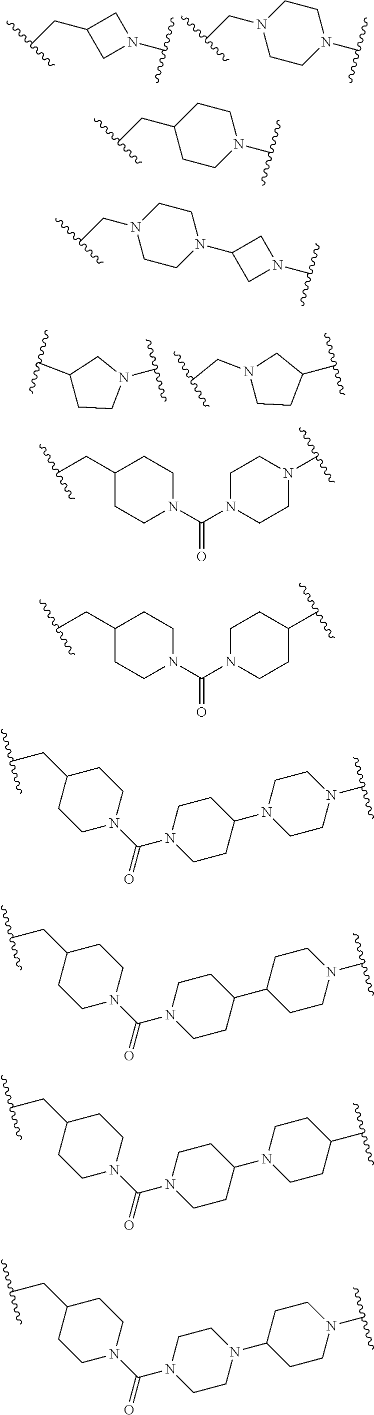

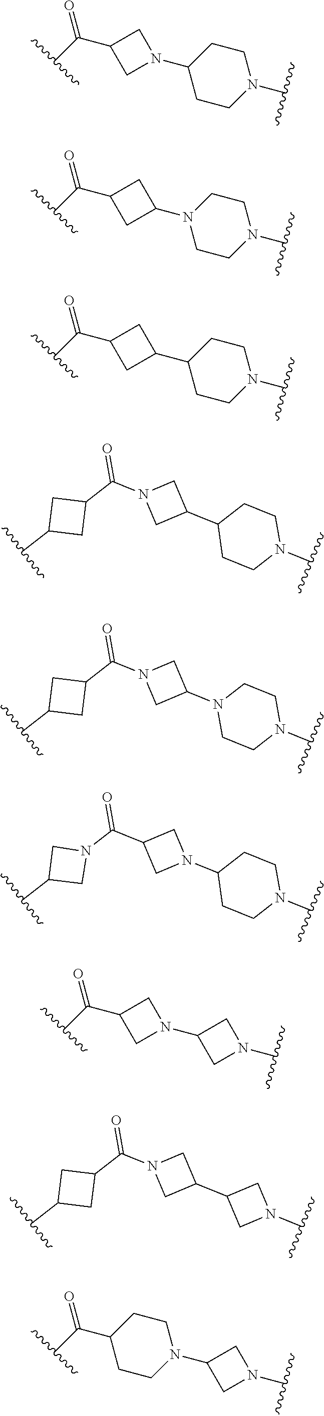

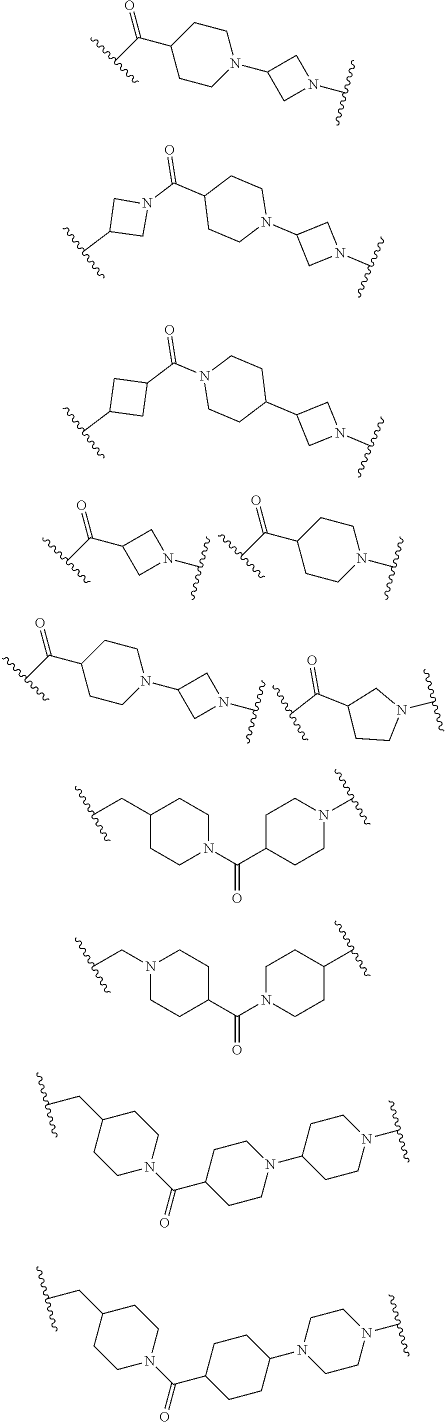

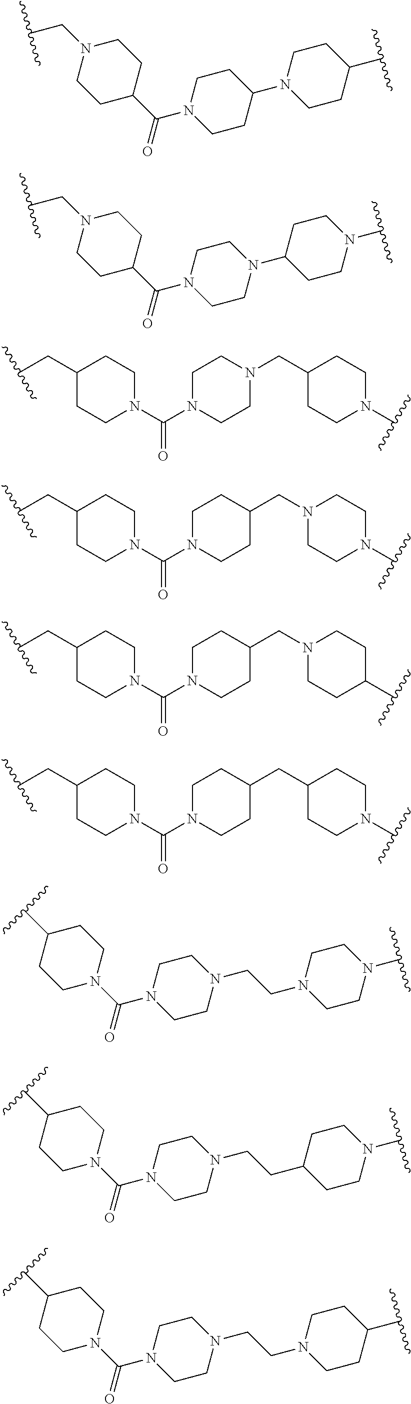

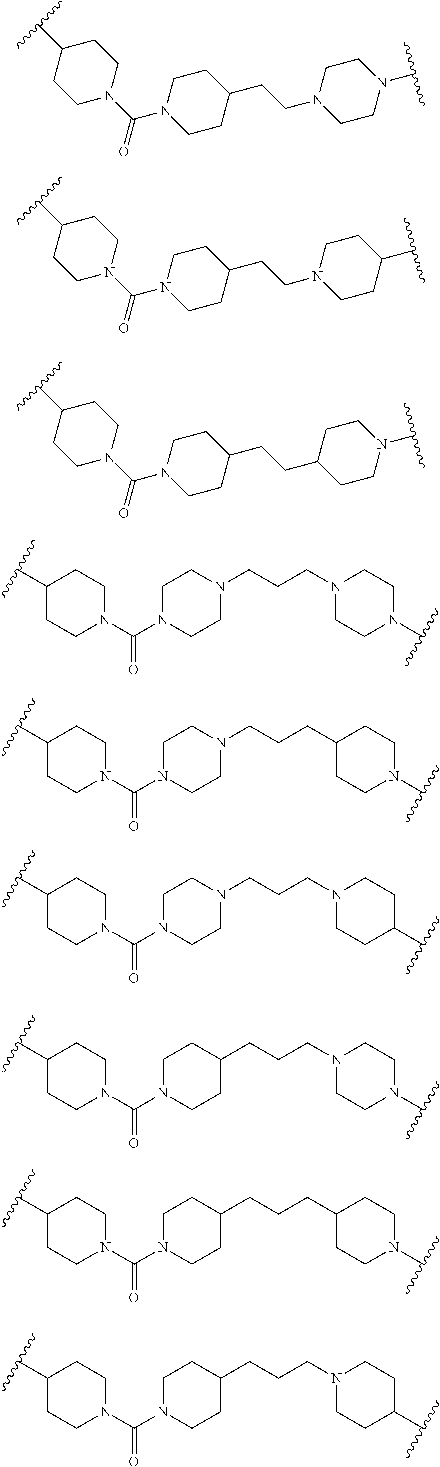

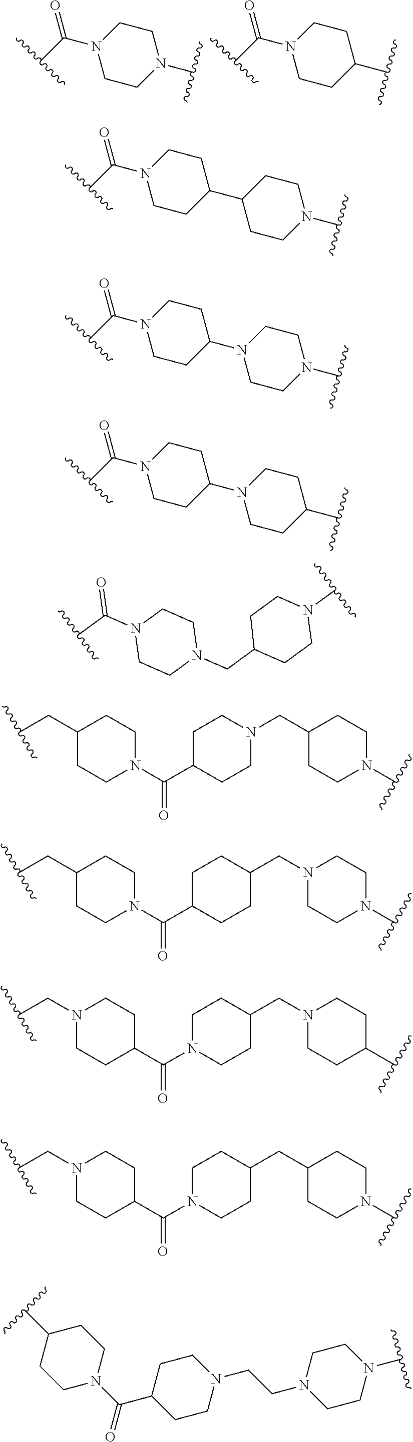

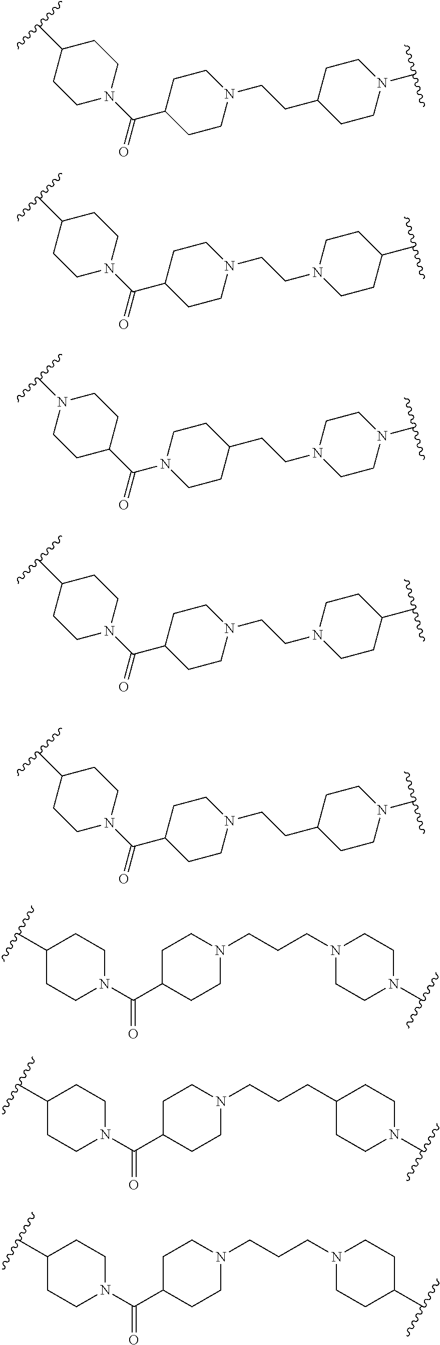

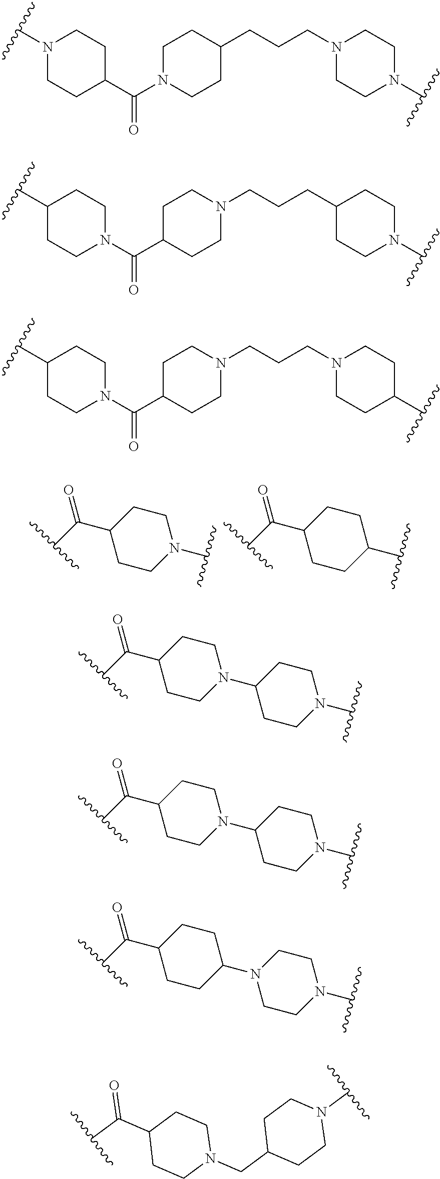

3. The compound of claim 1, wherein the Degron is selected from the group consisting of:

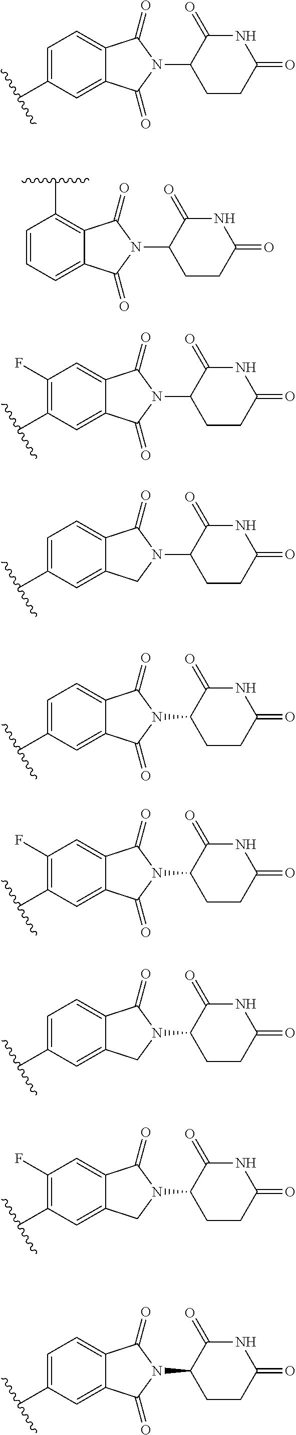

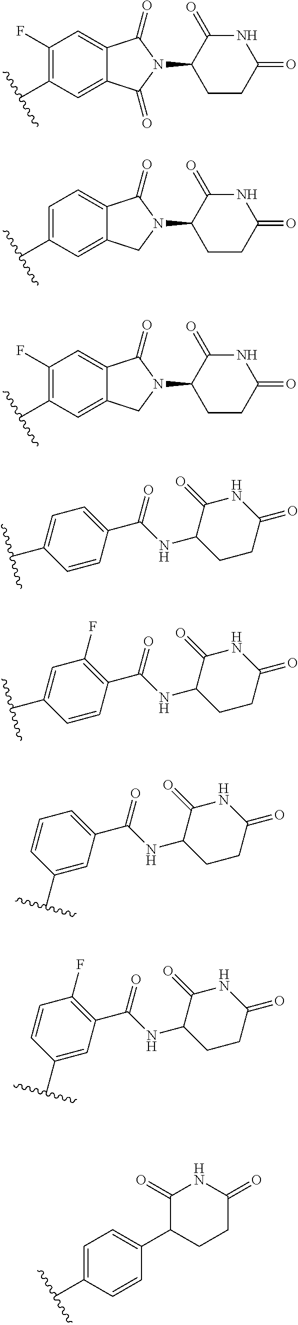

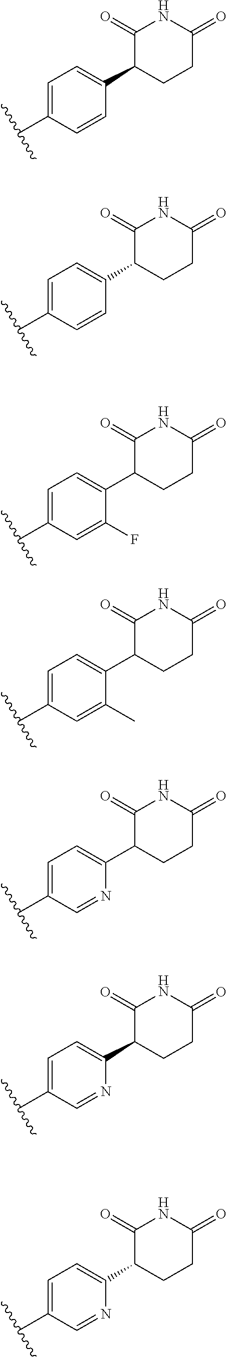

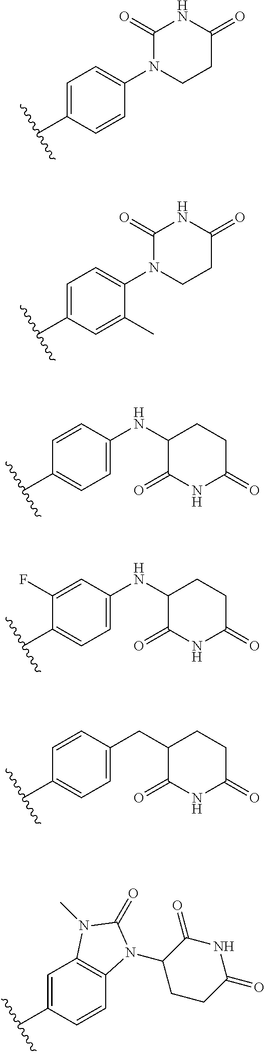 [SMILES: *c1ccc(N2CCC(=O)NC2=O)cc1 ; *c1ccc(N2CCC(=O)NC2=O)c(C)c1 ; *c1ccc(NC2CCC(=O)NC2=O)cc1 ; *c1ccc(NC2CCC(=O)NC2=O)cc1F ; *c1ccc(CC2CCC(=O)NC2=O)cc1 ; *c1ccc2c(c1)n(C)c(=O)n2C1CCC(=O)NC1=O]

4. The compound according to claim 1, wherein the compound represented by Formula (I) is selected from the group consisting of:
5-(4-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperazin-1-yl)-2-(2,6-dioxopiperidin-3-yl)isoindoline-1,3-dione;
3-(5-(4-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperazin-1-yl)-1-oxoisoindolin-2-yl)piperidine-2,6-dione;
5-(4-(1-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-4-yl)piperazin-1-yl)-2-(2,6-dioxopiperidin-3-yl)isoindoline-1,3-dione;
3-(5-(4-(1-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-4-yl)piperazin-1-yl)-1-oxoisoindolin-2-yl)piperidine-2,6-dione;
5-(4-(4-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperazin-1-yl)piperidin-1-yl)-2-(2,6-dioxopiperidin-3-yl)isoindoline-1,3-dione;
3-(5-(4-(4-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperazin-1-yl)piperidin-1-yl)-1-oxoisoindolin-2-yl)piperidine-2,6-dione;
5-(1′-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)-[1,4′-bipiperidin]-4-yl)-2-(2,6-dioxopiperidin-3-yl)isoindoline-1,3-dione;
3-(5-(1′-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)-[1,4′-bipiperidin]-4-yl)-1-oxoisoindolin-2-yl)piperidine-2,6-dione;
5-(1′-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)-[4,4′-bipiperidin]-1-yl)-2-(2,6-dioxopiperidin-3-yl)isoindoline-1,3-dione;
3-(5-(1′-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)-[4,4′-bipiperidin]-1-yl)-1-oxoisoindolin-2-yl)piperidine-2,6-dione;
5-(4-((1-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-4-yl)methyl)piperazin-1-yl)-2-(2,6-dioxopiperidin-3-yl)isoindoline-1,3-dione;
3-(5-(4-((1-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-4-yl)methyl)piperazin-1-yl)-1-oxoisoindolin-2-yl)piperidine-2,6-dione;
5-(4-((1-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-4-yl)methyl)piperidin-1-yl)-2-(2,6-dioxopiperidin-3-yl)isoindoline-1,3-dione;
3-(5-(4-((1-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-4-yl)methyl)piperidin-1-yl)-1-oxoisoindolin-2-yl)piperidine-2,6-dione;
5-(4-((4-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperazin-1-yl)methyl)piperidin-1-yl)-2-(2,6-dioxopiperidin-3-yl)isoindoline-1,3-dione;
3-(5-(4-((4-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperazin-1-yl)methyl)piperidin-1-yl)-1-oxoisoindolin-2-yl)piperidine-2,6-dione;
5-(1-((1-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-4-yl)methyl)piperidin-4-yl)-2-(2,6-dioxopiperidin-3-yl)isoindoline-1,3-dione;
3-(5-(1-((1-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-4-yl)methyl)piperidin-4-yl)-1-oxoisoindolin-2-yl)piperidine-2,6-dione;
5-(4-(2-(1-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-4-yl)ethyl)piperazin-1-yl)-2-(2,6-dioxopiperidin-3-yl)isoindoline-1,3-dione;
3-(5-(4-(2-(1-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-4-yl)ethyl)piperazin-1-yl)-1-oxoisoindolin-2-yl)piperidine-2,6-dione;
5-(4-(2-(4-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperazin-1-yl)ethyl)piperazin-1-yl)-2-(2,6-dioxopiperidin-3-yl)isoindoline-1,3-dione;
3-(5-(4-(2-(4-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperazin-1-yl)ethyl)piperazin-1-yl)-1-oxoisoindolin-2-yl)piperidine-2,6-dione;
5-(4-(2-(4-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperazin-1-yl)ethyl)piperidin-1-yl)-2-(2,6-dioxopiperidin-3-yl)isoindoline-1,3-dione;
3-(5-(4-(2-(4-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperazin-1-yl)ethyl)piperidin-1-yl)-1-oxoisoindolin-2-yl)piperidine-2,6-dione;
5-(4-(2-(1-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-4-yl)ethyl)piperidin-1-yl)-2-(2,6-dioxopiperidin-3-yl)isoindoline-1,3-dione;
3-(5-(4-(2-(1-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-4-yl)ethyl)piperidin-1-yl)-1-oxoisoindolin-2-yl)piperidine-2,6-dione;
5-(1-(2-(4-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperazin-1-yl)ethyl)piperidin-4-yl)-2-(2,6-dioxopiperidin-3-yl)isoindoline-1,3-dione;
3-(5-(1-(2-(4-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperazin-1-yl)ethyl)piperidin-4-yl)-1-oxoisoindolin-2-yl)piperidine-2,6-dione;
5-(1-(2-(1-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-4-yl)ethyl)piperidin-4-yl)-2-(2,6-dioxopiperidin-3-yl)isoindoline-1,3-dione;
3-(5-(1-(2-(1-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-4-yl)ethyl)piperidin-4-yl)-1-oxoisoindolin-2-yl)piperidine-2,6-dione;
5-(4-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carbonyl)piperazin-1-yl)-2-(2,6-dioxopiperidin-3-yl)isoindoline-1,3-dione;
3-(5-(4-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carbonyl)piperazin-1-yl)-1-oxoisoindolin-2-yl)piperidine-2,6-dione;
5-(1-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carbonyl)piperidin-4-yl)-2-(2,6-dioxopiperidin-3-yl)isoindoline-1,3-dione;
5-(1-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carbonyl)piperidin-4-yl)-2-(2,6-dioxopiperidin-3-yl)isoindoline-1,3-dione;
5-(4-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carbonyl)piperazin-1-yl)-2-(2,6-dioxopiperidin-3-yl)isoindoline-1,3-dione;
3-(5-(4-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carbonyl)piperazin-1-yl)-1-oxoisoindolin-2-yl)piperidine-2,6-dione;
5-(1-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carbonyl)piperidin-4-yl)-2-(2,6-dioxopiperidin-3-yl)isoindoline-1,3-dione;
3-(5-(1-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carbonyl)piperidin-4-yl)-1-oxoisoindolin-2-yl)piperidine-2,6-dione;
4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-(2-(2,6-dioxopiperidin-3-yl)-1,3-dioxoisoindolin-5-yl)piperidine-1-carboxamide;
4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-(2-(2,6-dioxopiperidin-3-yl)-1-oxoisoindolin-5-yl)piperidine-1-carboxamide;
4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)-N-(2-(2,6-dioxopiperidin-3-yl)-1,3-dioxoisoindolin-5-yl)piperidine-1-carboxamide;
4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)-N-(2-(2,6-dioxopiperidin-3-yl)-1-oxoisoindolin-5-yl)piperidine-1-carboxamide
4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)-N-(2-(2,6-dioxopiperidin-3-yl)-1,3-dioxoisoindolin-5-yl)piperazine-1-carboxamide;
4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)-N-(2-(2,6-dioxopiperidin-3-yl)-1-oxoisoindolin-5-yl)piperazine-1-carboxamide;
4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)-N-(2-(2,6-dioxopiperidin-3-yl)-1,3-dioxoisoindolin-5-yl)piperidine-1-carboxamide;
4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)-N-(2-(2,6-dioxopiperidin-3-yl)-1-oxoisoindolin-5-yl)piperidine-1-carboxamide;
5-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)amino)-2-(2,6-dioxopiperidin-3-yl)isoindoline-1,3-dione;
3-(5-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)amino)-1-oxoisoindolin-2-yl)piperidine-2,6-dione;
5-((3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propyl)amino)-2-(2,6-dioxopiperidin-3-yl)isoindoline-1,3-dione;
3-(5-((3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propyl)amino)-1-oxoisoindolin-2-yl)piperidine-2,6-dione;
5-((3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propyl)amino)-2-(2,6-dioxopiperidin-3-yl)-6-fluoroisoindoline-1,3-dione;
3-(5-((3-(4-(6-((6-acetyl-8-cyclopentyl-5-methyl-7-oxo-7,8-dihydropyrido[2,3-d]pyrimidin-2-ylamino)pyridin-3-yl)piperazin-1-yl)propyl)amino)-6-fluoro-1-oxoisoindolin-2-yl)piperidine-2,6-dione;
5-((4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butyl)amino)-2-(2,6-dioxopiperidin-3-yl)isoindoline-1,3-dione;
3-(5-((4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butyl)amino)-1-oxoisoindolin-2-yl)piperidine-2,6-dione;
5-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)(methyl)amino)-2-(2,6-dioxopiperidin-3-yl)isoindoline-1,3-dione;
3-(5-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)(methyl)amino)-1-oxoisoindolin-2-yl)piperidine-2,6-dione;
5-((3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propyl)(methyl)amino)-2-(2,6-dioxopiperidin-3-yl)isoindoline-1,3-dione;
3-(5-((3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propyl)(methyl)amino)-1-oxoisoindolin-2-yl)piperidine-2,6-dione;
5-((4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butyl)(methyl)amino)-2-(2,6-dioxopiperidin-3-yl)isoindoline-1,3-dione;
3-(5-((4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butyl)(methyl)amino)-1-oxoisoindolin-2-yl)piperidine-2,6-dione;
4-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)(methyl)amino)-2-(2,6-dioxopiperidin-3-yl)isoindoline-1,3-dione;
4-((3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propyl)amino)-2-(2,6-dioxopiperidin-3-yl)isoindoline-1,3-dione;
5-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidin-1-yl)-2-(2,6-dioxopiperidin-3-yl)isoindoline-1,3-dione;
3-(5-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidin-1-yl)-1-oxoisoindolin-2-yl)piperidine-2,6-dione;
5-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidin-1-yl)-2-(2,6-dioxopiperidin-3-yl)isoindoline-1,3-dione;
3-(5-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidin-1-yl)-1-oxoisoindolin-2-yl)piperidine-2,6-dione;
5-(4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)piperazin-1-yl)-2-(2,6-dioxopiperidin-3-yl)isoindoline-1,3-dione;
3-(5-(4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)piperazin-1-yl)-1-oxoisoindolin-2-yl)piperidine-2,6-dione
5-(4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)piperidin-1-yl)-2-(2,6-dioxopiperidin-3-yl)isoindoline-1,3-dione;
3-(5-(4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)piperidin-1-yl)-1-oxoisoindolin-2-yl)piperidine-2,6-dione;
5-(1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)piperidin-4-yl)-2-(2,6-dioxopiperidin-3-yl)isoindoline-1,3-dione;
3-(5-(1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)piperidin-4-yl)-1-oxoisoindolin-2-yl)piperidine-2,6-dione;
2-(2,6-dioxopiperidin-3-yl)-5-((3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)propyl)amino)isoindoline-1,3-dione;
3-(5-((3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)propyl)amino)-1-oxoisoindolin-2-yl)piperidine-2,6-dione;
7-cyclopentyl-2-((5-(4-(3-((2-(2,6-dioxopiperidin-3-yl)-1,3-dioxoisoindolin-5-yl)amino)propyl)piperazin-1-yl)pyridin-2-yl)amino)-N,N-dimethyl-7H-pyrrolo[2,3-d]pyrimidine-6-carboxamide;
7-cyclopentyl-2-((5-(4-(3-((2-(2,6-dioxopiperidin-3-yl)-1-oxoisoindolin-5-yl)amino)propyl)piperazin-1-yl)pyridin-2-yl)amino)-N,N-dimethyl-7H-pyrrolo[2,3-d]pyrimidine-6-carboxamide;
4-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidin-1-yl)-2-(2,6-dioxopiperidin-3-yl)isoindoline-1,3-dione;
4-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidin-1-yl)-2-(2,6-dioxopiperidin-3-yl)isoindoline-1,3-dione
4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)azetidin-1-yl)-2-(2,6-dioxopiperidin-3-yl)isoindoline-1,3-dione;
5-(3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)azetidin-1-yl)-2-(2,6-dioxopiperidin-3-yl)isoindoline-1,3-dione;
4-(3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)azetidin-1-yl)-2-(2,6-dioxopiperidin-3-yl)isoindoline-1,3-dione;
5-(1′-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carbonyl)-[4,4′-bipiperidin]-1-yl)-2-(2,6-dioxopiperidin-3-yl)isoindoline-1,3-dione;
5-(4-(2-(1-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carbonyl)piperidin-4-yl)ethyl)piperazin-1-yl)-2-(2,6-dioxopiperidin-3-yl)isoindoline-1,3-dione;
5-(4-((1-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carbonyl)piperidin-4-yl)methyl)piperazin-1-yl)-2-(2,6-dioxopiperidin-3-yl)isoindoline-1,3-dione;
5-(1′-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carbonyl)-[4,4′-bipiperidin]-1-yl)-2-(2,6-dioxopiperidin-3-yl)isoindoline-1,3-dione;
5-(4-((1-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carbonyl)piperidin-4-yl)methyl)piperazin-1-yl)-2-(2,6-dioxopiperidin-3-yl)isoindoline-1,3-dione;
5-(4-(2-(1-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carbonyl)piperidin-4-yl)ethyl)piperazin-1-yl)-2-(2,6-dioxopiperidin-3-yl)isoindoline-1,3-dione;
5-(4-(1-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carbonyl)piperidin-4-yl)piperazin-1-yl)-2-(2,6-dioxopiperidin-3-yl)isoindoline-1,3-dione;
5-(4-(1-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidine-1-carbonyl)piperidin-4-yl)piperazin-1-yl)-2-(2,6-dioxopiperidin-3-yl)isoindoline-1,3-dione;
2-(2,6-dioxopiperidin-3-yl)-5-((2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)ethyl)amino)isoindoline-1,3-dione;
3-(6-((2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)ethyl)amino)-1-oxoisoindolin-2-yl)piperidine-2,6-dione;
2-(2,6-dioxopiperidin-3-yl)-4-((2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)ethyl)amino)isoindoline-1,3-dione;
3-(4-((2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)ethyl)amino)-1-oxoisoindolin-2-yl)piperidine-2,6-dione;
2-(2,6-dioxopiperidin-3-yl)-5-(3-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)azetidin-1-yl)isoindoline-1,3-dione;
3-(5-(3-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)azetidin-1-yl)-1-oxoisoindolin-2-yl)piperidine-2,6-dione;
N-(2,6-dioxopiperidin-3-yl)-4-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)azetidin-1-yl)benzamide;
3-(5-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)azetidin-1-yl)-1-oxoisoindolin-2-yl)piperidine-2,6-dione;
2-(2,6-dioxopiperidin-3-yl)-4-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)azetidin-1-yl)isoindoline-1,3-dione;
3-(4-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)azetidin-1-yl)-1-oxoisoindolin-2-yl)piperidine-2,6-dione;
2-(2,6-dioxopiperidin-3-yl)-4-((3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)propyl)amino)isoindoline-1,3-dione;
2-(2,6-dioxopiperidin-3-yl)-4-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)isoindoline-1,3-dione;
4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)azetidin-1-yl)-N-(2,6-dioxopiperidin-3-yl)benzamide;
4-((3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propyl)(methyl)amino)-2-(2,6-dioxopiperidin-3-yl)isoindoline-1,3-dione;
5-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethyl)amino)-2-(2,6-dioxopiperidin-3-yl)isoindoline-1,3-dione;
1-(4-((3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propyl)amino)phenyl)dihydropyrimidine-2,4(1H,3H)-dione;
1-(4-(3-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)azetidin-1-yl)phenyl)dihydropyrimidine-2,4(1H,3H)-dione;
1-(4-((3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)propyl)amino)phenyl)dihydropyrimidine-2,4(1H,3H)-dione;
1-(4-(4-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)piperidin-1-yl)phenyl)dihydropyrimidine-2,4(1H,3H)-dione;
1-(4-(4-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)piperidin-1-yl)phenyl)dihydropyrimidine-2,4(1H,3H)-dione;
1-(4-(3-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)piperidin-1-yl)phenyl)dihydropyrimidine-2,4(1H,3H)-dione;
1-(4-(3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)azetidin-1-yl)phenyl)dihydropyrimidine-2,4(1H,3H)-dione;
1-(4-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)piperidin-1-yl)phenyl)dihydropyrimidine-2,4(1H,3H)-dione;
1-(4-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidin-1-yl)phenyl)dihydropyrimidine-2,4(1H,3H)-dione;
5-(4-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-1-yl)-2-(2,6-dioxopiperidin-3-yl)isoindoline-1,3-dione;
3-(5-(4-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-1-yl)-1-oxoisoindolin-2-yl)piperidine-2,6-dione;
4-(4-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-1-yl)-N-(2,6-dioxopiperidin-3-yl)benzamide;
3-((4-(4-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)piperidin-1-yl)phenyl)amino)piperidine-2,6-dione;
3-(5-(3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)azetidin-1-yl)-1-oxoisoindolin-2-yl)piperidine-2,6-dione;
4-(3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)azetidin-1-yl)-N-(2,6-dioxopiperidin-3-yl)benzamide;
3-((4-(3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)azetidin-1-yl)phenyl)amino)piperidine-2,6-dione;
5-(3-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)azetidin-1-yl)-2-(2,6-dioxopiperidin-3-yl)isoindoline-1,3-dione;
3-(5-(3-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)azetidin-1-yl)-1-oxoisoindolin-2-yl)piperidine-2,6-dione;
4-(3-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)azetidin-1-yl)-N-(2,6-dioxopiperidin-3-yl)benzamide;
3-((4-(3-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)azetidin-1-yl)phenyl)amino)piperidine-2,6-dione;
3-(5-(3-((4-(6-((6-acetyl-8-cyclopentyl-5-methyl-7-oxo-7,8-dihydropyrido[2,3-d]pyrimidin-2-yl)amino)pyridin-3-yl)piperidin-1-yl)methyl)azetidin-1-yl)-1-oxoisoindolin-2-yl)piperidine-2,6-dione;
4-(3-((4-(6-((6-acetyl-8-cyclopentyl-5-methyl-7-oxo-7,8-dihydropyrido[2,3-d]pyrimidin-2-yl)amino)pyridin-3-yl)piperidin-1-yl)methyl)azetidin-1-yl)-N-(2,6-dioxopiperidin-3-yl)benzamide;
3-((4-(3-((4-(6-((6-acetyl-8-cyclopentyl-5-methyl-7-oxo-7,8-dihydropyrido[2,3-d]pyrimidin-2-yl)amino)pyridin-3-yl)piperidin-1-yl)methyl)azetidin-1-yl)phenyl)amino)piperidine-2,6-dione;
5-(3-(4-(6-((6-acetyl-8-cyclopentyl-5-methyl-7-oxo-7,8-dihydropyrido[2,3-d]pyrimidin-2-yl)amino)pyridin-3-yl)piperidine-1-carbonyl)azetidin-1-yl)-2-(2,6-dioxopiperidin-3-yl)isoindoline-1,3-dione;
3-(5-(3-(4-(6-((6-acetyl-8-cyclopentyl-5-methyl-7-oxo-7,8-dihydropyrido[2,3-d]pyrimidin-2-yl)amino)pyridin-3-yl)piperidine-1-carbonyl)azetidin-1-yl)-1-oxoisoindolin-2-yl)piperidine-2,6-dione;
4-(3-(4-(6-((6-acetyl-8-cyclopentyl-5-methyl-7-oxo-7,8-dihydropyrido[2,3-d]pyrimidin-2-yl)amino)pyridin-3-yl)piperidine-1-carbonyl)azetidin-1-yl)-N-(2,6-dioxopiperidin-3-yl)benzamide;
3-((4-(3-(4-(6-((6-acetyl-8-cyclopentyl-5-methyl-7-oxo-7,8-dihydropyrido[2,3-d]pyrimidin-2-yl)amino)pyridin-3-yl)piperidine-1-carbonyl)azetidin-1-yl)phenyl)amino)piperidine-2,6-dione;
N-(2,6-dioxopiperidin-3-yl)-4-(3-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)azetidin-1-yl)benzamide;
3-((4-(3-((4-((6-((5-fluoro-4-(4-fluoro-1-isopropyl-2-methyl-1H-benzo[d]imidazol-6-yl)pyrimidin-2-yl)amino)pyridin-3-yl)methyl) piperazin-1-yl)methyl)azetidin-1-yl) phenyl)amino)piperidine-2,6-dione;
2-(2,6-dioxopiperidin-3-yl)-5-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazine-1-carbonyl)azetidin-1-yl)isoindoline-1,3-dione;
3-(5-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazine-1-carbonyl)azetidin-1-yl)-1-oxoisoindolin-2-yl)piperidine-2,6-dione;
N-(2,6-dioxopiperidin-3-yl)-4-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazine-1-carbonyl)azetidin-1-yl)benzamide;
3-((4-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazine-1-carbonyl)azetidin-1-yl)phenyl)amino)piperidine-2,6-dione;
2-(2,6-dioxopiperidin-3-yl)-5-(3-((4-((6-((5-fluoro-4-(4-fluoro-1-isopropyl-2-methyl-1H-benzo[d]imidazol-6-yl)pyrimidin-2-yl)amino)pyridin-3-yl)methyl)piperidin-1-yl)methyl)azetidin-1-yl)isoindoline-1,3-dione;
3-(5-(3-((4-((6-((5-fluoro-4-(4-fluoro-1-isopropyl-2-methyl-1H-benzo[d]imidazol-6-yl)pyrimidin-2-yl)amino)pyridin-3-yl)methyl)piperidin-1-yl)methyl)azetidin-1-yl)-1-oxoisoindolin-2-yl)piperidine-2,6-dione;
N-(2,6-dioxopiperidin-3-yl)-4-(3-((4-((6-((5-fluoro-4-(4-fluoro-1-isopropyl-2-methyl-1H-benzo[d]imidazol-6-yl)pyrimidin-2-yl)amino)pyridin-3-yl)methyl)piperidin-1-yl)methyl)azetidin-1-yl)benzamide;
3-((4-(3-((4-((6-((5-fluoro-4-(4-fluoro-1-isopropyl-2-methyl-1H-benzo[d]imidazol-6-yl)pyrimidin-2-yl)amino)pyridin-3-yl)methyl)piperidin-1-yl)methyl)azetidin-1-yl)phenyl)amino)piperidine-2,6-dione;
2-(2,6-dioxopiperidin-3-yl)-5-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperidine-1-carbonyl)azetidin-1-yl)isoindoline-1,3-dione;
3-(5-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperidine-1-carbonyl)azetidin-1-yl)-1-oxoisoindolin-2-yl)piperidine-2,6-dione;
N-(2,6-dioxopiperidin-3-yl)-4-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperidine-1-carbonyl)azetidin-1-yl)benzamide;
3-((4-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperidine-1-carbonyl)azetidin-1-yl)phenyl)amino)piperidine-2,6-dione;
2-(2,6-dioxopiperidin-3-yl)-5-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)azetidin-1-yl)isoindoline-1,3-dione;
2-(2,6-dioxopiperidin-3-yl)-5-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperidin-1-yl)azetidin-1-yl)isoindoline-1,3-dione;
2-(2,6-dioxopiperidin-3-yl)-5-(4-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)piperidin-1-yl)isoindoline-1,3-dione;
N-(2,6-dioxopiperidin-3-yl)-4-(4-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)piperidin-1-yl)benzamide;
N-(2,6-dioxopiperidin-3-yl)-4-(4-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)piperidin-1-yl)benzamide;
3-((4-(4-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)piperidin-1-yl)phenyl)amino)piperidine-2,6-dione;
2-(2,6-dioxopiperidin-3-yl)-5-(4-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazine-1-carbonyl)piperidin-1-yl)isoindoline-1,3-dione;
3-(5-(4(4-((6-((5-fluoro-4-(4-fluoro-1-isopropyl-2-methyl-1H-benzo[d]imidazol-6-yl)pyrimidin-2-yl)amino)pyridin-3-yl)methyl)piperazine-1-carbonyl)piperidin-1-yl)-1-oxoisoindolin-2-yl)piperidine-2,6-dione;
N-(2,6-dioxopiperidin-3-yl)-4-(4-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazine-1-carbonyl)piperidin-1-yl)benzamide;
3-((4-(4-(4-((6-((5-fluoro-4-(4-fluoro-1-isopropyl-2-methyl-1H-benzo[d]imidazol-6-yl)pyrimidin-2-yl)amino)pyridin-3-yl)methyl) piperazine-1-carbonyl)piperidin-1-yl)phenyl)amino)piperidine-2,6-dione;
2-(2,6-dioxopiperidin-3-yl)-5-(4-((4-((6-((5-fluoro-4-(4-fluoro-1-isopropyl-2-methyl-1H-benzo[d]imidazol-6-yl)pyrimidin-2-yl)amino)pyridin-3-yl)methyl) piperazin-1-yl)methyl)piperidin-1-yl)isoindoline-1,3-dione;
3-(5-(4-((4-((6-((5-fluoro-4-(4-fluoro-1-isopropyl-2-methyl-1H-benzo[d]imidazol-6-yl)pyrimidin-2-yl)amino)pyridin-3-yl)methyl) piperidin-1-yl)methyl)piperidin-1-yl)-1-oxoisoindolin-2-yl)piperidine-2,6-dione;
N-(2,6-dioxopiperidin-3-yl)-4-(4-((4-((6-((5-fluoro-4-(4-fluoro-1-isopropyl-2-methyl-1H-benzo[d]imidazol-6-yl)pyrimidin-2-yl)amino)pyridin-3-yl)methyl)piperidin-1-yl)methyl)piperidin-1-yl)benzamide;
3-((4-(4-((4-((6-((5-fluoro-4-(4-fluoro-1-isopropyl-2-methyl-1H-benzo[d]imidazol-6-yl)pyrimidin-2-yl)amino)pyridin-3-yl)methyl) piperidin-1-yl)methyl)piperidin-1-yl)phenyl)amino)piperidine-2,6-dione;
2-(2,6-dioxopiperidin-3-yl)-5-(4-(4-((6-((5-fluoro-4-(4-fluoro-1-isopropyl-2-methyl-1H-benzo[d]imidazol-6-yl)pyrimidin-2-yl)amino)pyridin-3-yl)methyl) piperidine-1-carbonyl)piperidin-1-yl)isoindoline-1,3-dione;
3-(5-(4-(4-((6-((5-fluoro-4-(4-fluoro-1-isopropyl-2-methyl-1H-benzo[d] imidazol-6-yl)pyrimidin-2-yl)amino)pyridin-3-yl)methyl)piperidine-1-carbonyl)piperidin-1-yl)-1-oxoisoindolin-2-yl)piperidine-2,6-dione;
N-(2,6-dioxopiperidin-3-yl)-4-(4-(4-((6-((5-fluoro-4-(4-fluoro-1-isopropyl-2-methyl-1H-benzo[d]imidazol-6-yl)pyrimidin-2-yl)amino)pyridin-3-yl)methyl)piperidine-1-carbonyl)piperidin-1-yl)benzamide;
3-((4-(4-(4-((6-((5-fluoro-4-(4-fluoro-1-isopropyl-2-methyl-1H-benzo[d]imidazol-6-yl)pyrimidin-2-yl)amino)pyridin-3-yl)methyl)piperidine-1-carbonyl)piperidin-1-yl)phenyl)amino)piperidine-2,6-dione;
2-(2,6-dioxopiperidin-3-yl)-5-(4-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)piperidin-1-yl)isoindoline-1,3-dione;
3-(5-(4-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)piperidin-1-yl)-1-oxoisoindolin-2-yl)piperidine-2,6-dione;
N-(2,6-dioxopiperidin-3-yl)-4-(4-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)piperidin-1-yl)benzamide;
3-((4-(4-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)piperidin-1-yl)phenyl)amino)piperidine-2,6-dione;
2-(2,6-dioxopiperidin-3-yl)-5-(4-((6-((5-fluoro-4-(4-fluoro-1-isopropyl-2-methyl-1H-benzo[d]imidazol-6-yl)pyrimidin-2-yl)amino)pyridin-3-yl)methyl)-[1,4′-bipiperidin]-1′-yl)isoindoline-1,3-dione;
3-(5-(4-((6-((5-fluoro-4-(4-fluoro-1-isopropyl-2-methyl-1H-benzo[d]imidazol-6-yl)pyrimidin-2-yl)amino)pyridin-3-yl)methyl)-[1,4′-bipiperidin]-1′-yl)-1-oxoisoindolin-2-yl)piperidine-2,6-dione;
N-(2,6-dioxopiperidin-3-yl)-4-(4-((6-((5-fluoro-4-(4-fluoro-1-isopropyl-2-methyl-1H-benzo[d]imidazol-6-yl)pyrimidin-2-yl)amino)pyridin-3-yl)methyl)-[1,4′-bipiperidin]-1′-yl)benzamide;
3-((4-(4-((6-((5-fluoro-4-(4-fluoro-1-isopropyl-2-methyl-1H-benzo[d]imidazol-6-yl)pyrimidin-2-yl)amino)pyridin-3-yl)methyl)-[1,4′-bipiperidin]-1′-yl)phenyl)amino)piperidine-2,6-dione;
and combinations thereof.

5. A composition comprising at least one compound of claim 1.

6. A pharmaceutical composition comprising at least one compound of claim 1 and a pharmaceutically acceptable carrier.

7. The pharmaceutical composition of claim 6, wherein said pharmaceutical composition is suitable for oral administration.

8. The pharmaceutical composition of claim 6, when the pharmaceutical composition is suitable for parenteral administration.

9. A method of preparing at least one compound of claim 1 or composition or pharmaceutical composition thereof.

10. A method of treating a disease or disorder in a subject in need thereof, the method comprising administering at least one compound of claim 1 or composition or pharmaceutical composition thereof to the subject.

11. A method for treating cancer in a subject in need thereof, the method comprising administering an effective amount of a compound of claim 1 or composition or pharmaceutical composition thereof to the subject.

12. The method of claim 11, wherein said cancer expresses CDK4/6.

13. The method of claim 11, where said subject expresses a mutant CDK4/6 protein.

14. A method of reducing the level or activity of a target protein, the method comprising administering at least one compound of claim 1 or composition or pharmaceutical composition thereof.

15. A method of inhibiting a target protein, the method comprising administering at least one compound of claim 1 or composition or pharmaceutical composition thereof.

16. The method of claim 14, wherein the target protein is CDK4/6.

17. The method of claim 15, wherein the target protein is CDK4/6.
